(19) United States
(12) Patent Application Publication
(10) Pub. No.: US 2017/0184612 A1
(43) Pub. Date: Jun. 29, 2017

(54) USE OF LEUKOCYTES AND NOVEL BIOMARKERS IN THE DIAGNOSIS, CONFIRMATION, AND TREATMENT OF A NEUROLOGICAL DISORDER

(71) Applicants: David Sulzer, New York, NY (US); Alessandro Sette, La Jolla, CA (US)

(72) Inventors: David Sulzer, New York, NY (US); Alessandro Sette, La Jolla, CA (US)

(73) Assignees: The Trustees of Columbia University in the City of New York, New York, NY (US); La Jolla Institute for Allergy & Immunology, La Jolla, CA (US)

(21) Appl. No.: 15/300,713

(22) PCT Filed: Apr. 3, 2015
(86) PCT No.: PCT/US15/24310

§ 371 Date: Sep. 29, 2016

Related U.S. Application Data
(60) Provisional application No. 61/977,336, filed on Apr. 9, 2014.

(51) Int. Cl. G01N 33/68; A61K 39/00; G01N 33/569

(52) U.S. Cl. CPC G01N 33/6896; G01N 33/56972; A61K 39/0007; G01N 2800/2821; G01N 2800/2835; G01N 2800/50; G01N 2800/52; G01N 2800/2814; A61K 2039/577; A61K 2039/605

(57) ABSTRACT

The present invention provides methods for assessing whether a subject is at risk of developing a neurological disorder, diagnosing or confirming whether a subject is afflicted with a neurological disorder, assessing whether PD has progressed in a subject afflicted with PD, assessing whether a neurological disorder is developing in a subject who has been identified as being at risk of developing the neurological disorder, assessing whether a subject afflicted with a neurological disorder is likely to benefit from a therapy, assessing whether a subject afflicted with a neurological disorder has benefited from a therapy, treating a subject afflicted with a neurological disorder, and prophylactically treating a subject who has been identified as being at risk of developing a neurological disorder. The present invention also provides epitopes, compounds and compositions relating to these methods.

This application claims priority of U.S. Provisional Application No. 61/977,336, filed Apr. 9, 2014, the entire contents of which are hereby incorporated herein by reference.

This invention was made with government support under grant numbers R01NS064155, U01NS082157 and P01NS058793 awarded by the National Institutes of Health. The U.S. Government has certain rights in the invention.

This application incorporates-by-reference nucleotide and/or amino acid sequences which are present in the file named “150403_0575_85568-A-PCT_Sequence_Listing_REB.txt”, which is 75.6 kilobytes in size, and which was created Apr. 3, 2015 in the IBM-PC machine format, having an operating system compatibility with MS-Windows, which is contained in the text file filed Apr. 3, 2015 as part of this application.

Throughout this application, various publications are referenced, including referenced in parenthesis. Full citations for publications referenced in parenthesis may be found listed at the end of the specification immediately preceding the claims. The disclosures of all referenced publications in their entireties are hereby incorporated by reference into this application in order to more fully describe the state of the art to which this invention pertains.

### BACKGROUND OF INVENTION

Parkinson's disease (PD) affects about 1 of 250 people older than 40, about 1 of 100 people older than 65, and about 1 of 10 people older than 80. Merck Manual, Parkinson's Disease, last full review/revision August 2007 by David Eidelberg and Michael Pourfar, available at merckmanuals.com/home/brain_spinal_cord_and_nerve_disorders/movement_disorders/parkinsons_disease.html (hereinafter “Merck Manual”).

What causes PD is unclear. According to one theory, Parkinson's disease may result from abnormal deposits of synuclein (a protein in the brain that helps nerve cells communicate) (Merck Manual). These deposits, called Lewy bodies, can accumulate in several regions of the brain, particularly in the substantia nigra (deep within the cerebrum) and interfere with brain function (Merck Manual). Lewy bodies often accumulate in other parts of the brain and nervous system, suggesting that they may be involved in other disorders (Merck Manual). In Lewy body dementia, Lewy bodies form throughout the outer layer of the brain (cerebral cortex). Lewy bodies may also be involved in Alzheimer's disease (Merck Manual).

Improved and novel methods for diagnosing, confirming, providing biomarkers for, and treating PD are needed.

### SUMMARY OF THE INVENTION

The present invention provides methods for assessing whether a subject is at risk of developing an α-synucleinopathy, Parkinson's disease (PD), Lewy Body dementia (LBD), or Alzheimer's disease (AD) comprising
i) obtaining leukocytes from the subject;
ii) contacting the leukocytes with an epitope;
iii) determining whether the leukocytes have increased activation after contact with the epitope; and
iv) identifying the subject as at risk of developing PD, LBD or AD if in step iii) the leukocytes are determined to have increased activation after contact with the epitope, and identifying the subject as not at risk of developing the α-synucleinopathy, PD, LED or AD if in step iii) the leukocytes are determined to not have increased activation after contact with the epitope.

The present invention also provides in a process for assessing whether a subject is at risk of developing an α-synucleinopathy, Parkinson's disease (PD), Lewy Body dementia (LBD), or Alzheimer's disease (AD), which involves an array of testing, the improvement comprising including in the array of testing the steps of:
i) obtaining leukocytes from the subject;
ii) contacting the leukocytes with an epitope;
iii) determining whether the leukocytes have increased activation after contact with the epitope; and
iv) identifying the subject as at risk of developing PD, LBD or AD if in step iii) the leukocytes are determined to have increased activation after contact with the epitope, and identifying the subject as not at risk of developing the α-synucleinopathy, PD, LBD or AD if in step iii) the leukocytes are determined to not have increased activation after contact with the epitope.

The present invention further provides methods for assessing whether a subject is at risk of developing an α-synucleinopathy, Parkinson's disease (PD), Lewy Body dementia (LBD), or Alzheimer's disease (AD) comprising
i) obtaining leukocytes from the subject;
ii) separating the leukocytes into 2 or more pools of leukocytes and contacting each pool with an epitope, wherein each pool is contacted with a different epitope;
iii) determining whether each pool has increased activation after contact with the epitope; and
iv) identifying the subject as at risk of developing the α-synucleinopathy, PD, LBD, or AD if and only if in step iii) 1 or more pools is determined to have increased activation after contact with an epitope.

The present invention provides in a process for assessing whether a subject is at risk of developing an α-synucleinopathy, Parkinson's disease (PD), Lewy Body dementia (LBD), or Alzheimer's disease (AD), which involves an array of testing, the improvement comprising including in the array of testing the steps of:
i) obtaining leukocytes from the subject;
ii) separating the leukocytes into 2 or more pools of leukocytes and contacting each pool with an epitope, wherein each pool is contacted with a different epitope;
iii) determining whether each pool has increased activation after contact with the epitope; and
iv) identifying the subject as at risk of developing the α-synucleinopathy, PD, LBD, or AD if in step iii) 1 or more pools is determined to have increased activation after contact with an epitope.

The present invention also provides methods for diagnosing or confirming whether a subject is afflicted with an α-synucleinopathy, Parkinson's disease (PD), Lewy Body dementia (LBD), or Alzheimer's disease (AD) comprising
i) obtaining leukocytes from the subject;
ii) contacting the leukocytes with an epitope;
iii) determining whether the leukocytes have increased activation after contact with the epitope; and
iv) identifying the subject as afflicted with PD if in step iii) the leukocytes are determined to have increased activation after contact with the epitope, and identifying the subject as not afflicted with the α-synucleinopathy, PD, LBD, or AD if in step iii) the leukocytes are determined to not have increased activation after contact with the epitope.

The present invention provides in a process for diagnosing or confirming whether a subject is afflicted with an α-synucleinopathy, Parkinson's disease (PD), Lewy Body dementia (LBD), or Alzheimer's disease (AD), which involves an array of testing, the improvement comprising including in the array of testing the steps of:
i) obtaining leukocytes from the subject;
ii) separating the leukocytes into 1 or more pools of leukocytes and contacting each pool with an epitope, wherein each pool is contacted with a different epitope;
iii) determining whether each pool has increased activation after contact with the epitope; and
iv) identifying the subject as afflicted with the α-synucleinopathy, PD, LBD, or AD if and only if in step iii) 1 or more pools is determined to have increased activation after contact with an epitope.

The present invention provides methods for diagnosing or confirming whether a subject is afflicted with an α-synucleinopathy, Parkinson's disease (PD), Lewy Body dementia (LBD), or Alzheimer's disease (AD) comprising
i) obtaining leukocytes from the subject;
ii) separating the leukocytes into 1 or more pools of leukocytes and contacting each pool with an epitope, wherein each pool is contacted with a different epitope;
iii) determining whether each pool has increased activation after contact with the epitope; and
iv) identifying the subject as afflicted with the α-synucleinopathy, PD, LBD, or AD if and only if in step iii) 1 or more pools is determined to have increased activation after contact with an epitope.

The present invention provides in a process for diagnosing or confirming whether a subject is afflicted with an α-synucleinopathy, Parkinson's disease (PD), Lewy Body dementia (LBD), or Alzheimer's disease (AD), which involves an array of testing, the improvement comprising including in the array of testing the steps of:
i) obtaining leukocytes from the subject;
ii) separating the leukocytes into 1 or more pools of leukocytes and contacting each pool with an epitope, wherein each pool is contacted with a different epitope;
iii) determining whether each pool has increased activation after contact with the epitope; and
iv) identifying the subject as afflicted with the α-synucleinopathy, PD, LED, or AD if and only if in step iii) 1 or more pools is determined to have increased activation after contact with an epitope.

The present invention also provides methods for assessing whether an α-synucleinopathy, Parkinson's disease (PD), Lewy Body dementia (LBD), or Alzheimer's disease (AD) has progressed in a subject afflicted with the α-synucleinopathy, PD, LBD, or AD comprising performing each of the following steps i) to iii):
i) obtaining leukocytes from the subject;
ii) contacting the leukocytes with an epitope that was previously identified to increase activation of the leukocytes; and
iii) determining the level of activation of the leukocytes after contact with the epitope,

at a first and a second point in time, and then
iv) concluding that the α-synucleinopathy, PD, LBD, or AD has progressed in the subject if the leukocytes are determined to be more activated in step iii) performed at the second point in time compared to the level of activation in step iii) performed at the first point in time.

The present invention further provides methods for assessing whether an α-synucleinopathy, Parkinson's disease (PD), Lewy Body dementia (LBD), or Alzheimer's disease (AD) has progressed in a subject afflicted with the α-synucleinopathy, PD, LBD, or AD comprising performing each of the following steps i) to iii):
i) obtaining leukocytes from the subject;
ii) separating the leukocytes into two or more pools of leukocytes and contacting each pool with an epitope, wherein each pool is contacted with a different epitope;
iii) determining whether each pool has increased activation after contact with the epitope;

at a first and a second point in time, and then
iv) concluding that the α-synucleinopathy, PD, LBD, or AD has progressed in the subject if more pools of leukocytes are determined to be activated in step iii) performed at the second point in time compared to the number of pools that are determined to be activated in step iii) performed at the first point in time.

The present invention provides methods for assessing whether an α-synucleinopathy, Parkinson's disease (PD), Lewy Body dementia (LBD), or Alzheimer's disease (AD) is progressing in a subject afflicted with the α-synucleinopathy, PD, LBD, or AD comprising periodically performing the method according to the method of the invention.

The present invention also provides methods for treating a subject afflicted with an α-synucleinopathy, Parkinson's disease (PD), Lewy Body dementia (LBD), or Alzheimer's disease (AD), comprising administering to the subject a compound that is approved for use in treating subjects afflicted with the α-synucleinopathy, PD, LBD, or AD, wherein the subject has been diagnosed or confirmed to be afflicted with PD according to a method of the invention.

The present invention further provides methods for treating a subject afflicted with the α-synucleinopathy, Parkinson's disease (PD), Lewy Body dementia (LBD), or Alzheimer's disease (AD), comprising diagnosing or confirming the subject to be afflicted with the α-synucleinopathy, PD, LBD, or AD according to a method of the invention, and administering to the subject a compound that is approved for use in treating subjects afflicted with PD, LBD, or AD.

The present invention also provides methods for assessing whether an α-synucleinopathy, Parkinson's disease (PD), Lewy Body dementia (LBD), or Alzheimer's disease (AD) is developing in a subject who has been identified as being at risk of developing the α-synucleinopathy, PD, LBD, or AD comprising performing each of the following steps i) to iii):
i) obtaining leukocytes from the subject;
ii) contacting the leukocytes with an epitope that was previously identified to increase activation of the leukocytes; and
iii) determining the level of activation of the leukocytes after contact with the epitope,

at a first and a second point in time, and then
iv) concluding that the α-synucleinopathy, PD, LBD, or AD is developing in the subject if the leukocytes are determined to be more activated in step iii) performed at the second point in time compared to the level of activation in step iii) performed at the first point in time.

The present invention further provides methods for assessing whether an α-synucleinopathy, Parkinson's disease (PD), Lewy Body dementia (LBD), or Alzheimer's disease (AD) is developing in a subject who has been identified as being at risk of developing the α-synucleinopathy, PD, LSD, or AD comprising performing each of the following steps i) to iii):
i) obtaining leukocytes from the subject;
ii) separating the leukocytes into two or more pools of leukocytes and contacting each pool with an epitope, wherein each pool is contacted with a different epitope;
iii) determining whether each pool has increased activation after contact with the epitope;

at a first and a second point in time, and then
iv) concluding that that PD, LBD, or AD is developing in the subject if more pools of leukocytes are determined to be activated in step iii) performed at the second point in time compared to the number of pools that are determined to be activated in step iii) performed at the first point in time.

The present invention also provides methods for assessing whether a subject afflicted with an α-synucleinopathy, Parkinson's disease (PD), Lewy Body dementia (LBD), or Alzheimer's disease (AD) is likely to benefit from a therapy, wherein the therapy is directed to leukocytes that are activated by an epitope, the method comprising
i) obtaining leukocytes from the subject;
ii) contacting the leukocytes with the epitope;
iii) determining whether the leukocytes have increased activation after contact with the epitope; and
iv) identifying the subject as likely to benefit from the therapy if in step iii) the leukocytes are determined to have increased activation after contact with the epitope, and identifying the subject as unlikely to benefit from the therapy if in step iii) the leukocytes are determined to not have increased activation after contact with the epitope.

The present invention further provides methods for producing a written or electronic report identifying whether a subject afflicted with an α-synucleinopathy, Parkinson's disease (PD), Lewy Body dementia (LBD), or Alzheimer's disease (AD) is likely to benefit from a therapy, wherein the therapy is directed to leukocytes that are activated by an epitope, comprising
i) obtaining leukocytes from the subject;
ii) contacting the leukocytes with the epitope;
iii) determining whether the leukocytes have increased activation after contact with the epitope;
iv) identifying the subject as likely to benefit from the therapy if in step iii) the leukocytes are determined to have increased activation after contact with the epitope, and identifying the leukocytes of the subject as unlikely to benefit from the therapy if in step iii) the leukocytes are determined to not have increased activation after contact with the epitope; and
v) preparing the written or electronic report identifying whether the subject is likely to benefit from the therapy based on the identification in step iv).

The present invention provides methods for assessing whether a subject afflicted with an α-synucleinopathy, Parkinson's disease (PD), Lewy Body dementia (LBD), or Alzheimer's disease (AD) has benefited from a therapy, wherein the therapy is directed to leukocytes that are activated by an epitope, the method comprising
i) obtaining leukocytes from the subject;
ii) contacting the leukocytes with the epitope;
iii) determining whether the leukocytes have increased activation after contact with the epitope; and
iv) identifying the subject as having benefited from the therapy if in step iii) if the leukocytes are determined to have increased activation after contact with the epitope, and identifying the subject as not having benefited from the therapy if in step iii) the leukocytes are determined to not have increased activation after contact with the epitope.

The present invention also provides methods for treating a subject afflicted with an α-synucleinopathy, Parkinson's disease (PD), Lewy Body dementia (LBD), or Alzheimer's disease (AD) who has been identified as being likely to benefit from a therapy directed to leukocytes that are activated by an epitope, comprising administering the therapy that is specific for leukocytes that are activated by the epitope to the subject.

The present invention further provides methods for prophylactically treating a subject who has been identified as being at risk of developing an α-synucleinopathy, Parkinson's disease (PD), Lewy Body dementia (LBD), or Alzheimer's disease (AD) comprising administering to the subject a therapy that is directed to leukocytes that are activated by an epitope, wherein leukocytes of the subject have been determined to have increased activation after contact with the epitope.

The present invention provides methods for treating a subject afflicted with an α-synucleinopathy, Parkinson's disease (PD), Lewy

Body dementia (LBD), or Alzheimer's disease (AD) comprising administering to the subject a therapy that is directed to leukocytes that are activated by an epitope, wherein leukocytes of the subject have been determined to have increased activation after contact with the epitope.

The present invention also provides methods for treating a subject afflicted with an α-synucleinopathy, Parkinson's disease (PD), Lewy Body dementia (LBD), or Alzheimer's disease (AD), comprising administering an immunosuppressant therapy to the subject.

The present invention also provides methods for treating, such as prophylactically treating, a subject afflicted with an α-synucleinopathy, Parkinson's disease (PD), Lewy Body dementia (LED), or Alzheimer's disease (AD), comprising administering to the subject an immunosuppressant therapy.

The present invention further provides methods for assessing whether leukocytes of a subject afflicted with an α-synucleinopathy, Parkinson's disease (PD), Lewy Body dementia (LBD), or Alzheimer's disease (AD) are activated by an epitope, comprising
i) obtaining leukocytes from the subject;
ii) contacting the leukocytes with the epitope;
iii) determining whether the leukocytes have increased activation after contact with the epitope; and
iv) identifying the leukocytes of the subject as activated by the epitope if in step iii) the leukocytes are determined to have increased activation after contact with the epitope, and identifying the leukocytes of the subject as not activated by the epitope if in step iii) the leukocytes are determined to not have increased activation after contact with the epitope.

The present invention provides compounds for treating an α-synucleinopathy, Parkinson's disease (PD), Lewy Body dementia (LBD), or Alzheimer's disease (AD), comprising i) a major histocompatibility complex (MHC) Tetramer having four MHC molecules, wherein each MHC molecule is associated with an epitope, and ii) a toxin.

The present invention also provides pharmaceutical compositions comprising one or more compounds or epitopes of the invention.

### BRIEF DESCRIPTION OF THE DRAWINGS

FIGS. 1A-G. Human SN and LC express MHC-I. (A) Fluorescent images showing double immunolabel for the neuronal nuclei marker, Fox-3 (green) and human MHC-I HLA (A, B and C, red) in human postmortem hippocampal/entorhinal cortex sections and striatal sections from control individuals. White circles demonstrate that there is no overlap between neurons and HLA⁺ structures. Scale bar, 60 μm. (B,C) Brightfield and immunofluorescence images of SN stained for (B) HLA (A, B and C, red) and TH (green) and (C) β2m (red) and TH (green). NM was identified under brightfield illumination. Encircled neurons demonstrate that TH⁺ neurons display HLA immunolabel, overlapping in particular with NM. Scale bar, 50 μm in b and 75 μm in c. (D) Confocal immunofluorescent label of TH (green) and HLA (A, B and C, red) in SN and LC control and PD samples. The first row shows a representative example of an SN DA neuron that does not express HLA. When HLA was observed in SN and LC neurons (examples in the second through fifth rows), it was often associated with NM. Scale bar, 25 μm. (E and F) Proportion of TH⁺ neurons with HLA (A, B and C) immunolabel in the SN (E) and the LC (F). Cell counts were performed in three sections per brain of eight control individuals and eight PD patients (SN) and eight control individuals and nine PD patients (LC). Data are presented as the mean±s.e.m. P>0.05 (NS) in E (Mann-Whitney U-test) and *P<0.05 in F (Mann-Whitney U-test). (G) Percentage of TH⁺ and TH⁻ neurons in the SN and LC (control and PD) labelled for HLA. The arrows in all panels point to blood vessels. Each experiment was repeated at least in triplicate. BF, brightfield; CTRL, control; NS, nonsignificant.

FIGS. 2A-F. Human SN and LC express MHC-1 with local CTLs. (A) V-VIP immunostain (purple) indicates HLA (A, B and C) in control and PD SN and LC samples. NM appears as brown precipitate. Arrows indicate labelled cells in which the chromogen fills and outlines cell bodies and occasional dendrites. The arrowheads indicate NM⁺ neurons devoid of cytosolic immunocytochemical label. Scale bar, 25 μm in the left panel and 15 μm in the right panel. (B) Immunoelectron microscopy images demonstrating antigenicity to HLA (A, B and C) within NM granules of control SN and LC (white arrows). Blood vessel endothelium was used as a positive control for the HLA antibody and showed immunolabel (black arrows); the erythrocyte showed very little staining (white outlined arrowhead). Lipid bodies (asterisk) within NM organelles did not exhibit HLA immunolabel. The black outlined arrowhead depicts a lysosome with HLA label in an LC neuron. Scale bar, 250 nm. (C) MS/MS spectrum (parent ion m/z 788.2=[M+3H]3⁺ corresponding to the peptide NTQTDRESLRNLRCYYNQS (SEQ ID NO: 58) observed in the analysis of NM isolated from SN tissue. See Table 1 for details. (D) β2m, HLA-A and HLA-C genes are robustly expressed in laser-captured NM SN neurons of control individuals. Data are presented as the mean±s.e.m. Samples from 9 control subjects were analyzed. (E) Double immunofluorescence in the SN of human of a postmortem control sample. NM was observed under brightfield microscopy. An arrow indicates a CD8⁺ T cell in contact with a NM⁺ neuron. Scale bar, 15 μm. (F) Immunofluorescence in hES-derived DA neurons, showing HLA (A, B and C) immunolabel in the cell body (upper panel) and dendrites (lower panel) of TH⁺ neurons exposed to human IFN-γ, but not in neurons treated with the vehicle. Asterisks indicate cell bodies and arrows point at dendrites. Scale bar, 10 μm. Each experiment was repeated at least in triplicate, and within each experiment, each condition was also performed at least three times. BF, brightfield; CTRL, control.

FIGS. 3A-E. Induced MHC-I by murine catecholamine neurons. (A) MHC-I immunolabel in postnatally-derived cultured SN DA neurons from wild type and β2m KO mice imaged by confocal microscopy. The upper row shows untreated DA (TH: green) neurons. The bottom row shows MHC-I (red) expressing DA neurons exposed to IFN-γ. The arrow indicates a MHC-I expressing astrocyte (bottom row). Scale bar, 20 μm. (B) Dose response of MHC-I induction by IFN-γ in neurons obtained from various brain regions. Data are presented as the mean±s.e.m (NS; ** P<0.01; *** P<0.001, Two-way analysis of variance (ANOVA) test). (C) IFN-γ released by microglia stimulated with LPS, NM or α-syn. Data are presented as the mean±s.e.m. (** P<0.01; *** P<0.001, One-way ANOVA with Tukey post-hoc test). (D) Expression of MHC-I after exposing primary VM neurons to medium from microglia pre-stimulated with LPS, NM or α-syn (wild type, nitrated or A53T mutant). Data are presented as the mean±s.e.m. (* P<0.05; ** P<0.01; *** P<0.001, One-way ANOVA with Tukey post hoc test). (E) Percentage of VM neurons that expressed MHC-I after exposure to microglial conditioned as in C), with or without a neutralizing antibody for IFN-γ. Data are presented as the mean±s.e.m. (NS; *P<0.05; ** P<0.01, two-tailed Student's T-test). Each experiment was repeated at least in triplicate, and within each experiment, each condition was also performed at least in triplicate, and within each experiment, each condition was also performed at least three times. For each condition, neurons on n=24 fields at 20× were quantified. ab=antibody; CTRL=control; NS=nonsignificant; WT=wild type.

FIGS. 4A-C. MHC-I induction in VM DA neurons is dependent on oxidative stress. (A) Example of TH/MHC-I double immunolabel of primary cultures of VM neurons after treatment with L-DOPA, which induced the presence of both NM (arrow) and MHC-I. Scale bar, 30 μm. (B) Fraction of TH⁺ and TH⁻ neurons that displayed NM following L-DOPA. Data are presented as mean±s.e.m. (NS, two-tailed Student's t-test). (C) The fraction of TH⁺ and TH⁻ neurons that displayed plasma membrane MHC-I following L-DOPA. Data are presented as mean±s.e.m. (* p<0.05, two-tailed Student's t-test). Each experiment was repeated at least in triplicate and within each experiment, each condition was also performed at least three times. For each condition, neurons on n=24 fields at 20× were quantified. BF=brightfield; CTRL=control; NS=nonsignificant.

FIGS. 5A-B. VM DA neurons load and display antigen. (A) VM DA neurons immunolabeled for TH and SIINFEKL-MHC-I. (8) The fraction of TH⁺ neurons that were labeled for SIINFEKL-MHC-I following vehicle, OVA, IFN-γ+SIINFEKL (SEQ ID NO: 9), and IFN-γ+OVA. Data are presented as the mean±s.e.m. (*** P<0.001, one-way ANOVA with Tukey post-hoc test). Each experiment was repeated at least in triplicate and within each experiment, each condition was also performed at least three times. For each condition, neurons on n=24 fields at 20× were quantified. Scale bar, 30 μm. IFN=interferon gamma; SIIN=SIINFEKL (SEQ ID NO: 9); Veh=vehicle.

FIGS. 6A-C. VM DA neurons that display antigen are killed by CD8⁺ T cells. (A) The fraction of VM DA neurons surviving in the presence of SIINFEKL (SEQ ID NO: 9), OT-1 cells pre-pulsed with SIINFEKL (SEQ ID NO: 9), and IFN-γ (100 ng/ml) in cultures obtained from wild type and β2m KO mice. Data are presented as the mean±s.e.m. (NS; ** P<0.01, One-way analysis of variance (ANOVA) with Tukey post-hoc test). (B) Survival of VM DA neurons incubated with IFN-7, SIINFEKL (SEQ ID NO: 9) peptide, and OT-1 cells with the pan-caspase inhibitor Z-VAD-FMK, the Fas/Fas ligand antagonist, Kp7-6, the perforin/granzyme antagonist, concanamycin A, or the combination of Kp7-6 and concanamycin A. Data are presented as the mean±s.e.m. (NS; * P<0.05, *** P<0.001, one-way ANOVA with Tukey post-hoc test). (C) VM DA neuron survival with SIINFEKL (SEQ ID NO: 9), OT-1 cells pre-pulsed with SIINFEKL (SEQ ID NO: 9), and microglial medium previously exposed to LPS, α-syn or NM. Data are presented as the mean±s.e.m. (NS; * P<0.05, ** P<0.01; *** P<0.001, one-way ANOVA with Tukey post-hoc test). Each experiment was repeated at least in triplicate and within each experiment, each condition was also performed at least three times. For each condition, neurons on n=24 fields at 20× were quantified. ConA=concanamycin A; CTRL=control; IFN=interferon gamma; NS=non significant; SIIN=SIINFEKL (SEQ ID NO: 9); Veh=vehicle.

FIGS. 7A-B. Series of low magnification brightfield and immunofluorescence images (Fox-3 in green/HLA A, B and C in red). Circles indicate neurons that have NM (see brightfield images) and are immunoreactive for both Fox-3 (green) and HLA (red). Arrows indicate blood vessels. Squares indicate the presence of nuclei of NM⁻ neurons, which are negative for HLA. Photomicrographs illustrate both a region with NM⁺ neurons (top row) and an area within the same section with NM⁻ (bottom row) of the SN. Consistent with TH staining results, HLA immunolabel appears mainly in blood vessels and in NM⁺ cells, while neurons devoid of NM are also devoid of HLA, both in the SN (A) and in the LC (B). The intraneuronal label of Fox-3 in SN neurons is consistent with previous reports (Cannon, J. R., & Greenamyre, J. T. NeuN is not a reliable marker of dopamine neurons in rat substantia nigra. Neurosci. Lett. 464, 14-7. (2009)). Scale=50 μm. BF=brightfield; CTRL=control.

FIGS. 8A-D. (A) Brightfield and immunofluorescence images of a VTA neuron (NM⁻) with HLA (red) and TH (green). Scale=10 μm. (B) V-VIP immunostaining of a SN section devoid of NM⁺ neurons showing HLA in blood vessels (arrows) but not in glia or neurons. Scale=50 μm (C) Additional immunoelectron microscopy images demonstrating antigenicity to HLA within NM granules of SN and LC (white arrows). Scale=500 nm. (D) Double immunofluorescence in the SN of human of a postmortem PD sample. NM was observed under brightfield microscopy. HLA appears in red and CD8⁺ T cells in green. Scale=50 μm. Each experiment was repeated at least in triplicate. Within each individual experiment, each condition was also performed at least three times; BF=brightfield.

FIGS. 9A-F. (A) Cultured motor neurons derived from hES treated with either the vehicle (PBS) or human IFN-γ. No MHC-I was observed in any condition. Green=synapsin, blue=choline acetyltransferase, a marker for motor neurons and red=MHC-I. Scale=50 μm. (B) Double TH/MHC-I immunolabel in unfixed postnatally-derived cultured SN DA neurons imaged by confocal microscopy. Control DA neurons exhibited no plasma membrane MHC-I immunolabel. DA neuron (TH: green) exposed to IFN-γ presented MHC-I. Scale=30 μm. (C) Comparison of the expression of MHC-I after exposing primary VM, cortical and striatal neurons to medium from microglia pre-stimulated with LPS, NM or α-syn. Data are presented as the mean±SEM (ns=non significant; ** p<0.01, One-way ANOVA with Tukey post-hoc test). (D) OT-1 cell proliferation as measured by BrdU incorporation. SIINFEKL (SEQ ID NO: 9) peptide alone did not activate OT-1 cell proliferation in the absence of other cells, but SIINFEKL (SEQ ID NO: 9) peptide activated OT-1 cell proliferation in the presence of either DCs, or with IFN-γ exposed VM neuronal cultures. Data are presented as the mean±SEM (ns=non significant; *** p<0.001, One-way ANOVA with Tukey post-hoc test). (E) Neuronal survival after incubating VM DA neurons with or without OT-1 cell conditioned medium after seven days of culture. Data are presented as the mean±SEM (ns=non significant, two-tailed Student's T-test). (F) Neuronal survival of wild type VM DA neurons plated on astrocytes from wild type of β2m KO mice after the addition of IFN-γ, SIINFEKL (SEQ ID NO: 9) and OT-1 cells. Data are presented as the mean±SEM (ns=non significant, two-tailed Student's T-test). Each experiment was repeated at least in triplicate and within each experiment, each condition was also performed at least three times. For each condition, neurons on n=24 fields at 20× were quantified. CTRL control; IFN=interferon gamma; SIIN=SIINFEKL (SEQ ID NO: 9) peptide; Veh=vehicle.

FIGS. 10A-E (A) Brightfield and fluorescence images in the human SN showing double immunostaining for HLA A, B, and C and TH and β2m and TH after pre-adsorbing each primary antibody with its correspondent neutralizing peptide. NM was identified under brightfield illumination. The staining for HLA was fully blocked within the neurons and partially blocked in the blood vessels, where the levels of HLA are much higher than in the neurons. β2m labelling was totally blocked in neurons and in blood vessels, and only some inespecific precipitate could be observed in the form of dots. Scale=100 μm. (B) Representative western blots of HLA and β2m expression in samples from the SN of a human control individual. A single band was observed at 43 KDa for HLA and at 12 KDa for β2m. (C) HLA blot (upper panel): the first lane shows the positive control, second and third lanes show our sample blotted with the primary antibody and fourth lane shows our sample blotted with the primary antibody pre-adsorbed with the blocking peptide. In the β2m blot (lower panel), the first lane shows the positive control, second lane shows our sample blotted with the primary antibody and third lane show our sample blotted with the primary antibody pre-adsorbed with the blocking peptide. (D) Immunofluorescence images of CD8⁺ expression in human spleen sections at different magnifications and the correspondent negative control (omission of primary antibody). Calibration bars=50 μm (small zoom) and 10 μm (large zoom). (E) Representative western blot of MHC-I expression in samples from brain homogenates from wild type versus β2m KO mice. Positive control was loaded on both sides of the gel. Left panel shows the uncropped film. BF=brightfield; BP=blocking peptide; C+=positive control; S1=sample.

FIGS. 11A-C. 11 (A) IFNg Reactivity to Synuclein; (B) IL-5 Reactivity to Synuclein; (C) IFNg and IL-5 Reactivity to Synuclein.

FIGS. 12A-B. 12 (A) IFNg—Graph of SFC for Donors; (B) IL-5—Graph of SFC for Donors.

FIG. 13. PD patients (black) are far more reactive to α-syn derived peptides than age matched control (green). (Top) PD patients show far more response to 15 aa length peptides. In the region containing Y39 (left yellow shaded area) (green) p<0.004, Mann Whitney test vs. controls. For phosphorylated S129 regions, PD patients also had far higher response than controls (p=0.006). For combined response to 15-mer epitopes from both regions, p<0.005, with 12/44 PD patients responding, and 3/23 controls (p=0.03). (Bottom) T cell response against short (8-11 mer) peptides (bottom) was seen only in PD patients (12/44) with no control subjects responding (0/24. p<0.005). Altogether for long and short peptides, 50% of PD patients showed antigenic responses to α-syn epitopes and 12.5% of controls (p<0.01 by Chi square) and the difference in magnitude response is p<0.005.

FIG. 14. rAAV2-GFP (top row) and rAAV2-SYN (bottom) adenoviruses injected in the SN of WT mice show high signals in the DA neurons by 2 weeks after the injection.

FIG. 15. Westerns blot of samples obtained from the SN dissected from n=5 WT mice injected with rAAV2-GFP or rAAV2-SYN. rAAV2-SYN mice exhibit increased of MHC-I (400%), the MHC-I subunit β2m (200%) and MHC-II (400%) more than the GFP control virus: each comparison is p>0.01.

FIG. 16. MHC-I label in SN neurons (TH+) is higher when injected with rAAV2-SYN than rAAV2-GFP.

FIG. 17. ELISA for TNF-α shows far higher proinflammatory cytokine in the striatum of WT rAAV2-SYN SN injected mice than WT rAAV2-GFP. MHC-I KO mice had even lower TNF-α levels (not shown).

### DETAILED DESCRIPTION OF THE INVENTION

The present invention provides methods for assessing whether a subject is at risk of developing an α-synucleinopathy, Parkinson's disease (PD), Lewy Body dementia (LBD), or Alzheimer's disease (AD) comprising i) obtaining leukocytes from the subject;
ii) contacting the leukocytes with an epitope;
iii) determining whether the leukocytes have increased activation after contact with the epitope; and
iv) identifying the subject as at risk of developing PD, LBD or AD if in step iii) the leukocytes are determined to have increased activation after contact with the epitope, and identifying the subject as not at risk of developing the α-synucleinopathy, PD, LBD or AD if in step iii) the leukocytes are determined to not have increased activation after contact with the epitope.

In some embodiments, the method is for assessing whether a subject is at risk of developing Parkinson's disease (PD) and comprises
i) obtaining leukocytes from the subject;
ii) contacting the leukocytes with an epitope;
iii) determining whether the leukocytes have increased activation after contact with the epitope; and
iv) identifying the subject as at risk of developing PD if in step iii) the leukocytes are determined to have increased activation after contact with the epitope, and identifying the subject as not at risk of developing PD if in step iii) the leukocytes are determined to not have increased activation after contact with the epitope.

The present invention provides in a process for assessing whether a subject is at risk of developing an α-synucleinopathy, Parkinson's disease (PD), Lewy Body dementia (LBD), or Alzheimer's disease (AD), which involves an array of testing, the improvement comprising including in the array of testing the steps of:
i) obtaining leukocytes from the subject;
ii) contacting the leukocytes with an epitope;
iii) determining whether the leukocytes have increased activation after contact with the epitope; and
iv) identifying the subject as at risk of developing PD, LBD or AD if in step iii) the leukocytes are determined to have increased activation after contact with the epitope, and identifying the subject as not at risk of developing the α-synucleinopathy, PD, LBD or AD if in step iii) the leukocytes are determined to not have increased activation after contact with the epitope.

The present invention provides methods for assessing whether a subject is at risk of developing an α-synucleinopathy, Parkinson's disease (PD), Lewy Body dementia (LBD), or Alzheimer's disease (AD) comprising
i) obtaining leukocytes from the subject;
ii) separating the leukocytes into 2 or more pools of leukocytes and contacting each pool with an epitope, wherein each pool is contacted with a different epitope;
iii) determining whether each pool has increased activation after contact with the epitope; and
iv) identifying the subject as at risk of developing the α-synucleinopathy, PD, LBD, or AD if and only if in step iii) 1 or more pools is determined to have increased activation after contact with an epitope.

The present invention provides in a process for assessing whether a subject is at risk of developing an α-synucleinopathy, Parkinson's disease (PD), Lewy Body dementia (LBD), or Alzheimer's disease (AD), which involves an array of testing, the improvement comprising including in the array of testing the steps of:
i) obtaining leukocytes from the subject;
ii) separating the leukocytes into 2 or more pools of leukocytes and contacting each pool with an epitope, wherein each pool is contacted with a different epitope;
iii) determining whether each pool has increased activation after contact with the epitope; and
iv) identifying the subject as at risk of developing the α-synucleinopathy, PD, LBD, or AD if in step iii) 1 or more pools is determined to have increased activation after contact with an epitope.

In some embodiments, the method is for assessing whether a subject is at risk of developing Parkinson's disease (PD) and comprises
i) obtaining leukocytes from the subject;
ii) separating the leukocytes into 2 or more pools of leukocytes and contacting each pool with an epitope, wherein each pool is contacted with a different epitope;
iii) determining whether each pool has increased activation after contact with the epitope; and
iv) identifying the subject as at risk of developing PD if and only if in step iii) 1 or more pools is determined to have increased activation after contact with an epitope.

In some embodiments, in step ii) the leukocytes are separated into 2, 3, 4, 5, 6, 7, 8, 9, 10, 11-50 or more pools, and in step iv) the subject is identified as at risk of developing the α-synucleinopathy, PD, LBD or AD if and only if in step iii) 1, 2, 3, 4, 5, 6, 7, 8, 9, 10, 11-50 or more pools is determined to have increased activation after contact with an epitope.

In some embodiments, the subject is at least about 35, 40, 45, 50, 55, 60, 65, 70, 75 or 80 years of age. In some embodiments, the subject is less than about 35, 40, 45, 50, 55, 60, 65, 70, 75 or 80 years of age.

In some embodiments, the subject has a symptom that has preceded the onset of the α-synucleinopathy, PD, LBD or AD in subjects who have developed the α-synucleinopathy, PD, LBD or AD. In some embodiments, the symptom has preceded the onset of the α-synucleinopathy, PD, LBD or AD in the subjects by at least about 5, 10, 15, 20, 25, 30 or 5-30 years.

In some embodiments, the subject is afflicted with cognitive decline, constipation or orthostatic hypotension. In some embodiments, the subject is afflicted with cognitive decline, and the cognitive decline is reduced spatial reasoning ability and/or reduced memory ability.

In some embodiments, the process further comprises directing the subject to
(a) be monitored more frequently for the α-synucleinopathy, PD, LBD, or AD; or
(b) receive additional diagnostic testing for the α-synucleinopathy, PD, LBD, or AD,

if the subject is identified as at risk of developing the α-synucleinopathy, PD, LBD, or AD.

In some embodiments, the method further comprises determining the presence of at least one human leukocyte antigen (HLA) allele in the subject. In some embodiments, the subject is identified as at risk of developing the α-synucleinopathy, PD, LBD, or AD if
(a) the leukocytes are determined to have increased activation after contact with the epitope, or 1 or more pools is determined to have increased activation after contact with an epitope, and
(b) subject has the HLA allele DRB5*01:01 or DRB1*15:01.

In some embodiments, determining the presence of at least one HLA allele in the subject comprises obtaining genomic DNA from the subject and assaying with a probe or a primer, wherein if the assaying is with a primer, then the assaying comprises
(1) hybridizing a primer to a nucleic acid having the sequence of a region proximal to the HLA allele or
(2) hybridizing a primer to a nucleic acid having the sequence within the HLA allele, and

if the assaying is with a probe, then the assaying comprises hybridizing a probe to a nucleic acid having the sequence within the HLA allele. In some embodiments, the assaying is with a probe, and the probe is part of a probe array.

The present invention provides methods for diagnosing or confirming whether a subject is afflicted with an α-synucleinopathy, Parkinson's disease (PD), Lewy Body dementia (LBD), or Alzheimer's disease (AD) comprising
i) obtaining leukocytes from the subject;
ii) contacting the leukocytes with an epitope;
iii) determining whether the leukocytes have increased activation after contact with the epitope; and
iv) identifying the subject as afflicted with PD if in step iii) the leukocytes are determined to have increased activation after contact with the epitope, and identifying the subject as not afflicted with the α-synucleinopathy, PD, LBD, or AD if in step iii) the leukocytes are determined to not have increased activation after contact with the epitope.

The present invention provides in a process for diagnosing or confirming whether a subject is afflicted with an α-synucleinopathy, Parkinson's disease (PD), Lewy Body dementia (LBD), or Alzheimer's disease (AD), which involves an array of testing, the improvement comprising including in the array of testing the steps of:
i) obtaining leukocytes from the subject;
ii) separating the leukocytes into 1 or more pools of leukocytes and contacting each pool with an epitope, wherein each pool is contacted with a different epitope;
iii) determining whether each pool has increased activation after contact with the epitope; and
iv) identifying the subject as afflicted with the α-synucleinopathy, PD, LBD, or AD if and only if in step iii) 1 or more pools is determined to have increased activation after contact with an epitope.

In some embodiments, the method is for diagnosing or confirming whether a subject is afflicted with Parkinson's disease (PD) and comprises
i) obtaining leukocytes from the subject;
ii) contacting the leukocytes with an epitope;
iii) determining whether the leukocytes have increased activation after contact with the epitope; and
iv) identifying the subject as afflicted with PD if in step iii) the leukocytes are determined to have increased activation after contact with the epitope, and identifying the subject as not afflicted with PD if in step iii) the leukocytes are determined to not have increased activation after contact with the epitope.

The present invention provide methods for diagnosing or confirming whether a subject is afflicted with an α-synucleinopathy, Parkinson's disease (PD), Lewy Body dementia (LBD), or Alzheimer's disease (AD) comprising
i) obtaining leukocytes from the subject;
ii) separating the leukocytes into 1 or more pools of leukocytes and contacting each pool with an epitope, wherein each pool is contacted with a different epitope;
iii) determining whether each pool has increased activation after contact with the epitope; and
iv) identifying the subject as afflicted with the α-synucleinopathy, PD, LBD, or AD if and only if in step iii) 1 or more pools is determined to have increased activation after contact with an epitope.

The present invention provides in a process for diagnosing or confirming whether a subject is afflicted with an α-synucleinopathy, Parkinson's disease (PD), Lewy Body dementia (LED), or Alzheimer's disease (AD), which involves an array of testing, the improvement comprising including in the array of testing the steps of:
i) obtaining leukocytes from the subject;
ii) separating the leukocytes into 1 or more pools of leukocytes and contacting each pool with an epitope, wherein each pool is contacted with a different epitope;
iii) determining whether each pool has increased activation after contact with the epitope; and
iv) identifying the subject as afflicted with the α-synucleinopathy, PD, LED, or AD if and only if in step iii) 1 or more pools is determined to have increased activation after contact with an epitope.

In some embodiments, the method is for diagnosing or confirming whether a subject is afflicted with Parkinson's disease (PD) and comprises
i) obtaining leukocytes from the subject;
ii) separating the leukocytes into 2 or more pools of leukocytes and contacting each pool with an epitope, wherein each pool is contacted with a different epitope;
iii) determining whether each pool has increased activation after contact with the epitope; and
iv) identifying the subject as afflicted with PD if and only if in step iii) 1 or more pools is determined to have increased activation after contact with an epitope.

In some embodiments, in step ii) the leukocytes are separated into 2, 3, 4, 5, 6, 7, 8, 9, 10, 11-50 or more pools, and in step iv) the subject is identified as afflicted with the α-synucleinopathy, PD, LBD or AD if and only if in step iii) 1, 2, 3, 4, 5, 6, 7, 8, 9, 10, 11-50 or more pools is determined to have increased activation after contact with an epitope.

In some embodiments, the method further comprises determining the presence of at least one human leukocyte antigen (HLA) allele in the subject. In some embodiments, the subject is identified as afflicted with the α-synucleinopathy, PD, LBD, or AD if
(a) the leukocytes are determined to have increased activation after contact with the epitope, or 1 or more pools is determined to have increased activation after contact with an epitope, and
(b) the subject has the HLA allele DRB5*01:01 or DRB1*15:01.

In some embodiments, determining the presence of at least one HLA allele in the subject comprises obtaining genomic DNA from the subject and assaying with a probe or a primer, wherein if the assaying is with a primer, then the assaying comprises
(1) hybridizing a primer to a nucleic acid having the sequence of a region proximal to the HLA allele or
(2) hybridizing a primer to a nucleic acid having the sequence within the HLA allele, and

if the assaying is with a probe, then the assaying comprises hybridizing a probe to a nucleic acid having the sequence within the HLA allele. In some embodiments, the assaying is with a probe, and the probe is part of a probe array.

The present invention provides methods for assessing whether an α-synucleinopathy, Parkinson's disease (PD), Lewy Body dementia (LBD), or Alzheimer's disease (AD) has progressed in a subject afflicted with the α-synucleinopathy, PD, LBD, or AD comprising performing each of the following steps i) to iii):
i) obtaining leukocytes from the subject;
ii) contacting the leukocytes with an epitope that was previously identified to increase activation of the leukocytes; and
iii) determining the level of activation of the leukocytes after contact with the epitope,
   at a first and a second point in time, and then
iv) concluding that the α-synucleinopathy, PD, LBD, or AD has progressed in the subject if the leukocytes are determined to be more activated in step iii) performed at the second point in time compared to the level of activation in step iii) performed at the first point in time.

In some embodiments, the method is for assessing whether Parkinson's disease (PD) has progressed in a subject afflicted with PD and comprises performing each of the following steps i) to iii):
i) obtaining leukocytes from the subject;
ii) contacting the leukocytes with an epitope that was previously identified to increase activation of the leukocytes; and
iii) determining the level of activation of the leukocytes after contact with the epitope,

at a first and a second point in time, and then
iv) concluding that the PD has progressed in the subject if the leukocytes are determined to be more activated in step iii) performed at the second point in time compared to the level of activation in step iii) performed at the first point in time.

The present invention provides methods for assessing whether an α-synucleinopathy, Parkinson's disease (PD), Lewy Body dementia (LBD), or Alzheimer's disease (AD) has progressed in a subject afflicted with the α-synucleinopathy, PD, LBD, or AD comprising performing each of the following steps i) to iii):
i) obtaining leukocytes from the subject;
ii) separating the leukocytes into two or more pools of leukocytes and contacting each pool with an epitope, wherein each pool is contacted with a different epitope;
iii) determining whether each pool has increased activation after contact with the epitope;

at a first and a second point in time, and then
iv) concluding that the α-synucleinopathy, PD, LBD, or AD has progressed in the subject if more pools of leukocytes are determined to be activated in step iii) performed at the second point in time compared to the number of pools that are determined to be activated in step iii) performed at the first point in time.

In some embodiments, the method is for assessing whether Parkinson's disease (PD) has progressed in a subject afflicted with PD and comprises performing each of the following steps i) to iii):
i) obtaining leukocytes from the subject;
ii) separating the leukocytes into two or more pools of leukocytes and contacting each pool with an epitope, wherein each pool is contacted with a different epitope;
iii) determining whether each pool has increased activation after contact with the epitope;

at a first and a second point in time, and then
iv) concluding that the PD has progressed in the subject if more pools of leukocytes are determined to be activated in step iii) performed at the second point in time compared to the number of pools that are determined to be activated in step iii) performed at the first point in time.

The present invention also provides methods for assessing whether an α-synucleinopathy, Parkinson's disease (PD), Lewy Body dementia (LBD), or Alzheimer's disease (AD) is progressing in a subject afflicted with the α-synucleinopathy, PD, LBD, or AD comprising periodically performing the method according to the method of the invention.

The present invention also provides methods for treating a subject afflicted with an α-synucleinopathy, Parkinson's disease (PD), Lewy Body dementia (LBD), or Alzheimer's disease (AD), comprising administering to the subject a compound that is approved for use in treating subjects afflicted with the α-synucleinopathy, PD, LBD, or AD, wherein the subject has been diagnosed or confirmed to be afflicted with PD according to a method of the invention.

The present invention further provides methods for treating a subject afflicted with the α-synucleinopathy, Parkinson's disease (PD), Lewy Body dementia (LBD), or Alzheimer's disease (AD), comprising diagnosing or confirming the subject to be afflicted with the α-synucleinopathy, PD, LBD, or AD according to a method of the invention, and administering to the subject a compound that is approved for use in treating subjects afflicted with PD, LBD, or AD. In some embodiments, the subject has been diagnosed or confirmed to be afflicted with PD according to a method of the invention.

The present invention provides methods for assessing whether an α-synucleinopathy, Parkinson's disease (PD), Lewy Body dementia (LBD), or Alzheimer's disease (AD) is developing in a subject who has been identified as being at risk of developing the α-synucleinopathy, PD, LBD, or AD comprising performing each of the following steps i) to iii):
i) obtaining leukocytes from the subject;
ii) contacting the leukocytes with an epitope that was previously identified to increase activation of the leukocytes; and
iii) determining the level of activation of the leukocytes after contact with the epitope,

at a first and a second point in time, and then
iv) concluding that the α-synucleinopathy, PD, LBD, or AD is developing in the subject if the leukocytes are determined to be more activated in step iii) performed at the second point in time compared to the level of activation in step iii) performed at the first point in time.

In some embodiments, the method is for assessing whether Parkinson's disease (PD) is developing in a subject who has been identified as being at risk of developing PD, and comprises performing each of the following steps i) to iii):
i) obtaining leukocytes from the subject;
ii) contacting the leukocytes with an epitope that was previously identified to increase activation of the leukocytes; and
iii) determining the level of activation of the leukocytes after contact with the epitope,

at a first and a second point in time, and then
iv) concluding that PD is developing in the subject if the leukocytes are determined to be more activated in step iii) performed at the second point in time compared to the level of activation in step iii) performed at the first point in time.

The present invention provides methods for assessing whether an α-synucleinopathy, Parkinson's disease (PD), Lewy Body dementia (LBD), or Alzheimer's disease (AD) is developing in a subject who has been identified as being at risk of developing the α-synucleinopathy, PD, LBD, or AD comprising performing each of the following steps i) to iii):
i) obtaining leukocytes from the subject;
ii) separating the leukocytes into two or more pools of leukocytes and contacting each pool with an epitope, wherein each pool is contacted with a different epitope;
iii) determining whether each pool has increased activation after contact with the epitope;

at a first and a second point in time, and then
iv) concluding that that PD, LBD, or AD is developing in the subject if more pools of leukocytes are determined to be activated in step iii) performed at the second point in time compared to the number of pools that are determined to be activated in step iii) performed at the first point in time.

In some embodiments, the method is for assessing whether Parkinson's disease (PD) is developing in a subject who has been identified as being at risk of developing PD, and comprises performing each of the following steps i) to iii):
i) obtaining leukocytes from the subject;
ii) separating the leukocytes into two or more pools of leukocytes and contacting each pool with an epitope, wherein each pool is contacted with a different epitope;
iii) determining whether each pool has increased activation after contact with the epitope;

at a first and a second point in time, and then
iv) concluding that that PD is developing in the subject if more pools of leukocytes are determined to be activated in step iii) performed at the second point in time compared to the number of pools that are determined to be activated in step iii) performed at the first point in time.

In some embodiments, the subject has been identified as being at risk of developing the α-synucleinopathy, PD, LBD, or AD according to a method of the invention.

In some embodiments, the second point in time is at least about 1, 2, 3, 4, 5, 6, 7, 8, 9, 10, 20, 30, 40, 50, 52, or 104 weeks after the first point in time.

The present invention provides methods for assessing whether a subject afflicted with an α-synucleinopathy, Parkinson's disease (PD), Lewy Body dementia (LBD), or Alzheimer's disease (AD) is likely to benefit from a therapy, wherein the therapy is directed to leukocytes that are activated by an epitope, the method comprising
i) obtaining leukocytes from the subject;
ii) contacting the leukocytes with the epitope;
iii) determining whether the leukocytes have increased activation after contact with the epitope; and
iv) identifying the subject as likely to benefit from the therapy if in step iii) the leukocytes are determined to have increased activation after contact with the epitope, and identifying the subject as unlikely to benefit from the therapy if in step iii) the leukocytes are determined to not have increased activation after contact with the epitope.

In some embodiments, the method is for assessing whether a subject afflicted with Parkinson's disease (PD) is likely to benefit from a therapy, wherein the therapy is directed to leukocytes that are activated by an epitope, the method comprising
i) obtaining leukocytes from the subject;
ii) contacting the leukocytes with the epitope;
iii) determining whether the leukocytes have increased activation after contact with the epitope; and
iv) identifying the subject as likely to benefit from the therapy if in step iii) the leukocytes are determined to have increased activation after contact with the epitope, and identifying the subject as unlikely to benefit from the therapy if in step iii) the leukocytes are determined to not have increased activation after contact with the epitope.

The present invention provides methods for producing a written or electronic report identifying whether a subject afflicted with an α-synucleinopathy, Parkinson's disease (PD), Lewy Body dementia (LED), or Alzheimer's disease (AD) is likely to benefit from a therapy, wherein the therapy is directed to leukocytes that are activated by an epitope, comprising
i) obtaining leukocytes from the subject;
ii) contacting the leukocytes with the epitope;
iii) determining whether the leukocytes have increased activation after contact with the epitope;
iv) identifying the subject as likely to benefit from the therapy if in step iii) the leukocytes are determined to have increased activation after contact with the epitope, and identifying the leukocytes of the subject as unlikely to benefit from the therapy if in step iii) the leukocytes are determined to not have increased activation after contact with the epitope; and
v) preparing the written or electronic report identifying whether the subject is likely to benefit from the therapy based on the identification in step iv).

In some embodiments, the method further comprises determining the presence of at least one human leukocyte antigen (HLA) allele in the subject. In some embodiments, the subject is identified as likely to benefit from the therapy if
(a) the leukocytes are determined to have increased activation after contact with the epitope, or 1 or more pools is determined to have increased activation after contact with an epitope, and
(b) the subject has the HLA allele DRB5*01:01 or DRB1*15:01.

In some embodiments, determining the presence of at least one HLA allele in the subject comprises obtaining genomic DNA from the subject and assaying with a probe or a primer, wherein if the assaying is with a primer, then the assaying comprises
(1) hybridizing a primer to a nucleic acid having the sequence of a region proximal to the HLA allele or
(2) hybridizing a primer to a nucleic acid having the sequence within the HLA allele, and

if the assaying is with a probe, then the assaying comprises hybridizing a probe to a nucleic acid having the sequence within the HLA allele. In some embodiments, the assaying is with a probe, and the probe is part of a probe array.

The present invention provide methods for assessing whether a subject afflicted with an α-synucleinopathy, Parkinson's disease (PD), Lewy Body dementia (LBD), or Alzheimer's disease (AD) has benefited from a therapy, wherein the therapy is directed to leukocytes that are activated by an epitope, the method comprising
i) obtaining leukocytes from the subject;
ii) contacting the leukocytes with the epitope;
iii) determining whether the leukocytes have increased activation after contact with the epitope; and
iv) identifying the subject as having benefited from the therapy if in step iii) if the leukocytes are determined to have increased activation after contact with the epitope, and identifying the subject as not having benefited from the therapy if in step iii) the leukocytes are determined to not have increased activation after contact with the epitope.

The present invention also provide methods for treating a subject afflicted with an α-synucleinopathy, Parkinson's disease (PD), Lewy Body dementia (LBD), or Alzheimer's disease (AD) who has been identified as being likely to benefit from a therapy directed to leukocytes that are activated by an epitope, comprising administering the therapy that is specific for leukocytes that are activated by the epitope to the subject. In some embodiments, the subject has been identified as being likely to benefit from a therapy directed to leukocytes that are activated by an epitope according to a method of the invention.

The present invention provides methods for prophylactically treating a subject who has been identified as being at risk of developing an α-synucleinopathy, Parkinson's disease (PD), Lewy Body dementia (LBD), or Alzheimer's disease (AD) comprising administering to the subject a therapy that is directed to leukocytes that are activated by an epitope, wherein leukocytes of the subject have been determined to have increased activation after contact with the epitope. In some embodiments, the subject has been identified as being at risk of developing the α-synucleinopathy, PD, LBD, or AD according a method of the invention.

The present invention also provides methods for treating a subject afflicted with an α-synucleinopathy, Parkinson's disease (PD), Lewy Body dementia (LBD), or Alzheimer's disease (AD) comprising administering to the subject a therapy that is directed to leukocytes that are activated by an epitope, wherein leukocytes of the subject have been determined to have increased activation after contact with the epitope. In some embodiments, the subject has been diagnosed or confirmed to be afflicted with the α-synucleinopathy, PD, LBD, or AD according to a method of the invention.

In some embodiments, the therapy is tolerization therapy, and the tolerization therapy is specific for leukocytes that are activated by the epitope. In some embodiments, administering the tolerization therapy comprises administering to the subject the epitope in an amount that is effective to reduce activation of leukocytes in the subject by the epitope. In some embodiments, the epitope is administered orally, intranasally, or subcutaneously. In some embodiments, the epitope is administered together with an adjuvant. In some embodiments, the adjuvant is aluminum hydroxide.

In some embodiments, the therapy comprises selectively killing the leukocytes that are activated by the epitope in the subject. In some embodiments, selectively killing the leukocytes that are activated by the epitope in the subject comprises administering to the subject an effective amount of a compound comprising a major histocompatibility complex (MHC) Tetramer and a toxin to the subject, wherein the MHC Tetramer comprises the epitope. In some embodiments, the MHC tetramer is a MHC Class I tetramer or a MHC Class II tetramer. In some embodiments, the toxin is a ribosome-inactivating protein. In some embodiments, the ribosome-inactivating protein is saporin. In some embodiments, the MHC Tetramer comprises four H2-D^{b} peptides.

The present invention provides a method for treating a subject afflicted with an α-synucleinopathy, Parkinson's disease (PD), Lewy Body dementia (LBD), or Alzheimer's disease (AD), comprising administering an immunosuppressant therapy to the subject. In some embodiments, the subject has been identified as being likely to benefit from a therapy directed to leukocytes that are activated by an epitope according to a method of the invention.

The present invention also provides methods for prophylactically treating a subject who has been identified as being at risk of developing an α-synucleinopathy, Parkinson's disease (PD), Lewy Body dementia (LED), or Alzheimer's disease (AD), comprising administering to the subject an immunosuppressant therapy. In some embodiments, the subject has been identified as being at risk of developing the α-synucleinopathy, PD, LBD, or AD according to a method of the invention.

The present invention provides methods for treating a subject afflicted with an α-synucleinopathy, Parkinson's disease (PD), Lewy Body dementia (LBD), or Alzheimer's disease (AD), comprising administering to the subject an immunosuppressant therapy. In some embodiments, the subject has been diagnosed or confirmed to be afflicted with the α-synucleinopathy, PD, LBD, or AD according to a method of the invention.

In some embodiments, the immunosuppressant therapy comprises administering an effective amount of an immunosuppressive compound to the subject. Non-limiting examples of immunosuppressive compounds include a calcineurin inhibitor, a compound that blocks a chemokine receptor that is expressed by a leukocyte, a glucocorticoid, a mTOR inhibitor, an anti-metabolic compound, a phosphodiesterase-5 inhibitor, an antibody, or a leukocyte function antigen-3 (LFA-3)/Fc fusion protein.

In some embodiments, the immunosuppressive compound is a calcineurin inhibitor, and the calcineurin inhibitor is cyclosporine or tacrolimus. In some embodiments, the calcuneurin inhibitor is cyclosporine. In some embodiments, the immunosuppressive compound is a compound that blocks a chemokine receptor that is expressed by a leukocyte. In some embodiments, the chemokine receptor that is expressed by a leukocyte is C-C chemokine receptor type 5 (CCR5). In some embodiments, the immunosuppressive compound is approved for use in the treatment of subjects afflicted with human immunodeficiency virus (HIV). In some embodiments, the immunosuppressive compound is maraviroc. In some embodiments, the immunosuppressive compound is a glucocorticoid, and the glucocorticoid is prednisone or prednisolone.

In some embodiments, the immunosuppressive compound is a mTOR inhibitor, and the mTOR inhibitor is rapamaycin. In some embodiments, the immunosuppressive compound is an anti-metabolic compound, and the anti-metabolic compound is azathioprine, micophenolate or mofetil. In some embodiments, the immunosuppressive compound is a phosphodiesterase-5 inhibitor, and the phosphodiesterase-5 inhibitor is sildenafil or paclitaxel. In some embodiments, the immunosuppressive compound is a LFA-3/Fc fusion protein, and the LFA-3/Fc fusion protein is a CD2-directed LFA-3/Fc fusion protein. In some embodiments, the CD2-directed LFA-3/Fc fusion protein is alefacept.

The present invention provides a method for assessing whether leukocytes of a subject afflicted with an α-synucleinopathy, Parkinson's disease (PD), Lewy Body dementia (LBD), or Alzheimer's disease (AD) are activated by an epitope, comprising
i) obtaining leukocytes from the subject;
ii) contacting the leukocytes with the epitope;
iii) determining whether the leukocytes have increased activation after contact with the epitope; and
iv) identifying the leukocytes of the subject as activated by the epitope if in step iii) the leukocytes are determined to have increased activation after contact with the epitope, and identifying the leukocytes of the subject as not activated by the epitope if in step iii) the leukocytes are determined to not have increased activation after contact with the epitope.

In some embodiments, the epitope is or comprises part of a compound that is produced by neurons in subjects afflicted with the α-synucleinopathy, PD, LBD, or AD. In some embodiments, the neurons are in the ventral midbrain, the substantia nigra, the locus coeruleus, or the ventral tegmental area. In some embodiments, the neurons are catecholamine neurons.

In some embodiments, the epitope comprises consecutive amino acids that are identical to a stretch of consecutive amino acids in a protein that is produced by the neurons. In some embodiments, the epitope comprises consecutive amino acids that are identical to a stretch of consecutive amino acids in α-synuclein (α-syn), tau, leucine-rich repeat kinase 2 (LRRK2) or glucocerebrosidase. In some embodiments, the epitope comprises consecutive amino acids that are identical to a stretch of consecutive amino acids in an α-syn, tau, leucine-rich repeat kinase 2 (LRRK2) or glucocerebrosidase mutant.

In some embodiments, the epitope comprises consecutive amino acids that are identical to a stretch of consecutive amino acids in an α-syn mutant. In some embodiments, the α-syn mutant is an α-syn A53T or A30P mutant. In some embodiments, the epitope comprises about 15, at least 15, 5-50, 8-11, or 8-14 amino acids. In some embodiments, the epitope comprises 5-50 amino acids. In some embodiments, the epitope comprises 8-14 amino acids.

In some embodiments, the epitope is phosphorylated, nitrated, or dopamine modified. In some embodiments, the epitope comprises a phosphorylated serine or a phosphorylated tyrosine. In some embodiments, the epitope comprises a phosphorylated serine. In some embodiments, the phosphorylated serine or phosphorylated tyrosine is within a stretch of consecutive amino acids that is identical to a stretch of consecutive amino acids comprising the serine at position 129 of α-syn or the tyrosine at position 39 of α-syn.

In some embodiments, the epitope comprises consecutive amino acids in the sequence set forth as VFMKGLSKA (SEQ ID NO: 10), DVFMKGLSKA (SEQ ID NO: 11), GVVAAAEKTK (SEQ ID NO: 12), VAAAEKTKQGVAEAP (SEQ ID NO: 13), VAAAEKTKQGVAEAA (SEQ ID NO: 14), AGKTKEGVL (SEQ ID NO: 15), PGKTKEGVL (SEQ ID NO: 16), AGKTKEGVLY (SEQ ID NO: 17), APGKTKEGVL (SEQ ID NO: 18), GVAEAAGKTK (SEQ ID NO: 19), KQGVAEAPGKTKEGV (SEQ ID NO: 20), PGKTKEGVLYVGSKT (SEQ ID NO: 21), KTKEGVLYVGSKTKK (SEQ ID NO: 22), KQGVAEAAGKTKEGV (SEQ ID NO: 23), AGKTKEGVLYVGSKT (SEQ ID NO: 24), VLYVGSKTK (SEQ ID NO: 25), LYVGSKTKK (SEQ ID NO: 26), YVGSKTKEGV (SEQ ID NO: 27), VLYVGSKTKK (SEQ ID NO: 28), GVLYVGSKTK (SEQ ID NO: 29), LYVGSKTKEG (SEQ ID NO: 30), KTKKGVVHGV (SEQ ID NO: 31), KTKKGVVHG (SEQ ID NO: 32), YVGSKTKKGVVHGVA (SEQ ID NO: 33), KTKEGVLYVGSKTKE (SEQ ID NO: 34), VTNVGGAVV (SEQ ID NO: 35), GVVHGVTTV (SEQ ID NO: 36), EEGAPQEGI (SEQ ID NO: 37), GSIAAATGFV (SEQ ID NO: 38), SIAAATGFVK (SEQ ID NO: 39), AGSIAAATGF (SEQ ID NO: 40), IAAATGFVKK (SEQ ID NO: 41), APQEGILEDM (SEQ ID NO: 42), EEGAPQEGIL (SEQ ID NO: 43), VFMKGLSKAK (SEQ ID NO: 44), AEAAGKTKEG (SEQ ID NO: 45), YVGSKTKEGVVHGVT (SEQ ID NO: 46), or IAAATGFVK (SEQ ID NO: 47). In some embodiments, the epitope comprises at least 8 consecutive amino acids having a sequence within the amino acid sequence set forth as KTKEGVLYVGSKTKE (SEQ ID NO: 34), GKTKEGVLYVGSKTK (SEQ ID NO: 59) or DNEAYEMPSEEGYQDY (SEQ ID NO: 48). In some embodiments, the epitope comprises at least 8 consecutive amino acids having a sequence within the amino acid sequence set forth as DNEAYEMPSEEGYQDY (SEQ ID NO: 48).

In some embodiments, the epitope comprises consecutive amino acids in the sequence set forth as PSEEGYQDY (SEQ ID NO: 49), YEMPSEEGY (SEQ ID NO: 50), MPSEEGYQD (SEQ ID NO: 51), AYEMPSEEGY (SEQ ID NO: 52), MPSEEGYQDY (SEQ ID NO: 53), EMPSEEGYQD (SEQ ID NO: 54), DNEAYEMPSE (SEQ ID NO: 55), YEMPSEEGYQ (SEQ ID NO: 56), or SEEGYQDYEP (SEQ ID NO: 57). In some embodiments, the serine in the sequence set forth as PSEEGYQDY (SEQ ID NO: 49), YEMPSEEGY (SEQ ID NO: 50), MPSEEGYQD (SEQ ID NO: 51), AYEMPSEEGY (SEQ ID NO: 52), MPSEEGYQDY (SEQ ID NO: 53), EMPSEEGYQD (SEQ ID NO: 54), DNEAYEMPSE (SEQ ID NO: 55), YEMPSEEGYQ (SEQ ID NO: 56), or SEEGYQDYEP (SEQ ID NO: 57) is phosphorylated.

In some embodiments, the epitope comprises a non-amino acid polymer that is produced by the neurons. In some embodiments, the epitope is neuromelanin or a portion thereof.

In some embodiments, in step iii) the leukocytes are determined to have increased activation after contact with the epitope if the leukocytes express or release more of at least one cytokine compared to corresponding leukocytes not contacted with the epitope. In some embodiments, in step iii) the leukocytes are determined to have increased activation after contact with the epitope if the leukocytes release at least one cytokine. In some embodiments, in step iii) the leukocytes are determined to have released the at least one cytokine if there are over 20 spot-forming cells (SFC) per million cells as measured by an ELISpot assay comprising the colorimetric detection of the at least one cytokine.

In some embodiments, the at least one cytokine is at least interferon-gamma (IFN-γ) or IL-5. In some embodiments, the at least one cytokine is at least TNFα, IL-4, IL-17, IL-10, or IL-21. In some embodiments, the at least one cytokine is at least interferon-gamma (IFN-7). In some embodiments, the at least one cytokine is at least IL-5. In some embodiments, the at least one cytokine is two or more cytokines, wherein the two or more cytokines are at least IFN-γ and IL-5.

In some embodiments, the leukocytes are T cells. In some embodiments, the T cells are CD4+ T cells, CD8+ T cells, and/or CD4+CD8+ T cells. In some embodiments, the at least one cytokine is at least IFN-γ. In some embodiments, the at least one cytokine is IFN-γ. In some embodiments, the T cells are CD4+ T cells. In some embodiments, the at least one cytokine is at least IL-5.

In some embodiments, the leukocytes in step i) are in a blood sample taken from the subject, and contacting the leukocytes with the epitope in step ii) comprises contacting the blood with the epitope. In some embodiments, contacting the blood with the epitope comprises adding the epitope to the blood.

In some embodiments, in step iii) the level of the at least one cytokine that is released from the leukocytes is assayed through a process comprising an enzyme-linked immunosorbent assay (ELISA). In some embodiments, in step iii) the level of the at least one cytokine that is expressed or released from the leukocytes is assayed through a process comprising an enzyme-linked immunosorbent assay (ELISA), intracellular cytokine staining (ICS), or quantitative RT-PCR.

In some embodiments, determining whether the leukocytes have increased activation comprises
(a) contacting the leukocytes with compound comprising a major histocompatibility complex (MHC) Tetramer having four MHC molecules, wherein each MHC molecule is associated with an epitope;
(b) identifying leukocytes that become bound to the compound as activated.

In some embodiments, each of the four MHC molecules of the MHC Tetramer are associated with each other via a tetramerization agent. In some embodiments, the tetramerization agent is streptavidin or avidin. In some embodiments, a label is covalently bound to the tetramerization agent. In some embodiments, the MHC Tetramer comprises four MHC monomer fusion proteins, wherein each MHC monomer fusion protein comprises a MHC molecule and biotin. In some embodiments, the MHC molecule is encoded by the DRB1*15:01 or DRB5*01:01 HLA allele.

The present invention provides methods for assessing whether a test compound is an epitope to which leukocytes of a subject suffering from a neurological disorder are responsive comprising
i) obtaining leukocytes from the subject;
ii) contacting the leukocytes with the test compound;
iii) determining whether the leukocytes has increased activation after contact with the test compound; and
iv) identifying the test compound as an epitope to which the leukocytes are responsive if in step iii) the leukocytes are determined to have increased activation after contact with the test compound, and identifying the test compound as not an epitope to which the leukocytes are responsive if in step iii) the leukocytes are determined to not have increased activation after contact with the test compound.

In some embodiments, the test compound is or comprises part of a compound that is produced by neurons in subjects afflicted with an α-synucleinopathy, Parkinson's disease (PD), Lewy Body dementia (LBD), or Alzheimer's disease (AD). In some embodiments, the neurons are in the ventral midbrain, the substantia nigra, the locus coeruleus, or the ventral tegmental area. In some embodiments, the neurons are catecholamine neurons.

In some embodiments, the test compound is a test polypeptide comprising consecutive amino acids that are identical to a stretch of consecutive amino acids in a protein that is produced by the neurons. In some embodiments, the test polypeptide comprises consecutive amino acids that are identical to a stretch of consecutive amino acids in α-synuclein (α-syn), tau, leucine-rich repeat kinase 2 (LRRK2) or glucocerebrosidase. In some embodiments, the test polypeptide comprises consecutive amino acids that are identical to a stretch of consecutive amino acids in an α-syn, tau, leucine-rich repeat kinase 2 (LRRK2) or glucocerebrosidase mutant. In some embodiments, the test polypeptide comprises consecutive amino acids that are identical to a stretch of consecutive amino acids in an α-syn mutant. In some embodiments, the α-syn mutant is an α-syn A53T or A30P mutant. In some embodiments, the test polypeptide comprises 5-50 amino acids. In some embodiments, the test polypeptide comprises 8-14 amino acids. In some embodiments, the test polypeptide comprises about 15, at least 15, 5-50, 8-11, or 8-14 amino acids. In some embodiments, the test polypeptide is phosphorylated, nitrated, or dopamine modified.

In some embodiments, the test compound comprises a non-amino acid polymer that is produced by the neurons. In some embodiments, the test compound is neuromelanin, or a portion thereof.

In some embodiments, in step iii) the leukocytes are determined to have increased activation after contact with the test compound if the leukocytes release at least one cytokine. In some embodiments, in step iii) the leukocytes are determined to have released the at least one cytokine if there are over 20 spot-forming cells (SFC) per million cells as measured by an ELISpot assay comprising the colorimetric detection of the at least one cytokine.

In some embodiments, the at least one cytokine is at least interferon-gamma (IFN-7) or IL-5. In some embodiments, the at least one cytokine is at least TNFα, IL-4, IL-17, IL-10, or IL-21. In some embodiments, the at least one cytokine is at least interferon-gamma (IFN-7). In some embodiments, the at least one cytokine is at least IL-5. In some embodiments, the at least one cytokine is two or more cytokines, wherein the two or more cytokines are at least IFN-γ and IL-5.

In some embodiments, the leukocytes are T cells. In some embodiments, the T cells are CD4+ T cells, CD8+ T cells, and/or CD4+CD8+ T cells. In some embodiments, the at least one cytokine is at least IFN-7. In some embodiments, the at least one cytokine is IFN-γ. In some embodiments, the T cells are CD4+ T cells. In some embodiments, the at least one cytokine is at least IL-5.

In some embodiments, the leukocytes in step i) are in a blood sample taken from the subject, and contacting the leukocytes with the test compound in step ii) comprises contacting the blood with the test compound. In some embodiments, contacting the blood with the test compound comprises adding the test compound to the blood.

In some embodiments, in step iii) the level of the at least one cytokine that is released from the leukocytes is assayed through a process comprising an enzyme-linked immunosorbent assay (ELISA).

The present invention provides kits for use in methods of the invention, comprising an epitope of the invention. In some embodiments, the kit further comprises an anti-IFN-γ antibody or an anti-IFN-γ antibody.

The present invention also provides compounds for treating an α-synucleinopathy, Parkinson's disease (PD), Lewy Body dementia (LBD), or Alzheimer's disease (AD), comprising i) a major histocompatibility complex (MHC) Tetramer having four MHC molecules, wherein each MHC molecule is associated with an epitope, and ii) a toxin. In some embodiments, each of the four MHC molecules of the MHC Tetramer are associated with each other via a tetramerization agent. In some embodiments, the tetramerization agent is streptavidin or avidin. In some embodiments, the toxin is covalently bound to the streptavidin or the avidin. In some embodiments, the MHC Tetramer comprises four MHC monomer fusion proteins, wherein each MHC monomer fusion protein comprises a MHC molecule and biotin. In some embodiments, the MHC tetramer is a MHC Class I tetramer or a MHC Class II tetramer. In some embodiments, the toxin is a ribosome-inactivating protein such as saporin. In some embodiments, the MHC Tetramer comprises four H2-D^{b} peptides.

The present invention also provides pharmaceutical compositions comprising one or more compounds or epitopes of the invention.

In some embodiments, the leukocytes are lymphocytes. In some embodiments, the lymphocytes are B cells or T cells. In some embodiments, the subject is a human subject. PD is often diagnosed by a neurologist who can evaluate symptoms and their severity. National Institute of Neurological Disorders and Stroke, Parkinson's Disease Backgrounder, available at www.ninds.nih.gov/disorders/parkinsons_disease/parkinsons_disease_back grounder.htm, last updated Oct. 18, 2004 (hereinafter “NINDS Backgrounder”. According to the National Institute of Neurological disorders and Stroke, there is currently no test that can clearly identify the disease. (NINDS Backgrounder). Sometimes people with suspected PD are given anti-Parkinson's drugs to see if they respond (NINDS Backgrounder). Other tests, such as brain scans, can help doctors decide if a patient has true PD or some other disorder that resembles it (NINDS Backgrounder). Microscopic brain structures called Lewy bodies, which can be seen only during an autopsy, are regarded as a hallmark of classical PD (NINDS Backgrounder). Autopsies have uncovered Lewy bodies in a surprising number of older persons without diagnosed PD—8% of people over 50, almost 13% of people over 70, and almost 16% of those over 80, according to one study (NINDS Backgrounder). As a result, some experts believe PD is something of an “iceberg; phenomenon,” lurking undetected in as many as 20 people for each known Parkinson's patient (NINDS Backgrounder). Without wishing to be bound by any scientific theory, a few researchers contend that almost everyone would develop Parkinson's eventually if they lived long enough (NINDS Backgrounder).

The present invention provides methods for identifying subjects afflicted with PD that would previously have remained undetected. Aspects of the present invention enable the detection of PD in presymptomatic stages. Additionally, the present invention provides methods for identifying those who might eventually develop PD. PD has an increased prevalence with age. See, for example, the NINDS Backgrounder; and Van Den Eeden et al., (2003) Incidence of Parkinson's Disease: Variation by Age, Gender, and Race/Ethnicity, Am. J. Epidemiol. 157 (11): 1015-1022, the entire content of each of which is hereby incorporated herein by reference. PD ranks among the most common late-life neurodegenerative diseases, affecting approximately 1.5% to 2.0% of people aged 60 years and older (Patrick Sweeney, Parkinson's Disease, Cleveland Clinic, available at clevelandclinicmeded.com/medicalpubs/diseasemanagement/neurology/parkinsons-disease/). Without wishing to be bound by any scientific theory, the peripheral immune response of PD may begin in a subject decades before PD may be diagnosed by a neurologist, and subjects having the peripheral immune response may be identified for earlier therapy using methods of the invention. There are a number of peripheral symptoms associated with PD including orthostatic hypotension and constipation. The present invention provides methods for diagnosing causes of these symptoms, and be used to identify subjects who may benefit from prophylactic treatment for PD.

Aspects of the present invention provide an epitope useful as a test/biomarker for PD, which could include identifying patients in preclinical stages or in danger of PD, and to measure disease progression and/or response. As described herein, aspects of the invention provide means to detect these T cells in patient blood. Similar approaches for identifying tuberculosis patients will be known to those skilled in the art. For use of a similar test in TB diagnosis, see en.wikipedia.org/wiki/QuantiFERON, the entire contents of which are hereby incorporated by reference.

Additionally, aspects of the invention define which precise T cells and antigens individual patients have, and provide individualized therapy that spare other important immune functions. The test/biomarker test for PD has already been conducted and is effective, as described in the Examples disclosed herein.

Without wishing to be bound by any scientific theory, the T cells identified in embodiments of the invention may kill the neurons in PD, and thus be a step in the disease. The present invention provides means to treat PD, as blocking these T cells arrest the disease progression. An example would be tolerization: particular epitopes the T cells recognize are determined, and patients are exposed to the epitope in a form that alters the immune system to recognize it as self, and halt or reduce making killer T cells. In addition to α-synuclein, there are additional mutant proteins that cause PD, including LRRK2 and glucocerebrosidase. These markers are similarly useful for diagnosing, confirming, providing biomarkers for, and treating PD.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg” is a disclosure of 0.2 mg/kg, 0.3 mg/kg, 0.4 mg/kg, 0.5 mg/kg, 0.6 mg/kg etc. up to 5.0 mg/kg.

### Abbreviations

α-syn, alpha synuclein; β2m, beta 2 microglobulin; BF, brightfield; BrdU, 5-bromo-2-deoxyuridine; BSA, bovine seroalbumin; ConA, concananycin A; CNS, central nervous system; CTLs, cytotoxic T cells CTRL, control; DA, dopamine/dopaminergic; DCs, dendritic cells; ELISA, enzyme-linked immunosorbent assay; hES, human stem cells; HLA, human leukocyte antigen; IFN-γ, interferon gamma; KO, knocked out; LC, locus coeruleus; LGN, lateral geniculate nucleus; LPS, lipopolysaccharide; MAP-2, microtubule associated protein-2; MHC, major histocompatibility complex; MHC-I, major histocompatibility complex class I; MHC-II, major histocompatibility complex class II; MN, spinal motor neurons; Mut-α-syn, mutated (A53T) alpha synuclein; NE, norepinephrine/norepinephrinergic; Nit-α-syn, nitrated alpha synuclein; NM, neuromelanin; OVA, ovalbumin; SEM, standard error of the mean; SIIN, SIINFEKL (SEQ ID NO: 9) peptide; SIINFEKL-MHC-I, the MHC-I complex when occupied by SIINFEKL (SEQ ID NO: 9) peptide; SN, substantia nigra; TH, tyrosine hydroxylase; PD, Parkinson's disease Veh, vehicle; VM, ventral midbrain; VTA, ventral tegmental area

### Terms

Unless otherwise defined, all technical and scientific terms used herein have the same meaning as commonly understood by a person of ordinary skill in the art to which this invention belongs. As used herein, and unless stated otherwise or required otherwise by context, each of the following terms shall have the definition set forth below.

As used herein, “about” in the context of a numerical value or range means±10% of the numerical value or range recited or claimed, unless the context requires a more limited range.

As used herein, a “subject afflicted with” a condition, e.g. PD, LBD, or AD, means a subject who was been affirmatively diagnosed to have the condition.

Embodiments of the present invention relate to determining whether leukocytes have increase activation after contact with an epitope or test compound. It will be understood that the “increased activation” of the leukocytes is in response to contact with the epitope or the test compound. General methods for assaying whether a leukocyte has increased activation will be known to those of ordinary skill in the art. Additionally, assays for determining increased activation that are described for particular epitopes or test compounds in the Examples herein may be applied to other epitopes and test compounds of the invention.

In some embodiments, the leukocytes are determined to have increased activation after contact with the epitope if the leukocytes release a cytokine. In some embodiments, the leukocytes are determined to have increased activation after contact with an epitope or test compound if the leukocytes release a cytokine that is not released by corresponding leukocytes not contacted with the epitope or test compound. In some embodiments, a cytokine is determined to be released if there is a minimum of about 5, 10, 15, 20, 25, 30, 35, 40, 45, 50, 100, or about 10-50 spot-forming cells (SFC) per million cells are measured using an ELISpot assay comprising the colorimetric detection of the cytokine. In some embodiments, the leukocytes are determined to have released the at least one cytokine if there are over 20 spot-forming cells (SFC) per million cells as measured by an ELISpot assay comprising the colorimetric detection of the at least one cytokine. A person having ordinary skill in the art will readily be able to perform the ELISpot assay in embodiments of the invention using the disclosures herein. ELISpot assays are described in Czerkinsky et al. (1988) Reverse ELISpot Assay for Clonal Analysis of Cytokine Production. I. Enumeration of gamma-Interferon-Secreting Cells. J. Immunol. Methods, 110:29; Sedgwick and Holt, (1983) A Solid-Phase Immunoenzymatic technique for the Enumeration of Specific Antibody-Secreting Cells. J. Immunol. Methods, 57: 301; and Czerkinsky et al. (1983) A Solid-Phase Enzyme-Linked Immunospot (ELISPOT) Assay for Enumeration of Specific Antibody Secreting Cells. J. Immunol. Methods, 65:109, the entire content of each of which is incorporated herein by reference.

In some embodiments, the leukocytes are determined to have increased activation after contact with an epitope or test compound if the leukocytes release more of a cytokine than corresponding leukocytes not contacted with the epitope or test compound. In some embodiments, the leukocytes are determined to have increased activation after contact with the epitope or test compound if the leukocytes release about 1, 2, 3, 4, 5, 10, 15, 20, 25, 30, 35, 40, 45, 50, 55, 60, 65, 70, 80, 90, 100, 150, 200, 300, 400, 500, 750, 1,000, or 1-5,000% more of a cytokine than corresponding leukocytes not contacted with the epitope or test compound. Methods for assaying increased cytokine release include but are not limited to the ELISpot assay Western Blot Analysis and ELISA, which will be well understood to those in the art.

In some embodiments, the leukocytes are determined to have increased activation after contact with the epitope or test compound if the leukocytes proliferate, and corresponding leukocytes not contacted with the epitope or test compound do not proliferate. In some embodiments, the leukocytes are determined to have increased activation after contact with the epitope or test compound if the leukocytes proliferate more than corresponding leukocytes not contacted with the epitope or test compound. In some embodiments, the leukocytes are determined to have increased activation after contact with the epitope or test compound if the leukocytes proliferate about 1, 2, 3, 4, 5, 10, 15, 20, 25, 30, 35, 40, 45, 50, 55, 60, 65, 70, 80, 90, 100, 150, or 200% more than corresponding leukocytes not contacted with the epitope or test compound. In some embodiments the leukocytes are determined to have increased activation after contact with the epitope or test compound if the leukocytes become differentiated after contact with the epitope or test compound, and corresponding leukocytes not contacted with the epitope or test compound do not become differentiated. In some embodiments the leukocytes are determined to have increased activation after contact with the epitope or test compound if the leukocytes are more differentiated than corresponding leukocytes not contacted with the epitope or test compound. Methods for assaying increased proliferation and differentiation are well known in the art, and include cell counting and fluorescence-activated cell sorting (FACS).

In some embodiments, the leukocytes are determined to have increased activation after contact with the epitope or test compound if the leukocytes express a gene at a higher or lower level than corresponding leukocytes not contacted with the epitope or test compound. In some embodiments, the expression is about 1, 2, 3, 4, 5, 10, 15, 20, 25, 30, 35, 40, 45, 50, 55, 60, 65, 70, 80, 90, 100, 150, 200, 300 400, 500, 750, 1,000, or 1-5,000% higher or lower than in the corresponding leukocytes. In some embodiments, the gene encodes a cytokine. Examples of genes which are differentially expressed in activated T cells are described in Teague et al. (1999) Activation changes the spectrum but not the diversity of genes expressed by T cells. PNAS Vol. 96, No. 22, 12691-12696, the entire content of which is hereby incorporated herein by reference. Methods for assaying gene expression are well known in the art, and include PCR, RT-PCR, Northern Blot Analysis, and microarray analysis.

The release, proliferation, differentiation or change in expression may be measured at, for example, about 0.5, 1, 2, 3, 4, 5, 6, 10, 12, 18, 24, 30, 36, 42, 48, or 72 hours after the leukocytes are contacted with the epitope or test compound.

As used herein, a stretch of “consecutive amino acids” means a plurality of amino acids arranged in a chain, each of which is joined to a preceding amino acid by a peptide bond, excepting that the first amino acid in the chain may optionally not be joined to a preceding amino acid. The amino acids of the chain may be naturally or non-naturally occurring, or may comprise a mixture thereof. The amino acids, unless otherwise indicated, may be genetically encoded, naturally-occurring but not genetically encoded, or non-naturally occurring, and any selection thereof.

As used herein a therapy that is “directed to leukocytes that are activated by an epitope” is a therapy that selectively reduces or prevents the activation of the leukocytes by the epitope. It will be understood that selectively reducing or preventing the activation of leukocytes by an epitope includes killing the leukocytes or reducing the viability or proliferation of leukocytes that are capable of becoming activated when they are contacted with the epitope. Non-limiting examples of therapies that are directed to leukocytes that are activated by an epitope include administration of a compound that selectively kills leukocytes that are capable of becoming activated when they are contacted with the epitope, and tolerization therapy.

As used herein, “tolerization therapy” or “antigen-specific tolerization” comprises exposing a subject with an epitope in a way that alters the subject's immune system to have reduced activation by the epitope. Tolerization therapy results in a decrease in the activation of a leukocyte of the subject, such as a T cell, by the epitope. Tolerization therapy is discussed in Coppieters et al. (2013) Clinical Immunology, 149, 345-355; Billetta et al. (2012) Clin Immunol, 145(2):94-101; and Lutterotti and Martin (2014) Expert Opinion on Investigational Drugs, Vol. 23, No. 1, pages 9-20, the entire content of each of which is hereby incorporated herein by reference.

Aspects of the present invention relate to a compound comprising a major histocompatibility complex (MHC) Tetramer and a toxin. MHC Tetramers are complexes of four Major Histocompatibility Complex (MHC) molecules which are each associated with a specific molecule. The specific molecule may be an epitope of the invention. In some embodiments, the four MHC molecules are associated with each other via a tetramerization agent. In some embodiments, the MHC Tetramer comprises four MHC monomer fusion proteins, wherein each MHC monomer fusion protein comprises a MHC molecule and biotin. In some embodiments, the tetramerization agent is streptavidin or avidin. In the compound, a MHC Tetramer may be coupled to a toxin by, e.g., a covalent bond, a linker, a streptavidin-biotin interaction or a streptavidin-avidin interaction. In some embodiments, the toxin is covalently bound to the streptavidin or the avidin. There are two types of Tetramers, Class I and Class II. In some embodiments, the MHC molecules of a Class I Tetramer are mutated to minimize binding of the MHC molecule to CD8⁺ cell surfaces. These Class I Tetramers show diminished CD8-mediated binding to the general CD8⁺ lymphocyte population, but retain MHC peptide-specific binding to TCR thus facilitating targeting of specific T cells that are activated by the epitope. MHC Tetramers are described in U.S. Patent Application Publication No. 2004/0137642 A1, published Jul. 15, 2004; Hess et al., (2007) Selective deletion of antigen-specific CD8+ T cells by MHC class I tetramers coupled to the type I ribosome-inactivating protein saporin. Blood, 106:3300-3307; and at www.beckmancoulter.com/wsrportal/wsr/research-and-discovery/products-and-services/flow-cytometry/class-I-itag-mhc-tetramers/index.htm, the entire content of each of which is hereby incorporated by reference.

It will be understood that by “treating” a subject there are multiple possible outcomes. For instance, treating a subject may comprise substantially reducing, slowing, stopping, preventing or reversing the progression of a disease, particularly a neurological disorder such as PD, LSD, or AD. Additionally, treating a subject may comprise substantially reducing, slowing, stopping, preventing or reversing a symptom of a disease. In the most favorable case, reduction is equivalent to prevention.

As used herein, a “symptom” associated with PD, LSD, or AD includes any clinical or laboratory manifestation associated with PD, LBD, or AD and is not limited to what the subject can feel or observe. Common symptoms of PD include but are not limited to tremors, muscle stiffness, difficulty maintaining balance, difficulty maintaining posture, bradykinesia, akinesia, rigid limbs, a shuffling gait, and a stooped posture. Other symptoms of PD include but are not limited to depression, personality changes, dementia, sleep disturbances, speech impairments, and sexual difficulties.

As used herein, “effective” when referring to an amount of a compound administered to a subject for the treatment of a neurological disease refers to the quantity of the compound that is sufficient to yield a desired therapeutic response without undue adverse side effects (such as toxicity, irritation, or allergic response) commensurate with a reasonable benefit/risk ratio when used in the manner of this invention.

Amino acid sequences of α-syn are accessible in public databases by the accession numbers NP_000336 and NP_009292, which are set forth herein as SEQ ID NOs: 1 and 2 respectively. Nucleotide sequences for α-syn is accessible in public databases by the accession numbers NM_000345 and NM_007308, which are set forth herein as SEQ ID NOs: 3 and 4 respectively. The amino acid sequence of leucine-rich repeat kinase 2 (LRRK2) is accessible in public databases by the accession number NP_940980 and is set forth herein as SEQ ID NO: 5. A nucleotide sequence for LRRK2 is accessible in public databases by the accession number NM_198578, which is set forth herein as SEQ ID NO: 6. The amino acid sequence of glucocerebrosidase is accessible in public databases by the accession number BAA02545 and is set forth herein as SEQ ID NO: 7. A nucleotide sequence for glucocerebrosidase is accessible in public databases by the accession number D13286, which is set forth herein as SEQ ID NO: 8. Amino acid and nucleotide sequences of tau are accessible in public databases by the NCBI Gene ID: 4137. The name of the tau gene is microtubule-associated protein tau (MAPT).

Aspects of the present invention relate to the HLA alleles DRB5*01:01 and DRB1*15:01. The amino acid sequence for the DRB5*01:01 protein sequence is set forth herein as SEQ ID NO: 120 and the amino acid sequence for the DRB1*15:01 protein sequence is set forth herein as SEQ ID NO: 121. Additional information about these and other HLA alleles is available in Wissemann et al. (2013) Association of Parkinson disease with structural and regulatory variants in the HLA region. Am J Hum Genet, 93:984-993, PMC3824116, the entire content of which is incorporated herein by reference. Additional information, including sequence information, relating to these alleles and other alleles disclosed herein is available at www.ebi.ac.uk/ipd/imgt/hla/allele.html. It will be understood that persons skilled in the art are able to identify and obtain sequences and genomic locations for the HLA alleles disclosed herein using knowledge in the art.

Non-limiting examples of compounds which may be used in the treatment of PD in embodiments of the invention include dopamine precursors (e.g., levodopa and carbidopa), dopamine agonists (e.g., bromocriptine, pramipexole, ropinirole, apomorphine, and rotigotine), MAO-B inhibitors (e.g., rasagiline and selegiline), COMT inhibitors (e.g., entacapone and tolcapone), anticholinergic compounds (e.g., trihexyphenidyl, benztropine, amitiriptyline and diphenhydramine) antiviral compounds (e.g., amantadine), and beta-blockers (e.g., propranolol).

Non-limiting examples of compounds which may be used in the treatment of PD in embodiments of the invention also include immunosuppressive compounds. In some embodiments, an immunosuppressive compound targets an autoimmune component in PD, for example T cell activation or function. Non-limiting examples of approaches for suppressing the immune system, or a component thereof, in embodiments of the subject invention include:
1. Blocking receptors of chemokines such as CCR5 present on cytotoxic T cells. This can be achieved by using antagonist drugs such as maraviroc. It will be understood that CCR5 is one of the HIV-1 receptors and such drugs have been in use for years to treat HIV patients.
2. Administering a glucocorticoid such as prednisone or prednisolone, which are effective immunosuppressive agents. They inhibit the activation of cytotoxic T cells. Additionally, they cross the blood brain barrier and are used to treat multiple sclerosis (MS).
3. Administering a calcineurin inhibitor such as cyclosporine or tacrolimus, which are potent immunosuppressive agents. They inhibit calcineurin, which blocks phosphatase activity, and thus T cell activation. They are used to inhibit transplant rejection.
4. Administering an inhibitor of mTOR such as rapamaycin, which blocks cell cycle at G1>S phase. Rapamaycin inhibits T cell activation and proliferation. It is used to treat transplant rejection.
5. Administering an anti-metabolic drug including azathioprine, micophenolate or mofetil to block killer T cells.
6. Administration of antibodies for LFA-3Ig1 fusion protein, which interferes with T cell activation. This has been used in psoriasis.
7. Administering a phosphodiesterase-5 inhibitor such as sildenafil or paclitaxel, which have been used in melanoma to lead to cell-mediated T cell immunosuppression.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

All publications and other references mentioned herein are incorporated by reference in their entirety, as if each individual publication or reference were specifically and individually indicated to be incorporated by reference. Publications and references cited herein are not admitted to be prior art.

This invention will be better understood by reference to the Experimental Details which follow, but those skilled in the art will readily appreciate that the specific experiments detailed are only illustrative of the invention as defined in the claims which follow thereafter.

### Experimental Details

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Example 1. MHC-I in Human Catecholamine Neurons

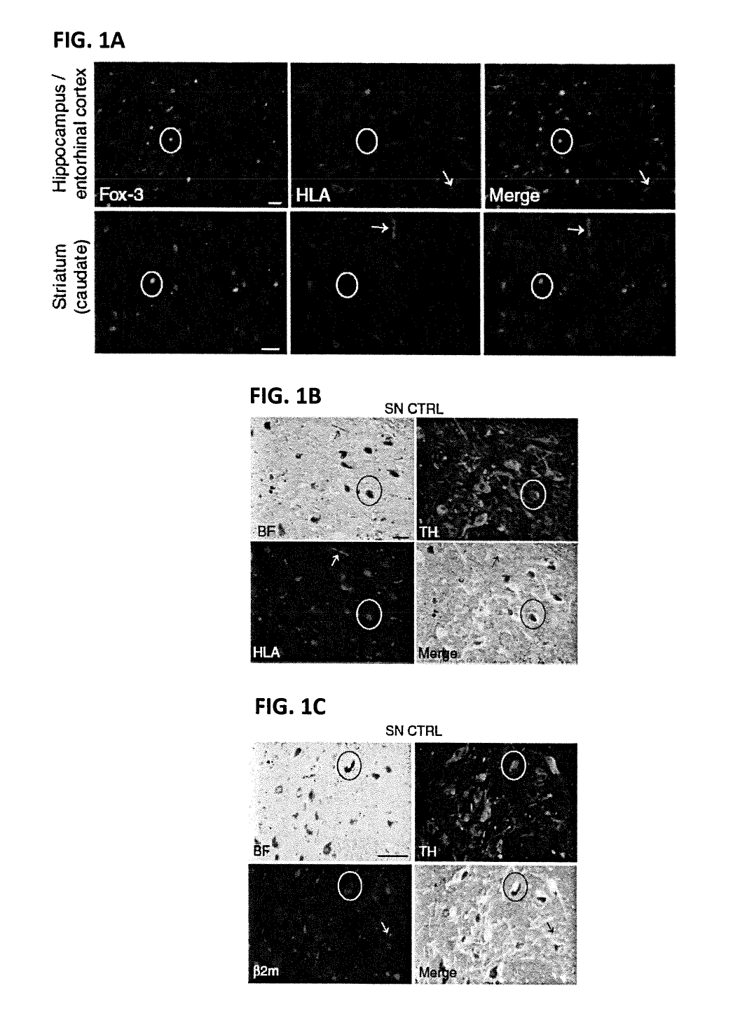

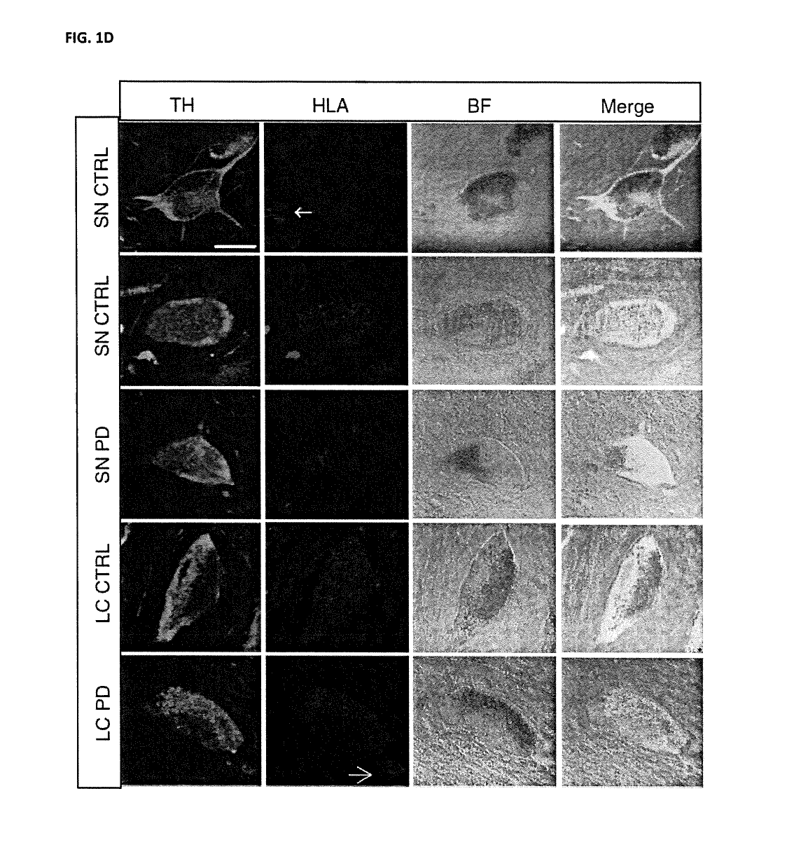

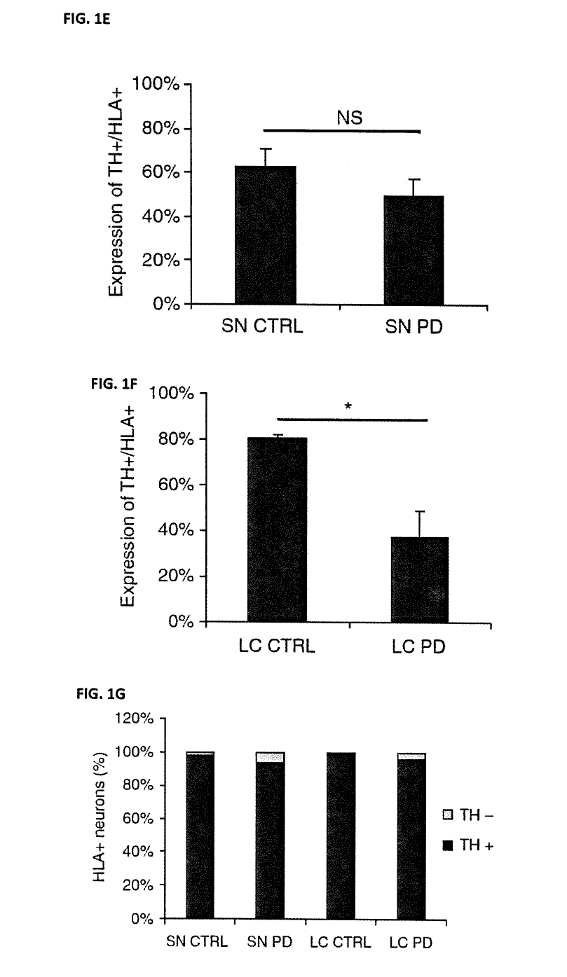

Human MHC-I consists of an α chain encoded by human leukocyte antigen (HLA) genes (HLA-A, HLA-B and HLA-C) and a noncovalently associated β2m subunit. A variety of techniques were used to assess the presence of MHC-I in human postmortem samples. First, MHC-I was examined by immunofluorescence using a primary antibody against human HLA a chain (amino acids 63-362) that detects an epitope common in HLA-A, B and C, together with an antibody against Fox-3, a neuronal nuclei marker. Sections of hippocampus with adjacent entorhinal cortex from human controls (59-85 years old) were initially examined. Consistent with previous reports, blood vessels in hippocampus and cortex were labeled for HLA, but no staining was observed in neurons or glia (FIG. 1A). MHC-II was never observed in neurons. Similarly, HLA immunolabel in sections of the caudate nucleus from control postmortem samples in the same age range was found only in blood vessels and not in glia or neurons (FIG. 1A).

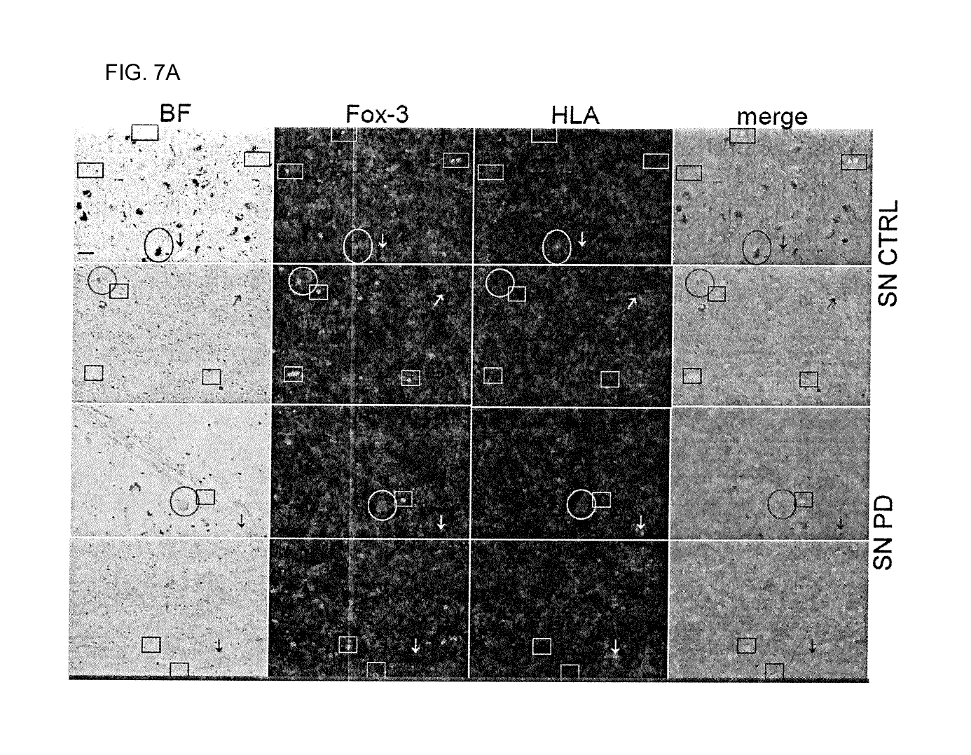

Double immunolabeling for HLA and Fox-3, or HLA and the catecholamine marker, tyrosine hydroxylase (TH) in the SN and LC of control samples and PD samples was then performed. In sharp contrast to the other brain regions examined, HLA immunolabel was present selectively in many of the TH+ NM containing neurons, as well as blood vessels (FIG. 1B and FIG. 7). The presence of MHC-I in SN neurons was independently confirmed by double immunofluorescence label for TH and an antibody against amino acids 1-120 of human β2m, the β chain component of MHC-I (FIG. 1C). The β2m cellular distribution and neuronal pattern of staining appeared identical to that of the HLA a chain (compare FIGS. 1B and 1C). Immunolabel for both MHC-I components was observed throughout the cytosol and often at particularly high levels in regions associated with NM in both SN and LC catecholamine neurons (FIG. 1B, C, D, and FIG. 7). The specificity of the neuronal MHC-I immunolabel was confirmed by the absence of staining when the respective primary antibodies were pre-adsorbed by recombinant HLA or β2m peptides (see Example 8).

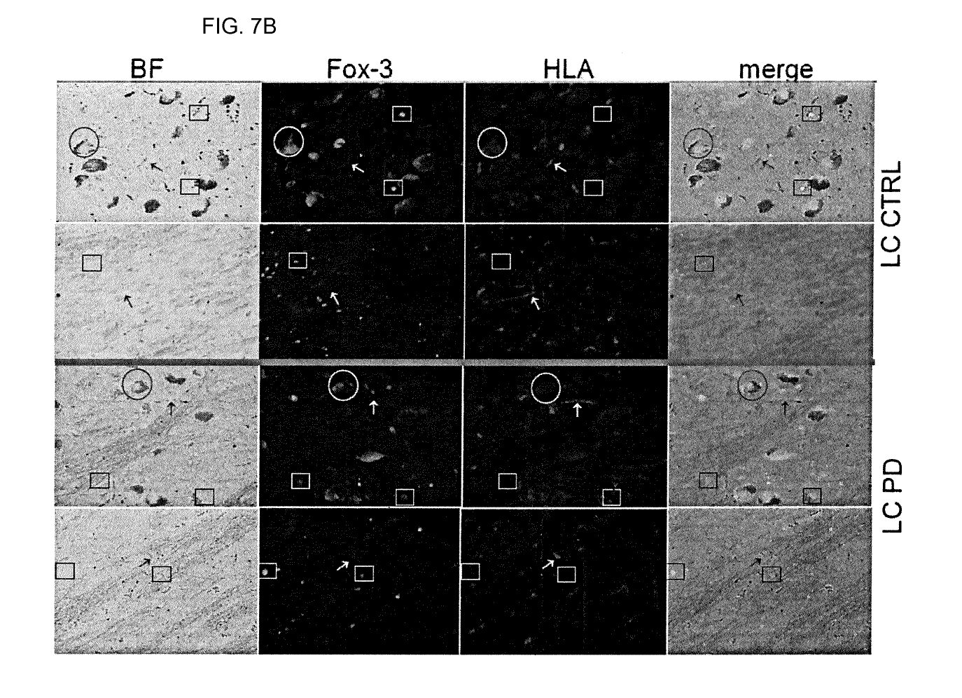

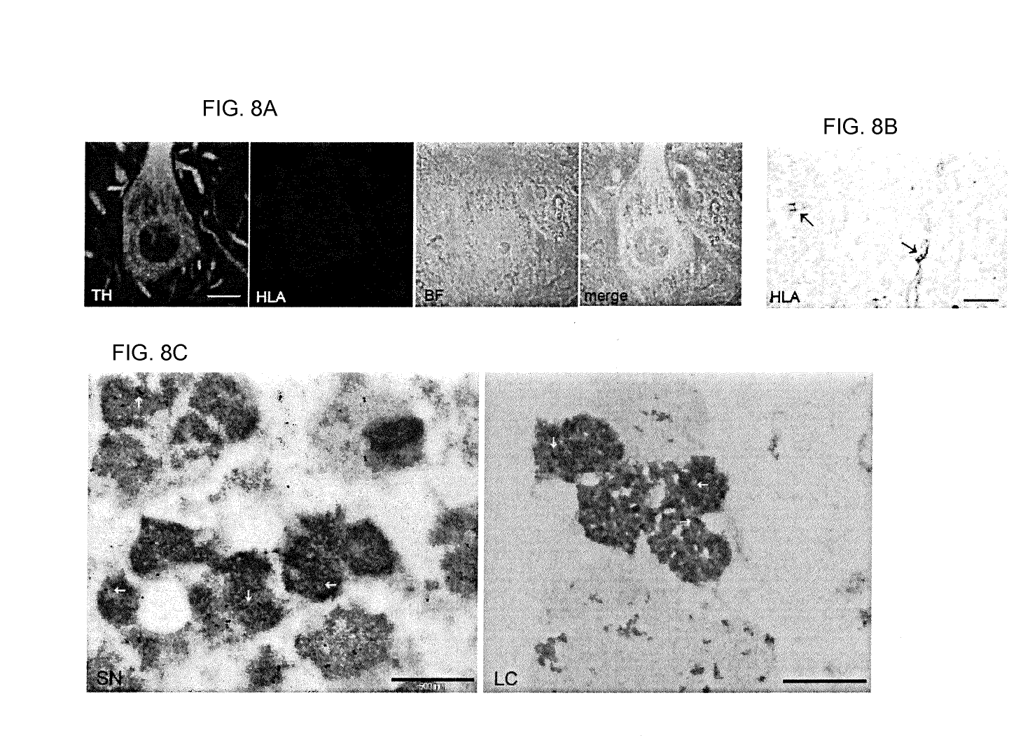

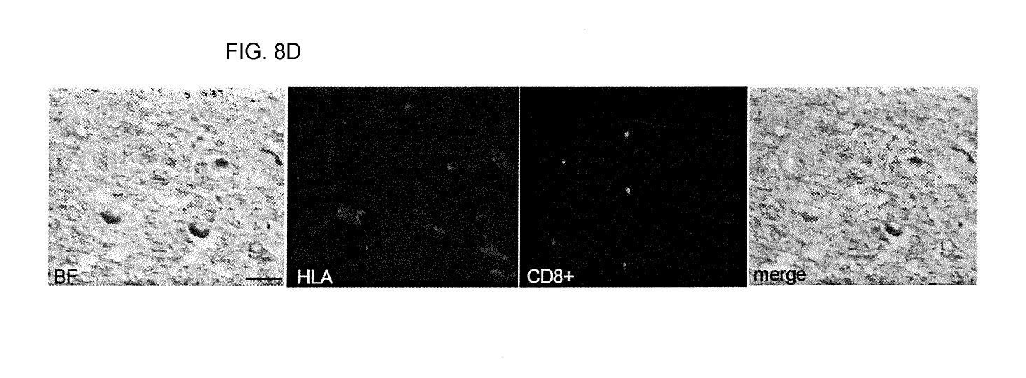

In SN postmortem material from 8 control individuals, 62.53±8.6% (mean±s.e.m.) of TH⁺ neurons displayed HLA⁺ label, whereas 49.03±8.7% (mean±s.e.m.) were labeled in 8 age-matched PD patients (FIG. 1E). Only <3% of TH⁻ SN control neurons and <7% of TH⁻ SN PD patient neurons expressed HLA (FIG. 1G). In the same sections, no DA neurons were observed in the ventral tegmental area (VTA) that expressed MHC-I (FIG. 8A). In the LC, 80.3±1.8% (mean±s.e.m.) of TH⁺ neurons were HLA⁺ from 8 control individuals, and 37.3±11.8% of TH⁺ were HLA⁺ in neurons of 9 age-matched PD patients; this difference was statistically significant (FIG. 1F). No TH⁻ neurons expressed HLA label in LC control samples, and less than 5% TH⁻ did in LC PD (FIG. 1G).

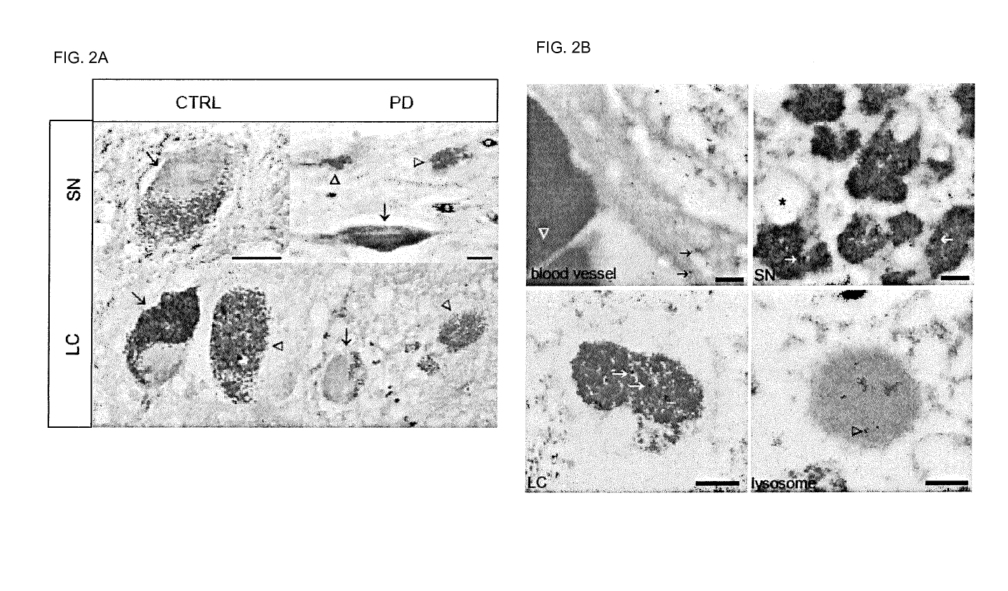

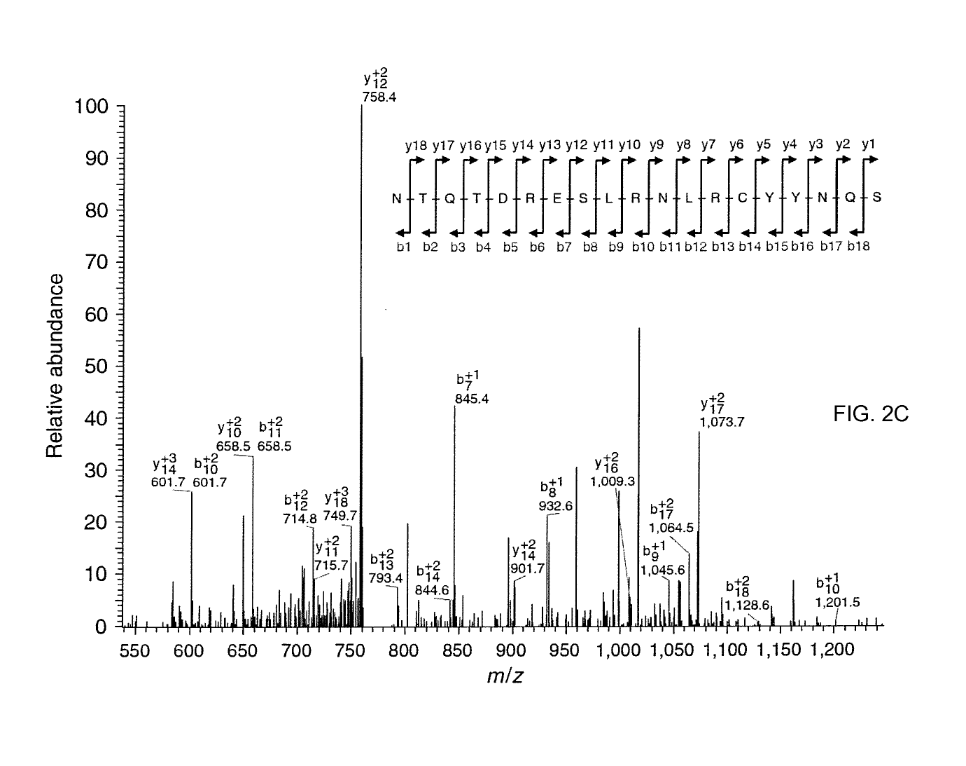

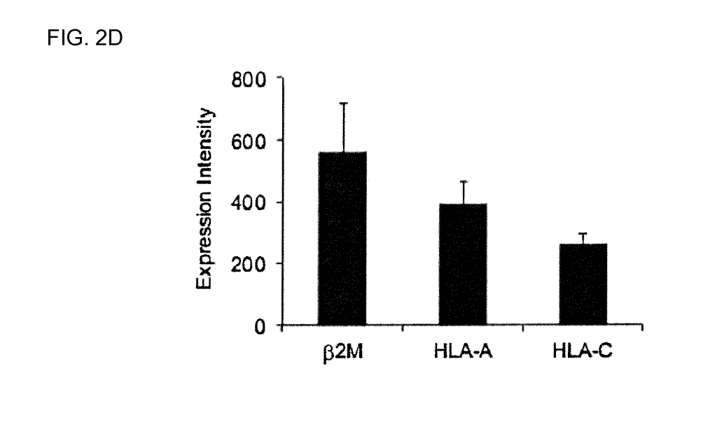

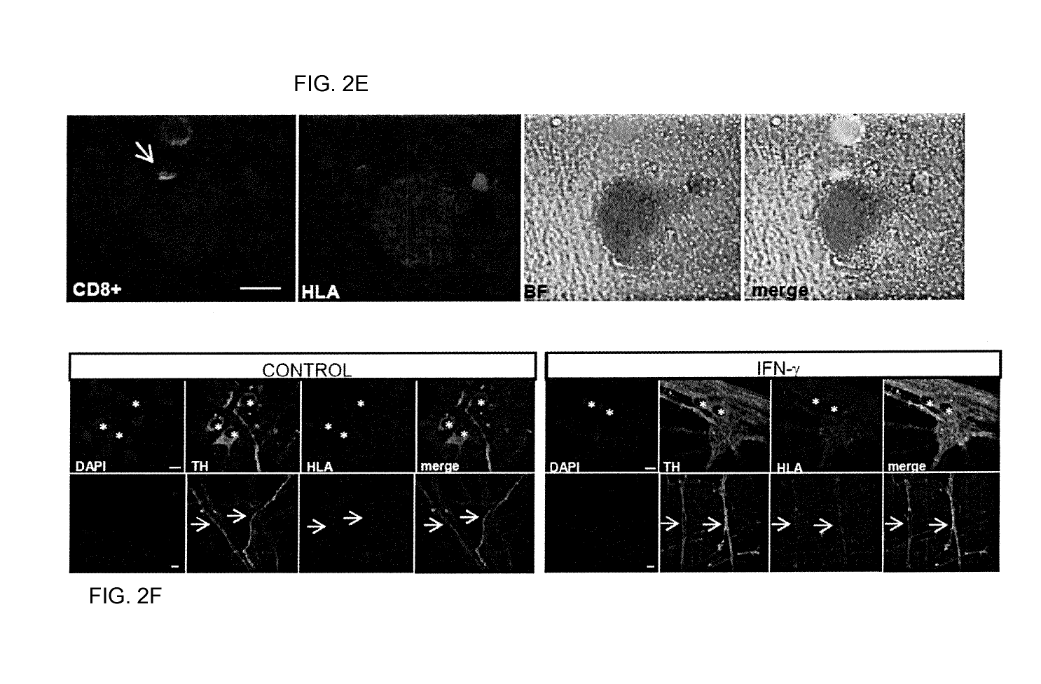

Multiple approaches were then used to corroborate the identification of MHC-I expression in SN neurons obtained by immunofluorescence. To discard artifacts due to autofluorescence, the presence of MHC-I was assessed via immunoperoxidase label. HLA label was clearly present in blood vessels, and negligible in microglia, astrocytes, and NM-neurons (FIG. 8B). In contrast, clear HLA immunoperoxidase label was observed in both NM+ SN and LC neurons, both in control and PD samples (FIG. 2A). MHC-II was never observed in neurons. HLA immunolabel was then examined in LC and SN from control samples by immunoelectron microscopy. Consistent with the immunofluorescent images of MHC-I, immunogold label for MHC-I was associated with blood vessels and was clearly present in NM organelles in LC and SN neurons (FIG. 2B, FIG. 8C).

As NM organelles can be efficiently isolated from human SN (Sulzer et al., 2000; Zecca et al., 2008), mass spectrometry was used to detect the presence of MHC proteins in isolated NM organelles and purified NM. A variety of MHC-I components, including peptides corresponding to alleles of HLA-A, HLA-B, HLA-C and β2m genes (FIG. 2C, Table 1 and Table 2), were present in both NM organelles and purified NM; in contrast, no MHC-II peptides were identified in any human SN NM samples.

The abundance of MHC-I mRNA in human NM+SN neurons from control samples was then evaluated. Robust expression signals were confirmed for the β2M and HLA-A and HLA-C genes in a laser-captured NM+SN neuron microarray data set (Zheng et al., 2010), comprising nine (FIG. 2D) control individuals without neurodegenerative diseases (mean age at death (range), 75 years (61-80).

It is impossible to determine if neuronal MHC-I in postmortem tissue was immunologically competent, but the presence of CTLs in SN and LC would indicate that such interactions could occur. CD8+ and CD4+ cells have been identified previously in human SN in close vicinity to NM+ neurons and are present at particularly high levels in PD (Brochard et al., 2009). Using double immunolabel assay for HLA and CD8+ CTLs in SN and LC control samples, CD8+ CTLs were observed in close proximity to HLA-expressing NM+ neurons in both control (FIG. 2E) and PD specimens (FIG. 8D); in some cases, catecholaminergic HLA⁺ neurons and CTLs appeared to be in contact (FIG. 2E).

### Example 2. MHC-I Induction hES-Derived DA Neurons

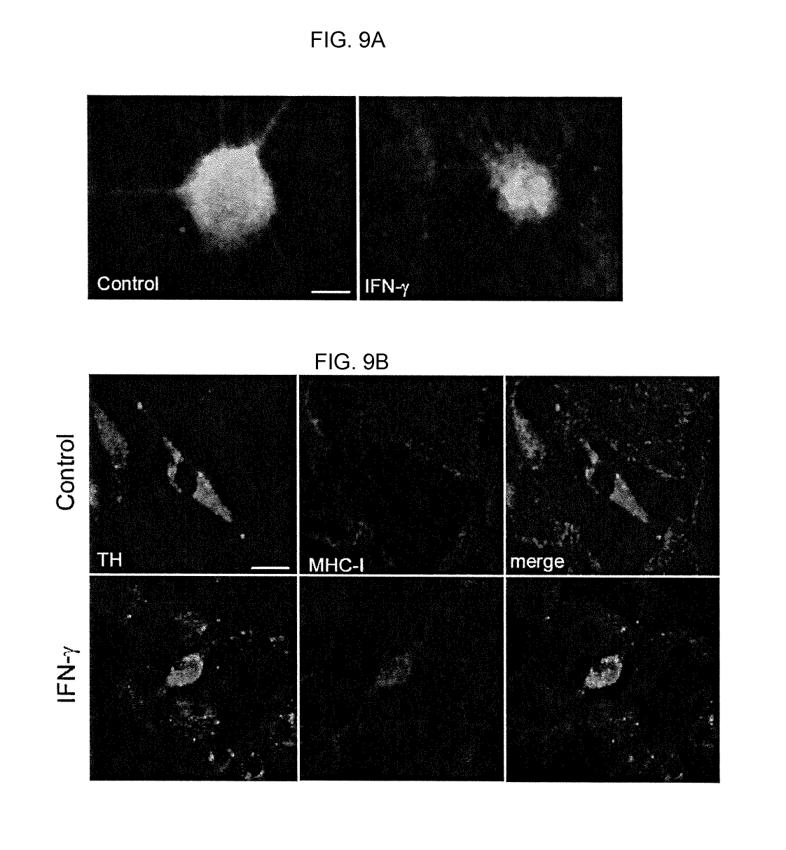

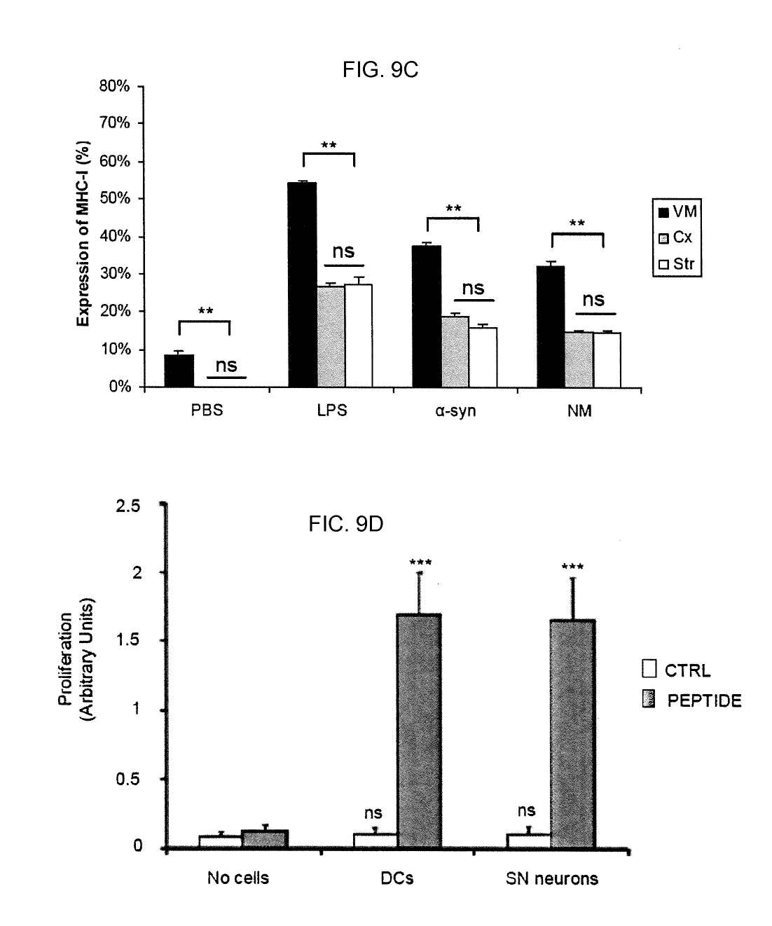

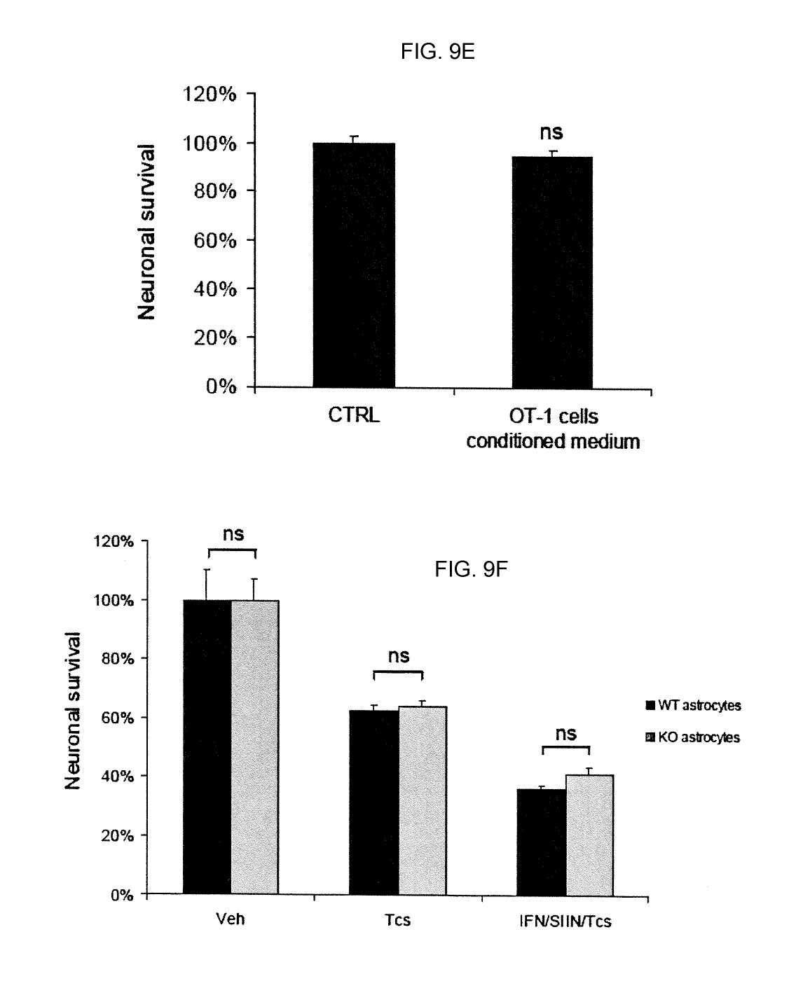

CNS neurons in postmortem samples do not retain intact plasma membranes (Lewis, 2002). To confirm that MHC-I can be localized in the plasma membrane of human DA neurons, neuronal cultures derived from hES that exhibit a range of properties of authentic SN DA neurons were examined (Kriks et al., 2011). Mature midbrain DA neurons (150 days in culture) displayed a dense TH+ fiber network that extended over distances of several millimeters. IFN-γ is a proinflammatory cytokine found at high levels in cerebrospinal fluid and blood of patients with neurodegenerative disorders (Solerte et al., 2000; Mogi et al., 2007; Kallaur et al., 2013) that induces MHC-I expression in antigen presenting cells by activating a regulatory element of the MHC-I promoter (van den Elsen et al., 2004). Untreated DA neurons exhibited no MHC-I; however, human IFN-γ induced plasma membrane expression of MHC-I by virtually all TH+ neurons (FIG. 2F). In control experiments to assess neuronal specificity, mature motor neurons derived from hES were examined after exposure to human IFN-γ, but no MHC-I expression was observed (FIG. 9A).

TABLE 1

| Sample Type | Peptide | HLA Gene |
|---|---|---|
| NM-Organelles 1 | YWDLQTRNVKAHSQTDRA | HLA-A |
| (1 subject) | RNTQIFKTNTQTHRENLRIALRY | HLA-B |
| | FLPTGGKGGSCSQAAS | HLA-C |
| | QRMEPRAPWIEQERPAYW | HLA-A |
| | VYSRHPAENGKSNFLNCYVSGFHPSDIE | (B2M) Beta-2- |
| | | microglobulln |
| | PDGRLLRGYDDDAYDG | HLA-B/HLA-A |
| NM-Organelles 2 | GHVRGVAPDIPGERE | HLA-A |
| (1 subject) | HDADVGGGPCGGAVESLPGGHVRG | HLA-A |
| | FLPTGGKGGSCSDAAS | HLA-C |
| NM Isolated from | RKLEAAGVAEDLRAYLEGECV | HLA-B |
| SN tissues | DVGSDGRFLR | HLA-A |
| (5 subjects) | NTDTDRESLRNLRCYYNDS | HLA-8 |
| | SLPGGRVRGVAPQIPGE | FILA-B |
| | KWEAARVAEQLRAYLEGLCVEWLRRH | FILA-B |
| | DRETRDLTGNGKD | MICA & PERB11.1 |
| | LSSWTAADTAAQITQRKLEAA | HLA-B/HLA-C |
| | ILRWEPSSQPTIPIVGIIAGLVL | HLA-A |
| NM isolated from | GHVRGVAPQIPGERE | HLA-A |
| NM organelles | HQAQVGGGPCGGAVESLPGGHVRG | HLA-A |
| (1 subject) | FLPTGGKGGSCSQAAS | HLA-C |
| | FLPTGGKGGSCSQAASSNSAQGSD | HLA-C |

Table 1: MHC-I Peptides Associated with NM were Identified by Mass Spectrometry in Samples Isolated from Human SN of Normal Subjects.

NM-Organelles 1 and NM-Organelles 2 were each prepared from two different single subjects. NM isolated from SN tissues was isolated from a pool of SN samples obtained from five subjects. An additional NM isolated from NM organelles was prepared from a single subject. For each peptide the corresponding gene(s) is reported. Peptides, alleles, and accession numbers are detailed in Table 2.

The SEQ ID Numbers for the sequences shown in Table 1 from the top to the bottom of the table are SEQ ID NOS. 60-80, respectively.

Table 2 (Below): MHC-I Peptides Associated with NH and Identified by Mass Spectrometry Analysis of Samples Isolated from Human SN.

ND: the nucelotidic sequence of this allele corresponds to an HLA gene but is not deposited in the IMGT/HLA alleles database (Robinson et al., 2011; www.ebi.ac.uk/ipd/imgt/hla/). (a, b, c, d): Both peptides (a) and (b) are components of nine different MHC-I proteins. In our samples, from 1 to 9 of these different MHC-I proteins can be present. Both peptides (c) and (d) are components of five different MHC-I proteins. In our sample, from 1 to 5 of these different MHC-I proteins can be present. NM-organelles 1 and NM-organelles 2 were prepared from two different individuals. NM isolated from SN tissues was isolated from a pool of SN samples obtained from five individuals. Another NM sample was isolated from NM organelles prepared from one individual. For each peptide the corresponding gene(s) and the number of different alleles that may encode the peptide is reported. *MICA & PERB11.1 are HLA related genes.

The SEQ ID Numbers for the sequences shown in Table 1 from the top to the bottom of the table are SEQ ID NOS. 60-80, respectively.

TABLE 2

| | | | Number | | | Accession |
|---|---|---|---|---|---|---|
| | | | of MHC-I | | | Number |
| | | | sequences | | | (European |
| Sample | | | containing | HLA | Alleles encoding | Nucleotide |
| Type | # | Peptide | the peptide | Gene | the peptide | Archive) |
| NM-Organelles 1 | 1 | YWDLQTRNVKAHSQTDRA | 2 | HLA-A | HLA-A*43:01 | X61703 |
| (1 subject) | | | | | HLA-A*29:20 | FJ222559 |
| | 2 | RNTQIFKTNTQTHRENLRIALRY | 1 | HLA-B | HLA-B*51:52 | AM906166 |
| | 3 | FLPTGGKGGSCSQAAS | 5 | HLA-C | HLA-C*03.04:01:01 | CR759828 |
| | | | | | HIA-C*05:01:01:02 | 8X248310 |
| | | | | | HLA-C*06:02:01:01 | CR388229 |
| | | | | | HLA-C*16:01:01 | BX927178 |
| | | | | | n.d. | BC008457 |
| | 4 | ORMEPRAPWIEOERPAYW | 1 | HLA-A | HLA-A*31:22 | EU580147 |
| | 5 | VYSRHPAENGKSNFINCYVSGFH | 5 | B2M (Beta-2- | not HLA gene | — |
| | | PSDIE | | microglobulin) | | |
| | 6 | PDGRLLRGYOCIDAYOG | 105 | HLA-A/HLA-B | This peptide can be encoded by | |
| | | | | | the allele HLA-B*74:11 (accession | |
| | | | | | number: DQ888175) and by 104 | |
| | | | | | alleles belonging to HLA-A*74 | |
| | | | | | and HLA-A*32 allele families | |
| NM-Organelles 2 | 1 | GHVRGVAPQIPGERE | 9 | HLA-A | HLA-A*02:20:02 | AJ276069 |
| (1 subject) | | | | | HLA-A*02:08 | X94571 |
| | | | | | n.d. | AJ429615 |
| | | | | | n.d. | AY918166 |
| | | | | | HLA-A*02:17:01 | X89707 |
| | | | | | HLA-A*02:01:01:01/ | CR388220 |
| | | | | | HLA-H*01:01:01:01/ | |
| | | | | | HLA-K*01:02 | |
| | | | | | HLA-A*02:250N | FN806794 |
| | | | | | HLA-A*2new | AM905047 |
| | | | | | HLA-X68:13 | AM072964 |
| | 2 | HQAQVGGGPCGGAVESLPGGHVRG | 9 | HLA-A | The same of above | |
| | 3 | FLPTGGKGGSCSQAAS | 5 | HLA-C | HLA-C*03:04:01:01 | CR759828 |
| | | | | | HLA-C*05:01:01:02 | BX248310 |
| | | | | | HLA-C*06:02:01:01 | CR388229 |
| | | | | | HLA-C*16:01:01 | BX927178 |
| | | | | | n.d. | BC008457 |
| NM isolated | 1 | RKLEAAGVAEQLRAYLEGECV^{(a)} | 1 | HLA-B | HLA-B*40:67 | Af3257503 |
| from SN | 2 | DVGSDGRFLR | >1000 | HLA-A | This peptide can be encoded | |
| tissues | | | | | by >1000 different alleles | |
| (5 subjects) | | | | | of HLA-A gene | |
| | 3 | NTQTDRESLRNLRCYYNQS | 1 | HLA-B | HLA-B*08:34 | AM778129 |
| | 4 | SLPGGRVRGVAPQIPGE | 1 | HLA-B | HLA-B*27:12 | Y14582 |
| | 5 | KWEAARVAEQLRAYLEGLCVEWL | 27 | HLA-B | This peptide can be encoded by | |
| | | RRH | | | 27 alleles belonging to HLA-B*15, | |
| | | | | | B*35,B*51, B*52, B*57, B*58 | |
| | | | | | alleles families | |
| | 6 | DRETRDLTGNGKD | 56 | MICA & | MICA*001 | |
| | | | | PERB11.1 (s) | | |
| | 7 | LSSWTAADTAAQITQRKLEAA | 4 | HLA-B/HLA-C | HLA-B*40:77 | EF521874 |
| | | | | | HLA-B*48new | DQ238865 |
| | | | | | HLA-C*07:168 | HQ589100 |
| | | | | | HLA-C*07:81 | FJ618920 |
| | 8 | ILRWEPSSQPTIPIVGIIAGLVL | 1 | HLA-A | HLA-A*02:134 | AM746482 |
| NM isolated | 1 | GHVRGVAPQIPGERE ^{(a)} | 9 | HLA-A | HLA-A*02:20:02 | AJ276069 |
| from NM | | | | | HLA-A*02:08 | X94571 |
| Organellos | | | | | n.d. | AJ429615 |
| (1 subtect) | | | | | n.d. | AY918166 |
| | | | | | HLA-A*02:17:01 | X89707 |
| | | | | | HLA-A*02:01:01:01/ | |
| | | | | | HLA-H*01:01:01:01/ | CR388220 |
| | | | | | HLA-K*01:02 | |
| | | | | | HLA-A*02:250N | FN806794 |
| | | | | | HLA-A*2new | AM905047 |
| | | | | | HLA-A*68:13 | AM072964 |
| | 2 | HQAQVGGGPCGGAVESLPGGHV | 9 | HLA-A | The same of above | |
| | | RG ^{(b)} | | | | |
| | 3 | FLPTGGKGGSCSQAAS ^{(c)} | 5 | HLA-C | HLA-C*03:04:01:01 | CR759828 |
| | | | | | HLA-C*05:01:01:02 | 6X248310 |
| | | | | | HLA-C*06:02:01:01 | CR368229 |
| | | | | | HLA-C*16:01:01 | BX927178 |
| | | | | | n.d | BC008457 |

| | | | | |
|---|---|---|---|---|
| 4 | FLPTGGKGGSCSQAASSNSAQG | 5 | HLA-C | The same of above |
| | SD ^{(d)} | | | |

### Example 3. Induction of Neuronal MHC-I in Murine Catecholamine Neurons

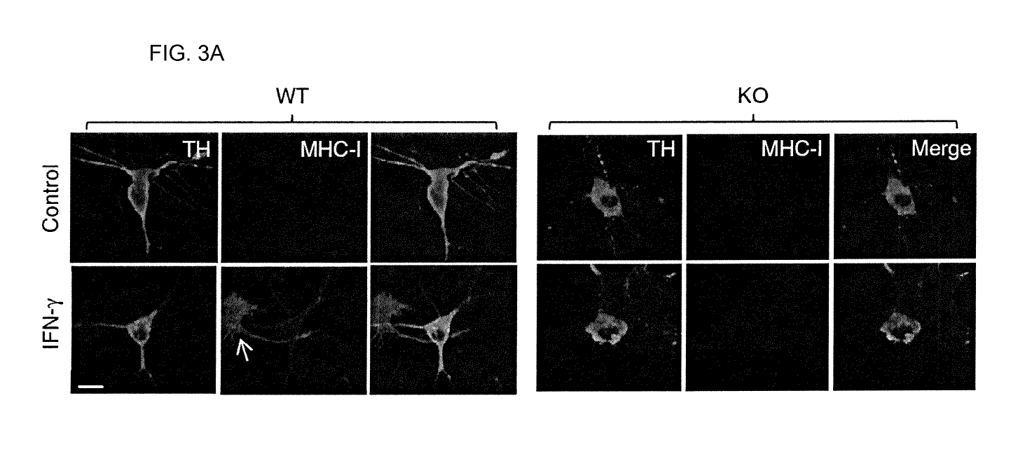

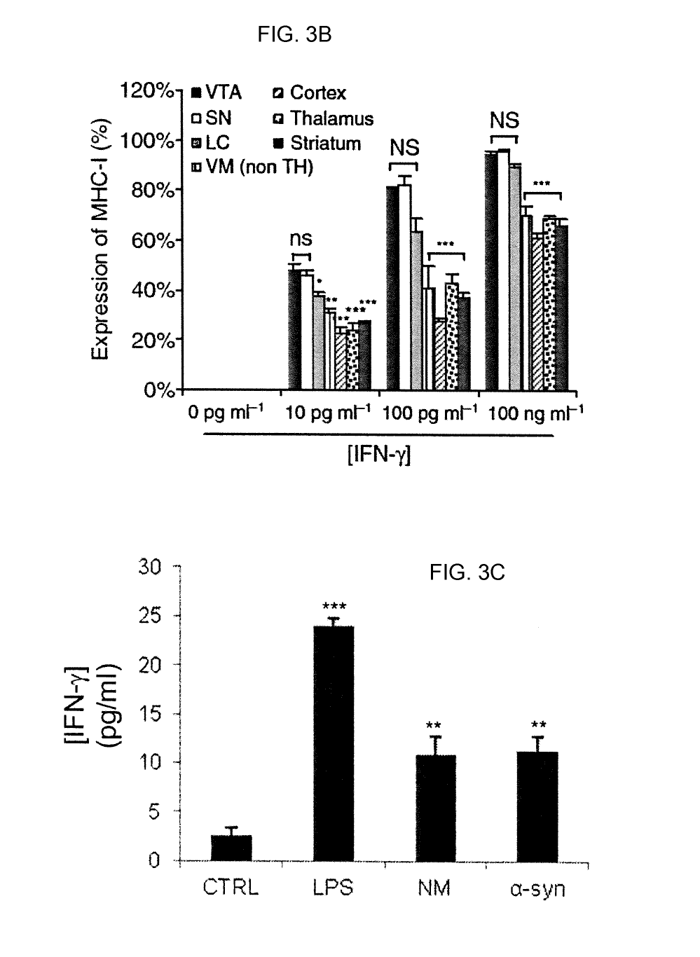

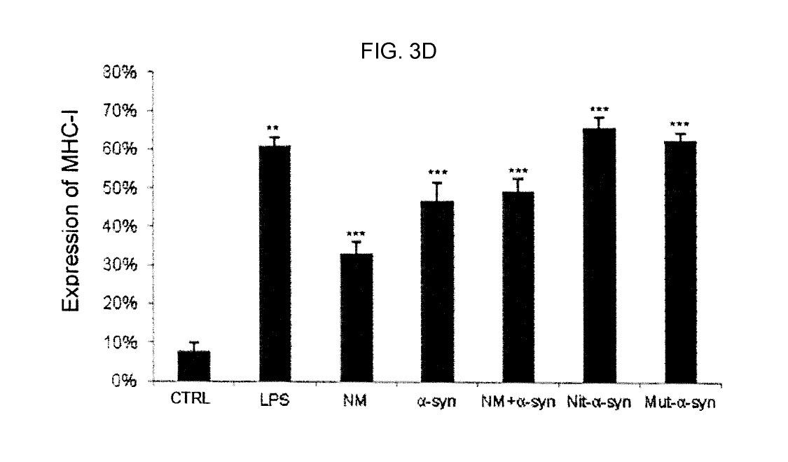

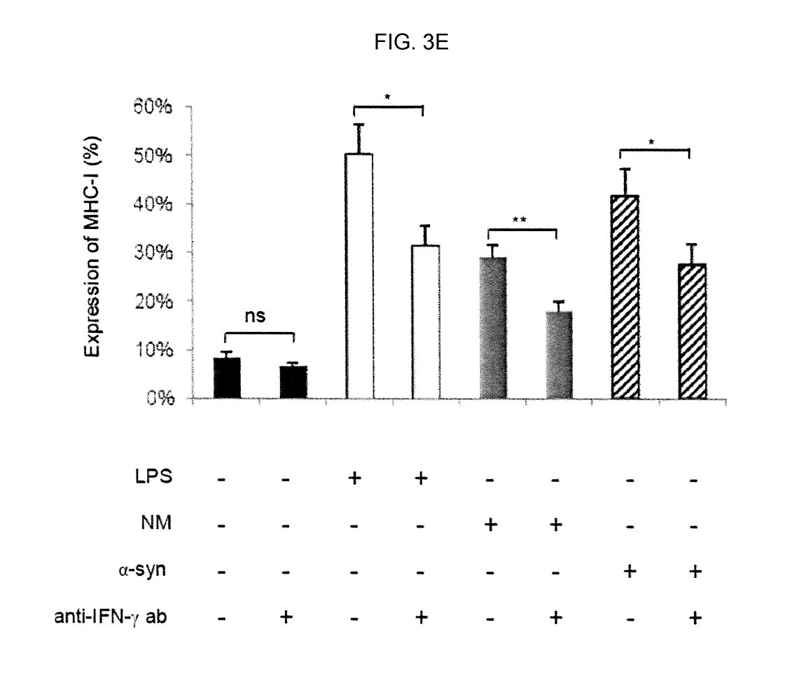

To study the mechanisms underlying the preferential display of MHC-I by catecholamine neurons, primary neuronal cultures of >7 days in vitro prepared from 0-3 day old C57BL/6 mice were examined in the studies herein (Sulzer et al., 2008). DA neurons from VTA and SN and NE neurons of the LC were identified by immunolabel for TH. MHC-I staining was examined using an antibody raised against the H-2K^{b} and H-2D^{b} class I mouse alloantigens. Untreated TH+ neurons exhibited no MHC-I immunolabel (FIG. 3A). The response to recombinant mouse IFN-γ was then examined, which induced robust plasma membrane neuronal MHC-I expression on TH+ neurons (FIG. 3A).

Detection of MHC-I immunolabel can be influenced by fixation, and therefore labeling of living neurons was also examined. Immunolabel for MHC-I was absent in control neuronal cultures and was observed in unfixed neurons only following IFN-γ (FIG. 9B), indicating that detection of neuronal MHC-I was not an artifact of fixation.

To confirm that the immunolabel was specific for MHC-I, neurons cultured from MHC-I null (“knockout”: KO) mice which are deficient for β2m (B6.129P2-B2mtm1Unc/J) were examined. As expected, IFN-γ did not induce MHC-I (H-2K^{b} and H-2D^{b}) immunolabel in these mutant neurons (FIG. 3A). A series of control experiments was further conducted, in which wild type and KO astrocytes were used combinatorially with wild-type and KO neurons. For each combination, wild type neurons and astrocytes were induced to express MHC-I by IFN-γ, while in each case the KO neurons and astrocytes did not express MHC-I.

The response to IFN-γ of LC and ventral midbrain (VM: SN and VTA) catecholamine neurons was compared to cortical, thalamic, striatal and non-DA SN cultured neurons that were identified by label for the neuron specific cytoskeleton protein, microtubule-associated protein-2 (MAP-2). Catecholamine neurons of the SN, VTA and LC were far more responsive to IFN-γ than were non-catecholamine neurons: even 1000-fold excesses of IFN-γ did not induce non-catecholamine neurons to express MHC-I at the level of catecholamine neurons (compare 100 pg/ml to 100 ng/ml, FIG. 3B).

### Example 4. Regulation of MHC-I Expression by Activated Microglia

Without wishing to be bound by any scientific theory, the induction of MHC-I in the SN neuronal culture experiments described herein, as well as in prior reports from other neurons (Medana et al., 2000; Meuth et al., 2009), was triggered by IFN-γ. This cytokine is considered to be a product of lymphoid cells, but CNS microglia provide an alternate source of this proinflammatory signal (Kawanokuchi et al., 2006). Activated microglia have long been recognized in a variety of neurodegenerative disorders including PD (Foix and Nicolesco, 1925). Extracellular NM, which is a remnant of dead SN neurons (Foix and Nicolesco, 1925), and various forms of extracellular α-syn can activate microglia (Zhang et al., 2007; Volpicelli-Daley et al., 2011). Using an enzyme-linked immunosorbent assay (ELISA), it was found that primary microglial cultures exposed to human NM or native α-syn for 72 h secreted 5-10 fold more IFN-γ than untreated cells (FIG. 3C). Control experiments revealed that lipopolysaccharide (LPS), an inflammatory component from Gram-negative bacteria, also induced IFN-γ secretion in primary microglial culture (FIG. 3C).

Both overexpression and multiple mutations of α-syn are implicated in PD (Polymeropoulos et al., 1997; Singleton et al., 2003). The effects of conditioned media from microglial cultures exposed to NM or a variety of modified α-syn proteins on neuronal MHC-I expression were examined. The conditioned medium obtained from microglia cultures incubated for 72 hours with NM, α-syn, NM with α-syn, nitrated α-syn (Nit-α-syn), A53T mutant α-syn (Mut-α-syn), or LPS, elicited cell surface MHC-I presentation by VM neurons (FIG. 3D). This induction of MHC-I expression was in part mediated by IFN-γ, as the addition of IFN-γ neutralizing antibody to the conditioned medium decreased MHC-I expression by 30-50% (FIG. 3E). In contrast, there was no induction of neuronal MHC-I when cultures were directly exposed to equivalent levels of LPS, α-syn, or NM in the absence of microglial cells. These results indicate that substances including NM and α-syn can activate microglia, as demonstrated by the release of IFN-γ, as well as that additional inflammatory factors induce MHC-I display by VM neurons.

Primary cortical and striatal neurons were also analyzed for MHC-I expression after exposure to microglial medium pre-incubated for 72 hours with LPS, NM or α-syn under the same conditions used for VM DA cultures. Under these conditions, cortical and striatal neurons also expressed MHC-I, but in a significantly lower proportion than VM DA neurons (FIG. 9C).

### Example 5. Regulation of Expression in Response to Oxidative Stress

Protein oxidation has been suggested to provide a “global signal” that induces MHC-I expression (Teoh and Davies, 2004). DA-derived oxidative stress has been associated with neurodegenerative diseases including PD (Fahn and Sulzer, 2004; Hwang, 2013), in part because cytosolic DA, which is also the precursor to NE in LC neurons, can be metabolized to a quinone that reacts with proteins including α-syn (Conway et al., 2001; Norris et al., 2005). DA-modified α-syn leads to cellular toxicity and autophagy dysfunction (Martinez-Vicente et al., 2008; Muñoz et al., 2012). Moreover, oxidation of cytosolic DA triggers NM synthesis as an autophagic cellular stress response (Sulzer et al., 2000). Without wishing to be bound by any scientific theory, it was thus hypothesized that conditions which promote oxidative stress via high cytosolic DA (Mosharov et al., 2009) may account for the selectivity of neuronal MHC-I expression by catecholamine neurons.

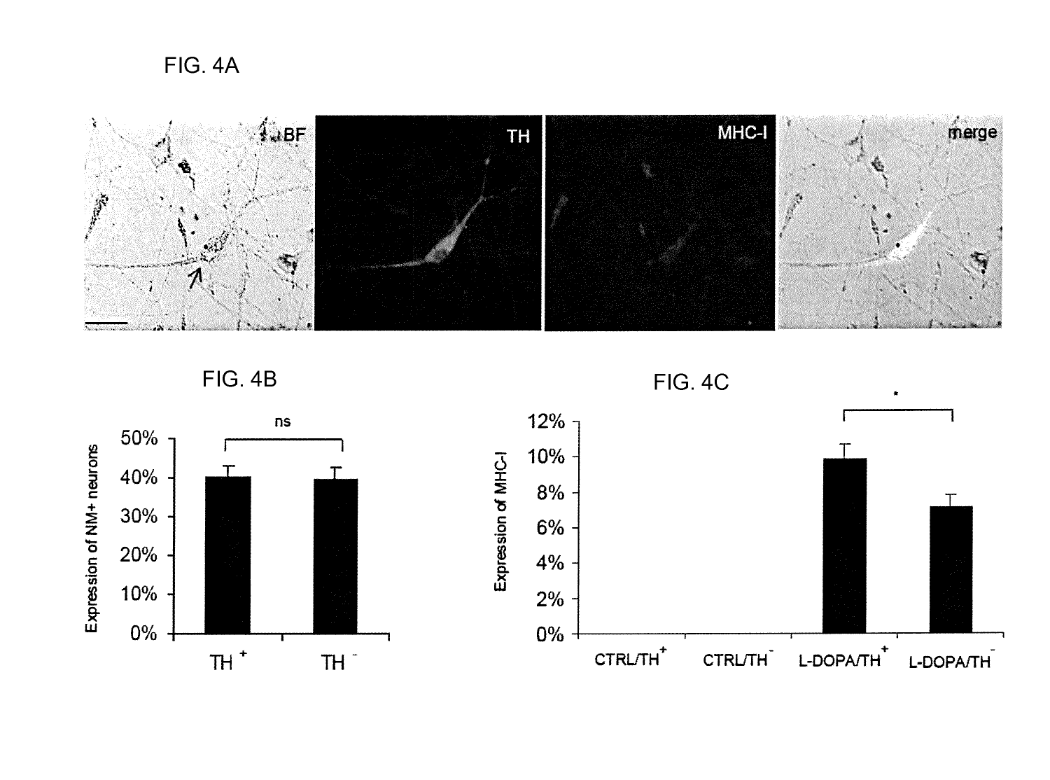

To enhance cytosolic DA, the DA precursor, L-DOPA, was used, which is accumulated by amino acid transport and converted into DA in the cytosol. Exposure to high levels of L-DOPA can produce NM in both catecholaminergic and nominally non-catecholaminergic cells (Sulzer et al., 2000). L-DOPA is used therapeutically in PD treatment, and while there is no clear evidence indicating that L-DOPA is toxic in humans including PD patients, the compound can be toxic to cultured cells (Pardo et al., 1995; Mosharov et al., 2009). SN murine neuronal cultures were exposed to L-DOPA under conditions that substantially increase cytosolic DA as measured by intracellular patch electrochemistry (Mosharov et al., 2009) and promote NM production as observed by brightfield microscopy (Sulzer et al., 2000). Control SN neurons exhibited no visible NM, while the addition of L-DOPA induced NM in 40% of the neurons (FIGS. 4A and 4B), confirming the formation of non-degradable oxidized catecholamine products within autophagic organelles (Sulzer et al., 2000). In these experiments L-DOPA was found to stimulate neuronal MHC-I display in ˜10% of TH+ neurons and ˜7% of TH− neurons (FIG. 4C).

Altogether, the data herein indicate that high levels of neuronal DA can induce the formation of pigmented NM organelles and the presentation of neuronal MHC-I even in the absence of microglia or exogenously added IFN-γ. This suggests that redox stress in neuronal cytosol due to cytosolic catecholamines could underlie the particular expression of MHC-I by catecholamine neurons. This is also consistent with the observation that TH− neurons can display MHC-I when treated with L-DOPA, which induces accumulation of catechols in cytosol followed by their subsequent oxidation. L-DOPA exposure in patients is noted to be substantially lower than in the culture system, and may not be sufficient to enhance oxidation or MHC-I display, but the findings herein suggest that a history of cytosolic catecholamine could provide a signal leading to the MHC-I display specific to catecholamine neurons in adult human.

### Example 6. SN Neurons are Antigen Presenting Cells

A characteristic of “professional” antigen cells such as dendritic cells (DCs) is their capacity to process and load antigen onto the MHC-I groove. Although previous in vitro studies show hippocampal neurons display MHC-I upon exposure to IFN-γ and present small peptides exogenously added to the culture (Medana et al., 2000; Meuth et al., 2009), there are to our knowledge no reports examining whether neurons can internalize, process and load antigens onto the MHC-I as other cells do.

To examine antigen presentation by cultured neurons, 1) all sources of bovine serum albumin (BSA) were removed from the media, and 2) VM neuronal cultures were exposed to chicken OVA. OVA is a 385 amino acid foreign protein (Nisbet et al., 1981) that can be cleaved to an 8 amino acid SIINFEKL (SEQ ID NO: 9) peptide by DCs and other “professional” antigen presenting cells; the SIINFEKL (SEQ ID NO: 9) peptide is then loaded and presented in their MHC-I groove (Falk et al., 1993).

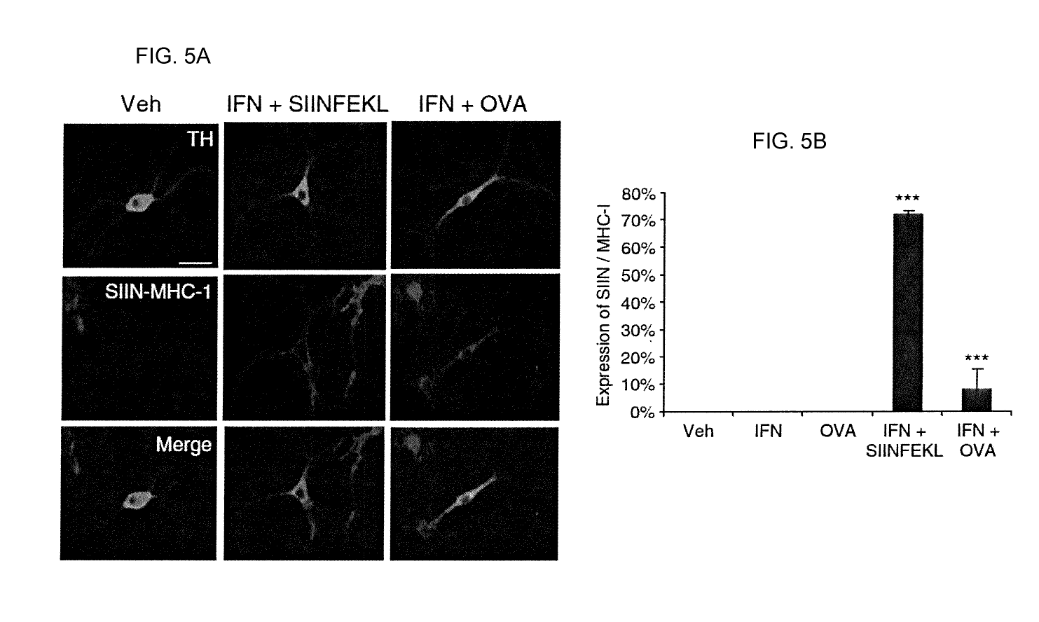

Following exposure of SN neuronal cultures to OVA or vehicle for 7 days, neurons were exposed to IFN-γ or saline for 72 h (note that these cultures were never exposed to SIINFEKL (SEQ ID NO: 9)). The cultures were then double immunolabeled for TH and an antibody that recognizes only the MHC-I complex when is occupied by SIINFEKL (SEQ ID NO: 9) (SIINFEKL-MHC-I). Occasional label of astrocytes, but not neurons, for SIINFEKL-MHC-I was observed when the cultures were exposed to the vehicle, IFN-γ, or OVA alone (FIG. 5A). In contrast, ˜10% of TH+ neurons exposed to both OVA and IFN-γ were immunolabeled for SIINFEKL-MHC-I (P<0.001, one-way ANOVA; FIG. 5A), which was present throughout the cytoplasm, indicating that SIINFEKL (SEQ ID NO: 9) was loaded onto MHC-I within the neuron. In contrast, when cultures were exposed to IFN-γ with extracellular SIINFEKL (SEQ ID NO: 9) as a positive control, ˜70% of TH+ neurons exhibited SIINFEKL-MHC-I immunolabel selectively on the plasma membrane and not in the cytosol (P<0.001, one-way ANOVA, FIG. 5A,B). The results herein indicate that OVA had been processed to SIINFEKL (SEQ ID NO: 9) within these mixed neuron/astrocyte cultures and loaded into the MHC-I groove within neuronal cytosol, and that the resulting complex was presented on the neuronal plasma membrane.

### Example 7. VM DA Neuronal Killing by CTLs

As mentioned above, MHC-I display by CNS neurons has to date mostly been implicated in synaptic plasticity (Huh et al., 2000; Oliveira et al., 2004; Goddard et al., 2007; Corriveau et al., 1998; Shatz, 2009; Needleman et al., 2010; Glynn et al., 2011; Elmer and McAllister, 2012).

The capacity of CTLs to respond to MHC-I induced SN neurons was examined first, by following CTL proliferation using 5-bromo-2-deoxyuridine (BrdU) incorporation. The induction of CTL proliferation by DCs and VM neuronal cultures was compared using OT-1 CTL cells that constitutively recognize and respond to SIINFEKL (SEQ ID NO: 9) (Budhu et al., 2010), and it was found that the combination of IFN-γ and SIINFEKL (SEQ ID NO: 9) induced similar CTL proliferation by both DC and VM neuronal cultures (FIG. 9D). In contrast, no neuronally induced proliferation of another clonal CD4+ T cell line that specifically recognizes MHC-II was observed.

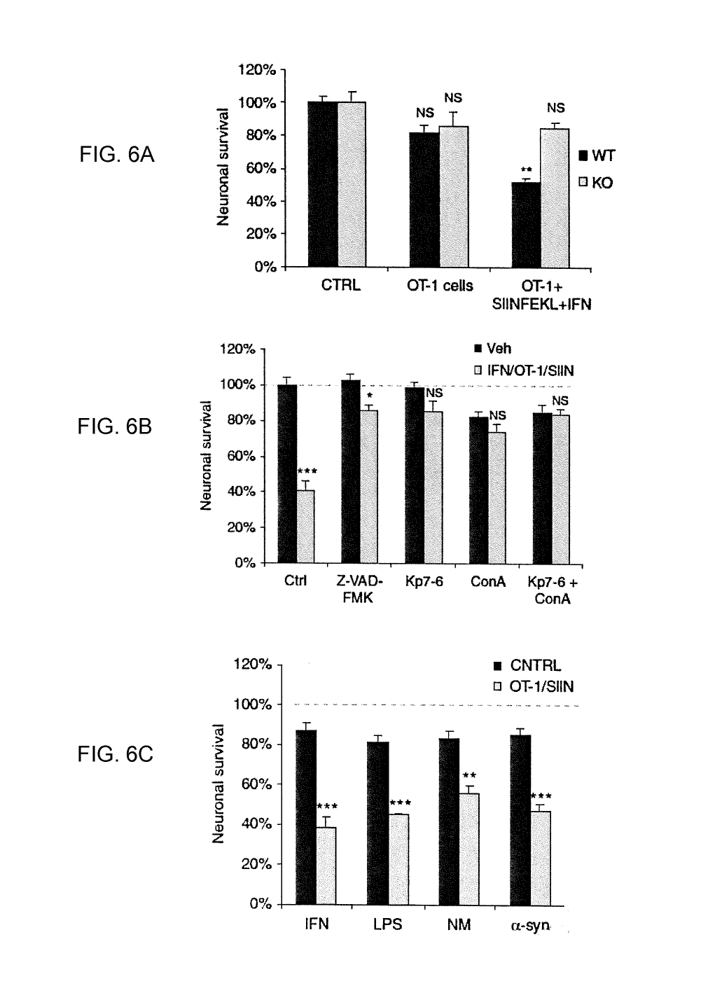

These results led to the examination of whether neuronal antigen-loaded MHC-I was competent to trigger CTL mediated cell death. The OT-1 CTL line was used as effector cells and SIINFEKL (SEQ ID NO: 9) peptide-pulsed SN neurons were used as the target cells. The combination of CTLs, IFN-γ, and SIINFEKL (SEQ ID NO: 9) killed 55% of TH+ neurons; as expected, no neuronal death was triggered in similarly treated cultures of MHC-I null (knockout: KO) SN neurons (FIG. 6A). The presence of CTLs was required to elicit neuronal death, as medium conditioned by SIINFEKL (SEQ ID NO: 9)-activated CTLs but with the CTLs themselves omitted, did not kill neurons (FIG. 9E). To determine whether the astrocyte monolayer played a role in the CTL-mediated neuronal death, cultures in which wild type or KO astrocytes were plated under wild-type ventral midbrain neurons, were compared. MHC-I was induced by IFN-γ, and then SIINFEKL (SEQ ID NO: 9) and OT-1 cells were added to the culture. Different levels of neuronal death between neurons plated on wild type astrocytes or KO astrocytes was not observed, indicating that the ability of astrocytes in culture to express MHC-I is irrelevant to OT-1 mediated neuronal death (FIG. 9F).

The mechanism of induced neuronal death by CTLs was then explored. The Fas/Fas ligand antagonist, Kp7-6, the caspase inhibitor, Z-VAD-FMK, and the perforin/granzyme antagonist, concanamycin A, each were found to protect against CTL mediated neuronal death, and that the combination of Kp7-6 and concanamycin A was found to completely block neuronal death (FIG. 6B). These results indicate that both Fas/Fas ligand and perforin/granzyme pathways play a role in SN neuron CTL-mediated death.

Finally, as predicted from MHC-I induction by activated microglia (FIG. 3D), the combination of SIINFEKL (SEQ ID NO: 9), CTLs and medium conditioned by microglia exposed to LPS, α-syn, or NM caused extensive neuronal death (FIG. 6C). Thus, SN neuronal MHC-I can be triggered by secretion of IFN-γ from microglia activated by proinflammatory compounds, or two substances, NM and α-syn, present in the extracellular milieu of the SN in PD.

### Example 8. Materials and Methods for Examples 1-7

Tissue Obtaining and Handling

This study was approved by the Institutional Review Board of: the Institute of Biomedical Technologies—National Research Council of Italy (Segrate, Milano, Italy), the New York Brain Bank (Columbia University) and the Center for Neurologic Diseases (Harvard Medical School). Informed consent was obtained from all subjects. Brain samples from 34 normal subjects and 9 PD patients were obtained during autopsy within 5-42 h after death of male and female subjects without evidence of neuropsychiatric and other degenerative disorders. At histological examination, control samples showed no macroscopic neurological, vascular alterations, Lewy bodies or other pathological markers. Tissues from PD subjects were examined histologically by routine staining to confirm loss of SN and LC pigmented neurons, the presence of Lewy bodies and extraneuronal NM. Subjects ages were between 47 and 94 years old, and each PD sample was paired with an age-matched control. Samples from 8 control and 9 PD individuals were used for immunohistochemistry and immunofluorescence; samples from 1 control subject was used for electron microscopy; samples from 8 control subjects for mass spectrometry; two existing laser-captured microdissected microarray data sets of 17 controls were analyzed for mRNA expression.

Each experiment described below was performed at least in triplicate.

Immunohistochemistry and Immunofluorescence

SN/VTA, LC, hippocampal with adjacent entorhinal cortex, and striatum (caudate) tissues blocks for histology and histochemistry were fixed in 10% formalin (pH 7.2-7.4) and embedded in paraffin (Zecca et al., 2004). Tissue sections were incubated with citric acid at 95° C. for 30 minutes to retrieve antigen and blocked in normal serum; those sections designated for immunohistochemistry were treated with H₂O₂ to inactivate endogenous peroxidase. Primary antibodies for immunohistochemistry and immunofluorescence were the following: for human MHC-I, we used a mouse monoclonal antibody raised against amino acids 63-362 of the human HLA, 1:100, Santa Cruz Biotechnology (catalogue number: sc-55582; this epitope is shared by HLA-A, HLA-B and HLA-C and so detects all three), and β2m (mouse monoclonal antibody raised against amino acids 1-120 of the human β2m, 1:100, Santa Cruz Biotechnology, catalogue number: sc-13565). A second antibody against human β2m was used (mouse monoclonal antibody raised against amino acids 1-119 of human β2m, 1; 100, Abnova, catalogue number: H00000567-M01), that provided the same pattern of staining as the HLA and the original β2m antibodies. For human MHC-II: a mouse monoclonal antibody raised against HLA-DR/DP/DQ/DX (CR3/43), 1:100, Santa Cruz Biotechnology, catalogue number: sc-53302. For TH: rabbit polyclonal, 1:2000, Millipore, catalogue number: AB152. For Fox-3: rabbit polyclonal, 1:500, Abcam, catalogue number: ab104225. For CD8⁺: rabbit polyclonal, raised against a synthetic peptide sequence comprising the 13 C-terminal amino acids of the cytoplasmic domain of α-chain of the CD8 molecule, 1:50, ThermoScientific, catalogue number: RB-9009-P1.

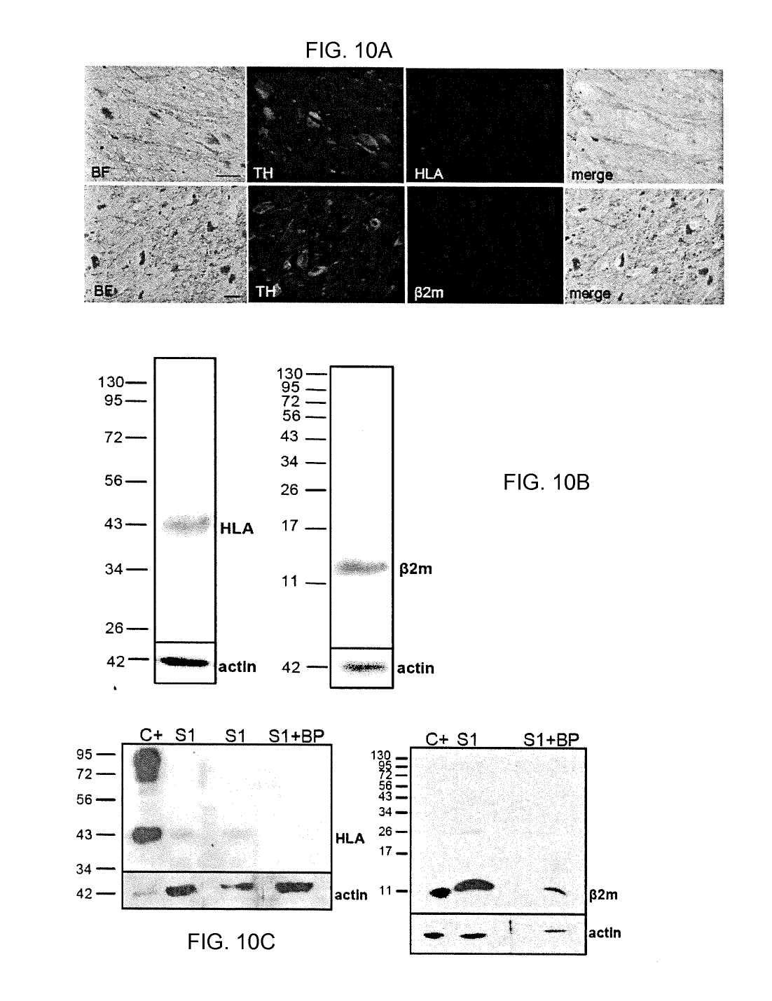

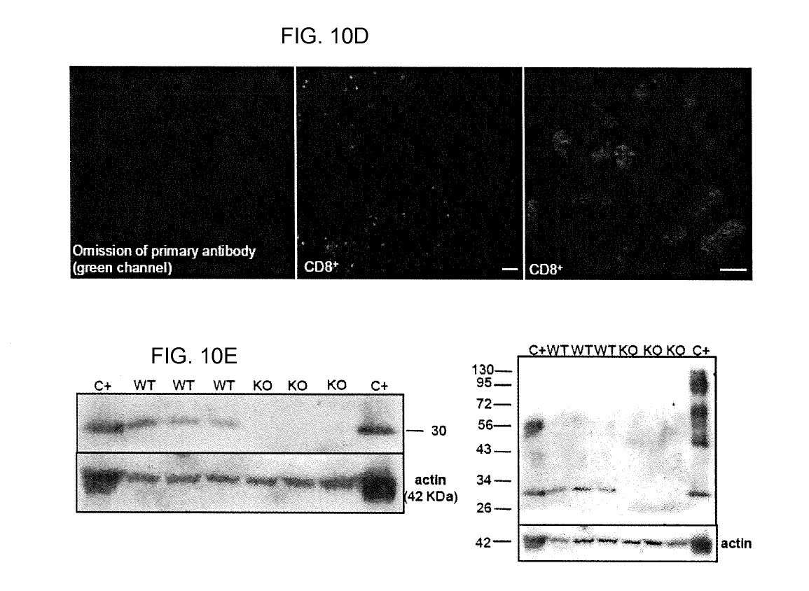

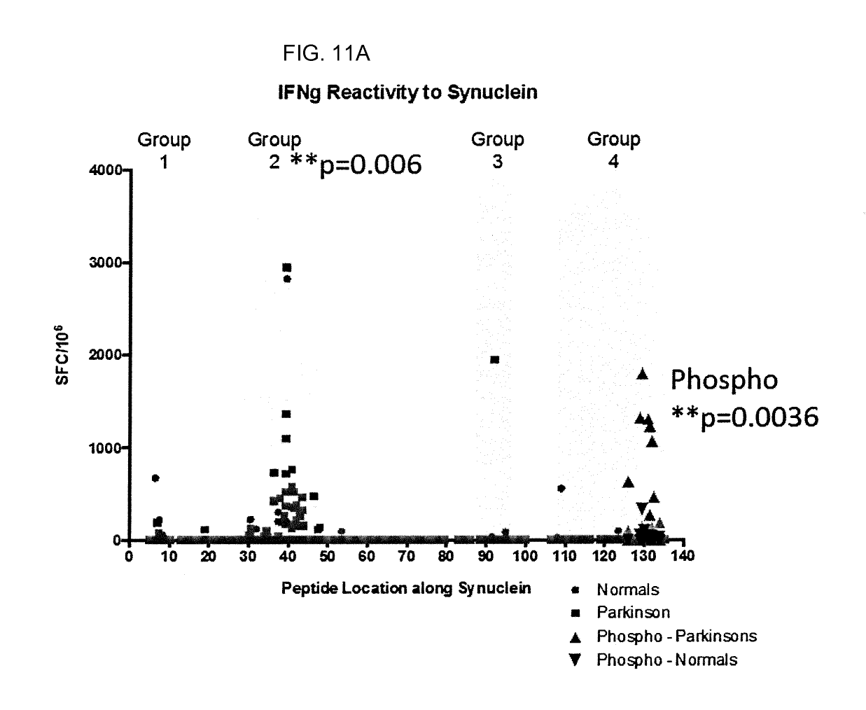

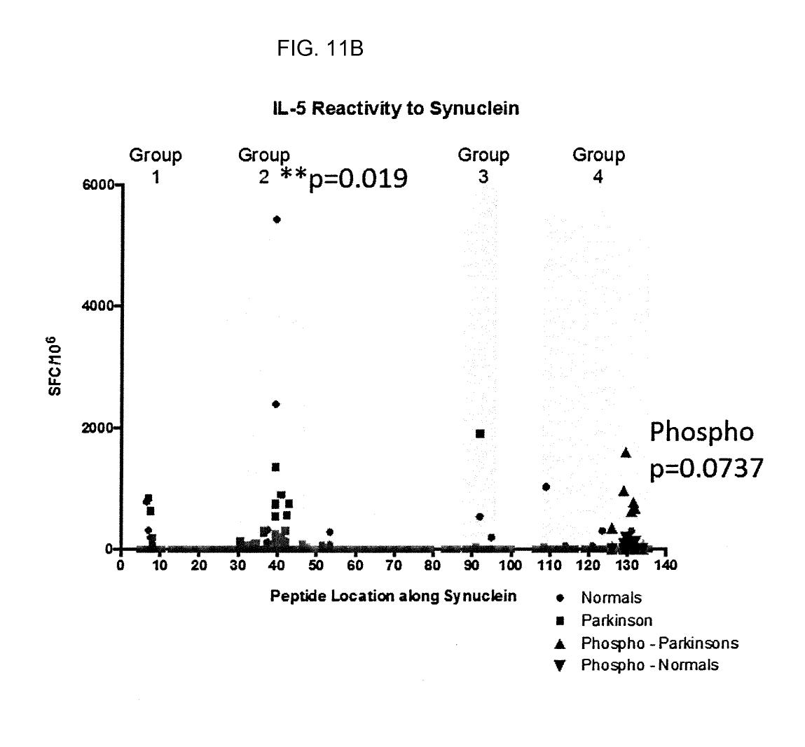

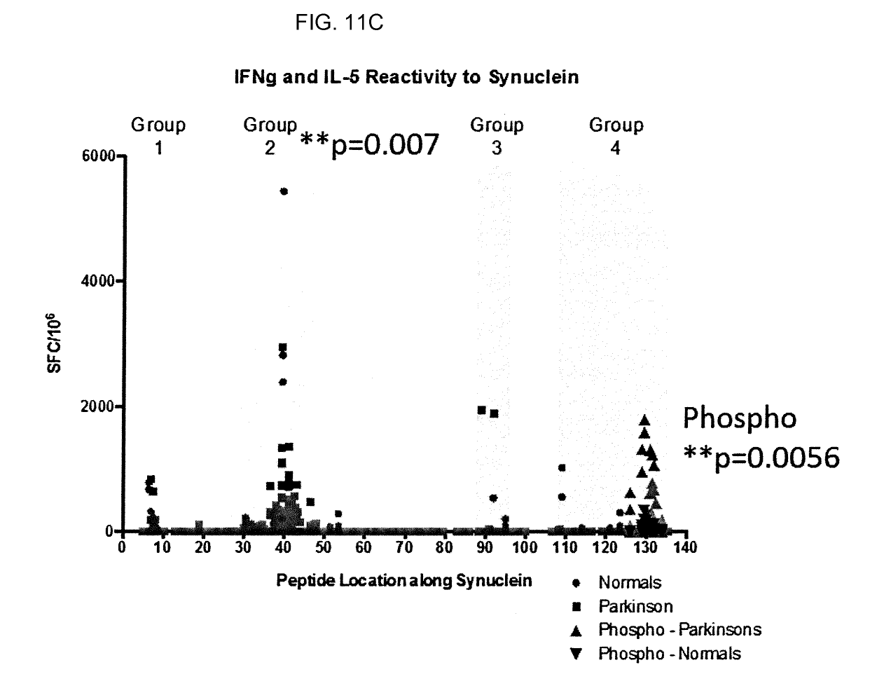

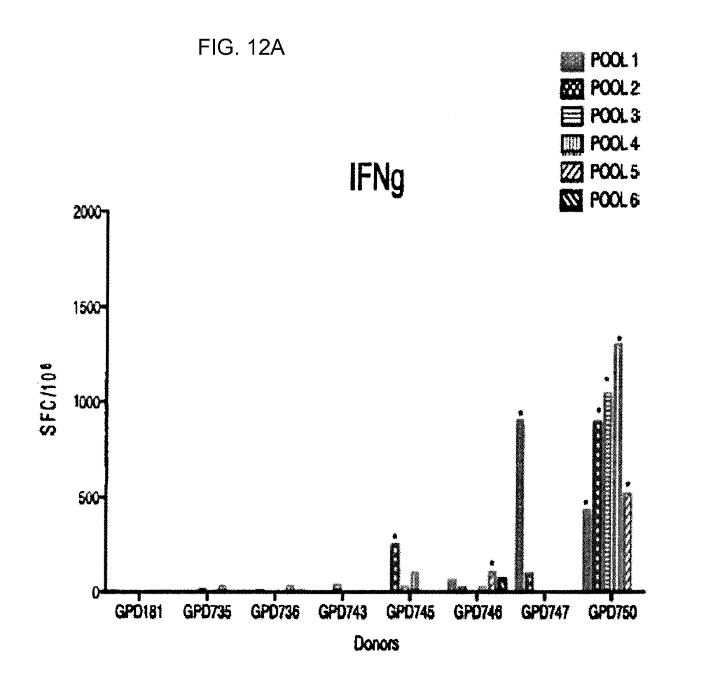

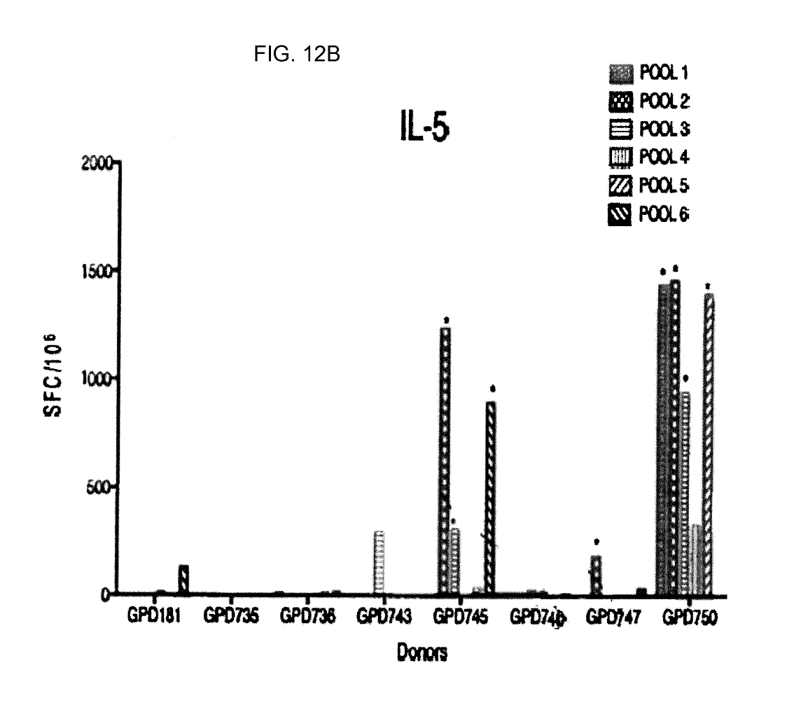

Preliminary control experiments in which the primary antibodies were omitted or HLA (A, B and C) and β2m antibodies were pre-absorbed with the corresponding neutralizing peptide (293T cell lysate transfected with the sequence corresponding to the HLA epitope shared by HLA A, B and C and human full-length β2m peptide respectively), were performed in order to confirm the specificity of the primary antibodies (FIG. 10A).

The HLA (A, B and C) and β2m primary antibodies produced a pattern of label similar to well established MHC-I⁺ structures in human. The immunolabel of blood vessels within the same sections was used as an internal positive control to confirm that the labeling techniques were successful. As the pre-adsorption control does not prove that the monoclonal antibody does not bind another protein, but rather that it binds to the protein of interest, additional controls for HLA and β2m primary antibodies were carried out. From one control brain, the SN of the opposite hemisphere that was processed for immunostaining was homogenated for western blots. Both antibodies produced a single, clear band that matched with the appropriate molecular weight (46 KDa for HLA and 12 KDa for β2m, see FIG. 10 B, C). As an additional control for CD8⁺ antibody, human spleen (paraffin sections obtained from Cell Marque) was used to test specificity of this antibody, as well as absence of labeling when omitting the primary antibody (FIG. 10 D).

Sections designated for immunohistochemistry were incubated with a biotinylated secondary antibody (1:200, VectorLabs) followed by avidin-biotin complex (VectorLabs); V-VIP was used as the chromogen (VectorLabs). Sections designated for immunofluorescence were incubated with secondary fluorescent antibodies (Alexa Fluor-488 or Alexa Fluor-594, 1:400, Invitrogen) and mounted with a water-based medium. Images were captured with a Leica SP5 microscope. There was no fluorescence bleedthrough of secondary antibodies under the conditions used. Counting of NM⁺, TH⁺ and HLA⁺ neurons was performed in three sections per patient (n=8 or 9 patients per condition) using a fluorescent microscope at 20× magnification. Quantification was based on the absolute presence of HLA and rated as positive or negative.

Electron Microscopy

Immunoelectron microscopy experiments were carried out on tissue blocks fixed in 4% paraformaldehyde/0.25% glutaraldehyde in cacodylate buffer (0.12 M, pH 7.4) and embedded in LRW resin. Ultrathin sections were incubated with an HLA (A, B and C) antibody (1:200, Santa Cruz Biotechnologies) and then with a gold particle (15 nm)-conjugated secondary antibody (1:100, British Biocell International). Micrographs were acquired by a transmission electron microscope LEO 912 (Advanced Light and Electron Microscopy BioImaging Center—San Raffaele Scientific Institute).

Mass Spectrometry

MHC-I peptides were identified by mass spectrometry in NM pigment isolated from SN, organelles containing NM, and NM separated from organelles obtained as reported (Zecca et al., 2008). Digested peptides were separated by two-dimensional micro-liquid chromatography coupled to an ion trap mass spectrometer (2DC-MS/MS). The SEQUEST database search algorithm was used to match experimental spectra to peptide sequences in the database. Peptide search was executed against an updated non-redundant human protein sequence database and MHC-I isoform (HLA gene) database from National Center for Biotechnology Information (NCBI) (www.ncbi.nlm.nih.gov). Laser capture microdissection and bioinformatics analysis Affymetrix CEL files for the National Brain Databank data set (NBD) were normalized to “all probe sets” using standard procedures, and scaled to 100 by the MAS5 algorithm implemented in the Bioconductor package (Zheng et al., 2008; Gentleman et al., 2004). Laser-capture microdissection and microarray methods used to generate the NBD data set are described (Simunovic et al., 2009).

Western Blotting

Tissue for western blotting was obtained from the New York Brain Bank frozen, and then rapidly homogenated at 4° C. in lysis buffer containing 50 mM of Tris-HCl, 150 mM of NaCl, 5 mM of EDTA and 1% of Triton-Tx. Protein concentration of the total homogenate was measured using the BCA assay (Thermo Scientific). 60 μg of protein per lane were run on a 10% polyacrylamide gel for HLA antibody and on a 15% polyacrylamide gel for β2m antibody. Proteins were transferred to PVDF membranes (Immobilon, Millipore Corporation, Bedford, Mass., USA). After blocking in 5% dry milk, primary antibodies were applied overnight at 4° C., then rinsed 3 times in Tris-buffered saline containing 0.1% Tween-20 (Fisher Scientific). Peroxidase-labeled secondary antibodies were applied for 1 h at RT, blots rinsed in Tris-buffered saline-Tween as before, developed with enhanced chemiluminescence (Immobilon HRP substrate reagents, Millipore), and then exposed to film (Crystalgen). HLA (1:100, from Santa Cruz Biotechnologies) and β2m (1:100, from Abnova) primary antibodies were used. Actin (1:500,000, from Sigma Aldrich) primary antibody was used as a loading control.

hES Derived DA Neuronal Cultures

hES derived DA neuronal cultures were obtained as described (Kriks et al., 2011). Briefly, H9 human ES cells were subjected to dual SMAD-inhibition followed by exposure to sonic hedgehog, purmorphamine, FGF8 and CHIR99021. Neurons were passaged on day 20, replated on glass-bottom dishes on day 30 at a density of 200.000 cells per 20 μl droplet, and then matured in Neurobasal media with the addition of B27 supplement, GDNF, BDNF, ascorbic acid, cAMP, TGFbeta and DAPT. hES derived spinal motor neuron (MN) cultures expressing EYFP under control of the human synapsin promoter were generated by exposure to dual SMAD-inhibition followed by caudalization with retinoid acid and ventralization through the hedgehog pathway. MN aggregates were replated on day 20 and matured in Neurobasal media with the addition of B27 supplement, GDNF, BDNF, ascorbic acid, cAMP and DAPT.

Cultures were treated with human IFN-γ (eBioscience) at a non-toxic concentration for hES (100 ng/ml, 72h). Cultures were then incubated with the MHC-I antibody prior to fixation in 4% paraformaldehyde and double immunofluorescence for TH and MHC-I as above. Micrographs were acquired using a Leica TCS SP5-II inverted confocal microscope.

Mice

Animal protocols were approved by the Columbia University Institutional Animal Care and Use Committee and in accordance with the National Institutes of Health Guide to the Care and Use of Laboratory Animals guidelines. Mouse lines including wild type C57BL/6, OT-1, OT-2 and B6.129P2-B2mtm1Unc/J mice were maintained under pathogen-free conditions and had access to food and water ad libitum. Each experiment described below was performed at least in triplicate.

Primary Cultures

Primary cultures of microglial cells, SN, VTA, VM (including SN and VTA), LC, cortical, striatal and thalamic neurons were derived from brains of newborn C57/516 or B6.129P2-B2m^{tm1Unc}/J mice (0-3 days, either sex)(Sulzer et al., 2008). B6.129P2-B2m^{tm1Unc}/J mice were generated by a targeted disruption of the β2m gene, and have little or no MHC-I protein expression on the cell surface⁶⁸. Briefly, for microglial cultures cells dissociated from 5 cortexes of 0-3 days old C57/B16 mice were seeded onto 75 cm² cell culture flasks in microglial culture medium (MEM supplemented with calf serum, glucose 45%, penicillin/streptomycin, insulin (25 mg/ml) and 200 mM glutamine) in a 5% CO² incubator at 37° C. (Sulzer et al., 2008). Two weeks later, microglia were detached from flasks by mild shaking and plated on 12 well-plates at a density of 45,000-50,000 microglial cells/well. For neuronal cultures, the area of interest was dissected and neurons were dissociated and plated at a density of 80,000/cm′ onto a layer of rat cortical glial cells grown on round glass coverslips and maintained in culture medium without added GDNF (glial cell line-derived neurotrophic factor) in a 5% CO² incubator at 37° C. for 5-9 days)(Sulzer et al., 2008).

Primary DCs were obtained from mouse (either sex) femur hematopoietic stem cells. In brief, mouse femur hematopoietic stem cells from bone marrow were harvested from the hind legs of 8 to 12-week old male wild-type C57BL/6J and plated at 2×10⁶ cells/ml density in DC culture medium (DMEM supplemented with 10% FBS, lx non-essential amino acids, 2 mM L-glutamine, 1 mM sodium pyruvate and 20 mM HEPES). For differentiation to dendritic cells, 10 ng/ml of recombinant mouse GM-CSF was added to media. Cells were fed every 2 days with fresh DMEM containing GM-CSF, and the cultures were trypsinized after 8 days and plated at 4×10⁵ cells/cm² density to be used for experiments the following day (Tallóczy et al., 2008). OT-1 CD8⁺ and OT-2 CD4⁺ T cell lines, a kind gift from Dr. Raphael Clynes (Department of Medicine and Microbiology, Columbia University Medical Center), express a transgene encoding T cell receptor that specifically recognizes SIINFEKL (SEQ ID NO: 9) peptide (derived from OVA) bound to MHC-I H-2Kb or to MHC-II I-A/I-E (Hogquist et al., 1994). Activated primary OT-1 and OT-2 T cells were generated by the addition of SIINFEKL (SEQ ID NO: 9) to the cultures (Harris et al., 2012). Briefly, OT-1/OT-2 mouse spleens were dissected and homogenized, the released cells were pelleted and resuspended in 5 ml ACK buffer (0.15 M NH4Cl, 1 mM KHCO3, and 0.1 mM EDTA) for 1 min to lyse red blood cells, and the splenocytes were pelleted, washed, resuspended at 5×10⁶ cells/ml in OT-1 growth medium containing 0.75 μg/ml SIINFEKL (SEQ ID NO: 9) peptide, and incubated at 37° C. in a 95% air/5% CO′ humidified atmosphere. On days 3 and 5, 25 ml of fresh OT-1/OT-2 growth medium containing 10 U/ml of mouse recombinant IL-2 was added to the cultures. On day 7, the cells were harvested and OT-1/OT-2 cells were purified by centrifugation at 400 g for 30 min at room temperature over a Histopaque gradient (density=1.083) (Budhu et al., 2010).

Cellular Treatments

IFN-γ (R&D Systems) was used at a range of non-toxic concentrations for neurons or OT-1 cells (10 μg/ml-100 ng/ml, 72h). 12-well cell culture plates were pre-treated with LPS (500 ng/ml, Sigma-Aldrich), NM (5 μg/ml), α-syn, Nit-α-syn or Mut-α-syn (200 μg/ml, kindly provided by Peter Lansbury, Harvard University) for 72 h before plating 45,000-50,000 microglial cells/well for an additional 24h. A neutralizing IFN-γ antibody (1:500, PBL InterferonSource) was used to block IFN-γ released from microglia. VM neurons were incubated for 72 h with microglial medium pre-stimulated by LPS, NM, α-syn, Nit-α-syn or Mut-α-syn. As a control experiment, we directly incubated VM neurons with the same compounds in the absence of microglia. In another set of experiments, VM, cortical and striatal primary neurons were incubated for 72 h with microglial medium pre-stimulated by LPS, NM, α-syn. NM was induced in SN cultured neurons by using L-DOPA (Sulzer et al., 2000) (Sigma Aldrich). For foreign protein processing and subsequent antigen presentation, VM cultures were maintained for 7 days in either 1) BSA/serum-free neuronal medium, 2) BSA/serum-free supplemented with OVA (2.5 mg/ml), adding IFN-γ (100 ng/ml) or saline for an additional 72h.

ELISA

The concentration of IFN-γ released by microglia (plated as above) was measured by an ELISA kit (eBioscience) that detected the amount of this proinflammatory cytokine in a range of 0.7-100 μg/ml.

Immunofluorescence

Cell cultures were double-labeled for MHC-I using a specific antibody raised against the H-2K^{b} and H-2D^{b} class I alloantigens (mouse monoclonal, 1:100, BD Biosciences, catalogue number: 553575), MHC-II (by using an mouse monoclonal antibody that reacts with a polymorphic determinant shared by the I-A[b], I-A[d], I-A[q], I-E[d], and I-E[k] MHC-II alloantigens, 1:100, BD Biosciences, catalogue number: 556999) or SIINFEKL-MHC-I (mouse monoclonal, 1:100, eBioscience, catalogue number 14-5743-81) and TH (rabbit polyclonal, 1:2000, Millipore, catalogue number: AB152) or MAP-2 (rabbit polyclonal, 1:1000, Millipore, catalogue number: AB5622), fixed with freshly prepared 4% paraformaldehyde (Electron Microscopy Sciences), blocked with normal serum (Jackson Immunoresearch) and incubated with fluorescent secondary antibodies (Alexa Fluor-594 or Alexa Fluor-488, 1:400, Invitrogen). Images were captured using a Leica SP5 microscope system. There was no fluorescence bleed-through of secondary antibodies under the conditions used. MHC-I label observed in the neuronal cultures obtained from wild type mice was absent in neurons from β2m KO mice (FIG. 3A).

In order to assess the proportion of TH⁺ and MAP-2⁺ neurons that expressed MHC-I⁺, first TH⁺ and MAP-2⁺ neurons were counted in 24 fields surrounding the border of the well at 20× magnification. The number of TH⁺ or MAP-2⁺ neurons with intact soma and two or more neurites equal to or greater than the diameter of the cell soma was counted. Then, the fluorescent filter was inserted for each field to determine how many of these neurons also expressed MHC-I.

To further analyze the specificity of the MHC-I antibody, a western blot was run of wild type versus β2m KO brain tissue (see western blot paragraph below for detailed method). A single band (˜30 KDa) was observed in lanes loaded with brain tissue from wild type mice, whereas there were no visible bands in the lanes that contained brain tissue from β2m KO. A positive control (mouse spleen homogenate) displayed a band at the same molecular weight (FIG. 10E).

Western Blotting

The brains of 3 C57/BL6 and 3 B6.129P2-B2m^{tm1Unc}/J were dissected on ice and then rapidly homogenated at 4° C. in lysis buffer containing 50 mM of Tris-HCl, 150 mM of NaCl, 5 mM of EDTA and 1% of Triton-Tx. Protein concentration of the total homogenate was measured using the BCA assay (Thermo Scientific). 20 μg of protein per lane were run on a 12% polyacrylamide gel. Proteins were transferred to PVDF membranes (Immobilon, Millipore Corporation, Bedford, Mass., USA). After blocking in 5% dry milk, primary antibody (MHC-I antibody raised against the H-2K^{b} and H-2D^{b} class I alloantigens, mouse monoclonal, 1:100, BD Biosciences), was applied overnight at 4° C., then rinsed 3 times in Tris-buffered saline containing 0.1% Tween-20 (Fisher Scientific). Peroxidase-labeled secondary antibodies were applied for 1 h at RT, blots rinsed in Tris-buffered saline-tween as before, developed with enhanced chemiluminescence (Immobilon HRP substrate reagents, Millipore), and then exposed to film (Crystalgen). Actin (1:500,000, from Sigma Aldrich) primary antibody was used as a loading control. 20 μg of mouse spleen homogenate was loaded as a positive control for the MHC-I antibody.

Proliferation and Killing Assays

OT-1 CD8⁺ cell proliferation was assessed by BrdU incorporation. For OT-1 T cell cytotoxicity assays, DC and VM (from C57BL/6 and 56.129P2-B2mtm1Unc/J mice) cell cultures were incubated with neuronal medium or neuronal medium containing 100 ng/ml of IFN-γ (R&D Systems). Neurons were loaded with or without SIINFEKL (SEQ ID NO: 9) peptide (1 μM), and, subsequently in vitro activated OT-1 cells were added at a density of 100,000 cells/well and co-incubated for 24 h.

We used the same conditions for VM cell cultures with microglial medium previously stimulated with LPS, NM or α-syn instead of IFN-γ. Neurons were pre-incubated with IFN-γ and SIINFEKL (SEQ ID NO: 9) peptide as above, and then with: 1) a pan-caspase general inhibitor (Z-VAD-FMK, 20 μM, 24h, IMGENEX); 2) a specific granzyme/perforin inhibitor (concanamycin A, 100 nM, 24h, Sigma Aldrich); 3) a specific Fas/FasL antagonist (Kp7-6, 1 mM, 24h, EMD Biosciences) and 4) concanamycin A and Kp7-6 together. OT-1 cells were then added to the culture (100,000 cells/well) and maintained for 24 h.

Following immunocytochemical staining of VM neurons, we assessed the proportion of surviving DA (TH⁺) and non-DA cells (MAP-2⁺, TH⁻). Total numbers of TH⁺ and MAP-2⁺ neurons were counted in 24 fields surrounding the border of the well at 20× magnification. The number of surviving TH+ neurons with an intact soma and two or more neurites equal to or greater than the diameter of the cell soma was counted and normalized to the number in untreated cultures.

Statistics

Results are presented as mean±standard error of the mean (SEM). Each experiment was repeated at least 3 times, and each condition was also examined in three subjects or cultures within each experiment. Two-way ANOVA was used to analyze the IFN-γ dose-response experiments. Two-tailed Student's T-test (or Man Whitney U test for non-parametric cases) or one-way ANOVA with Tukey post-hoc tests were used to analyze the other experiments.

### Example 9. Cytokine Release in Controls and PD Patients

Blood from age matched controls and PD patients were obtained and mononuclear cells were isolated by gradient centrifugation.

Release of the cytokines gamma-interferon, which measures activation of CD4+ and/or CD8+ T cells, and the interleukin, IL-5, which measures activation of CD4+ T cells was measured by ELISpot assay. Briefly, the isolated cells were plated in wells that have colorimetric detection of gamma-interferon and IL-5, and were stimulated with pools of 95 epitopes of a-synuclein that the Sette lab determined would potentially be displayed by MHC-I or MHC-II antigen-presenting proteins in humans. After 22 hours of stimulation at 37 C, the cells were removed and release of cytokines was measured by colorimetric detection of spot-forming cells (SFC). Confirmed release of cytokine is determined by the presence of a minimum of 20 SFC per million cells.

Cytokine release was detected in only 15 of 160 stimulations to a-synuclein epitopes across controls (n=12), while 43 of 248 stimulations in PD patients had antigenic responses, yielding a probability of p=0.029 that PD patients are a different population (two-tailed Fisher exact test). Thus, the data shows that PD patients are more likely to have T cells in blood that recognize and are activated by a-synuclein than unaffected individuals.

TABLE 3

| | | | | Patients Summary | | | | | | | | | | | | | | | |
|---|---|---|---|---|---|---|---|---|---|---|---|---|---|---|---|---|---|---|---|
| | | | | Pool Stimulated | | | | | | | | | | | | | | | |
| | | | | IFNg | | | | | | | | IL-g | | | | | | | |
| | | | | | | | Donors | | | | Res- | | | | Donors | | | | Re- |
| | | | | | | | test | Pep- | | | ponse | | | | test | Pep- | | | sponse |
| | Pooling | | Sequence | Donors | Pool | SFC | (pep- | tide | Total | Avg | Fre- | Donors | Pool | SFC | (pep- | tide | Total | Avg | Fre- |
| Peptide ID | sort | Pool | SEQ ID NO | tested | + | Pool | tide) | + | SFC | SFC | quency | tested | + | Pool | tide) | + | SFC | SFC | quency |
| 3521.001 | 1 | 1 | VFMKGLSKA | 19 | 2 | 1340 | 3 | 1 | 75 | 75 | 33% | 19 | 2 | 1907 | 3 | 1 | 634 | 634 | 33% |
| | | | SEQ ID NO: 10 | | | | | | | | | | | | | | | | |
| 3521.0012 | 2 | 1 | KTKQGVAEA | 19 | 2 | 1340 | 3 | 0 | 0 | | 0% | 19 | 2 | 1907 | 3 | 0 | 0 | | 0% |
| | | | SEQ ID NO: 81 | | | | | | | | | | | | | | | | |
| 3521.0016 | 3 | 1 | DVFMKGLSK | 19 | 2 | 1340 | 3 | 0 | 0 | | 0% | 19 | 2 | 1907 | 3 | 0 | 0 | | 0% |
| | | | SEQ ID NO: 82 | | | | | | | | | | | | | | | | |
| 3521.0017 | 4 | 1 | FMKGLSKAK | 19 | 2 | 1340 | 3 | 0 | 0 | | 0% | 19 | 2 | 1907 | 3 | 0 | 0 | | 0% |
| | | | SEQ ID NO: 83 | | | | | | | | | | | | | | | | |
| 3521.0039 | 5 | 1 | DVFMKGLSKA | 19 | 2 | 1340 | 3 | 1 | 189 | 189 | 33% | 19 | 2 | 1907 | 3 | 1 | 838 | 838 | 33% |
| | | | SEQ ID NO: 11 | | | | | | | | | | | | | | | | |
| 3521.0043 | 6 | 1 | VFMKGLSKAK | 19 | 2 | 1340 | 3 | 0 | 0 | | 0% | 19 | 2 | 1907 | 3 | 1 | 191 | 191 | 33% |
| | | | SEQ ID NO: 44 | | | | | | | | | | | | | | | | |
| 3521.0046 | 7 | 1 | FMKGLSKAKE | 19 | 2 | 1340 | 3 | 0 | 0 | | 0% | 19 | 2 | 1907 | 3 | 0 | 0 | | 0% |
| | | | SEQ ID NO: 84 | | | | | | | | | | | | | | | | |
| 3521.0052 | 8 | 1 | KTKQGVAEAA | 19 | 2 | 1340 | 3 | 0 | 0 | | 0% | 19 | 2 | 1907 | 3 | 0 | 0 | | 0% |
| | | | SEQ ID NO: 85 | | | | | | | | | | | | | | | | |
| 3521.0053 | 9 | 1 | KAKEGVVAAA | 19 | 2 | 1340 | 3 | 0 | 0 | | 0% | 19 | 2 | 1907 | 3 | 0 | 0 | | 0% |
| | | | SEQ ID NO: 86 | | | | | | | | | | | | | | | | |
| 3521.0066 | 10 | 1 | KAKEGVVAA | 19 | 2 | 1340 | 3 | 0 | 0 | | 0% | 19 | 2 | 1907 | 3 | 0 | 0 | | 0% |
| | | | SEQ ID NO: 87 | | | | | | | | | | | | | | | | |
| 3521.007 | 11 | 1 | GVVAAAEKTK | 19 | 2 | 1340 | 3 | 2 | 225 | 113 | 67% | 19 | 2 | 1907 | 3 | 1 | 23 | 23 | 33% |
| | | | SEQ ID NO: 12 | | | | | | | | | | | | | | | | |
| 3521.0074 | 12 | 1 | VAAAEKTKQGVAEAP | 19 | 2 | 1340 | 3 | 1 | 67 | 67 | 33% | 19 | 2 | 1907 | 3 | 0 | 0 | | 0% |
| | | | SEQ ID NO: 13 | | | | | | | | | | | | | | | | |
| 3521.008 | 13 | 1 | VAAAEKTKQGVAEAA | 19 | 2 | 1340 | 3 | 1 | 150 | 150 | 33% | 19 | 2 | 1907 | 3 | 0 | 0 | | 0% |
| | | | SEQ ID NO: 14 | | | | | | | | | | | | | | | | |
| 3521.0004 | 14 | 2 | KTKEGVLYV | 19 | 8 | 8206 | 8 | 0 | 0 | | 0% | 19 | 6 | 3908 | 8 | 0 | 0 | | 0% |
| | | | SEQ ID NO: 88 | | | | | | | | | | | | | | | | |
| 3521.0019 | 15 | 2 | APGKTKEGV | 19 | 8 | 8206 | 8 | 0 | 0 | | 0% | 19 | 6 | 3908 | 8 | 0 | 0 | | 0% |
| | | | SEQ ID NO: 89 | | | | | | | | | | | | | | | | |
| 3521.002 | 16 | 2 | AGKTKEGVL | 19 | 8 | 8206 | 8 | 1 | 98 | 98 | 13% | 19 | 6 | 3908 | 8 | 1 | 106 | 106 | 13% |
| | | | SEQ ID NO: 15 | | | | | | | | | | | | | | | | |
| 3521.0021 | 17 | 2 | PGKTKEGVL | 19 | 8 | 8206 | 8 | 1 | 36 | 36 | 13% | 19 | 6 | 3908 | 8 | 0 | 0 | | 0% |
| | | | SEQ ID NO: 16 | | | | | | | | | | | | | | | | |
| 3521.0042 | 18 | 2 | AGKTKEGVLY | 19 | 8 | 8206 | 8 | 1 | 31 | 31 | 13% | 19 | 6 | 3908 | 8 | 0 | 0 | | 0% |
| | | | SEQ ID NO: 17 | | | | | | | | | | | | | | | | |
| 3521.005 | 19 | 2 | APGKTKEGVL | 19 | 8 | 8206 | 8 | 1 | 44 | 44 | 13% | 19 | 6 | 3908 | 8 | 2 | 137 | 67.5 | 25% |
| | | | SEQ ID NO: 18 | | | | | | | | | | | | | | | | |
| 3521.0059 | 20 | 2 | AEAAGKTKEG | 19 | 8 | 8206 | 8 | 0 | 0 | | 0% | 19 | 6 | 3908 | 8 | 1 | 71 | 71 | 13% |
| | | | SEQ ID NO: 45 | | | | | | | | | | | | | | | | |
| 3521.0068 | 21 | 2 | GVALAPGKTK | 19 | 8 | 8206 | 8 | 0 | 0 | | 0% | 19 | 6 | 3908 | 8 | 0 | 0 | | 0% |
| | | | SEQ ID NO: 90 | | | | | | | | | | | | | | | | |
| 3521.0069 | 22 | 2 | GVALAAGKTK | 19 | 8 | 8206 | 8 | 1 | 53 | 53 | 13% | 19 | 6 | 3908 | 8 | 0 | 0 | | 0% |
| | | | SEQ ID NO: 19 | | | | | | | | | | | | | | | | |
| 3521.0075 | 23 | 2 | KQGVAEAPGKTKEGV | 19 | 8 | 8206 | 8 | 3 | 209 | 70 | 38% | 19 | 6 | 3908 | 8 | 1 | 58 | 58 | 13% |
| | | | SEQ ID NO: 20 | | | | | | | | | | | | | | | | |
| 3521.0076 | 24 | 2 | PGKTKEGVLYVGSKT | 19 | 8 | 8206 | 8 | 2 | 80 | 40 | 25% | 19 | 6 | 3908 | 8 | 1 | 89 | 89 | 13% |
| | | | SEQ ID NO: 21 | | | | | | | | | | | | | | | | |
| 3521.0077 | 25 | 2 | KTKEGVLYVGSKTKK | 19 | 8 | 8206 | 8 | 5 | 3505 | 701 | 63% | 19 | 6 | 3908 | 8 | 6 | 3565 | 595 | 75% |
| | | | SEQ ID NO: 22 | | | | | | | | | | | | | | | | |
| 3521.0081 | 26 | 2 | KQGVAEAAGKTKEGV | 19 | 8 | 8206 | 8 | 2 | 167 | 83 | 25% | 19 | 6 | 3908 | 8 | 2 | 186 | 93 | 25% |
| | | | SEQ ID NO: 23 | | | | | | | | | | | | | | | | |
| 3521.0082 | 27 | 2 | AGKTKEGVLYVGSKT | 19 | 8 | 8206 | 8 | 1 | 27 | 27 | 13% | 19 | 6 | 3908 | 8 | 0 | 0 | | 0% |
| | | | SEQ ID NO: 24 | | | | | | | | | | | | | | | | |
| 3521.0006 | 28 | 3 | VLYVGSKTK | 19 | 6 | 6068 | 6 | 5 | 1519 | 304 | 83% | 19 | 3 | 1526 | 6 | 2 | 168 | 84 | 33% |
| | | | SEQ ID NO: 25 | | | | | | | | | | | | | | | | |
| 3521.0009 | 29 | 3 | LYVGSKTKK | 19 | 6 | 6068 | 6 | 2 | 326 | 163 | 33% | 19 | 3 | 1526 | 6 | 1 | 559 | 559 | 17% |
| | | | SEQ ID NO: 26 | | | | | | | | | | | | | | | | |
| 3521.0031 | 30 | 3 | YVGSKTKEGV | 19 | 6 | 6068 | 6 | 1 | 153 | 153 | 17% | 19 | 3 | 1526 | 6 | 0 | 0 | | 0% |
| | | | SEQ ID NO: 27 | | | | | | | | | | | | | | | | |
| 3521.0033 | 31 | 3 | VLYVGSKTEK | 19 | 6 | 6068 | 6 | 5 | 1626 | 325 | 83% | 19 | 3 | 1526 | 6 | 3 | 528 | 176 | 50% |
| | | | SEQ ID NO: 28 | | | | | | | | | | | | | | | | |
| 3521.0034 | 32 | 3 | GVLYVGSKTK | 19 | 6 | 6068 | 6 | 5 | 2260 | 452 | 83% | 19 | 3 | 1526 | 6 | 2 | 284 | 142 | 33% |
| | | | SEQ ID NO: 29 | | | | | | | | | | | | | | | | |
| 3521.0037 | 33 | 3 | LYVGSKTKEG | 19 | 6 | 6068 | 6 | 1 | 248 | 248 | 17% | 19 | 3 | 1526 | 6 | 1 | 754 | 754 | 17% |
| | | | SEQ ID NO: 30 | | | | | | | | | | | | | | | | |
| 3521.0045 | 34 | 3 | KTKKGVVHGV | 19 | 6 | 6068 | 6 | 1 | 133 | 133 | 17% | 19 | 3 | 1526 | 6 | 0 | 0 | | 0% |
| | | | SEQ ID NO: 31 | | | | | | | | | | | | | | | | |
| 3521.0062 | 35 | 3 | KTKKGVVHG | 19 | 6 | 6068 | 6 | 1 | 113 | 113 | 17% | 19 | 3 | 1526 | 6 | 1 | 40 | 40 | 17% |
| | | | SEQ ID NO: 32 | | | | | | | | | | | | | | | | |
| 3521.0067 | 36 | 3 | KTKEGVVHG | 19 | 6 | 6068 | 6 | 0 | 0 | | 0% | 19 | 3 | 1526 | 6 | 0 | 0 | | 0% |
| | | | SEQ ID NO: 91 | | | | | | | | | | | | | | | | |
| 3521.0071 | 37 | 3 | YVGSKTKEGVVHGVT | 19 | 6 | 6068 | 6 | 0 | 0 | | 0% | 19 | 3 | 1526 | 6 | 1 | 35 | 35 | 17% |
| | | | SEQ ID NO: 46 | | | | | | | | | | | | | | | | |
| 3521.0078 | 38 | 3 | YVGSKTKKGVVHGVA | 19 | 6 | 6068 | 6 | 1 | 474 | 474 | 17% | 19 | 3 | 1526 | 6 | 1 | 93 | 93 | 17% |
| | | | SEQ ID NO: 33 | | | | | | | | | | | | | | | | |
| 3521.0083 | 39 | 3 | KTKEGVLYVGSKTKE | 19 | 6 | 6068 | 6 | 5 | 5544 | 1109 | 83% | 19 | 3 | 1526 | 6 | 5 | 1933 | 387 | 83% |
| | | | SEQ ID NO: 34 | | | | | | | | | | | | | | | | |
| 3521.0084 | 40 | 3 | YVGSKTKEGVVHGVA | 19 | 6 | 6068 | 6 | 0 | 0 | | 0% | 19 | 3 | 1526 | 6 | 0 | 0 | | 0% |
| | | | SEQ ID NO: 92 | | | | | | | | | | | | | | | | |
| 3521.0005 | 41 | 4 | AVVTGVTAV | 19 | 3 | 2587 | 2 | 0 | 0 | | 0% | 19 | 1 | 327 | 2 | 0 | 0 | | 0% |
| | | | SEQ ID NO: 93 | | | | | | | | | | | | | | | | |
| 3521.0011 | 42 | 4 | QVTNVGGAVV | 19 | 3 | 2587 | 2 | 0 | 0 | | 0% | 19 | 1 | 327 | 2 | 0 | 0 | | 0% |
| | | | SEQ ID NO: 94 | | | | | | | | | | | | | | | | |
| 3521.0013 | 43 | 4 | VTNVGGAVV | 19 | 3 | 2587 | 2 | 1 | 209 | 209 | 50% | 19 | 1 | 327 | 2 | 0 | 0 | | 0% |
| | | | SEQ ID NO: 35 | | | | | | | | | | | | | | | | |
| 3521.0015 | 44 | 4 | KTKEQVTNV | 19 | 3 | 2587 | 2 | 0 | 0 | | 0% | 19 | 1 | 327 | 2 | 0 | 0 | | 0% |
| | | | SEQ ID NO: 95 | | | | | | | | | | | | | | | | |
| 3521.0029 | 45 | 4 | NVGGAVVTGV | 19 | 3 | 2587 | 2 | 0 | 0 | | 0% | 19 | 1 | 327 | 2 | 0 | 0 | | 0% |
| | | | SEQ ID NO: 96 | | | | | | | | | | | | | | | | |
| 3521.0032 | 46 | 4 | GAVVTGVTAV | 19 | 3 | 2587 | 2 | 0 | 0 | | 0% | 19 | 1 | 327 | 2 | 0 | 0 | | 0% |
| | | | SEQ ID NO: 97 | | | | | | | | | | | | | | | | |
| 3521.0044 | 47 | 4 | GVTAVAQKTV | 19 | 3 | 2587 | 2 | 0 | 0 | | 0% | 19 | 1 | 327 | 2 | 0 | 0 | | 0% |
| | | | SEQ ID NO: 98 | | | | | | | | | | | | | | | | |
| 3521.0048 | 48 | 4 | EQVTNVGGAV | 19 | 3 | 2587 | 2 | 0 | 0 | | 0% | 19 | 1 | 327 | 2 | 0 | 0 | | 0% |
| | | | SEQ ID NO: 99 | | | | | | | | | | | | | | | | |
| 3521.006 | 49 | 4 | VATVAEKTKE | 19 | 3 | 2587 | 2 | 0 | 0 | | 0% | 19 | 1 | 327 | 2 | 0 | 0 | | 0% |
| | | | SEQ ID NO: 100 | | | | | | | | | | | | | | | | |
| 3521.0063 | 50 | 4 | GVVHGVTTV | 19 | 3 | 2587 | 2 | 1 | 143 | 143 | 50% | 19 | 1 | 327 | 2 | 0 | 0 | | 0% |
| | | | SEQ ID NO: 36 | | | | | | | | | | | | | | | | |
| 3521.0064 | 51 | 4 | GVVHGVATV | 19 | 3 | 2587 | 2 | 0 | 0 | | 0% | 19 | 1 | 327 | 2 | 0 | 0 | | 0% |
| | | | SEQ ID NO: 101 | | | | | | | | | | | | | | | | |
| 3521.0072 | 52 | 4 | EGVVHGTTVAEKTK | 19 | 3 | 2587 | 2 | 0 | 0 | | 0% | 19 | 1 | 327 | 2 | 0 | 0 | | 0% |
| | | | SEQ ID NO: 102 | | | | | | | | | | | | | | | | |
| 3521.0073 | 53 | 4 | TTVAEKTKEQVTNVG | 19 | 3 | 2587 | 2 | 0 | 0 | | 0% | 19 | 1 | 327 | 2 | 0 | 0 | | 0% |
| | | | SEQ ID NO: 103 | | | | | | | | | | | | | | | | |
| 3521.0079 | 54 | 4 | KGVVHGVATVAEKTK | 19 | 3 | 2587 | 2 | 0 | 0 | | 0% | 19 | 1 | 327 | 2 | 0 | 0 | | 0% |
| | | | SEQ ID NO: 104 | | | | | | | | | | | | | | | | |
| 3521.0085 | 55 | 4 | EGVVHGVATVAEKTK | 19 | 3 | 2587 | 2 | 0 | 0 | | 0% | 19 | 1 | 327 | 2 | 0 | 0 | | 0% |
| | | | SEQ ID NO: 105 | | | | | | | | | | | | | | | | |
| 3521.0086 | 56 | 4 | ATVAEKTKEQVTNVG | 19 | 3 | 2587 | 2 | 0 | 0 | | 0% | 19 | 1 | 327 | 2 | 0 | 0 | | 0% |
| | | | SEQ ID NO: 106 | | | | | | | | | | | | | | | | |
| 3521.0003 | 57 | 5 | SIAAATGFV | 19 | 3 | 2670 | 4 | 0 | 0 | | 0% | 19 | 2 | 1670 | 4 | 0 | 0 | | 0% |
| | | | SEQ ID NO: 107 | | | | | | | | | | | | | | | | |
| 3521.0007 | 58 | 5 | AAATGFVKK | 19 | 3 | 2670 | 4 | 0 | 0 | | 0% | 19 | 2 | 1670 | 4 | 0 | 0 | | 0% |
| | | | SEQ ID NO: 108 | | | | | | | | | | | | | | | | |
| 3521.0008 | 59 | 5 | IAAATGFVK | 19 | 3 | 2670 | 4 | 0 | 0 | | 0% | 19 | 2 | 1670 | 4 | 1 | 177 | 177 | 25% |
| | | | SEQ ID NO: 47 | | | | | | | | | | | | | | | | |
| 3521.0014 | 60 | 5 | KTVEGAGSI | 19 | 3 | 2670 | 4 | 0 | 0 | | 0% | 19 | 2 | 1670 | 4 | 0 | 0 | | 0% |
| | | | SEQ ID NO: 109 | | | | | | | | | | | | | | | | |
| 3521.0022 | 61 | 5 | GSIAAATGF | 19 | 3 | 2670 | 4 | 0 | 0 | | 0% | 19 | 2 | 1670 | 4 | 0 | 0 | | 0% |
| | | | SEQ ID NO: 110 | | | | | | | | | | | | | | | | |
| 3521.0025 | 62 | 5 | EEGAPQEGI | 19 | 3 | 2670 | 4 | 1 | 290 | 290 | 25% | 19 | 2 | 1670 | 4 | 2 | 172 | 86 | 50% |
| | | | SEQ ID NO: 37 | | | | | | | | | | | | | | | | |
| 3521.003 | 63 | 5 | GSIAAATGFV | 19 | 3 | 2670 | 4 | 0 | 0 | | 0% | 19 | 2 | 1670 | 4 | 1 | 40 | 40 | 25% |
| | | | SEQ ID NO: 38 | | | | | | | | | | | | | | | | |
| 3521.0035 | 64 | 5 | SIAAATGFVK | 19 | 3 | 2670 | 4 | 1 | 1946 | 1946 | 25% | 19 | 2 | 1670 | 4 | 1 | 1898 | 1898 | 25% |
| | | | SEQ ID NO: 39 | | | | | | | | | | | | | | | | |
| 3521.0036 | 65 | 5 | AGSIAAATGF | 19 | 3 | 2670 | 4 | 0 | 0 | | 0% | 19 | 2 | 1670 | 4 | 0 | 0 | | 0% |
| | | | SEQ ID NO: 40 | | | | | | | | | | | | | | | | |
| 3521.0047 | 66 | 5 | IAAATGFVKK | 19 | 3 | 2670 | 4 | 0 | 0 | | 0% | 19 | 2 | 1670 | 4 | 0 | 0 | | 0% |
| | | | SEQ ID NO: 41 | | | | | | | | | | | | | | | | |
| 3521.0049 | 67 | 5 | APQEGILEDM | 19 | 3 | 2670 | 4 | 0 | 0 | | 0% | 19 | 2 | 1670 | 4 | 0 | 0 | | 0% |
| | | | SEQ ID NO: 42 | | | | | | | | | | | | | | | | |
| 3521.0051 | 68 | 5 | EEGAPQEGIL | 19 | 3 | 2670 | 4 | 1 | 427 | 427 | 25% | 19 | 2 | 1670 | 4 | 0 | 0 | | 0% |
| | | | SEQ ID NO: 43 | | | | | | | | | | | | | | | | |
| 3521.0057 | 69 | 5 | NEEGAPQEGI | 19 | 3 | 2670 | 4 | 0 | 0 | | 0% | 19 | 2 | 1670 | 4 | 0 | 0 | | 0% |
| | | | SEQ ID NO: 111 | | | | | | | | | | | | | | | | |
| 3521.0061 | 70 | 5 | AATGFVKKDQ | 19 | 3 | 2670 | 4 | 0 | 0 | | 0% | 19 | 2 | 1670 | 4 | 0 | 0 | | 0% |
| | | | SEQ ID NO: 112 | | | | | | | | | | | | | | | | |
| 3521.0065 | 71 | 5 | FVKKDQLGK | 19 | 3 | 2670 | 4 | 0 | 0 | | 0% | 19 | 2 | 1670 | 4 | 0 | 0 | | 0% |
| | | | SEQ ID NO: 113 | | | | | | | | | | | | | | | | |
| 3521.0001 | 72 | 6 | PVDPDNEAY | 19 | 3 | 457 | 2 | 0 | 0 | | 0% | 19 | 2 | 970 | 2 | 0 | 0 | | 0% |
| | | | SEQ ID NO: 114 | | | | | | | | | | | | | | | | |
| 3521.0002 | 73 | 6 | PSEEGYQDY | 19 | 3 | 457 | 2 | 0 | 0 | | 0% | 19 | 2 | 970 | 2 | 0 | 0 | | 0% |
| | | | SEQ ID NO: 49 | | | | | | | | | | | | | | | | |
| 3521.0018 | 74 | 6 | MPVDPDNEA | 19 | 3 | 457 | 2 | 0 | 0 | | 0% | 19 | 2 | 970 | 2 | 0 | 0 | | 0% |
| | | | SEQ ID NO: 115 | | | | | | | | | | | | | | | | |
| 3521.0023 | 75 | 6 | YEMPSEEGY | 19 | 3 | 457 | 2 | 0 | 0 | | 0% | 19 | 2 | 970 | 2 | 0 | 0 | | 0% |
| | | | SEQ ID NO: 50 | | | | | | | | | | | | | | | | |
| 3521.0024 | 76 | 6 | DPPDNEAYEM | 19 | 3 | 457 | 2 | 0 | 0 | | 0% | 19 | 2 | 970 | 2 | 0 | 0 | | 0% |
| | | | SEQ ID NO: 116 | | | | | | | | | | | | | | | | |
| 3521.0026 | 77 | 6 | MPSEEGYQD | 19 | 3 | 457 | 2 | 0 | 0 | | 0% | 19 | 2 | 970 | 2 | 0 | 0 | | 0% |
| | | | SEQ ID NO: 51 | | | | | | | | | | | | | | | | |
| 3521.0027 | 78 | 6 | AYEMPSEEGY | 19 | 3 | 457 | 2 | 0 | 0 | | 0% | 19 | 2 | 970 | 2 | 0 | 0 | | 0% |
| | | | SEQ ID NO: 52 | | | | | | | | | | | | | | | | |
| 3521.0028 | 79 | 6 | MPSEEGYQDY | 19 | 3 | 457 | 2 | 0 | 0 | | 0% | 19 | 2 | 970 | 2 | 0 | 0 | | 0% |
| | | | SEQ ID NO: 53 | | | | | | | | | | | | | | | | |
| 3521.0038 | 80 | 6 | EMPSEEGYQD | 19 | 3 | 457 | 2 | 0 | 0 | | 0% | 19 | 2 | 970 | 2 | 0 | 0 | | 0% |
| | | | SEQ ID NO: 54 | | | | | | | | | | | | | | | | |
| 3521.004 | 81 | 6 | DNEAYEMPSE | 19 | 3 | 457 | 2 | 0 | 0 | | 0% | 19 | 2 | 970 | 2 | 0 | 0 | | 0% |
| | | | SEQ ID NO: 55 | | | | | | | | | | | | | | | | |
| 3521.0041 | 82 | 6 | EGILEDMPVD | 19 | 3 | 457 | 2 | 0 | 0 | | 0% | 19 | 2 | 970 | 2 | 0 | 0 | | 0% |
| | | | SEQ ID NO: 117 | | | | | | | | | | | | | | | | |
| 3521.0054 | 83 | 6 | MPVDPDNEAY | 19 | 3 | 457 | 2 | 0 | 0 | | 0% | 19 | 2 | 970 | 2 | 0 | 0 | | 0% |
| | | | SEQ ID NO: 118 | | | | | | | | | | | | | | | | |
| 3521.0055 | 84 | 6 | QEGILEDMPV | 19 | 3 | 457 | 2 | 0 | 0 | | 0% | 19 | 2 | 970 | 2 | 0 | 0 | | 0% |
| | | | SEQ ID NO: 119 | | | | | | | | | | | | | | | | |
| 3521.0056 | 85 | 6 | YEMPSEEGYQ | 19 | 3 | 457 | 2 | 0 | 0 | | 0% | 19 | 2 | 970 | 2 | 0 | 0 | | 0% |
| | | | SEQ ID NO: 56 | | | | | | | | | | | | | | | | |
| 3521.0058 | 86 | 6 | SEEGYQDYEP | 19 | 3 | 457 | 2 | 0 | 0 | | 0% | 19 | 2 | 970 | 2 | 0 | 0 | | 0% |
| | | | SEQ ID NO: 57 | | | | | | | | | | | | | | | | |
| 2 | 3521.0002 | Phospho | PSEEGYQDY | 10 | 2 | 1660 | 10 | 1 | 460 | 460 | 10% | 10 | 2 | 1026 | 10 | 0 | 0 | | 0% |
| | | | SEQ ID NO: 49 | | | | | | | | | | | | | | | | |
| 23 | 3521.0023 | | YEMPSEEGY | 10 | 2 | 1660 | 10 | 3 | 2039 | 680 | 30% | 10 | 2 | 1026 | 10 | 1 | 1593 | 1593 | 10% |
| | | | SEQ ID NO: 50 | | | | | | | | | | | | | | | | |
| 26 | 3521.0026 | | MPSEEGYQD | 10 | 2 | 1660 | 10 | 4 | 1627 | 407 | 40% | 10 | 2 | 1026 | 10 | 1 | 773 | 773 | 10% |
| | | | SEQ ID NO: 51 | | | | | | | | | | | | | | | | |
| 27 | 3521.0027 | | AYEMPSEEGY | 10 | 2 | 1660 | 10 | 2 | 1400 | 700 | 20% | 10 | 2 | 1026 | 10 | 2 | 1060 | 530 | 20% |
| | | | SEQ ID NO: 52 | | | | | | | | | | | | | | | | |
| 28 | 3521.0028 | | MPSEEGYQDY | 10 | 2 | 1660 | 10 | 3 | 1260 | 420 | 30% | 10 | 2 | 1026 | 10 | 2 | 700.3 | 350 | 20% |
| | | | SEQ ID NO: 53 | | | | | | | | | | | | | | | | |
| 38 | 3521.0038 | | EMPSEEGYQD | 10 | 2 | 1660 | 10 | 3 | 1507 | 502 | 30% | 10 | 2 | 1026 | 10 | 1 | 620 | 620 | 10% |
| | | | SEQ ID NO: 54 | | | | | | | | | | | | | | | | |
| 40 | 3521.004 | | DNEAYEMPSE | 10 | 2 | 1660 | 10 | 2 | 727 | 364 | 20% | 10 | 2 | 1026 | 10 | 2 | 406.3 | 203 | 20% |
| | | | SEQ ID NO: 55 | | | | | | | | | | | | | | | | |
| 56 | 3521.0056 | | YEMPSEEGYQ | 10 | 2 | 1660 | 10 | 0 | 0 | | 0% | 10 | 2 | 1026 | 10 | 0 | 0 | | 0% |
| | | | SEQ ID NO: 56 | | | | | | | | | | | | | | | | |
| 58 | 3521.0058 | | SEEGYQDYEP | 10 | 2 | 1660 | 10 | 1 | 213 | 107 | 20% | 10 | 2 | 1026 | 10 | 1 | 73 | 73 | 10% |
| | | | SEQ ID NO: 57 | | | | | | | | | | | | | | | | |
| | | | | Normals Summary | | | | | | | | | | | | | | | |
| | | | | Pool Stimulated | | | | | | | | | | | | | | | |
| | | | | IFNg | | | | | | | | IL-g | | | | | | | |
| | | | | | | | Donors | | | | Re- | | | | Donors | | | | Re- |
| | | | | | | | test | Pep- | | | sponse | | | | test | Pep- | | | sponse |
| | Pooling | | Sequence | Donors | Pool | SFC | (pep- | tide | Total | Avg | Fre- | Donors | Pool | SFC | (pep- | tide | Total | Avg | Fre- |
| Peptide ID | sort | Pool | SEQ ID NO | tested | + | Pool | tide) | + | SFC | SFC | quency | tested | + | Pool | tide) | + | SFC | SFC | quency |
| 3521.001 | 1 | 1 | VFMKGLSKA | 12 | 1 | 1550 | 1 | 1 | 220 | 220 | 100% | 12 | 1 | 906.7 | 1 | 1 | 193 | 193 | 100% |
| | | | SEQ ID NO: 10 | | | | | | | | | | | | | | | | |
| 3521.0012 | 2 | 1 | KTKQGVAEA | 12 | 1 | 1550 | 1 | 0 | 0 | | 0% | 12 | 1 | 906.7 | 1 | 0 | 0 | | 0% |
| | | | SEQ ID NO: 81 | | | | | | | | | | | | | | | | |
| 3521.0016 | 3 | 1 | DVFMKGLSK | 12 | 1 | 1550 | 1 | 1 | 673 | 673 | 100% | 12 | 1 | 906.7 | 1 | 1 | 780 | 180 | 100% |
| | | | SEQ ID NO: 82 | | | | | | | | | | | | | | | | |
| 3521.0017 | 4 | 1 | FMKGLSKAK | 12 | 1 | 1550 | 1 | 1 | 46.7 | 46.7 | 100% | 12 | 1 | 906.7 | 1 | 0 | 0 | | 0% |
| | | | SEQ ID NO: 83 | | | | | | | | | | | | | | | | |
| 3521.0039 | 5 | 1 | DVMFKGLSKA | 12 | 1 | 1550 | 1 | 1 | 187 | 187 | 100% | 12 | 1 | 906.7 | 1 | 1 | 313 | 313 | 100% |
| | | | SEQ ID NO: 11 | | | | | | | | | | | | | | | | |
| 3521.0043 | 6 | 1 | VFMKGLSKAK | 12 | 1 | 1550 | 1 | 1 | 66.7 | 66.7 | 100% | 12 | 1 | 906.7 | 1 | 1 | 93.3 | 93.3 | 100% |
| | | | SEQ ID NO: 44 | | | | | | | | | | | | | | | | |
| 3521.0046 | 7 | 1 | FMKGLSKAKE | 12 | 1 | 1550 | 1 | 0 | 0 | | 0% | 12 | 1 | 906.7 | 1 | 0 | 0 | | 0% |
| | | | SEQ ID NO: 84 | | | | | | | | | | | | | | | | |
| 3521.0052 | 8 | 1 | KTKQGVAEAA | 12 | 1 | 1550 | 1 | 0 | 0 | | 0% | 12 | 1 | 906.7 | 1 | 0 | 0 | | 0% |
| | | | SEQ ID NO: 85 | | | | | | | | | | | | | | | | |
| 3521.0053 | 9 | 1 | KAKEGVVAAA | 12 | 1 | 1550 | 1 | 0 | 0 | | 0% | 12 | 1 | 906.7 | 1 | 0 | 0 | | 0% |
| | | | SEQ ID NO: 86 | | | | | | | | | | | | | | | | |
| 3521.0066 | 10 | 1 | KAKEGVVAA | 12 | 1 | 1550 | 1 | 0 | 0 | | 0% | 12 | 1 | 906.7 | 1 | 0 | 0 | | 0% |
| | | | SEQ ID NO: 87 | | | | | | | | | | | | | | | | |
| 3521.007 | 11 | 1 | GVVAAAEKTK | 12 | 1 | 1550 | 1 | 0 | 0 | | 0% | 12 | 1 | 906.7 | 1 | 0 | 0 | | 0% |
| | | | SEQ ID NO: 12 | | | | | | | | | | | | | | | | |
| 3521.0074 | 12 | 1 | VAAAEKTKQGVAEAP | 12 | 1 | 1550 | 1 | 0 | 0 | | 0% | 12 | 1 | 906.7 | 1 | 0 | 0 | | 0% |
| | | | SEQ ID NO: 13 | | | | | | | | | | | | | | | | |
| 3521.008 | 13 | 1 | VAAAEKTKQGVAEAA | 12 | 1 | 1550 | 1 | 0 | 0 | | 0% | 12 | 1 | 906.7 | 1 | 0 | 0 | | 0% |
| | | | SEQ ID NO: 14 | | | | | | | | | | | | | | | | |
| 3521.0004 | 14 | 2 | KTKEGYLYV | 12 | 1 | 1223 | 2 | 0 | 0 | | 0% | 12 | 2 | 5390 | 2 | 0 | 0 | | 0% |
| | | | SEQ ID NO: 88 | | | | | | | | | | | | | | | | |
| 3521.0019 | 15 | 2 | APGKTKEGV | 12 | 1 | 1223 | 2 | 0 | 0 | | 0% | 12 | 2 | 5390 | 2 | 0 | 0 | | 0% |
| | | | SEQ ID NO: 89 | | | | | | | | | | | | | | | | |
| 3521.002 | 16 | 2 | AGKTKEGVL | 12 | 1 | 1223 | 2 | 0 | 0 | | 0% | 12 | 2 | 5390 | 2 | 0 | 0 | | 0% |
| | | | SEQ ID NO: 15 | | | | | | | | | | | | | | | | |
| 3521.0021 | 17 | 2 | PGKTKEGVL | 12 | 1 | 1223 | 2 | 0 | 0 | | 0% | 12 | 2 | 5390 | 2 | 0 | 0 | | 0% |
| | | | SEQ ID NO: 16 | | | | | | | | | | | | | | | | |
| 3521.0042 | 18 | 2 | AGKTKEGVLY | 12 | 1 | 1223 | 2 | 0 | 0 | | 0% | 12 | 2 | 5390 | 2 | 0 | 0 | | 0% |
| | | | SEQ ID NO: 17 | | | | | | | | | | | | | | | | |
| 3521.005 | 19 | 2 | APGKTKEGVL | 12 | 1 | 1223 | 2 | 0 | 0 | | 0% | 12 | 2 | 5390 | 2 | 0 | 0 | | 0% |
| | | | SEQ ID NO: 18 | | | | | | | | | | | | | | | | |
| 3521.0059 | 20 | 2 | AEAAGKTKEG | 12 | 1 | 1223 | 2 | 1 | 120 | 120 | 50% | 12 | 2 | 5390 | 2 | 0 | 0 | | 0% |
| | | | SEQ ID NO: 45 | | | | | | | | | | | | | | | | |
| 3521.0068 | 21 | 2 | GVAEAPGKTK | 12 | 1 | 1223 | 2 | 0 | 0 | | 0% | 12 | 2 | 5390 | 2 | 0 | 0 | | 0% |
| | | | SEQ ID NO: 90 | | | | | | | | | | | | | | | | |
| 3521.0069 | 22 | 2 | GVAEAPGKTK | 12 | 1 | 1223 | 2 | 0 | 0 | | 0% | 12 | 2 | 5390 | 2 | 0 | 0 | | 0% |
| | | | SEQ ID NO: 19 | | | | | | | | | | | | | | | | |
| 3521.0075 | 23 | 2 | KQGVAEAPGKTKEGV | 12 | 1 | 1223 | 2 | 0 | 0 | | 0% | 12 | 2 | 5390 | 2 | 1 | 147 | 147 | 50% |
| | | | SEQ ID NO: 20 | | | | | | | | | | | | | | | | |
| 3521.0076 | 24 | 2 | PGKTKEGVLYVGSKT | 12 | 1 | 1223 | 2 | 1 | 300 | 300 | 50% | 12 | 2 | 5390 | 2 | 1 | 120 | 120 | 50% |
| | | | SEQ ID NO: 21 | | | | | | | | | | | | | | | | |
| 3521.0077 | 25 | 2 | KTKEGVLYVGSKTKK | 12 | 1 | 1223 | 2 | 2 | 3020 | 1510 | 100% | 12 | 2 | 5390 | 2 | 2 | 1814 | 3907 | 100% |
| | | | SEQ ID NO: 22 | | | | | | | | | | | | | | | | |
| 3521.0081 | 26 | 2 | KQGVAEAAGKTKEGV | 12 | 1 | 1223 | 2 | 1 | 220 | 220 | 50% | 12 | 2 | 5390 | 2 | 1 | 120 | 120 | 50% |
| | | | SEQ ID NO: 23 | | | | | | | | | | | | | | | | |
| 3521.0082 | 27 | 2 | AGKTKEGVLYVGSKT | 12 | 1 | 1223 | 2 | 1 | 200 | 200 | 50% | 12 | 2 | 5390 | 2 | 1 | 320 | 320 | 50% |
| | | | SEQ ID NO: 24 | | | | | | | | | | | | | | | | |
| 3521.0006 | 28 | 3 | VLYVGSKTK | 12 | 1 | 127 | 0 | 0 | 0 | | | 12 | 0 | 0 | 0 | 0 | 0 | | |
| | | | SEQ ID NO: 25 | | | | | | | | | | | | | | | | |
| 3521.0009 | 29 | 3 | LYVGSKTKK | 12 | 1 | 127 | 0 | 0 | 0 | | | 12 | 0 | 0 | 0 | 0 | 0 | | |
| | | | SEQ ID NO: 26 | | | | | | | | | | | | | | | | |
| 3521.0031 | 30 | 3 | YVGSLTLEG | 12 | 1 | 127 | 0 | 0 | 0 | | | 12 | 0 | 0 | 0 | 0 | 0 | | |
| | | | SEQ ID NO: 27 | | | | | | | | | | | | | | | | |
| 3521.0033 | 31 | 3 | VLYVGSKTKK | 12 | 1 | 127 | 0 | 0 | 0 | | | 12 | 0 | 0 | 0 | 0 | 0 | | |
| | | | SEQ ID NO: 28 | | | | | | | | | | | | | | | | |
| 3521.0034 | 32 | 3 | GVLYVGSKTK | 12 | 1 | 127 | 0 | 0 | 0 | | | 12 | 0 | 0 | 0 | 0 | 0 | | |
| | | | SEQ ID NO: 29 | | | | | | | | | | | | | | | | |
| 3521.0037 | 33 | 3 | LYVGSKTKEG | 12 | 1 | 127 | 0 | 0 | 0 | | | 12 | 0 | 0 | 0 | 0 | 0 | | |
| | | | SEQ ID NO: 30 | | | | | | | | | | | | | | | | |
| 3521.0045 | 34 | 3 | KTKKGVVHGV | 12 | 1 | 127 | 0 | 0 | 0 | | | 12 | 0 | 0 | 0 | 0 | 0 | | |
| | | | SEQ ID NO: 31 | | | | | | | | | | | | | | | | |
| 3521.0062 | 35 | 3 | KTKKGVVHGV | 12 | 1 | 127 | 0 | 0 | 0 | | | 12 | 0 | 0 | 0 | 0 | 0 | | |
| | | | SEQ ID NO: 32 | | | | | | | | | | | | | | | | |
| 3521.0067 | 36 | 3 | KTKEGVVHG | 12 | 1 | 127 | 0 | 0 | 0 | | | 12 | 0 | 0 | 0 | 0 | 0 | | |
| | | | SEQ ID NO: 91 | | | | | | | | | | | | | | | | |
| 3521.0071 | 37 | 3 | KTKEGVVHG | 12 | 1 | 127 | 0 | 0 | 0 | | | 12 | 0 | 0 | 0 | 0 | 0 | | |
| | | | SEQ ID NO: 46 | | | | | | | | | | | | | | | | |
| 3521.0078 | 38 | 3 | YVGSKTKEGVVHGVT | 12 | 1 | 127 | 0 | 0 | 0 | | | 12 | 0 | 0 | 0 | 0 | 0 | | |
| | | | SEQ ID NO: 33 | | | | | | | | | | | | | | | | |
| 3521.0083 | 39 | 3 | YVGSKTKKGVVHGVA | 12 | 1 | 127 | 0 | 0 | 0 | | | 12 | 0 | 0 | 0 | 0 | 0 | | |
| | | | SEQ ID NO: 34 | | | | | | | | | | | | | | | | |
| 3521.0084 | 40 | 3 | YVGSKTKEGVVHGVA | 12 | 1 | 127 | 0 | 0 | 0 | | | 12 | 0 | 0 | 0 | 0 | 0 | | |
| | | | SEQ ID NO: 92 | | | | | | | | | | | | | | | | |
| 3521.0005 | 41 | 4 | AVVTGVTAV | 12 | 2 | 3497 | 2 | 0 | 0 | | 0% | 12 | 2 | 1167 | 2 | 0 | 0 | | 0% |
| | | | SEQ ID NO: 93 | | | | | | | | | | | | | | | | |
| 3521.0011 | 42 | 4 | AVTNVGGAV | 12 | 2 | 3497 | 2 | 0 | 0 | | 0% | 12 | 2 | 1167 | 2 | 0 | 0 | | 0% |
| | | | SEQ ID NO: 94 | | | | | | | | | | | | | | | | |
| 3521.0013 | 43 | 4 | VTNVGGAVV | 12 | 2 | 3497 | 2 | 0 | 0 | | 0% | 12 | 2 | 1167 | 2 | 0 | 0 | | 0% |
| | | | SEQ ID NO: 35 | | | | | | | | | | | | | | | | |
| 3521.0015 | 44 | 4 | KTKLQVTNV | 12 | 2 | 3497 | 2 | 0 | 0 | | 0% | 12 | 2 | 1167 | 2 | 0 | 0 | | 0% |
| | | | SEQ ID NO: 95 | | | | | | | | | | | | | | | | |
| 3521.0029 | 45 | 4 | NVGGAVVTGV | 12 | 2 | 3497 | 2 | 0 | 0 | | 0% | 12 | 2 | 1167 | 2 | 0 | 0 | | 0% |
| | | | SEQ ID NO: 96 | | | | | | | | | | | | | | | | |
| 3521.0032 | 46 | 4 | GAVVTGVTAV | 12 | 2 | 3497 | 2 | 0 | 0 | | 0% | 12 | 2 | 1167 | 2 | 0 | 0 | | 0% |
| | | | SEQ ID NO: 97 | | | | | | | | | | | | | | | | |
| 3521.0044 | 47 | 4 | GVTAVAQKTV | 12 | 2 | 3497 | 2 | 0 | 0 | | 0% | 12 | 2 | 1167 | 2 | 0 | 0 | | 0% |
| | | | SEQ ID NO: 98 | | | | | | | | | | | | | | | | |
| 3521.0048 | 48 | 4 | EQVTNVGGAV | 12 | 2 | 3497 | 2 | 0 | 0 | | 0% | 12 | 2 | 1167 | 2 | 0 | 0 | | 0% |
| | | | SEQ ID NO: 99 | | | | | | | | | | | | | | | | |
| 3521.006 | 49 | 4 | VATVAEKTKE | 12 | 2 | 3497 | 2 | 0 | 0 | | 0% | 12 | 2 | 1167 | 2 | 0 | 0 | | 0% |
| | | | SEQ ID NO: 100 | | | | | | | | | | | | | | | | |
| 3521.0063 | 50 | 4 | GVVHGVTTV | 12 | 2 | 3497 | 2 | 0 | 0 | | 0% | 12 | 2 | 1167 | 2 | 0 | 0 | | 0% |
| | | | SEQ ID NO: 36 | | | | | | | | | | | | | | | | |
| 3521.0064 | 51 | 4 | GVVHGVATV | 12 | 2 | 3497 | 2 | 0 | 0 | | 0% | 12 | 2 | 1167 | 2 | 1 | 75 | 75 | 50% |
| | | | SEQ ID NO: 101 | | | | | | | | | | | | | | | | |
| 3521.0072 | 52 | 4 | EGVVHGVTTVAEKTK | 12 | 2 | 3497 | 2 | 1 | 93.3 | 93.3 | 50% | 12 | 2 | 1167 | 2 | 0 | 0 | | 0% |
| | | | SEQ ID NO: 102 | | | | | | | | | | | | | | | | |
| 3521.0073 | 53 | 4 | TTVAEKTKEQVTNVG | 12 | 2 | 3497 | 2 | 0 | 0 | | 0% | 12 | 2 | 1167 | 2 | 0 | 0 | | 0% |
| | | | SEQ ID NO: 103 | | | | | | | | | | | | | | | | |
| 3521.0079 | 54 | 4 | KGVVHGVATVAEKTK | 12 | 2 | 3497 | 2 | 0 | 0 | | 0% | 12 | 2 | 1167 | 2 | 1 | 284 | 284 | 50% |
| | | | SEQ ID NO: 104 | | | | | | | | | | | | | | | | |
| 3521.0085 | 55 | 4 | EGVVHGVATVAEKTK | 12 | 2 | 3497 | 2 | 0 | 0 | | 0% | 12 | 2 | 1167 | 2 | 1 | 80 | 80 | 50% |
| | | | SEQ ID NO: 105 | | | | | | | | | | | | | | | | |
| 3521.0086 | 56 | 4 | ATVAEKTKEQVTNVG | 12 | 2 | 3497 | 2 | 0 | 0 | | 0% | 12 | 2 | 1167 | 2 | 0 | 0 | | 0% |
| | | | SEQ ID NO: 106 | | | | | | | | | | | | | | | | |
| 3521.0003 | 57 | 5 | SIAAATGFV | 12 | 2 | 373 | 2 | 1 | 33.3 | 33.3 | 50% | 12 | 2 | 277 | 2 | 0 | 0 | | 0% |
| | | | SEQ ID NO: 107 | | | | | | | | | | | | | | | | |
| 3521.0007 | 58 | 5 | AAATGFVKK | 12 | 2 | 373 | 2 | 0 | 0 | | 0% | 12 | 2 | 277 | 2 | 0 | 0 | | 0% |
| | | | SEQ ID NO: 108 | | | | | | | | | | | | | | | | |
| 3521.0008 | 59 | 5 | IAAATGFVK | 12 | 2 | 373 | 2 | 0 | 0 | | 0% | 12 | 2 | 277 | 2 | 0 | 0 | | 0% |
| | | | SEQ ID NO: 47 | | | | | | | | | | | | | | | | |
| 3521.0014 | 60 | 5 | KTVEGAGSI | 12 | 2 | 373 | 2 | 0 | 0 | | 0% | 12 | 2 | 277 | 2 | 0 | 0 | | 0% |
| | | | SEQ ID NO: 109 | | | | | | | | | | | | | | | | |
| 3521.0022 | 61 | 5 | GSIAAATGF | 12 | 2 | 373 | 2 | 0 | 0 | | 0% | 12 | 2 | 277 | 2 | 0 | 0 | | 0% |
| | | | SEQ ID NO: 110 | | | | | | | | | | | | | | | | |
| 3521.0025 | 62 | 5 | EEGAPQEGI | 12 | 2 | 373 | 2 | 0 | 0 | | 0% | 12 | 2 | 277 | 2 | 0 | 0 | | 0% |
| | | | SEQ ID NO: 37 | | | | | | | | | | | | | | | | |
| 3521.003 | 63 | 5 | GSIAAATGFV | 12 | 2 | 373 | 2 | 0 | 0 | | 0% | 12 | 2 | 277 | 2 | 0 | 0 | | 0% |
| | | | SEQ ID NO: 38 | | | | | | | | | | | | | | | | |
| 3521.0035 | 64 | 5 | SIAAATGFVK | 12 | 2 | 373 | 2 | 0 | 0 | | 0% | 12 | 2 | 277 | 2 | 1 | 536 | 536 | 50% |
| | | | SEQ ID NO: 39 | | | | | | | | | | | | | | | | |
| 3521.0036 | 65 | 5 | AGSIAAATGF | 12 | 2 | 373 | 2 | 0 | 0 | | 0% | 12 | 2 | 277 | 2 | 0 | 0 | | 0% |
| | | | SEQ ID NO: 40 | | | | | | | | | | | | | | | | |
| 3521.0047 | 66 | 5 | IAAATGFVKK | 12 | 2 | 373 | 2 | 0 | 0 | | 0% | 12 | 2 | 277 | 2 | 0 | 0 | | 0% |
| | | | SEQ ID NO: 41 | | | | | | | | | | | | | | | | |
| 3521.0049 | 67 | 5 | APQEGILEDM | 12 | 2 | 373 | 2 | 0 | 0 | | 0% | 12 | 2 | 277 | 2 | 0 | 0 | | 0% |
| | | | SEQ ID NO: 42 | | | | | | | | | | | | | | | | |
| 3521.0051 | 68 | 5 | EEGAPQEGIL | 12 | 2 | 373 | 2 | 1 | 554 | 554 | 50% | 12 | 2 | 277 | 2 | 1 | 1028 | 1028 | 50% |
| | | | SEQ ID NO: 43 | | | | | | | | | | | | | | | | |
| 3521.0057 | 69 | 5 | NEEGAPQEGI | 12 | 2 | 373 | 2 | 1 | 26.7 | 26.7 | 50% | 12 | 2 | 277 | 2 | 0 | 0 | | 0% |
| | | | SEQ ID NO: 111 | | | | | | | | | | | | | | | | |
| 3521.0061 | 70 | 5 | AATGFVKKDQ | 12 | 2 | 373 | 2 | 1 | 84 | 84 | 50% | 12 | 2 | 277 | 2 | 1 | 195 | 195 | 50% |
| | | | SEQ ID NO: 112 | | | | | | | | | | | | | | | | |
| 3521.0065 | 71 | 5 | FVKKDQLGK | 12 | 2 | 373 | 2 | 0 | 0 | | 0% | 12 | 2 | 277 | 2 | 0 | 0 | | 0% |
| | | | SEQ ID NO: 113 | | | | | | | | | | | | | | | | |
| 3521.0001 | 72 | 6 | PVDPDNEAY | 12 | 1 | 63 | 2 | 0 | | | 0% | 12 | 1 | 407 | 2 | 0 | 0 | | 0% |
| | | | SEQ ID NO: 114 | | | | | | | | | | | | | | | | |
| 3521.0002 | 73 | 6 | PSEEGYQDY | 12 | 1 | 63 | 2 | 0 | | | 0% | 12 | 1 | 407 | 2 | 0 | 0 | | 0% |
| | | | SEQ ID NO: 49 | | | | | | | | | | | | | | | | |
| 3521.0018 | 74 | 6 | MPVDPDNEA | 12 | 1 | 63 | 2 | 0 | | | 0% | 12 | 1 | 407 | 2 | 0 | 0 | | 0% |
| | | | SEQ ID NO: 115 | | | | | | | | | | | | | | | | |
| 3521.0023 | 75 | 6 | YEMPSEEGY | 12 | 1 | 63 | 2 | 0 | | | 0% | 12 | 1 | 407 | 2 | 1 | 58 | 58 | 50% |
| | | | SEQ ID NO: 50 | | | | | | | | | | | | | | | | |
| 3521.0024 | 76 | 6 | DPDNEAYEM | 12 | 1 | 63 | 2 | 93.3 | 93.3 | | 50% | 12 | 1 | 407 | 2 | 1 | 300 | 300 | 50% |
| | | | SEQ ID NO: 116 | | | | | | | | | | | | | | | | |
| 3521.0026 | 77 | 6 | MPSEEGYQD | 12 | 1 | 63 | 2 | 0 | | | 0% | 12 | 1 | 407 | 2 | 1 | 66 | 66 | 50% |
| | | | SEQ ID NO: 51 | | | | | | | | | | | | | | | | |
| 3521.0027 | 78 | 6 | AYEMPSEEGY | 12 | 1 | 63 | 2 | 0 | | | 0% | 12 | 1 | 407 | 2 | 0 | 0 | | 0% |
| | | | SEQ ID NO: 52 | | | | | | | | | | | | | | | | |
| 3521.0028 | 79 | 6 | MPSEEGYQDY | 12 | 1 | 63 | 2 | 0 | | | 0% | 12 | 1 | 407 | 2 | 0 | 0 | | 0% |
| | | | SEQ ID NO: 53 | | | | | | | | | | | | | | | | |
| 3521.0039 | 80 | 6 | EMPSEEGYQD | 12 | 1 | 63 | 2 | 0 | | | 0% | 12 | 1 | 407 | 2 | 1 | 297 | 297 | 50% |
| | | | SEQ ID NO: 54 | | | | | | | | | | | | | | | | |
| 3521.004 | 81 | 6 | DNEAYEMPSE | 12 | 1 | 63 | 2 | 0 | | | 0% | 12 | 1 | 407 | 2 | 0 | 0 | | 0% |
| | | | SEQ ID NO: 55 | | | | | | | | | | | | | | | | |
| 3521.0041 | 82 | 6 | EGILEDMPVD | 12 | 1 | 63 | 2 | 0 | | | 0% | 12 | 1 | 407 | 2 | 0 | 0 | | 0% |
| | | | SEQ ID NO: 117 | | | | | | | | | | | | | | | | |
| 3521.0054 | 83 | 6 | MPVDPDNEAY | 12 | 1 | 63 | 2 | 0 | | | 0% | 12 | 1 | 407 | 2 | 1 | 58 | 58 | 50% |
| | | | SEQ ID NO: 118 | | | | | | | | | | | | | | | | |
| 3521.0055 | 84 | 6 | QEGILEDMPV | 12 | 1 | 63 | 2 | 0 | | | 0% | 12 | 1 | 407 | 2 | 1 | 58 | 58 | 50% |
| | | | SEQ ID NO: 119 | | | | | | | | | | | | | | | | |
| 3521.0056 | 85 | 6 | YEMPSEEGYQ | 12 | 1 | 63 | 2 | 0 | | | 0% | 12 | 1 | 407 | 2 | 1 | 89 | 89 | 50% |
| | | | SEQ ID NO: 56 | | | | | | | | | | | | | | | | |
| 3521.0058 | 86 | 6 | SEEGYQDYEP | 12 | 1 | 63 | 2 | 0 | | | 0% | 12 | 1 | 407 | 2 | 1 | 58 | 58 | 50% |
| | | | SEQ ID NO: 57 | | | | | | | | | | | | | | | | |
| 2 | 3521.0002 | Poshpho | PSEEGYQDY | 8 | 1 | 307 | 8 | 0 | 0 | | 0% | 8 | 1 | 137 | 8 | 0 | 0 | | 0% |
| | | | SEQ ID NO: 49 | | | | | | | | | | | | | | | | |
| 23 | 3521.0023 | | YEMPSEEGY | 8 | 1 | 307 | 8 | 1 | 333 | 333 | 13% | 8 | 1 | 137 | 8 | 1 | 193 | 193 | 13% |
| | | | SEQ ID NO: 50 | | | | | | | | | | | | | | | | |
| 26 | 3521.0026 | | MPSEEGYQD | 8 | 1 | 307 | 8 | 1 | 27 | 27 | 13% | 8 | 1 | 137 | 8 | 1 | 93 | 93 | 13% |
| | | | SEQ ID NO: 51 | | | | | | | | | | | | | | | | |
| 27 | 3521.0027 | | AYEMPSEEGY | 8 | 1 | 307 | 8 | 1 | 53 | 53 | 13% | 8 | 1 | 137 | 8 | 1 | 87 | 87 | 13% |
| | | | SEQ ID NO: 52 | | | | | | | | | | | | | | | | |
| 28 | 3521.0028 | | MPSEEGYQDY | 8 | 1 | 307 | 8 | 1 | 40 | 40 | 13% | 8 | 1 | 137 | 8 | 1 | 113 | 113 | 13% |
| | | | SEQ ID NO: 53 | | | | | | | | | | | | | | | | |
| 39 | 3521.0038 | | EMPSEEGYQD | 8 | 1 | 307 | 8 | 1 | 40 | 40 | 13% | 8 | 1 | 137 | 8 | 1 | 33 | 33 | 13% |
| | | | SEQ ID NO: 54 | | | | | | | | | | | | | | | | |
| 40 | 3521.004 | | DNEAYEMPSE | 8 | 1 | 307 | 8 | 0 | 0 | | 0% | 8 | 1 | 137 | 8 | 0 | 0 | | 0% |
| | | | SEQ ID NO: 55 | | | | | | | | | | | | | | | | |
| 56 | 3521.0056 | | YEMPSEEGYQ | 8 | 1 | 307 | 8 | 1 | 100 | 100 | 13% | 8 | 1 | 137 | 8 | 1 | 167 | 167 | 13% |
| | | | SEQ ID NO: 56 | | | | | | | | | | | | | | | | |
| 58 | 3521.0058 | | SEEGYQDYEP | 8 | 1 | 307 | 8 | 1 | 26.7 | 26.7 | 13% | 8 | 1 | 137 | 8 | 0 | 0 | | 0% |
| | | | SEQ ID NO: 57 | | | | | | | | | | | | | | | | |

| | | | | Pool + in | Peptides + in |
|---|---|---|---|---|---|
| | Pooling | | Sequence | Patients & | Patients & |
| Peptide ID | sort | Pool | SEQ ID NO | Normals | Normals |
| 3521.001 | 1 | 1 | VFMKGLSKA | 1 | 1 |
| | | | SEQ ID NO: 10 | | |
| 3521.0012 | 2 | 1 | KTKQGVAEA | 1 | 0 |
| | | | SEQ ID NO: 81 | | |
| 3521.0016 | 3 | 1 | DVFMKGLSK | 1 | 0 |
| | | | SEQ ID NO: 82 | | |
| 3521.0017 | 4 | 1 | FMKGLSKAK | 1 | 0 |
| | | | SEQ ID NO: 83 | | |
| 3521.0039 | 5 | 1 | DVFMKGLSKA | 1 | 1 |
| | | | SEQ ID NO: 11 | | |
| 3521.0043 | 6 | 1 | VFMKGLSKAK | 1 | 1 |
| | | | SEQ ID NO: 44 | | |
| 3521.0046 | 7 | 1 | FMKGLSKAKE | 1 | 0 |
| | | | SEQ ID NO: 84 | | |
| 3521.0052 | 8 | 1 | KTKQGVAEAA | 1 | 0 |
| | | | SEQ ID NO: 85 | | |
| 3521.0053 | 9 | 1 | KAKEGVVAAA | 1 | 0 |
| | | | SEQ ID NO: 86 | | |
| 3521.0066 | 10 | 1 | KAKEGVVAA | 1 | 0 |
| | | | SEQ ID NO: 87 | | |
| 3521.007 | 11 | 1 | GVVAAAEKTK | 1 | 0 |
| | | | SEQ ID NO: 12 | | |
| 3521.0074 | 12 | 1 | VAAAEKTKQGVAEAP | 1 | 0 |
| | | | SEQ ID NO: 13 | | |
| 3521.008 | 13 | 1 | VAAAEKTKQGVAEAA | 1 | 0 |
| | | | SEQ ID NO: 14 | | |
| 3521.0004 | 14 | 2 | KTKEGVLYV | 1 | 0 |
| | | | SEQ ID NO: 88 | | |
| 3521.0019 | 15 | 2 | APGKTKEGV | 1 | 0 |
| | | | SEQ ID NO: 89 | | |
| 3521.002 | 16 | 2 | AGKTKEGVL | 1 | 0 |
| | | | SEQ ID NO: 15 | | |
| 3521.0021 | 17 | 2 | PGKTKEGVL | 1 | 0 |
| | | | SEQ ID NO: 16 | | |
| 3521.0042 | 18 | 2 | AGKTKEGGVL | 1 | 0 |
| | | | SEQ ID NO: 17 | | |
| 3521.005 | 19 | 2 | APGKTKEGVL | 1 | 0 |
| | | | SEQ ID NO: 18 | | |
| 3521.0059 | 20 | 2 | AEAAGKTKEG | 1 | 1 |
| | | | SEQ ID NO: 45 | | |
| 3521.0068 | 21 | 2 | GVAEAPGKTK | 1 | 0 |
| | | | SEQ ID NO: 90 | | |
| 3521.0069 | 22 | 2 | GVAEAAGKTK | | 0 |
| | | | SEQ ID NO: 19 | | |
| 3521.0075 | 23 | 2 | KQGVAEAPGTKEGV | 1 | 1 |
| | | | SEQ ID NO: 20 | | |
| 3521.0076 | 24 | 2 | PGKTKEGVLYVGSKT | 1 | 1 |
| | | | SEQ ID NO: 21 | | |
| 3521.0077 | 25 | 2 | KTKEGVLYVGSKTKK | 1 | 1 |
| | | | SEQ ID NO: 22 | | |
| 3521.0081 | 26 | 2 | KQGVAEAAGKTKEGV | 1 | 1 |
| | | | SEQ ID NO: 23 | | |
| 3521.0082 | 27 | 2 | AGKTKEGVLYVGSKT | 1 | 1 |
| | | | SEQ ID NO: 24 | | |
| 3521.0006 | 28 | 6 | VLYVGSKTK | 1 | 0 |
| | | | SEQ ID NO: 25 | | |
| 3521.0009 | 29 | 6 | LYVGSKTKK | 1 | 0 |
| | | | SEQ ID NO: 26 | | |
| 3521.0031 | 30 | 6 | YVGSKTKEGV | 1 | 0 |
| | | | SEQ ID NO: 27 | | |
| 3521.0033 | 31 | 6 | VLYVGSKTKK | 1 | 1 |
| | | | SEQ ID NO: 28 | | |
| 3521.0034 | 32 | 6 | GVLYVGSKTK | 1 | 1 |
| | | | SEQ ID NO: 29 | | |
| 3521.0037 | 33 | 6 | LYVGSKTKEG | 1 | 0 |
| | | | SEQ ID NO: 30 | | |
| 352100.45 | 34 | 6 | KTKKGVVHGV | 1 | 0 |
| | | | SEQ ID NO: 31 | | |
| 3521.0062 | 35 | 6 | KTKKGVVHG | 1 | 0 |
| | | | SEQ ID NO: 32 | | |
| 3521.0067 | 36 | 6 | KTKEGVVHG | 1 | 0 |
| | | | SEQ ID NO: 91 | | |
| 3521.0071 | 37 | 6 | YVGSKTKEGVVHGVT | 1 | 0 |
| | | | SEQ ID NO: 46 | | |
| 3521.0078 | 38 | 6 | YVGSKTKKGVVHGVA | 1 | 0 |
| | | | SEQ ID NO: 33 | | |
| 3521.0083 | 39 | 6 | KTKEGVLYVGSKTKE | 1 | 0 |
| | | | SEQ ID NO: 34 | | |
| 3521.0084 | 40 | 6 | YVGSKTKEGVVHGVA | 1 | 0 |
| | | | SEQ ID NO: 92 | | |
| 3521.0005 | 41 | 4 | AVVTGVTAV | 1 | 0 |
| | | | SEQ ID NO: 93 | | |
| 3521.0011 | 42 | 4 | QVTNVGGAV | 1 | 0 |
| | | | SEQ ID NO: 94 | | |
| 3521.0013 | 43 | 4 | VTNVGGAVV | 1 | 0 |
| | | | SEQ ID NO: 35 | | |
| 3521.0015 | 44 | 4 | KTKEQVTNV | 1 | 0 |
| | | | SEQ ID NO: 95 | | |
| 3521.0029 | 45 | 4 | NVGGAVVTGV | 1 | 0 |
| | | | SEQ ID NO: 96 | | |
| 3521.0032 | 46 | 4 | GAVVTGVTAV | 1 | 1 |
| | | | SEQ ID NO: 97 | | |
| 3521.0044 | 47 | 4 | GVTAVAQKTV | 1 | 0 |
| | | | SEQ ID NO: 98 | | |
| 3521.0048 | 48 | 4 | EQVTNVGGAV | 1 | 0 |
| | | | SEQ ID NO: 99 | | |
| 3521.006 | 49 | 4 | VATVAEKTKE | 1 | 1 |
| | | | SEQ ID NO: 100 | | |
| 3521.0063 | 50 | 4 | GVVHGVTTV | 1 | 1 |
| | | | SEQ ID NO: 36 | | |
| 3521.0064 | 51 | 4 | GVVHGVATV | 1 | 1 |
| | | | SEQ ID NO: 101 | | |
| 3521.0072 | 52 | 4 | EGVVHGVTTVAEKTK | 1 | 1 |
| | | | SEQ ID NO: 102 | | |
| 3521.0073 | 53 | 4 | TTVAEKTKEQVTNVG | 1 | 1 |
| | | | SEQ ID NO: 103 | | |
| 3521.0079 | 54 | 4 | KGVVHGVATVAEKTK | 1 | 1 |
| | | | SEQ ID NO: 104 | | |
| 3521.0085 | 55 | 4 | EGVVHGVATVAEKTK | 1 | 1 |
| | | | SEQ ID NO: 105 | | |
| 3521.0086 | 56 | 4 | ATVAEKTKEQVTNVG | 1 | 1 |
| | | | SEQ ID NO: 106 | | |
| 3521.0003 | 57 | 5 | SIAAATGFV | 1 | 0 |
| | | | SEQ ID NO: 107 | | |
| 3521.0007 | 58 | 5 | AAATGFVKK | 1 | 0 |
| | | | SEQ ID NO: 108 | | |
| 3521.0008 | 59 | 5 | IAAATGFVK | 1 | 0 |
| | | | SEQ ID NO: 47 | | |
| 3521.0014 | 60 | 5 | KTVEGAGSI | 1 | 0 |
| | | | SEQ ID NO: 109 | | |
| 3521.0022 | 61 | 5 | GSIAAATGF | 1 | 0 |
| | | | SEQ ID NO: 110 | | |
| 3521.0025 | 62 | 5 | EEGAPQEGI | 1 | 0 |
| | | | SEQ ID NO: 37 | | |
| 3521.003 | 63 | 5 | GSIAAATGFV | 1 | 0 |
| | | | SEQ ID NO: 38 | | |
| 3521.0035 | 64 | 5 | SIAAATGFVK | 1 | 1 |
| | | | SEQ ID NO: 39 | | |
| 3521.0036 | 65 | 5 | AGSIAAATGF | 1 | 0 |
| | | | SEQ ID NO: 40 | | |
| 3521.0047 | 66 | 5 | IAAATGFVKK | 1 | 0 |
| | | | SEQ ID NO: 41 | | |
| 3521.0049 | 67 | 5 | APQEGILEDM | 1 | 0 |
| | | | SEQ ID NO: 42 | | |
| 3521.0051 | 68 | 5 | EEGAPQEGIL | 1 | 1 |
| | | | SEQ ID NO: 43 | | |
| 3521.0057 | 69 | 5 | NEEGAPQEGI | 1 | 0 |
| | | | SEQ ID NO: 111 | | |
| 3521.0061 | 70 | 6 | AATGFVKKDQ | 1 | 0 |
| | | | SEQ ID NO: 112 | | |
| 3521.0065 | 71 | 6 | FVKKDQLGK | 1 | 0 |
| | | | SEQ ID NO: 113 | | |
| 3521.0001 | 72 | 6 | PVDPDNEAY | 1 | 0 |
| | | | SEQ ID NO: 114 | | |
| 3521.0002 | 73 | 6 | PSEEGYQDY | 1 | 0 |
| | | | SEQ ID NO: 49 | | |
| 3521.0018 | 74 | 6 | MPVDPDNEA | 1 | 0 |
| | | | SEQ ID NO: 115 | | |
| 3521.0023 | 75 | 6 | YEMPSEEGY | 1 | 0 |
| | | | SEQ ID NO: 50 | | |
| 3521.0024 | 76 | 6 | DPDNEAYEM | 1 | 0 |
| | | | SEQ ID NO: 116 | | |
| 3521.0026 | 77 | 6 | MPSEEGYQD | 1 | 0 |
| | | | SEQ ID NO: 51 | | |
| 3521.0027 | 78 | 6 | AYEMPSEEGY | 1 | 0 |
| | | | SEQ ID NO: 52 | | |
| 3521.0028 | 79 | 6 | MPSEEGYQDY | 1 | 0 |
| | | | SEQ ID NO: 53 | | |
| 3521.0038 | 80 | 6 | EMPSEEGYQD | 1 | 0 |
| | | | SEQ ID NO: 54 | | |
| 3521.004 | 81 | 6 | DNEAYEMPSE | 1 | 0 |
| | | | SEQ ID NO: 55 | | |
| 3521.0041 | 82 | 6 | EGILEDMPVD | 1 | 0 |
| | | | SEQ ID NO: 117 | | |
| 3521.0054 | 83 | 6 | MPVDPDNEAY | 1 | 0 |
| | | | SEQ ID NO: 118 | | |
| 3521.0055 | 84 | 6 | QEGILEDMPV | 1 | 0 |
| | | | SEQ ID NO: 119 | | |
| 3521.0056 | 85 | 6 | YEMPSEEGYQ | 1 | 0 |
| | | | SEQ ID NO: 56 | | |
| 3521.0058 | 86 | 6 | SEEGYQDYEP | 1 | 0 |
| | | | SEQ ID NO: 57 | | |
| 2 | 3521.0002 | Phospho | PSEEGYQDY | 1 | 0 |
| | | | SEQ ID NO: 49 | | |
| 23 | 3521.0023 | | YEMPSEEGY | 1 | 1 |
| | | | SEQ ID NO: 50 | | |
| 26 | 3521.0026 | | MPSEEGYQD | 1 | 1 |
| | | | SEQ ID NO: 51 | | |
| 27 | 3521.0027 | | AYEMPSEEGY | 1 | 1 |
| | | | SEQ ID NO: 52 | | |
| 28 | 3521.0028 | | MPSEEGYQDY | 1 | 1 |
| | | | SEQ ID NO: 53 | | |
| 38 | 3521.0038 | | EMPSEEGYQD | 1 | 1 |
| | | | SEQ ID NO: 54 | | |
| 40 | 3521.004 | | DNEAYEMPSE | 1 | 0 |
| | | | SEQ ID NO: 55 | | |
| 56 | 3521.0056 | | YEMPSEEGYQ | 1 | 0 |
| | | | SEQ ID NO: 56 | | |
| 58 | 3521.0058 | | SEEGYQDYEP | 1 | 1 |
| | | | SEQ ID NO: 57 | | |

### Example 10. Tolerization Therapy Specific for Epitopes of α-Syn Neuromelanin, Leucine-Rich Repeat Kinase 2 (LRRK2) and Glucocerebrosidase is Useful in Treating Subjects Afflicted with PD

Epitopes to which T cells are responsive in subjects afflicted with PD are identified by

i) obtaining T cells from each subject;
ii) contacting the T cells with a test compound;
iii) determining whether the T cells have increased activation after contact with the test compound; and
iv) identifying the test compound as an epitope to which the T cells are responsive if in step iii) the T cells are determined to have increased activation after contact with the test compound, and identifying the test compound as not an epitope to which the T cells are responsive if in step iii) the T cells are determined to not have increased activation after contact with the test compound. This method is repeated sequentially or in parallel for thousands of test compounds, each having an amino acid sequence identical to a stretch of consecutive amino acids in the α-syn, LRRK2 and glucocerebrosidase proteins, or being identical to a portion of neuromelanin. Epitopes for α-syn, neuromelanin, LRRK2 and glucocerebrosidase are identified in individual subjects.

The subjects afflicted with PD are then separated into one of two groups: 1) a test group that receives tolerization therapy, or 2) a control group that does not receive tolerization therapy.

Within the test group, an effective amount of an α-syn, neuromelanin, LRRK2 or glucocerebrosidase epitope is administered orally, nasally, or subcutaneously to each subject (i.e., tolerization therapy specific for the epitope). Within the control group, a polypeptide having a random sequence is administered to each subject.

Compared to the control group, subjects in the test group have a statistically significant reduction in symptoms of PD. Tremors are reduced and mobility is improved in a statistically significant proportion of the subjects. Additionally, a statistically significant proportion of the subjects have little or no progression of PD.

Less or no activation of T cells by the epitope is observed in subjects who receive and respond to tolerization therapy, but not in subjects who do not receive or who do not respond to tolerization therapy.

### Example 11. Autoimmune Features of Neurodegenerative Disorders Specific Aims

Neurodegenerative diseases are characterized by the misprocessing of specific proteins, but how and if this results in cell death has been unknown. This study supports collaborative research between immunology (Sette) and neuroscience (Sulzer) laboratories with disease experts (Alcalay, Warder), to pursue new findings that require such cross-disciplinary collaboration.

Joint Results disclosed herein provide the first direct evidence that Parkinson's disease (PD), which has long been known to feature prominent neuroinflammatory components, is for at least many patients in part an autoimmune disorder that features antigen presentation and specific T cell responses. The results demonstrate that PD shares fundamental features with classical autoimmune disorders including Type-1 diabetes (T1D), multiple sclerosis, and rheumatoid arthritis.

This new direction stems in part from a recent report of antigen presentation by substantia nigra (SN) dopamine and locus coeruleus norepinephrine neurons in adult human brain, and the accompanying demonstration of T cell mediated death in primary neuronal culture (Cebrian et al., 2014c). While indicating that the most targeted neurons in PD can present antigen, that study did not identify specific antigens. The Results disclosed herein now identify two disease-associated neoantigens (autoimmune self epitopes) that are expressed by human blood donors that are derived from alpha-synuclein (α-syn), a protein well established to be misprocessed in PD. These epitopes were found in 21 of 44 PD patients and comparatively little in age matched controls without neurodegenerative disease (p<0.01 patients versus controls). The α-syn epitopes are in two regions, one close to the pathogenic mutations that cause PD, and the other at a residue highly phosphorylated in PD. Two common HLA-DR alleles were identified in the patients with high binding affinity to these antigens; these alleles have been independently associated with PD by genome-wide association (GWAS) linkage studies, and so this process is a candidate for explaining at least forms of PD associated with these HLA-DR alleles. Finally, the Standaert group recently published that SN neuronal death in a viral α-syn overexpression model is absent in MHC-II null mice.

Without wishing to be bound by any scientific theory, the overall hypothesis in this study is that PD is associated with self-derived neoantigens that becomes increasingly expressed in aging or disease conditions. The overall aim in this study is to identify the antigenic responses associated with PD and Lewy Body disease (LBD). α-syn-derived neoantigens are identified in these patients and these profiles are compared in patients with additional Lewy Body dementia (LBD) as well as Alzheimer's disease (AD). As a control protein, phosphorylated-tau will be examined, which undergoes similar means of autophagic degradation to α-syn and is a PD rise factor. This study 1) identifies epitopes that act as neoantigens in PD, AD, and LBD; 2) characterizes the responsive T cells; and 3) characterizes the role of antigen presentation and T cell-mediated neuronal death by animal models that express an HLA allele implicated in PD with high affinity to an α-syn epitope.

The autoimmune features are confirmed, and therapies are used to treat other autoimmune disorders such as tolerization are used to treat PD.

Aim 1. Identify Antigenic Proteins and Epitopes in PD, LBD, and AD. Hypothesis:

Epitopes derived from native and modified α-syn and possibly tau protein in PD, AD, and LBD are recognized as neoantigens by patient T cells.

Plan:

Blood from patients and aged matched controls is examined to quantify the T cells that are responsive to potential neoantigens, particularly following phosphorylation, using in vitro assays. Epitopes, HLA restriction and cytokine profiles are defined for responsive patients. Specific epitope-HLA combinations are identified in patient populations.

Aim 2. Characterize the HLA Alleles the Epitopes and Responsive T Cells in PD, LBD, and AD.

Hypothesis:

T cells responsive to neoantigens in neurodegenerative disorders encompass memory T cell subsets that have undergone clonal expansion during pathogenesis.

Plan:

Subsets of responsive T cells from patients are identified and phenotyped by cytokine profiling and by using tetramers of specific HLA alleles and epitopes determined in Aim 1. This provides a means for diagnosis of subtypes of these disorders and potential biomarkers.

Aim 3. Determine the Role of HLA Restricted Alpha-Synuclein Antigen Presentation in a PD Mouse Model.

Hypothesis:

Overexpression of α-syn in DA neurons of the SN drives neuronal MHC and antigen expression, leading to T cell-mediated replication and neuronal death.

Plan:

A viral α-syn overexpression model (rAAV2-SYN) is adapted to humanized mutant mice that express HLA of high and low affinity for α-syn epitopes. This study examines MHC and cytokine expression, T cell infiltration to brain, PD model behaviors, alterations in DA neurotransmission, and SN neuronal death, which may be absent in MHC null mice and reinstated with human MHC alleles associated with PD and α-syn.

Research Strategy

(A) Significance

The causes of neuronal death in Parkinson's disease (PD) and related neurodegenerative disorders including Lewy Body dementia (LBD) and

Alzheimer's (AD) remain unknown. This study tests the novel hypothesis that PD is in part an autoimmune disorder of the acquired immune system in which T cells recognize proteins such as alpha-synuclein (α-syn) as neoantigens. This direction was initiated by the recent discovery of antigen presentation by adult human neurons of the substantia nigra (SN) and locus coeruleus (LC), and that cultured SN neurons induced to express a classical MHC-I presented antigen (the albumin-derived epitope, SIINFEKL) are killed by specific T cells that recognize that epitope (Cebrian et al., 2014c).

A primary neuropathological signature of PD is the presence of highly phosphorylated (at residue S129) α-syn aggregates known as Lewy bodies and Lewy neurites in surviving SN DA neurons. Lewy inclusions are also prominent in cortical neurons in LBD (Spillantini et al., 1998b; Spillantini et al., 1998a). Notably, in the synucleinopathies, PD with dementia (PDD), which is observed in a majority of long-term PD patients, and LBD, also feature aggregates of phosphorylated tau (phospho-tau), inclusions that are highly prominent in Alzheimer's disease (AD) (Arnold et al., 2013).

Classical autoimmune disorders such as Type-1 diabetes (T1D) are thought to result from an analogous misprocessing of self proteins to produce “neoantigens”, epitopes presented by major histocompatibility (MHC) proteins (Marrack and Kappler, 2012). These are often thought to have escaped self-recognition during thymic selection (Yin et al., 2013). Not only is the thymus lost during the third decade, but lysosomal degradation including chaperone-mediated autophagy (CMA) is less efficient with age (Cuervo and Macian, 2014), as apparent from the non-degraded lipofuscin or “aging pigment” and neuromelanin that accumulates in neurons of the SN and LC neurons that are most highly targeted in PD (Sulzer et al., 2008). A relevant finding from collaborations with the Cuervo laboratory is that the mutant forms of α-syn (A53T and A30P) and dopamine-modified α-syn both block CMA (Cuervo et al., 2004; Martinez-Vicente et al., 2008), while phosphorylated α-syn is not broken down by CMA (Martinez-Vicente et al., 2008). Thus, the appearance of novel breakdown products of α-syn during disease is expected, and could provide a source of neoantigens that could lead to PD.

As shown in the Results, neoantigens (Armstrong et al., 2014; Hall et al., 2014) derived from α-syn are found to be present in about 50% of cases of PD. Reports by others indicate that CD8⁺ and “helper” (CD4⁺) T cells are present at far higher than normal levels in the SN of PD brains than control subjects (Brochard et al., 2009). If antigens specific to PD were presented by these neurons in patients and responsive T cells were present, the neurons may be killed by immunological processes.

It is further found that α-syn provides two specific regions containing neoantigenic epitopes in ˜50% of PD patients. Both epitopes feature a region of the protein modified by phosphorylation. The first, Y39, is present in both controls and PD patients (Mahul-Mellier et al., 2014). The second, S129, exhibits much higher phosphorylation in PD (Foulds et al., 2013). Tau, which is implicated as a PD gene (Sharma et al., 2012), also a CMA substrate (Wang et al., 2009), highly phosphorylated in multiple disorders (PDD, LBD, AD), and produces aggregates that can overlap with Lewy aggregates particularly in PDD and LBD, is also a possible additional candidate neoantigen.

The identification of PD autoimmune features offers important implications including;
1. Therapeutic approaches being studied for classical autoimmune disorders such as tolerization therapy may be adopted for treatment.
2. Genetic PD models generally do not exhibit SN and LC neuronal death (Chesselet, 2008), perhaps as immune features are generally missing (Harms et al., 2013; Cebrian et al., 2014b), and so improved animal models could be developed.
3. Explanations for linkage between specific HLA genes and PD in GWAS studies would be explained (Lampe et al., 2003; Hamza et al., 2010; Hill-Burns et al., 2011; Ahmed et al., 2012; Wissemann et al., 2013; Zhao et al., 2013).
4. Screening for T cell response could provide a means for preclinical identification or a biomarker.
5. Insight may be provided on how α-syn pathology apparently “spreads” through the nervous system (Luk and Lee, 2014), particularly from the periphery (Shannon et al., 2012) where T cells replicate.
6. After age 50, the incidence of PD in identical and fraternal twins is identical (Tanner et al., 1999), and monogenic causes of the disease represent only a small fraction of the patients: this “sporadic” nature of PD might be explained by T cell receptors, which differ even in identical twins.

(B) Innovation

The project is to our knowledge the first to explore if proteins involved in the pathogenesis of neurodegenerative disorders produce neoantigens. This impetus is derived from a) our new study (Cebrian et al., 2014c) reporting that SN and LC neurons of adult humans, the major targeted neurons in PD, display the antigen presenting protein, MHC-I, a first demonstration of antigen presentation by adult human neurons. b) Results demonstrating that PD patients selectively possess T cells that recognize α-syn as a foreign protein. c) A study from David Standaert's laboratory showing that mice deficient for MHC-II are protected from a viral model of α-syn neurodegeneration (Harms et al., 2013).

This project clearly requires collaborative expertise in neuroscience, immunology and the clinic, and the gathering together of these skills has enabled this new direction. Together, a new insight is introduced, that PD is at least in part an autoimmune disease. This may be extended to LBD, which is also a synucleinopathy that features both phosorylated α-syn and phosphorylated tau aggregates, and AD which has high levels of tau aggregates but for which α-syn aggregates are also prominent in some individuals. Without wishing to be bound by any scientific theory, it is suggested, in analogy to T1D, that with disease processes, some individuals develop protein modifications, such as phosphorylated and dopamine-modified α-syn in PD or highly phosphorylated tau in AD, and that epitopes derived from the modified proteins are recognized as foreign. It is found that α-syn, while a “self protein”, may play a role similar to proinsulin or glutamic acid decarboxylase-derived neoantigens in T1D that lead to T cell mediated death of islet insulin-secreting cells in the pancreas (Roep and Peakman, 2012; Marrack and Kappler, 2012), or loss of oligodendrocytes in multiple sclerosis (Mars et al., 2012). This study and the present invention provides biomarkers, diagnostic and new therapeutic directions for treatment of PD.

(C) Approach

The Acquired Immune System and Autoimmune Disorders.

The concept of “neoantigens”, proteins that may undergo modification or disrupted normal degradation and become a source for novel antigenic epitopes, was developed from studies of autoimmune disease (Marrack and Kappler, 2012). For example, in T1D, the insulin precursor or glutamic acid decarboxylase enzyme is often presented as an antigen and mis-recognized as a foreign invader, so that the pancreatic beta cells are identified and killed by T cells. Other examples of classical T cell related autoimmune disorders are multiple sclerosis, in which glial cells are destroyed by T cells that mistake myelin protein as foreign (Traugott, 1987), lupus erythematosus (Sawla et al., 2012) and psoriatic arthritis (Diani et al., 2014). Particular MHC alleles are associated with particular neoantigen epitopes in these diseases: e.g., the HLA-DQ alleles DQA1*03:01/DQB1*03:02 are highly associated with T1D and present glutamic acid decarboxylase residues 121-140 and 250-266 (Chow et al., 2014).

The thymus participates in identifying proteins as self or non-self, but disappears in early adulthood. Thymus-derived T cells display an extremely high number of clones, each with different T cell receptors (TCR) on their surface. Millions of different TCRs are present in each individual; this is accomplished through a process of genetic recombination during development similar to antibody production by B cells. Within the thymus, the different T cells are “negatively selected” and deleted if they react with epitopes of intrinsic proteins, which protects against autoimmune response, and “positively selected” if they appropriately recognize foreign epitopes. In classical autoimmune disorders such as T1D, there are errors in this process, leading to identification of self-proteins, e.g., peptides derived from pre-proinsulin, as foreign even in young people (Marrack and Kappler, 2012; Roep and Peakman, 2012).

T cell types include “killer” cytotoxic CD8⁺ T cells and “helper” CD4⁺ T cells. The latter encompass subtypes involved in regulating immune responses, such as “T_{reg}” cells, and others that stimulate the acquired immune system, including recognition of “non-self” proteins that can stimulate killer T cells or antibody-producing B cells. Specific T cell clones, some of which are maintained after antigen exposure in low levels as “memory” T cells, are activated by particular MHC/epitope combinations, leading to cytokine release, clonal expansion, and acquired immune responses. A technology of specific HLA-epitope recombinant protein combinations known as “tetramers” is used to count the number of a particular T cell clone that participates in allergic reaction or autoimmune response (Kurtulus and Hildeman, 2013).

The activation of T cells requires the presentation of a peptide epitope by the heterodimeric MHC molecule expressed on the surface of antigen presenting cells. The inventors have shown this occurs in the specific neurons that are the most prone to neurodegeneration in PD (Cebrian et al., 2014c), a surprise to the field as it had been thought that mature neurons did not exhibit antigen. The HLA (human leukocyte antigen) genes that encode human MHC proteins are located in a region of chromosome 6. The HLA-A, B, and C genes encode alpha subunits of the dimer MHC-I that contain a groove that binds peptides, with an invariant beta microglobulin chain: there are thus typically 6 MHC-I alleles in human with heterozygous alleles. MHC-II binding of peptides is by products of the HLA-D genes, which are duplicated variously in the human population with as many as 6 alpha change genes and 6 beta chain genes, providing larger and variable numbers of MHC-II proteins in an individual (Cano et al., 2007).

MHC-I molecules are size restricted due to barriers on the edge of the groove and display peptides of 8-11 amino acids, and activate so-called cytotoxic or killer CD8⁺ T cells. MHC-II have a longer groove without barriers, and so binds peptide regions of ˜>15 amino acids, activating so-called helper CD4⁺ T cells. Individual HLA genes encompass a tremendous number of alleles, e.g., there are over 2,880 alleles in the human population for HLA-A, providing a broad range of response to infection between individuals. The binding of particular epitopes to the range of MHC are due to differences in amino acid residues in the alpha-(MHC-I) or beta-chain (MHC-II) binding grooves, and so different epitopes are “restricted” for presentation to different alleles. The presented epitope can be from an exogenous peptide loaded into the cell, which is known as “cross presentation” (Bevan, 2006).

An additional basis for this idea was suggested by studies showing that pathologically modified proteins block normal degradation, starting with dopamine-modified α-syn (Martinez-Vicente et al., 2008), which blocks the chaperone-mediated autophagy (CMA) pathway that appears to normally degrade α-syn (Cuervo et al., 2004) and tau (Wang et al., 2009). Studies by a long-term collaborator, Ana Maria Cuervo (Albert Einstein College of Medicine) have shown that CMA substrates can be shunted to an endosomal/lysosomal pathway known as endosomal-microautophagy in multivesicular bodies (MVBs) (Sahu et al., 2011). These organelles normally load antigen onto MHC-II molecules (Strawbridge and Blum, 2007; Crotzer and Blum, 2008; ten Broeke et al., 2013), and trigger an autoimmune cascade (Marrack and Kappler, 2012). This shift from CMA to MVB metabolism could explain the basis of the late stage induction of autoimmune response in PD.

Neurodegenerative Diseases Exhibit Specific Mishandled Proteins:

Neuropathologists identify disorders on the basis of immunolabel for misfolded proteins. These include AD (tau, APP), other forms of dementia (TDP-43), and ALS (TDP-43, FUS). In PD and LBD, the most prominent neuropathological features are the presence of intraneuronal abnormal aggregations of α-syn protein in the form of Lewy bodies and Lewy neurites, particularly in the phosphorylated 129S residue (Spillantini et al., 1997), but phospho-tau aggregates are also present.

There are additional posttranslational modifications that cause a neoantigenic response, including several already studied for inhibition of CMA (Martinez-Vicente et al., 2008), particularly nitrated Y39 (Danielson et al., 2009) and possibly Y129 (Hodara et al., 2004), and dopamine-modified α-syn, which interacts with aa125-129 (Norris et al., 2005). Phosphorylated S129 α-syn bound to lysosomes but was not accumulated by CMA, whereas dopamine-modified α-syn bound avidly but not accumulated and further blocked CMA for other peptides.

Tau, the protein product of the MAPT gene, in highly phosphorylated aggregates has long been associated with AD, progressive supranuclear palsy (PSD) and other dementias, but evidence strongly indicates a role in some PD. MAPT has been identified as a risk factor for PD by GWAS (Sharma et al., 2012), but not AD itself. Phospho-tau can also be high in PD and particularly LBD, while phospho-tau is very high in AD, and there is often significant overlap in patient brain pathology between the disorders. There are additional rarer diseases that also feature such overlap, such as PSP and FTDP-17 which are excluded from this study due to the low number of available patients, as well as multiple system atrophy, for which blood donations are problematic due to orthostatic and circulatory symptoms.

Phosphorylated candidate epitopes are important for tau, which has ˜40 potential phosphorylated sites (Sharma et al., 2012; Yin et al., 2013), of which 20 phosphorylated sites were identified in AD patients (Duka et al., 2013); 10 phosphorylated sites were identified in PD striata (S202, 235, 262, 356, 396/404, 409, 413, 422 and T205, 212); and seven sites in LBD (S214, 238, 396/404, 422 and T212, 217). Interestingly, in PD there are 3 clusters of phospho-tau (202,205, 212; 356, 396, 404; 409, 413, 422).

Tau and α-syn have many parallel features including association with PD by GWAS, phosphorylation under disease conditions, presence of protein (Hampel et al., 2010; Foulds et al., 2013; Zetterberg et al., 2013), and autoantibodies in blood (Bartos et al., 2012; Koehler et al., 2013), and similar degradation by CMA that is disturbed by mutation (Wang et al., 2009). They may even form joint oligomers in some patients (Sengupta et al., 2015). It is therefore considered important to characterize and compare the autoimmune features of these two proteins in these overlapping disorders.

While there are many hypotheses on how these proteins cause each disease, including from our own group (Petersen et al., 2001; Dauer et al., 2002; Cuervo et al., 2004; Martinez-Vicente et al., 2008; Mosharov et al., 2009; Martinez-Vicente et al., 2010; Orenstein et al., 2013), these efforts have not clearly uncovered the mechanisms of cell death. Moreover, in most animal models, demonstration of appropriate targeted neuronal death pathways has not been yet demonstrated. Without wishing to be bound by any scientific theory, it is suggested that this is because multiple “hits” must occur, similar to the idea that multiple genes must be mutated in cancer (Sulzer, 2007).

Without wishing to be bound by any scientific theory, it is suggested that neoantigens of diseases of aging arise because protein processing is altered, as is clear from the formation of the protein aggregates. The resulting epitopes of self proteins were not presented during thymic selection, but could be presented years later by specific HLAs. Some combinations of antigenic epitopes and HLA alleles have already been identified in PD, demonstrating an autoimmune feature in these patients. This is important, as PD has been linked by independent GWAS studies to the HLA alleles B*07:02, C*07:02, DRB5*01, DRB1*15:01, DQA1*01:02, and DQB1*06:02 (Wissemann et al., 2013), which comprise a relatively common linked set of HLA proteins. Remarkably, Data independently show that two of these alleles strongly bind α-syn derived epitopes in PD (see below).

Neuronal Antigen Presentation:

As detailed in reviews (Cebrian et al., 2014a, b) and others, studies starting in the 1920s confirmed the presence of high levels of neuroinflammation in PD and AD pathology, although this was essentially limited to microglial cells, with some evidence for “astroglyosis”. Microglial activation from resident brain inflammatory cells and peripheral macrophage and T-lymphocyte infiltration occur in SN of PD patients and animal models of PD (McGeer et al., 1988; McGeer et al., 2003; Brochard et a., 2009)(McGeer et al., 2003; Moehle and West, 2014). Moreover, cytokines including interferon gamma (IFN-γ), tumor necrosis factor alpha (TNF-α), interleukin 1-beta and interleukin 6 (IL-6) are elevated in the serum and cerebrospinal fluid of PD patients (Mogi et al., 2007; Hirsch and Hunot, 2009). MHC presentation by astrocytes in AD was reported 25 years ago (McGeer et al., 1988; Tooyama et al., 1990), although this insight remained dormant in the literature.

Antigen presentation by adult neurons, however, was discovered only in our recent study. The neurons most targeted in PD, i.e., adult human substantia nigra (SN) dopamine (DA) neurons and locus coeruleus (LC) neurons, present the antigen displaying protein, MHC-I, to date the only neurons in the adult brain identified to do so. This was confirmed by multiple independent techniques, including immunolabel, electron microscopy, proteomics and detection of mRNA. In addition, MHC-I induction was shown in DA neurons derived from human stem cells. CD8+ T cells were occasionally observed in association with the MHC-I DA neurons (Cebrian et al., 2014c). It was found that the combination of activated microglia and/or oxidative stress with neuronal antigen presentation and the presence of the appropriate T cells that recognize the antigen/MHC complex causes neuronal death (Cebrian et al., 2014c). These findings are the first suggesting that neuronal antigen presentation could play a role in adult neurodegenerative disorders.

As detailed in Aims 1 and 2, the activation of T cells to α-syn and possibly tau will be assessed by analyzing blood from patients. Neuronal antigen presentation and its role in neuronal death however, requires testing in animal models (Aim 3). The overall goal is to identify disease-associated antigens and MHC alleles that present them in neurodegenerative disorders.

Influenza and PD.

While not a central focus of this study, this work relates to evidence that some neurotropic viruses cause neurological disorders with a substantial delay after infection. Influenza, in particular, has been linked to encephalitis, parkinsonism and PD (Toovey, 2008), including delayed parkinsonism associated with the 1918 “Spanish flu” epidemic with associated von Economo's encephalitis, widely known via Oliver Sack's book, Awakenings. Some influenza strains are specifically neurotropic for SN DA neurons (Yamada et al., 1996), including neurovirulent influenza A virus (Takahashi et al., 1995; Mori and Kimura, 2000, 2001; Jang et al., 2009a). The highly pathogenic H5N1 influenza virus can enter the CNS and induce neuroinflammation, α-syn aggregation and phosphorylation and SN neuronal death (Jang et al., 2009b). These observations further endorse the possibility of an acquired immune response in some cases of parkinsonism.

Experimental Plans Aim 1. Identify Antigenic Proteins and Epitopes, HLA Alleles, and Responsive T Cells in PD and Other Disorders.

Hypothesis:

Epitopes derived from α-syn are recognized as neoantigens by PD and LBD patient T cells. Tau, which like α-syn is associated with PD, phosphorylated in affected brain, and degraded via the CMA pathway, is a potential candidate for T cell response in PD, LBD and AD and there may be overlap of response.

SubAim 1.1. Identify Possible Antigenic Epitopes:

Potential class I and II MHC epitopes derived from native α-syn and tau are identified by algorithmic protocols developed by Sette's lab and colleagues (Paul et al., 2013; Kim et al., 2014) that are widely used in immunology. Each possible amino acid sequence that can be derived from spanning the entirety of a particular protein, including its pathogenic alleles, is predicted for HLA binding for nearly the entire human population (Paul et al., 2013; Kim et al., 2014). The resulting peptides have been shown to successfully predict MHC-peptide binding of >90% of the human population (Calis et al., 2013). Following the analysis of the candidate antigens of α-syn and tau, each peptide with significant binding potential is manufactured. For α-syn, the 122 most antigenic peptides for MHC-I and MHC-II presentation were already synthesized (A and A, San Diego) and tested as below. The same algorithm is used to calculat 150 candidate tau epitopes that are synthesized.

As detailed above, modified residues, particularly by phosphorylation, are strong candidates to cause antigenic response. There are multiple such modifications, with the most clearly implicated phosphorylation. α-Syn is highly phosphorylated at S129 in PD compared to control both in the Lewy aggregates in brain and in blood, where levels are ˜300% higher in patients (Foulds et al., 2013). For α-syn, all 15 mer peptides containing phosphorylated S129 were thus manufactured. This is also done for phosphorylated Y39 and Y125, which have been reported in both controls and patients: at least three other sites (S87, Y133, Y136) are candidates (Taymans and Baekelandt, 2014), but to our knowledge these have not been identified in brain. This study examines for α-syn 15-mer derivatives of the nitrated residues and the sequences containing the dopamine-reacted sequence prepared as above (Martinez-Vicente et al., 2008). For tau, each candidate epitope of 15 mer peptides containing the 20 candidate phosphorylated residues, including the 10 phosporylated residues found in PD (Duka et al., 2013), are examined for T cell response.

SubAim 1.2: Identify Antigenic Responses Against Possible Epitopes in Patients and Controls.

The binding algorithms in SubAim 1 predict how well the peptides would bind to MHC if present, but not if they are present in patients. To determine this, the presence of T cell response against the candidate antigens by patient blood-derived peripheral blood mononuclear cells (PBMC) is assessed by ELISPOT assays upon stimulation with the peptides. This assay detects and quantifies cytokine release (in this case, IFNγ and IL-5) from specific activated T cells.

Blood from PD, AD, and LED patients and age-matched controls is obtained during diagnosis and checkups. The Columbia University (CU) IRB Blood is drawn from up to 150 controls patients with AD and LBD (PDD/DLB).

Data were obtained from blood samples (25 ml) from 44 PD patients and 24 age-matched controls. The experiments were conducted blindly. PBMCs from these samples were isolated and exposed to pools comprising the 122 candidate class I and class II antigens and phosphorylated S129 epitopes generated in Subaim 1. For the PBMCs responsive to the pooled peptides, the response was then deconvoluted to identify individual epitopes.

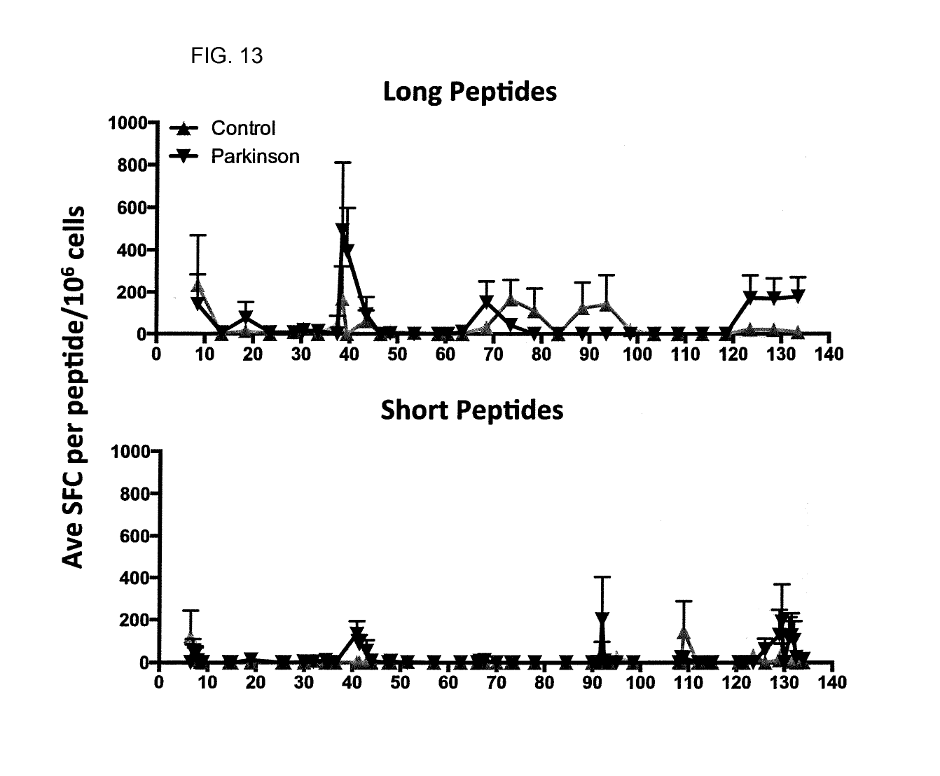

As shown in FIG. 13, there are two regions that contain epitopes in these patients: one includes Y39, which has two shorter antigenic epitopes (37VLYVGSKTK45 (SEQ ID NO: 25), 36GVLYVGSKTK45 (SEQ ID NO: 29)) and two longer antigenic epitopes that differ by one amino acid, 31GKTKEGVLYVGSKTK45 (SEQ ID NO: 59) and 32KTKEGVLYVGSKTKE46 (SEQ ID NO: 34). This region is very close to the several pathogenic mutations in the protein (A30P E46K H50Q G51D A53T) that in some cases were found to interfere with normal CMA degradation (Cuervo et al., 2004). Y39 has recently been discovered to be present in a phosphorylated state in both patients and controls (see Introduction). The two long epitopes have very high affinity for HLA-D subtypes identified in GWAS studies to cause PD (see Aim 2).

The second antigenic region is for regions containing S129 when phosphorylated, which as above is present at very high levels in PD patient blood as well as brain (Anderson et al., 2006). There is presently no restriction found for S129, and they could be recognized by multiple HLA alleles.

A correlation was not found between gender, age, or disease duration in the 44 patients. The strongest T cell response in PD patients to the two regions was observed in long (15-mer) peptides (class II specific). However, a response to the short peptides was only observed in PD patients in these two regions, with no response in controls (p<0.005), so both short and long peptides promise diagnostic and biomarker data.

In summary, 50% of PD patients examined respond to the antigens, with 32% responding to the Y39 epitopes and 23% to the phospho-S129 epitopes with a combined difference between PD patients (n=44) and controls (n=24) of p<0.005. Thus, PD patients possess specific T cells that recognize α-syn as a foreign protein. These Data indicate that PD has autoimmune features.

Note that while not all PD patients exhibit this autoimmune reaction, the level of response is variable in patients over the course of weeks in classical autoimmune disorders such as T1D or MS: e.g., these results already show higher levels of response than most with T1D, which are often ˜20% of individuals (Martinuzzi et al., 2008; Peakman, 2008; Petrich de Marquesini et al., 2010; Velthuis et al., 2010; Wu et al., 2012; Sarikonda et al., 2014). It is possible that the few control subjects who also possessed T cells that response to α-syn are in a pre-clinical stage or in danger of developing the disease.

Expected Outcomes and Limitations:

The Results demonstrate an autoimmune response in PD. The experiments performed 1) compare autoimmune response to α-syn and tau as features of PD, LBD and AD 2) identify the epitope regions responsible for the antigenic response 3) determine the effects of three candidate posttranslational modifications (phosphorylation, nitration, dopamine modification) that arise during neurodegenerative processes. The findings of T cell response to α-syn epitopes by PD patients vs. age-matched controls (p<0.005) should introduce the concept of an autoimmune component in PD.

Tau shows similar components. Tau is highly phosphorylated in these disorders, its aggregations overlap with α-syn to different extents in each disorders, mutations in MAPT are linked to PD, and it undergoes the same degradation pathway by CMA and blocks this pathway in modified forms (Wang et al., 2009). The particular forms of phospho-tau particular to each disease induce a T cell response particularly in patients with that disease, while reaction to α-syn may be less prominent in AD patients, but the overlap in these disorders makes these assays important to conduct. Tau and α-syn are only two candidates for autoimmune response, and for example LRRK2 is highly phosphorylated and may participate, as well as many additional neurodegeneration-linked proteins.

Methods. PD Subjects.

Age-matched PD patients and controls are recruited from the Center for Parkinson's Disease (CPD) at Columbia University. Control participants are usually the spouse or other non-blood related caregivers of PD patients in the Center. PD inclusion criteria is based on UK Brain Bank criteria for the clinical diagnosis of PD. These require 1) the presence of bradykinesia and either rest tremor or rigidity; 2) asymmetric onset; 3) progressive motor symptoms 4) age at onset 40-99 years, 5) patients with autoimmune disorders are excluded. Control inclusion criteria are similar but with lack of PD in first-degree blood relatives (an exception was made for one patient mentioned above), no evidence of PD on exam. AD subjects re recruited from the Aging and Dementia Center at Columbia University and examined Genetic, demographic and clinical data is collected. LED subjects arrive via both the CPD and Aging and Dementia Center. The criteria for diagnosis for AD are according to the NIA/NIH guidelines (McKhann et al., 2011), and for LBD, the guidelines of the DLB consortium (McKeith et al., 2005). The number of patients planned for testing is currently set at 50 per group, based on the incidence of T cell response to date in 50% of patients (see below).

TABLE 4
In cells expressing common HLA class II alleles, two HLA
alleles were found to bind with high affinity to the two antigens
(SEQ ID NO: 59 and 34, respectively) containing Y39, which was
produced high responses in PD patients (see FIG. 13).

| | GKTKEGINING5KTK | KIKEGVLYVGSICIXE |
|---|---|---|
| HLA Allele | IC₅₀ (nM) | IC₅₀ (nM) |
| DPB1*02:01 | 28691 | — |
| DPB1*03:01 | — | 4109 |
| DPB1*04:01 | — | — |
| DPB1*04:02 | 1698 | 2.8 |
| DPB1*05:01 | 1917 | — |
| DPB1*14:01 | 1537 | 16524 |
| DQB1*02:01 | 280 | 25952 |
| DQB1*03:02 | 21682 | 9245 |
| DQB1*C4:02 | — | 9642 |
| DQB1*05:01 | 24475 | 9635 |
| DQB1*06:02 | 30502 | — |
| DRB1*01:01 | 9371 | 23706 |
| DRB1*03:01 | 2944 | 5089 |
| DRB1*04:01 | 355 | 5140 |
| DRB1*04:05 | 1086 | 2450 |
| DRB1*07:01 | 1112 | 177 |
| DRB1*09:01 | 913 | — |
| DRB1*11:01 | 10348 | 22.85 |
| DRB1*12:01 | — | 7124 |
| DRB1*13:02 | — | — |
| DRB1*15:01 | 0.9 | 83.3 |
| DRB3*01:01 | 20717 | 718 |
| DRB3*02:02 | 30462 | 225 |
| DRB4*01:01 | 40440 | — |
| DRB5*01:01 | 56.2 | 8.1 |

Modified Peptide Candidates.

To determine whether the phosphorylated epitopes are major targets of recognition by T cells in patients, for each case, 15-mer chains, which are sufficient for MHC-II binding, are made encompassing phosphorylated residues. As the computational protocols above do not include analysis of phosphorylated sites, 15-mers that overlap by each 10 amino acids are produced, which is sufficient to determine particularly antigenic portions of the hyperphosphorylated protein. For α-syn, nitrated and dopamine modified residues above (aa39, 129, 125-129) are prepared as in our previous publication (Martinez-Vicente et al., 2008).

Immunological Methods.

PMBC fractions are recovered by standard methods with Ficoll gradient and T cells expansion (Oseroff et al., 2010). The methods for the analysis of T cell reactions and measurement of cytokines production using the enzyme-linked immunosorbent spot (ELISPOT) assay are published in analogous experiments to discover antigens in multiple sclerosis (Lolli et al., 2013) and in hay fever allergens (Oseroff et al., 2010). T cell responses are determined by measuring IFNγ and IL-5 release by ELISPOT. Statistical analysis of differences between populations is expected to be non-parametric and analyzed with the U-Mann-Whitney test. If there were a normal distribution of response, which is a priori unlikely as some patients have no T cell response, with an SD=1, 8 patients per group would be required to provide a p>90% that the study detect a >2 fold difference at a two-sided 0.05 significance level.

Aim 2. Characterize the Neoantigen-Responsive T Cells in AD, PD LSD. Hypothesis:

T cells responsive to neoantigens in neurodegenerative disorders encompass memory CD4⁺ and CD8⁺ T cell subsets that have undergone clonal expansion during the progression of the disease.

Plan:

This study identifies and characterizes the phenotype of subsets of responsive T cells from patients using intracellular cytokine staining (ICS) and tetramers of specific HLA alleles of epitopes determined in Aim 1. This may provide a means for diagnosis of PD and other neurodegenerative disorders, as well as biomarkers.

Sub-Aim 2.1. Determine HLA Restriction of T Cell Response.

Aim 1 shows if antigens are present in the patient blood, but particular antigens are restricted to presentation by specific HLA alleles. First, the allelic variants that are capable of presenting specific epitopes are determined for antigens identified in Aim 1 by standard HLA binding assays employing cell lines transfected with single HLA allelic variants. These experiments identify the HLA subtypes particularly prone to presenting each epitope. Using the Sette lab's standard in vitro MHC-peptide binding assays that examines binding in >90% of MHC-II alleles in the human population, it was found that two alleles, DRB1*15:01 and DRB5*01:01, which are typically linked, express very high binding to the antigenic epitopes containing α-syn Y39 (Table 4).

Sub-Aim 2.2. Determine HLA Restriction of T Cell Response.

Sub-Aim 2.1 indicates HLA alleles that can bind epitopes, but not that they do so in patients. Therefore, the HLA class I and/or class II restriction alleles of the responding T cells using the same PBMCs as in Aim 1 are demonstrated by inhibition with locus specific antibodies (anti HLA class I, anti DR, DP and DQ antibodies). The correspondence between HLA genotype for each PBMC donor and the results of identified antigenic peptides in Aim 1 predicts the HLA restriction for each epitope.

In 35 PD patients that were genotyped, it was confirmed that the same two common HLA-DR alleles (DRB1*15:01 and DRB5*01:01) that bound Y39 epitopes in the in vitro assay were that are highly associated with responses to the Y39 epitopes in the PD patients. These two alleles, which are in linkage disequilibrium and frequently expressed together, have been associated separately with PD by GWAS linkage studies (Wissemann et al., 2013) (Table 4). The odds ratio of the presence of these genotypes in 35 patients assayed which showed p values of <0.003, confirming that these two Y39 antigens are restricted to the two HLA alleles in PD patients (Table 5)

TABLE 5
Odds ratio of the presence of these genotypes in 35 patients.

| | A+ | A− | A+ | A− | Relative | Odds | |
|---|---|---|---|---|---|---|---|
| Allele | R+ | R+ | R− | R− | Frequency | Ratio | P-value |
| DRB5*01:01 | 9 | 2 | 5 | 19 | 2.1 | 15.4 | **0.0019 |
| DRB1*15:01 | 9 | 2 | 6 | 18 | 1.9 | 12.3 | **0.0028 |

A+, possesses allele; R+, T cell response.

Sub-Aim 2.3: Investigate the Functional Phenotype of Neoantigen Responsive T Cells.

The identification of the epitopes in Aim 1 allows the determination of the functional characterization of the responsive T cells, including the important issue of whether these are memory cells previously exposed to the antigens. To do so, epitope-specific T cells are stained to establish if they are CD4⁺ or CD8⁺. The phenotype identification is performed for each autoimmune reactive epitope using intracellular cytokine staining (ICS).

ICS to determine the T cell subtype will use cytokines including, but not limited to IFNγ, TNFα, IL-4, IL-17, IL-10, and IL-21. Additional staining for specific surface markers, including CD45RA, CCR7, and CD62L will classify whether the T cells are naïve or memory cells, and determine their activation/exhaustion states, which will indicate if the T cells have been active in the patients. If antigen-specific T cells are of a “memory phenotype”, it will mean that the T cells have been previously exposed and responded to the protein in the patient, and may therefore be involved in the development and/or progression of disease.

The responsive T cells to be analyzed are present in low amounts, and so identifying the functional phenotype requires 10-fold larger blood draws for this purpose (half of a typical 500 ml blood donation, i.e., 250 ml rather than 25 ml for all other blood draws in this proposal). This procedure has been approved for PD patients by CUMC IRB and is expanded to AD and LBD patients. The number of patients analyzed for T cell type is 15, based on the Results that ˜50% show response, yielding ˜7 opportunities to screen for memory cells, and as the alleles showing restriction occur in ˜15% of the general population, although 40% of the PD patients examined to date (Table 5).

Sub-Aim 2.3. Quantify and Further Characterize Responsive Patient T Cells by Using Tetramers.

The combination of allele and epitope is used to produce tetramers that quantify the reactive T cells in individuals (Cecconi et al., 2010). This technique is used as a new means to identify preclinical PD, and by extension, is used in other neurodegenerative disorders that show autoimmune features.

For proven HLA allele-epitope combinations, tetramers are produced to stain and phenotype expanded cells to quantify the autoimmune reactive T cells in patients. For example, we tetramers of DRB1*15:01 and DRB5*01:01 and their two restricted peptides shown in Table 5 are produced. While these T cells recognize this combination in many PD patients but not controls, this allows the quantification of T cells that recognize the restricted epitopes in individual patients; the numbers of specific T cells is correlated with UPDRS scores to determine if this assay might provide a progression biomarker. A battery of such tetramers could provide a general screening to identify “prodromal” individuals, or those that are in danger of developing particular diseases, and eventually could lead to individualized therapies.

Expected Outcomes and Limitations.

Without wishing to be bound by any scientific theory, robust results show HLA allele restriction in PD, provide an explanation for some causes of PD. The typing of T cells determines if the identified antigens are restricted to particular human HLA class I and/or class II phenotypes, while determining the state of responsive T cells provides insight into the biological role they play in neurodegenerative pathogenic processes. For example, if α-syn-specific T cells are memory cells, the T cells have already been exposed to that antigen in that individual, and thus they become suspects in the development and/or progression of disease. Finally, the use of tetramer technology allows individual patients to be screened to characterize their responsive T cells against specific neonantigens; this provides a new means for screening, individualized therapy, identification of prodromal patients, and the development of biomarkers for the autoimmune components of these diseases.

Methods.

PMBC fractions are obtained by standard gradient methods use Ficoll gradient and T cells are expanded as published (Oseroff et al., 2010). The methods for in vitro restriction and T cell ICS assay developed by Sette and collaborators have already been optimized for multiple sclerosis (Lolli et al., 2013) and hay fever (Oseroff et al., 2010) in analogous experiments. HLA typing is performed using standard methods (Oseroff et al., 2010).

Aim 3. Determine the Role of HLA Restricted Alpha-Synuclein Antigen Presentation in a PD Mouse Model.

An experimental question central to the Overall Hypothesis that cannot be tested in human, as it requires the induction of neuronal death, is if the presentation of these epitopes can lead to neurodegeneration. Neurons derived from pluripotent human stem cells could be used, and indeed have been used in publications including the demonstration that these present MHC-I following interferon-gamma (IFN-γ) (Cebrian et al., 2014c). However, components including blood, vasculature, astrocytes, microglia, and other neurons required to emulate the immune response in brain would be lacking. Thus, mouse models are used to address whether neuronal death be driven by α-syn epitopes presented by HLA.

Hypothesis:

Without wishing to be bound by any scientific theory, it is suggested that SN neurons in vivo undergo neuroinflammatory stress or death if the combination of a specific antigen by MHC and the appropriate responsive T cells are present. In Aim 2, such specific epitope and antigen combinations are identified to include specific epitopes restricted to two common HLA alleles, DRB1*15:01 and DRB5*01:01, that also have been found independently in GWAS studies to cause PD.

As mentioned in the above, David Standaert's lab recently showed that viral overexpression of α-syn in mice using AAV leads to SN dopamine neuron death but that neuronal death, is absent in mice null for MHC-II (Harms et al., 2013). Equally important, a prominent immunologist, Chilla David of the Mayo Clinic, has produced and donates a mouse line that expresses only human DRB1*15:01 on a background otherwise null for MHC-II. This line has already been used for studies on autoimmunity including for Rh factor (Bernardo et al., 2014).

Thus, it is tested whether this SN neuronal death and associated features of PD can be replaced in MHC-II null mice by specific reinstatement of the particular gene implicated in presentation of the identified epitopes. The results are compared with standard inbred strains, the MHC-II knockout, the MHC-I knockout, and a additional MHC-II line that does not show restriction for the antigenic epitopes identified in Aim 1.

Plans 1) Choice of α-Syn Mouse Model.

As neuronal antigen presentation and death cannot be assessed in living human, a mouse PD model is used. No “perfect” PD mouse model has been published despite decades of efforts. The classical models use neurotoxins specific for DA SN neurons, including MPTP and 60HDA, but while these are effective at determining the consequences of DA neuron loss, there is a strong and reasonable doubt that these exogenous toxins recapitulate important steps in PD pathogenesis.

Significant effort has therefore gone into developing α-syn animal models, as α-syn aggregation is a convergent pathology for nearly all PD patients (an exception is for some rare juvenile PD due to parkin mutations). The α-syn transgenic models have to date proven difficult to apply for this purpose, most of which do not display neuronal death in the SN (Magen and Chesselet, 2010). The only studies of α-syn transgenic mice that display extensive SN dopamine neuronal death involve the overexpression of the A53T mutant (Lin et al., 2012; Oliveras-Salva et al., 2014; Chen et al., 2015), which is extremely rare in PD and less applicable to a model of how α-syn may act in idiopathic PD.

Viral vectors that overexpress α-syn, in contrast, have successfully produced nigrostriatal degeneration (Kirik et al., 2002; St Martin et al., 2007). This may not be surprising, as in humans, overexpression of wild-type α-syn due to gene multiplication causes rare and virulent familial PD (Singleton et al., 2003). Adeno-associated virus vector, serotype 2 (AAV2-SYN) is considered, which was introduced in rat models, to provide the best available model to test this hypothesis, as it replicates several important features of PD: synucleinopathy (Kirik et al., 2003), age-dependent neuronal loss in the SN that progresses slowly (Yamada et al., 2004; St Martin et al., 2007), substantial suppression of tyrosine hydroxylase (TH) activity and striatal DA levels (Kirik et al., 2002) and mild motor impairment according to the apomorphine rotation and paw-reaching behavioral tests (Kirik et al., 2002).

An AAV2-SYN mouse PD model from the Standaert lab is adapted in which human α-syn is overexpressed by intracerebral injection (Theodore et al., 2008), and as mentioned, the model is mentioned to indicate a role for MHC-II expression in SN neuronal death (Harms et al., 2013). Injection of AAV2-SYN in the SN of WT mice results in 1) no immediate neuronal death after the injection, no visible neuronal loss after 3 months, and 30% SN neuronal death after 6 months; 2) proliferation of macrophages in the brain; 3) an increase in the levels of immunoglobulins and proinflammatory cytokines peaking 2 weeks post injection, 4) infiltration of B and T lymphocytes (Theodore et al., 2008).

Results.

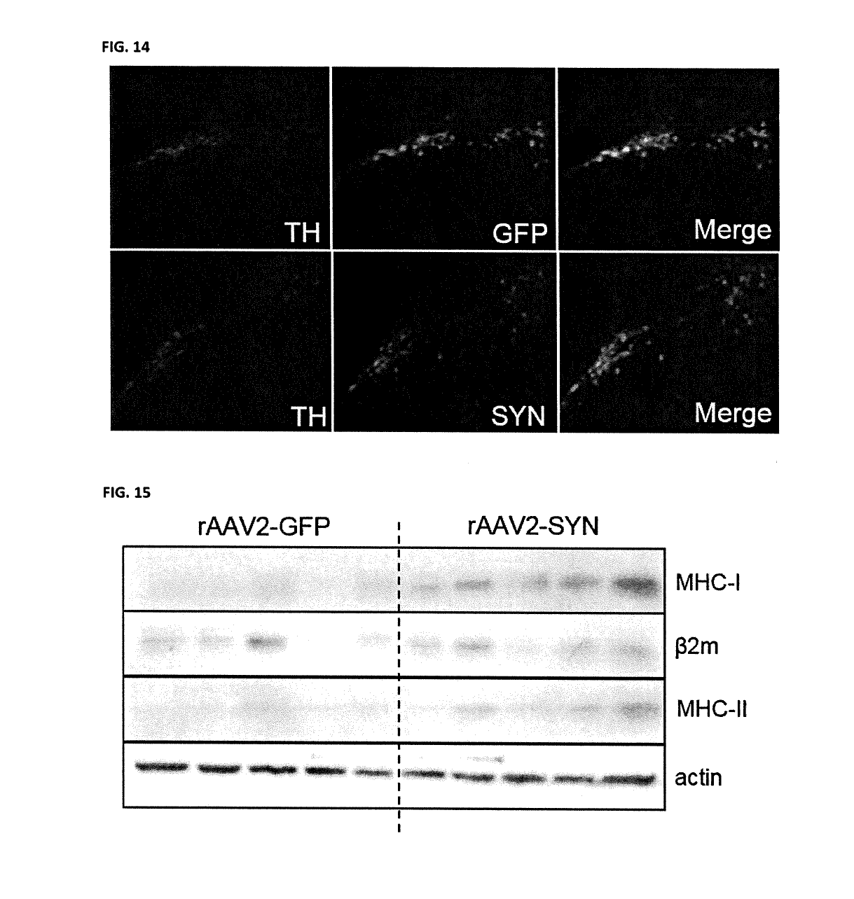

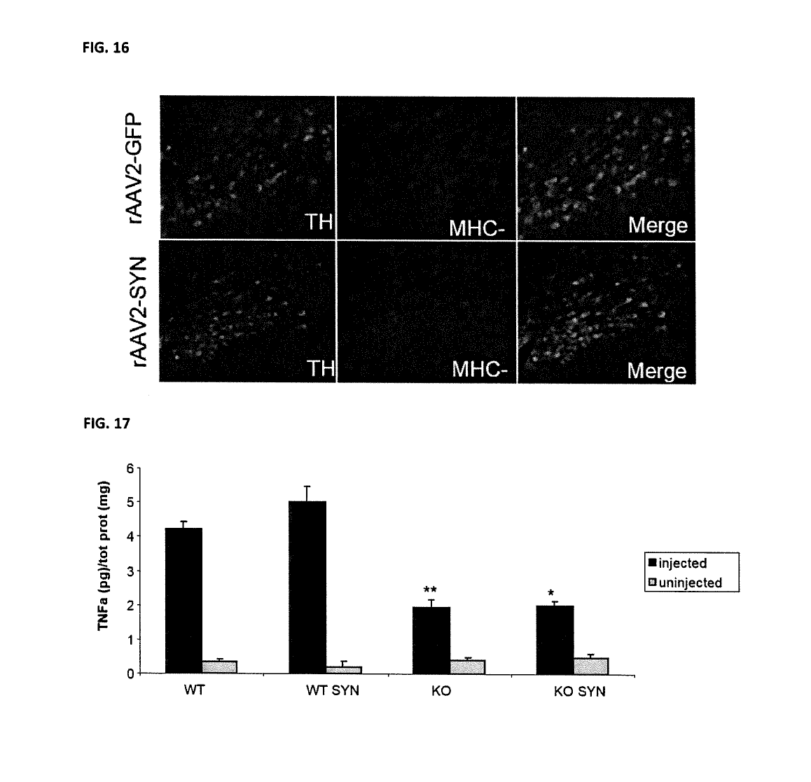

By 2 weeks post AAV, there is excellent colocalization of SN DA neurons and virally expressed human wild-type α-syn and GFP used as a control (FIG. 14).

Experimental Groups.

For each mouse line, AAV2-GFP and AAV2-SYN treated mice are compared. The genotypes compared are

1) wild type (WT: C57BL/6),

2) the same line deficient for MHC-I

3) or deficient for MHC-II

4) the DRB1*15:01 in the MHC-II background

5) a DRB1*04:01 line: while this allele is implicated in T1D and MS, it has no affinity for the epitopes examined, and may provide a useful negative control.

Without wishing to be bound by any scientific theory, it is hypothesizes that the MHC-I deficient lines may not show neurodegeneration, as shown for MHC-II deficient lines by the Standaert group (Harms et al., 2013). It is hypothesized that neurodegeneration should be reinstated following AAV injection in the DRB1*15:01, but not the DRB1*04:01 line.

MHC Response.

In WT mice, the α-syn expressing virus induces far more MHC-I and MHC-II (FIG. 15) protein in SN than the GFP control. MHC-I immunolabel in SN was also substantially higher, indicating that SN neuronal MHC-I is indeed inducible. MHC-I and II will be examined in each experimental group.

Neurodegeneration.

The AAV2-SYN PD model in WT mice causes a delayed neuronal loss in SN 3 to 6 months after the infection, when 30% of neuronal death is observed (St Martin et al., 2007; Theodore et al., 2008). Neuronal loss in response to α-syn and control GFP virus is compared 12 and 24 weeks post-injection in each line. Cell counting is performed with a computer assisted stereological toolbox software program (Stereo Investigator, MBF, USA) used in the Sulzer lab with the optical fractionator method.

Behavioral tests are performed in AAV2-GFP and AAV2-SYN treated mice of each genotype at 3 and 6 months after the injection. The corridor test is performed to address the lateralization of the lesion (Dowd et al., 2005; Fleming et al., 2013), and the beam traversal challenge test, the cylinder test and the adhesive test, to assess the motor abilities of the injected mice.

DA release and reuptake, which should be decreased with SN axonal degeneration, is measured in striatal slice using cyclic voltammetry methods standard, and in part developed, by the Sulzer lab, which have used in a variety of mouse models of PD (Abeliovich et al., 2000; Schmitz et al., 2013; Mosharov et al., 2014).

Cytokine Analysis.

Injection of AAV2-SYN in the WT mice caused a profound increase in several proinflammatory cytokines 2 weeks post-injection (Theodore et al., 2008). ELISAs are performed to detect the release of IFN-γ, TNF-α (FIG. 17), IL-1β (three major proinflammatory cytokines) and IL-17A (an indicator of activated T cells) in the SN of WT and MHC-I KO mice, 2 weeks post-injection.

Macrophage Label.

In WT animals, injection of AAV2-SYN produces a marked proliferation of macrophages in the brain (Theodore et al., 2008), which reaches maximum levels 4 weeks post-injection. 40 μm brain cryosections are immunolabelled 4 weeks after the injection for the macrophage/microglial markers, Iba-1 and CD11b. Astrogliosis are addressed by the levels of GFAP. Quantification of the levels of those proteins is assessed by western blot.

Infiltration of B and T Lymphocytes.

AAV2-SYN in the WT animals caused infiltration of B and T lymphocytes in the mouse SN (Theodore et al., 2008) 2 to 12 weeks after the injection. The presence, location and quantification of these two types of lymphocytes in the SN of each line is assessed. Adjacent SN sections of both groups of animals are immunolabeled for CD4 to mark helper T cells, CD8 to mark cytotoxic T cells, and CD45R (a marker for S cells). The quantification of infiltrated lymphocytes is assessed by western blot and unbiased stereology.

Expected Outcomes:

MHC-II null mice, as previously shown, are protected from AAV2-SYN injection, but neurodegeneration is reinstated in mice expressing DRB1*15:01, but not DRB1*04:01. Without wishing to be bound by any scientific theory, it is anticipated that AAV2-SYN injection of MHC-I null mice may trigger neuroinflammation (activated microglia, presence of proinflammatory cytokines, infiltration of lymphocytes), but that the downstream SN DA neuronal death may be blocked, although it may also be that MHC-I plays little role, which would also be important. These results provide an important in vivo test of the hypothesis that display of antigen restricted to a human MHC-II allele associated with PD leads to selective neurodgeneration in the disease. Together with the set of data in vitro and in human, this redefines the immunological component of PD, and provides evidence for a novel mechanism of neuronal death due to T-cell activity.

Methods:

Recombinant adeno-associated virus, serotype 2 (rAAV2) containing the gene for human αSYN (AAV2-SYN) or green fluorescent protein (AAV2-GFP) virus packaged as published (Zolotukhin et al., 1999; Theodore et al., 2008). 3.5×10¹² viral genome/ml for AAV2-SYN and 5.3×10¹² viral genome/ml for AAV2-GFP is injected stereotaxically into the right SN pars compacta of the mice (coordinates: anterior-posterior, −3.2 mm from bregma, medio-lateral, −1.2 from midline and dorso-ventral, −4.6 from the dura). Half of the animals on each group are transcardially perfused with 4% paraformaldehyde in phosphate buffered saline, and transferred to sucrose 30% for cryosectioning. 40 μm sections are serially collected in order to perform immunolabel. The SN of other the half is dissected, homogenated and quantified in the appropriate lysis buffer to be assayed by western blot or ELISA.

Plans.

The present study leads to new means to provide individualized therapy in neurodegeneration. One such possible therapy is “tolerization”, as performed for allergens, in which T cells are trained to recognize otherwise activating proteins as self. Tolerization therapy and T cell suppression may provide therapy for PD. Further potential additional PD-related antigens, such as LRRK2 and glucocerbrosidase, are examined.

There are important basic neuronal biology questions to address, particularly to elucidate why these proteins are loaded onto MHC, presumably when they are misfolded. This would intersect with the endosomal, lysosomal and proteosomal pathways responsible for the generation of antigenic fragments, and may would shed light on why this occurs with increased age and during the course of certain diseases. The phosphorylated S129 residue may be particularly antigenic due to “cross presentation”, and, if so, this should be replicatable on the responsible T cells isolated from patients.

Tetramer technology using particular HLA-epitope combinations can be used with high sensitivity and specificity to stain and track specific T cells in blood and other human tissue, including histological sections and autopsy samples. These reagents can be utilized to further probe the potential role of T cells that react specifically to α-syn. They can also be used to ascertain if individuals have α-syn-responsive T cells prior to the disease, and may provide a biomarker for its progression, which would be valuable for future therapy.

This study contributes to understanding the pathogenesis of PD. Without wishing to be bound by any scientific theory, T cells may be activated first by peripheral display of antigens prior to infiltrating and inducing damage in the CNS. This could explain, for example, how an infectious agent like influenza travels through the nervous system. Some investigations show that influenza virus elicits neuroinflammatory responses that persist long after the primary infection (Jang et al., 2009a).

### Discussion

Antigen presentation requires the expression of major histocompatibility complex (MHC) molecules. This involves the partial intracellular degradation of self and non-self proteins into 8-14 amino acid peptides, loading them to the antigen binding groove of the MHC class I (MHC-I) or class II (MHC-II), and translocating the complex to the cell surface for display (Chemali et al., 2011).

Although detection of MHC-I in the mature rodent central nervous system (CNS) was for many years confined to glial cells (Wong et al., 1984) a body of continuing reports demonstrates that MHC-I can be expressed by some neuronal populations, both in vitro, usually triggered by exposure to interferon gamma (IFN-γ), and in vivo. An initial study showed that MHC-I genes could be induced by IFN-γ in cultured rat hippocampal neurons (Neumann et al., 1995). Subsequently, mRNA for MHC-I was identified in multiple regions of the neonatal and adult rodent brain including the substantia nigra (SN), brainstem motor neurons (Lindå et al., 1999) the lateral geniculate nucleus (LGN), the cortex, the hippocampus (Huh et al., 2000) and cerebellum (Letellier et al., 2008). Subunits of the MHC-I molecule were detected by immunolabel in these regions (Needleman et al., 2010; Liu et al., 2012), with expression gradually decreasing as neonatal mice reached adulthood in regions including cingulate cortex and the hippocampus (Liu et al., 2012). These data suggest a potential role of MHC-I in early development that may be absent in maturity.

Neuronal MHC-I expression is thought to play a role in early developmental synaptic plasticity (Goddard et al., 1998; Corriveau et al., 1998; Glynn et al., 2011), in regeneration of neurons after axotomy (Oliveira et al., 2004) and in hippocampus-dependent memory (Nelson et al., 2013). MHC-I expression has also been shown in cultured hippocampal embryonic neurons that were targeted and killed by CD8⁺ cytotoxic T cells (CTLs) in response to exogenous viral or ovalbumin (OVA)-derived antigens (Medana et al., 2000; Meuth et al., 2009) Finally, neurons can be MHC-I-dependent targets for CTLs following neurotropic viral infections (Chevalier et al., 2011).

In humans, MHC-I has been reported in microglia and endothelia of the hippocampus in control individuals and Alzheimer's disease patients (Tooyama et al., 1990). MHC-II immunolabel of microglia, but not neurons, has been reported in the SN of patients with Alzheimer's and Parkinson's disease (PD) (McGeer et al., 1988) and in the hippocampus of patients with dementia with Lewy bodies (Imamura et al., 2005). Neuronal expression of MHC-I in the human brain has only been reported in a small number of studies. The first was a study of a childhood viral infection, Rasmussen's encephalitis, in which immunolabel for the MHC-I component, beta 2 microglobulin (β2m), was present in cortical and hippocampal neurons (Bien et al., 2002); more recently, MHC-I has also been observed in dysmorphic/dysphasic cortical neurons of focal cortical dysplasia, tuberous sclerosis complex and ganglioglioma cases (Prabowo et al., 2013) and in the embryonic LGN of the dorsal thalamus (Zhang et al., 2013a) and hippocampus (Zhang et al., 2013b). In the LGN, β2m was observed at 29-31 gestational weeks but was nearly absent by postnatal day 55, and was completely absent in the adult (Zhang et al., 2013a). MHC-I in the human visual cortex was not observed at any gestational or postnatal stage (Zhang et al., 2013a), while the expression of MHC-I was very low in the hippocampus at 20 gestational weeks and slowly increased during weeks 27-33. A rapid increase in MHC-I molecule expression was found in the subiculum that reached high levels at 31-33 gestational weeks, but no expression of MHC-I was found in the adult hippocampus (Zhang et al., 2013b). To date, there has been no evidence reported of neuronal MHC-I expression in the normal adult human brain.

Recent reports have demonstrated that some rodent neurons express major histocompatibility complex class I (MHC-I), but expression of this antigen-presenting protein has not been reported in normal adult human neurons. Evidence provided herein from immunolabel, RNA expression, and mass spectrometry in human postmortem samples demonstrates that the majority of human pigmented substantia nigra (SN) dopaminergic (DA) and locus coeruleus (LC) norepinephrinergic neurons express MHC-I. MHC-I expression was also induced in human stem cell derived DA neurons. Using murine primary neurons as a model, experiments in the Examples herein found that catecholamine neurons were far more responsive to induction of MHC-I by gamma-interferon than other neuronal populations examined. In addition, neuronal MHC-I was induced by factors released from microglia activated by neuromelanin or alpha-synuclein or by treating the neurons with the catecholamine precursor, L-dihydroxyphenylalanine, suggesting that cytosolic DA and/or oxidative stress may trigger selective MHC-I expression. The inventors observed that these neurons internalized foreign ovalbumin and displayed the ovalbumin-derived antigen SIINFEKL (SEQ ID NO: 9), and that the combination of antigen presentation with MHC-I expression triggered DA neuronal death by CD8+ cytotoxic T cells. These results indicate that T cell infiltration of the central nervous system observed in neurodegenerative disease coupled with antigen/MHC-I display may lead to death of SN and LC neurons.

Although the detection of MHC-I in the mature rodent central nervous system (CNS) was for many years confined to glial cells (Wong et al., 1984), a body of developing work demonstrates that MHC-I can be expressed by some neuronal populations, both in vitro, usually triggered by exposure to interferon gamma (IFN-γ), and in vivo. An initial study from Neumann et al. (1995) showed that MHC-I genes could be induced by IFN-γ in cultured rat hippocampal neurons.

Following that report, mRNA for MHC-I has been identified in several regions of the neonatal and adult rodent brain including the substantia nigra (SN), brainstem motor neurons (Lindå et al., 1999), the lateral geniculate nucleus (LGN), the cortex, the hippocampus (Huh et al., 2000) and cerebellum (Letellier et al., 2008). Subunits of the MHC-I molecule have also been detected by immunolabel in these regions (Needleman et al., 2010; Liu et al., 2012), with expression gradually decreasing in regions including cingulate cortex and the hippocampus as the newborn mice reached adulthood (Liu et al., 2012). These data suggest a potential role of MHC-I in early development that may be absent in maturity.

Neuronal MHC-I expression is thought to play a role in early developmental synaptic plasticity (Goddard et al., 2007; Corriveau et al., 1998; Shatz, 2009; Glynn et al., 2011; Elmer and McAllister, 2012), in the regeneration of neurons after axotomy (Oliveira et al., 2004; Sabha et al., 2008) and in hippocampus-dependent memory (Nelson et al., 2013). The expression of MHC-I has also been shown in hippocampal embryonic neurons in culture that were targeted and killed by CD8+ cytotoxic T cells (CTLs) in response to exogenous viral or ovalbumin (OVA)-derived antigens (Medana et al., 2000; Meuth et al., 2009). In addition, there is evidence showing that neurons can be MHC-I-dependent targets for CTLs upon neurotropic viral infection (Rall et al., 1995; Richter et al., 2009; Chevalier et al., 2011).

In humans, MHC-I has been reported in microglia and endothelia of the hippocampus in control individuals and Alzheimer's disease patients (Tooyama et al., 1990). MHC-II immunolabel of microglia, but not neurons, has been shown in the SN of patients with Alzheimer's and Parkinson's disease (PD) (McGeer et al., 1988), and in hippocampus of patients with dementia with Lewy bodies (Imamura et al., 2005). Neuronal expression of MHC-I in the human brain has only been reported in 1) a study of a childhood viral infection, Rasmussen's encephalitis, in which immunolabel for the MHC-I component, beta 2 microglobulin (β2m), was identified in cortical and hippocampal neurons (Bien et al., 2002), and 2) in the LGN of the dorsal thalamus (Zhang et al., 2013a) and the hippocampus (Zhang et al., 2013b) of embryos. In the LGN, β2m was observed at 29-31 gestational weeks but decreased with development and was nearly absent at postnatal day 55 and completely absent in adult LGN (Zhang et al., 2013a). MHC-I in the visual cortex was not observed at any gestational or postnatal stage (Zhang et al., 2013a). These authors also showed that the expression of MHC-I was very low in the hippocampus at 20 gestational weeks and slowly increased at 27-33. A rapid increase in MHC-I molecules expression was found in the subiculum that reached high levels at 31-33 gestational weeks, but no expression of MHC-I was found in the hippocampal formation of adults (Zhang et al., 2013b). To date, there has been no evidence reported of the presence of neuronal MHC-I expression in the normal adult human brain.

The Examples herein show that 1) in human postmortem samples from adult control individuals, MHC-I is expressed by SN dopaminergic (DA) and locus coeruleus (LC) norepinephrinergic (NE) neurons, 2) human stem cell (hES)—in vitro—derived DA neurons can be induced to express MHC-I in their membranes, 3) catecholamine murine neurons in primary culture are more prone to display MHC-I upon IFN-γ challenge than non-catecholamine neurons, 4) SN DA murine neurons in culture display MHC-I in response to microglia activated by neuromelanin (NM) or alpha-synuclein (α-syn), substances found extracellularly in postmortem PD brain, 5) in the absence of microglia, chronic exposure to the DA precursor, Ldihydroxyphenylalanine (L-DOPA), induces MHC-I in SN DA murine neurons, 6) SN murine neurons in culture can process and present foreign protein antigens by MHC-I, 7) in the presence of the appropriate antigen and CTLs, MHC-I expressing SN murine neurons are destroyed.

These findings suggest that neuronal MHC-I expression and antigen display in catecholamine neurons are triggered by microglial activation or high cytosolic DA, which, in the presence of the appropriate antigen and CTLs might play a critical role in neuronal death during diseases like PD in which CNS inflammation is robust.

Without wishing to be bound by any scientific theory, an immunologically-based mechanism that may link activated microglia, increased cytosolic oxidative stress and neuronal death of catecholamine neurons is proposed herein. Microglia activated by NM, native α-syn, modified α-syn, or mutant α-syn release IFN-γ that in turn induce MHC-I expression in neurons. Catecholamine neurons may be subject to additional oxidative stress due to the presence of cytosolic DA, and this could lead to their MHC-I induction. The capacity of catecholamine neurons to process and display antigens may render them selective targets for T cell mediated cell death.

Although CNS neurons have been classically considered “immunoprivilaged” and to not present antigen, several studies have identified neuronal MHC-I expression in rodents (Neumann et al., 1995; Huh et al., 2000; Medana et al., 2000; Oliveira et al., 2004; Goddard et al., 2007; Corriveau et al., 1998; Meuth et al., 2009; Needleman et al., 2010; Glynn et al., 2011). To our knowledge, the first data demonstrating that human adult SN and LC neurons express MHC-I, as confirmed by a variety of approaches, is provided herein. First, immunolabel for both MHC-I subunits, HLA and β2m, was detected in SN and LC neurons by fluorescent, histochemical and electron microscopic means. Second, mRNA for both subunits was identified in laser captured SN NM-containing neurons. Third, using mass spectrometry, β2m and specific HLA alleles were identified both in isolated NM organelles and purified NM from organelles and tissues. The human population expresses thousands of HLA-A, B and C alleles (Marsh et al., 2005), and remarkably, in samples of isolated NM organelles and purified NM obtained from a single subject's SN, mass spectrometry identified peptides that indicate the expression of specific HLA-A, HLA-B and HLA-C alleles (Table 1 and 2) Without wishing to be bound by any scientific theory, it is thus concluded that in contrast to a long-standing consensus, a subset of adult human neurons express MHC-I. In agreement with previous studies (Prabowo et al., 2013; Zhang et al., 2013a; Zhang et al., 2013b), the presence of neuronal MHC-I or β2m was not found in other regions of the adult brain examined including cortex, the hippocampus or the striatum. Human stem cell-derived DA neurons that exhibit properties of SN DA neurons (Kriks et al., 2011), were also induced to express MHC-I by human IFN-γ, a response analogous to that of mouse neurons. Thus, multiple lines of independent data demonstrate that human neurons, in particular catecholamine neurons, possess the ability to express MHC-I in their cell membrane.

When the expression of HLA in TH⁺ neurons was compared in postmortem samples from control versus PD individuals in Examples herein, the proportion of HLA⁺ in SN and LC neurons was found to be higher in control than in pathological specimens (FIGS. 1E and 1F). This would be expected if neurons that were HLA⁺ were selectively destroyed over the course of the disease. However, without wishing to be bound by any scientific theory, emphasis is made to interpret the results cautiously, as the initial proportion of HLA⁺ cannot be ascertained, nor can the duration that human neurons express the protein. In many cases, moreover, the total number of surviving NM⁺/TH⁺ neurons, especially in the LC PD sections, was very low and unlikely to reflect the proportion during early disease stages. It is further possible that the presence of HLA in human catecholamine neurons is related to another biological process and not to antigen presentation.

In Examples herein, an association of MHC-I and NM organelles was identified by mass specrometry and immunolabel. NM organelles are autophagic lysosomes that trap and concentrate cytosolic and organelle proteins, and NM synthesis is induced by cytosolic DA (Sulzer et al., 2000). These lysosomal compartments may participate in neuronal antigen presentation, either as an alternative to or following proteasomal processing (Kleijmeer et al., 2001; Chemali et al., 2011). New work demonstrates roles for autophagic lysosomes in the MHC-I response to bacterial infection (Fiegl et al., 2013), suggesting that uptake into lysosomes may participate in the normal handling and degradation of the protein.

Consistent with previous studies (Neumann et al., 1995; Medana et al., 2000; Meuth et al., 2009), the data herein show that the cultured murine neurons from multiple regions (VM, LC, cortex, striatum, thalamus) could be induced to display MHC-I. The data herein demonstrate that catecholamine neurons of the VM and LC were the most responsive to MHC-I induction. L-DOPA, which is converted to cytosolic DA in SN and LC neurons, triggered MHC-I expression, consistent with a role for oxidative stress in enhancing MHC-I (Teoh and Davies, 2004) via cytosolic catecholamine oxidation. Without wishing to be bound by any scientific theory, it may be that the presence of cytosolic catecholamine is responsible for the particular presence of MHC-I in human SN and LC catecholamine neurons. Without wishing to be bound by any scientific theory, it is noted that these findings should not be construed as suggesting that L-DOPA should not be used clinically; the high exposures used in this study are intended to load the cytosol with high DA levels and not to emulate therapeutic L-DOPA exposure in patients with PD. Additional oxidative stress in LC and SN neurons may result from tonic neuronal firing activity driven by L-type calcium currents (Mosharov et al., 2009; Chan et al., 2009).

The results with OT-I T cells shown herein, indicate that neuronal death ensues when neuronal MHC-I is expressed and displays a specific antigen recognized by a specific CTL. Previous studies of neuronal killing by CTLs (Medana et al., 2000; Meuth et al., 2009) used exogenous short viral and OVA-derived peptides in culture. It is shown herein that VM DA neurons can internalize and present foreign antigen, since addition of the large precursor protein OVA to VM cultures induced SIINFEKL-MHC-I expression on the plasma membrane and within neuronal cytosol. This neuronal MHC-I is immunologically competent, as demonstrated by the induction of CTL proliferation and both Fas and perforin-based CTL neuronal killing. While the loading of peptide onto MHC-I can apparently occur in neurons as well as extracellularly, proteolytic processing of the OVA might occur in non-neuronal compartments, including in astrocytes or by extracellular proteases.

There is a general consensus that many neurodegenerative diseases induce a robust inflammatory response, but it remains unclear how this inflammatory response is related to chronic neurodegeneration.

Without wishing to be bound by any scientific theory, the results shown herein suggest a selective upregulation of neuronal MHC-I, antigen presentation and cytolytic activity that may participate under some pathophysiological conditions. It has recently been demonstrated that microglia can be activated by substances released by degenerating neurons in PD, such as α-syn (Zhang et al., 2007; Beraud et al., 2013) or NM (Zhang et al., 2011; Zhang et al., 2013c), and that activated microglia can elicit neurotoxic responses (Block et al., 2007; Lull and Block, 2010; Zhao et al., 2013). Both NM and α-syn are found extracellularly in the postmortem brain of PD patients (Double, 2012), a disorder that features high levels of activated microglia in the SN (Foix and Nicolesco, 1925) and high levels of intracellular oxidative stress (Fahn and Sulzer, 2004). PD patients are reported to possess CNS chemotactic signals (Harris et al., 2012) and a compromised blood brain barrier (German et al., 2011; Farkas et al., 2000), which may explain why CTLs are found at substantially higher numbers in PD patients than age-matched controls (Brochard et al., 2009).

Catecholamine neuronal display of antigenic MHC-I could participate in a range of additional neurological disorders. For example, Japanese encephalitis virus can induce MHC-I expression in non-neuronal cells by interferon type 1 (Abraham et al., 2010), while in mice, IFN-γ plays a role in paraquat-induced neurodegeneration involving oxidative and proinflammatory pathways (Mangano et al., 2011). CNS-directed expression of IFN-γ produces basal ganglia calcification and nigrostriatal degeneration (Chakrabarty et al., 2011). In human case studies, a link is described between chronic hepatitis C patients who were treated with type 1 interferon and developed PD-like symptoms that reversed when the treatment was halted (Almeida et al., 2009). Thus, both clinical reports and the present results suggest reason to further explore roles for activated microglia, antigen presentation, neuronal MHC-I expression, and recruitment of CTLs in neurodegenerative diseases, including PD, that feature the presence of T cells, activated microglia, intracellular oxidative stress and aggregates of α-syn in the SN and LC.

Mutations or duplications in the gene α-synuclein cause Parkinson's disease (PD). Data herein show that normal people rarely have T cells against α-synuclein, whereas to date the majority of PD patients do. The present invention is useful to diagnose, confirm, provide a biomarker for, and treat PD.

As described herein, human neurons that are destroyed in PD, specifically the substnatia nigra and locus coeruleus, are to date the only adult human neurons that display the antigen presenting protein MHC-I. T cells capable of killing such neurons are already present particularly in PD brain. As described herein this system was replicated with mouse neurons, and the data herein showed that T cells indeed can kill these neurons. Without wishing to be bound by any scientific theory, these data suggest that at least some PD is in part an autoimmune disorder.

There is no data in the literature of what the T cells might recognize, and the data herein is the first to show that it recognizes α-synuclein.

Aspects of the present invention relate to the surprising discovery that epitopes that activate leukocytes are expressed on the surface of neurons in subjects afflicted with PD. Surprisingly, these epitopes are useful in diagnostic and treatment methods for PD. The present invention provides improved and novel methods for diagnosing, confirming, providing biomarkers for, and treating PD are needed. Additionally, specific treatments tailored for individual patients are provided herein.

### REFERENCES

Abeliovich et al. (2000) Mice lacking alpha-synuclein display functional deficits in the nigrostriatal dopamine system. Neuron 25:239-252.
Abraham et al. 2010. Japanese encephalitis virus utilizes the canonical pathway to activate NF-kappaB but it utilizes the type I interferon pathway to induce major histocompatibility complex class I expression in mouse embryonic fibroblasts. J. Virol. 84, 5485-93.
Ahmed et al. (2012) Association between Parkinson's disease and the HLA-DRB1 locus. Mov Disord 27:1104-1110.
Almeida et al. 2009. Interfereoninduced Parkinsonism in a patient with chronic hepatitis C. Arq. Neuropsiquiatr. 67, 715-6.
Álvarez-Erviti et al. 2011. Alphasynuclein release by neurons activates the inflammatory response in a microglial cell line. Neurosci Res. 69(4):337-42.
Anderson et al. (2006) Phosphorylation of Ser-129 is the dominant pathological modification of alpha-synuclein in familial and sporadic Lewy body disease. J Biol Chem 281:29739-29752.
Armstrong et al. (2014) A quantitative study of alpha-synuclein pathology in fifteen cases of dementia associated with Parkinson disease. J Neural Transm 121:171-181.Pmc4041534.
Arnold et al. (2013) Comparative survey of the topographical distribution of signature molecular lesions in major neurodegenerative diseases. J Comp Neurol 521:4339-4355.Pmc3872132.
Bartos et al. (2012) Patients with Alzheimer disease have elevated intrathecal synthesis of antibodies against tau protein and heavy neurofilament. J Neuroimmunol 252:100-105.
Beraud, et al. 2013. Microglial activation and antioxidant responses induced by the Parkinson's disease protein α-synuclein. J Neuroimmune Pharmacol. 8(1):94-117.
Bernardo et al. (2014) RhD Specific Antibodies Are Not Detectable in HLA-DRB1(*)1501 Mice Challenged with Human RhD Positive Erythrocytes. Advances in hematology 2014:470242.Pmc4297636.
Bevan M J (2006) Cross-priming. Nature immunology 7:363-365.
Bien et al. 2002. Destruction of neurons by cytotoxic T cells: a new pathogenic mechanism in Rasmussen's encephalitis. Ann. Neurol. 51, 311-8.
Block et al. 2007. Microglia-mediated neurotoxicity: uncovering the molecular mechanisms. Nat Rev Neurosci. 8(1):57-69.
Brochard et al. 2009. Infiltration of CD4+ lymphocytes into the brain contributes to neurodegeneration in a mouse model of Parkinson disease. J. Clin. Invest. 119, 182-192.2613467.
Budhu et al. 2010. CD8+ T cell concentration determines their efficiency in killing cognate antigen-expressing syngeneic mammalian cells in vitro and in mouse tissues. J. Exp. Med. 207, 223-235.
Calis et al. (2013) Properties of MHC class I presented peptides that enhance immunogenicity. PLoS Comput Biol 9:e1003266.3808449.
Cano et al. (2007) Common and well-documented HLA alleles: report of the Ad-Hoc committee of the american society for histocompatiblity and immunogenetics. Human immunology 68:392-417.
Cannon, J. R., and J. T. Greenamyre. 2009. NeuN is not a reliable marker of dopamine neurons in rat substantia nigra. Neurosci. Lett. 464, 14-7.
Chakrabarty et al. 2011. Interferon-γ induces progressive nigrostriatal degeneration and basal ganglia calcification. Nat. Neurosci. 14, 694-696.
Cebrian et al. (2014a) Neuronal MHC-I expression and its implications in synaptic function, axonal regeneration and Parkinson's and other brain diseases. Frontiers in neuroanatomy 8:114.Pmc4195363.
Cebrian et al. (2014b) Neuroinflammation in Parkinson's Disease Animal Models: A Cell Stress Response or a Step in Neurodegeneration? Curr Top Behav Neurosci.
Cebrian et al. (2014c) MHC-I expression renders catecholaminergic neurons susceptible to T-cell-mediated degeneration. Nat Commun 5:3633.Pmc4024461.
Cecconi et al. (2010) The CD4⁺ T-cell epitope-binding register is a critical parameter when generating functional HLA-DR tetramers with promiscuous peptides. Eur J Immunol 40:1603-1616.
Chan et al. 2009. Calcium homeostasis, selective vulnerability and Parkinson's disease. Trends Neurosci. 32, 249-256.
Chemali et al. 2011. Alternative pathways for MHC class I presentation: a new function for autophagy. Cell Mol Life Sci. 68, 1533-1541.
Chevalier et al. 2011. Neurons are MHC class I-dependent targets for CD8 T cells upon neurotropic viral infection. PLoS Pathog. 7(11):e1002393.
Chen et al. (2015) A53T human alpha-synuclein overexpression in transgenic mice induces pervasive mitochondria macroautophagy defects preceding dopamine neuron degeneration. 35:890-905.
Chesselet M F (2008) In vivo alpha-synuclein overexpression in rodents: a useful model of Parkinson's disease? Exp Neurol 209:22-27.
Chow et al. (2014) Assessment of CD4+ T cell responses to glutamic acid decarboxylase 65 using DQ8 tetramers reveals a pathogenic role of GAD65 121-140 and GAD65 250-266 in T1D development. PLoS One 9:e112882.Pmc4236121.
Conway et al. 2001. Kinetic stabilization of the alpha-synuclein protofibril by a dopamine-alpha-synuclein adduct. Science. 294(5545):1346-9.
Corriveau et al. 1998. Regulation of class I MHC gene expression in the developing and mature CNS by neural activity. Neuron. 21(3):505-20.
Crotzer V L, Blum J S (2008) Cytosol to lysosome transport of intracellular antigens during immune surveillance. Traffic 9:10-16.
Cuervo A M, Macian F (2014) Autophagy and the immune function in aging. Current opinion in immunology 29:97-104.Pmc4119564.
Cuervo et al. (2004) Impaired degradation of mutant alpha-synuclein by chaperone-mediated autophagy. Science 305:1292-1295.
Danielson et al. (2009) Preferentially increased nitration of alpha-synuclein at tyrosine-39 in a cellular oxidative model of Parkinson's disease. Anal Chem 81:7823-7828.Pmc2748813.
Dauer et al. (2002) Resistance of alpha-synuclein null mice to the parkinsonian neurotoxin MPTP. Proc Natl Acad Sci USA 99:14524-14529.
Diani et al. (2014) T cell responses in psoriasis and psoriatic arthritis. Autoimmunity reviews.
Double, K. L. 2012. Neuronal vulnerability in Parkinson's disease. Parkinsonism Relat Disord. 18 Suppl 1:S52-4.
Dowd et al. (2005) The Corridor Task: a simple test of lateralised response selection sensitive to unilateral dopamine deafferentation and graft-derived dopamine replacement in the striatum. Brain Res Bull 68:24-30.
Duka et al. (2013) Identification of the sites of tau hyperphosphorylation and activation of tau kinases in synucleinopathies and Alzheimer's diseases. PLoS One 8:e75025.Pmc3779212.
Elmer, B. M., and A. K. McAllister. 2012. Major histocompatibility complex class I proteins in brain development and plasticity. Trends Neurosci. 35(11):660-70.
Fahn S, and D. Sulzer. 2004. Neurodegeneration and neuroprotection in Parkinson disease. NeuroRx. 1(1):139-54.
Falk et al. 1993. Both human and mouse cells expressing H-2Kb and ovalbumin process the same peptide, SIINFEKL. Cell Immunol. 150(2):447-52.
Farkas, et al. 2000. Pathological features of cerebral cortical capillaries are doubled in Alzheimer's disease and Parkinson's disease. Acta Neuropathol. 100, 395-402.
Fiegl et al. 2013. Amphisomal route of MHC class I cross-presentation in bacteria-infected dendritic cells. J Immunol. 190(6):2791-806.
Fleming et al. (2013) Assessment of sensorimotor function in mouse models of Parkinson's disease. J Vis Exp.Pmc3727502.
Foix, C., and J. Nicolesco. 1925. Anatomie cérébrale. Les noyaux gris centraux et la región Mésencéphalo-sous-optique., Suivi d'un apéndice sur l'anatomie pathologique de la maladie de Parkinson. Masson et Cie, Eds (Paris), 508-538.
Foulds et al. (2013) A longitudinal study on alpha-synuclein in blood plasma as a biomarker for Parkinson's disease. Scientific reports 3:2540.Pmc3756331.
Gentleman et al. 2004. Bioconductor: open software development for computational biology and bioinformatics. Genome Biol. 5, R80.
German et al. 2011. Parkinson's Disease: A Role for the Immune System. Curr. Mol. Pharmacol. In press.
Glynn et al. 2011. MHCI negatively regulates synapse density during the establishment of cortical connections. Nat Neurosci. 14(4):442-51.
Goddard et al. 2007. Regulation of CNS synapses by neuronal MHC class I. Proc Natl Acad Sci USA. 2007 Apr. 17; 104(16):6828-33.
Hall et al. (2014) Hippocampal Lewy pathology and cholinergic dysfunction are associated with dementia in Parkinson's disease. Brain 137:2493-2508.
Hampel et al. (2010) Total and phosphorylated tau protein as biological markers of Alzheimer's disease. Experimental gerontology 45:30-40.Pmc2815003.
Hamza et al. (2010) Common genetic variation in the HLA region is associated with late-onset sporadic Parkinson's disease. Nat Genet 42:781-785.Pmc2930111.
Harms et al. (2013) MHCII is required for alpha-synuclein-induced activation of microglia, CD4 T cell proliferation, and dopaminergic neurodegeneration. J Neurosci 33:9592-9600.3903980.
Harris et al. 2012. Generalized Levy walks and the role of chemokines in migration of effector CD8+ T cells. Nature. 486, 545-8.
Hill-Burns et al. (2011) Evidence for more than one Parkinson's disease-associated variant within the HLA region. PLoS One 6:e27109.Pmc3212531.
Hirsch E C, Hunot S (2009) Neuroinflammation in Parkinson's disease: a target for neuroprotection? Lancet Neurol 8:382-397.
Hodara et al. (2004) Functional consequences of alpha-synuclein tyrosine nitration: diminished binding to lipid vesicles and increased fibril formation. J Biol Chem 279:47746-47753.
Hogquist et al. 1994. T cell receptor antagonist peptides induce positive selection. Cell. 76, 17-27.
Huh et al. 2000. Functional requirement for class I MHC in CNS development and plasticity. Science. 290, 2155-9.
Hwang, O. Role of oxidative stress in Parkinson's disease. 2013. Exp Neurobiol. 22(1):11-7.
Imamura et al. 2005. Cytokine production of activated microglia and decrease in neurotrophic factors of neurons in the hippocampus of Lewy body disease brains. Acta. Neuropathol. 109, 141-150.
Jang et al. (2009a) Viral parkinsonism. Biochim Biophys Acta 1792:714-721.
Jang et al. (2009b) Highly pathogenic H5N1 influenza virus can enter the central nervous system and induce neuroinflammation and neurodegeneration. Proc Natl Acad Sci USA 106:14063-14068.
Kallaur et al. 2013 Cytokine profile in relapsing-remitting multiple sclerosis patients and the association between progression and activity of the disease. Mol. Med. Report. In press.
Kawanokuchi et al. 2006. Production of interferon-gamma by microglia. Mult. Scler. 12, 558-64.
Kim et al. (2014) Dataset size and composition impact the reliability of performance benchmarks for peptide-MHC binding predictions. BMC Bioinformatics 15:241.Pmc4111843.
Kirik et al. (2002) Parkinson-like neurodegeneration induced by targeted overexpression of alpha-synuclein in the nigrostriatal system. J Neurosci 22:2780-2791.
Klegeris et al. 2008. Alpha-synuclein activates stress signaling protein kinases in THP-1 cells and microglia. Neurobiol Aging. 29(5):739-52.
Kleijmeer et al. 2001. Antigen loading of MID class I molecules in the endocytic tract. Traffic. 2, 124-137.
Koehler et al. (2013) Altered serum IgG levels to alpha-synuclein in dementia with Lewy bodies and Alzheimer's disease. PLoS One 8:e64649.Pmc3669378.
Kriks et al. 2011. Dopamine neurons derived from human ES cells efficiently engraft in animal models of Parkinson's disease. Nature. 480, 547-51.
Kurtulus S, Hildeman D (2013) Assessment of CD4(+) and CD8 (+) T cell responses using MHC class I and II tetramers. Methods Mol Biol 979:71-79.Pmc4265237.
Lampe et al. (2003) HLA typing and Parkinson's disease. European neurology 50:64-68.
Lanciego et al. 1997. Use of peroxidase substrate Vector VIP for multiple staining in light microscopy. J Neurosci Methods. 74(1):1-7.
Leaver et al. 2012. Effects of translocator protein (18 kDa) ligands on microglial activation and neuronal death in the quinolinic-acid-injected rat striatum. ACS Chem Neurosci. 3(2):114-9.
Lee et al. 2010. Alpha-synuclein activates microglia by inducing the expressions of matrix metalloproteinases and the subsequent activation of protease-activated receptor-1. J Immunol. 185(1):615-23.
Letellier et al. 2008. Normal adult climbing fiber monoinnervation of cerebellar Purkinje cells in mice lacking MHC class I molecules. Dev Neurobiol. 68(8):997-1006.
Lewis D. A. 2002 The Human Brain Revisited: Opportunities and Challenges in Postmortem Studies of Psychiatric Disorders. 2002 Neuropsychopharmacology.26:143-154.10.1038/S0893-133X(01)00393-.1
Lin et al. (2012) Conditional expression of Parkinson's disease-related mutant alpha-synuclein in the midbrain dopaminergic neurons causes progressive neurodegeneration and degradation of transcription factor nuclear receptor related 1. J Neurosci 32:9248-9264.Pmc3417246.
Lindå et al. 1999. Expression of MHC class I and beta2-microglobulin in rat spinal motoneurons: regulatory influences by IFNgamma and axotomy. Exp. Neurol. 150, 282-295.
Liu et al. 2012. The expression pattern of classical MHC class I molecules in the development of mouse central nervous system. Neurochem Res. 38(2):290-9.
Lolli et al. (2013) Increased CD8+ T cell responses to apoptotic T cell-associated antigens in multiple sclerosis. Journal of neuroinflammation 10:94.3734107.
Luk K C, Lee V M (2014) Modeling Lewy pathology propagation in Parkinson's disease. Parkinsonism Relat Disord 20 Suppl 1:S85-87.Pmc4034060.
Lull, M. E., and M. L. Block. 2010. Microglial activation and chronic neurodegeneration. Neurotherapeutics. 7(4):354-65.
Magen I, Chesselet M F (2010) Genetic mouse models of Parkinson's disease The state of the art. Prog Brain Res 184:53-87.
Mahul-Mellier et al. (2014) c-Abl phosphorylates alpha-synuclein and regulates its degradation: implication for alpha-synuclein clearance and contribution to the pathogenesis of Parkinson's disease. Hum Mol Genet 23:2858-2879.Pmc4014189.
Mangano et al. 2011. Interferon-7 plays a role in paraquat-induced neurodegeneration involving oxidative and proinflammatory pathways. Neurobiol. Aging. 33, 1411-26.
Marrack P, Kappler J W (2012) Do MHCII-presented neoantigens drive type 1 diabetes and other autoimmune diseases? Cold Spring Harbor perspectives in medicine 2:a007765.Pmc3426820.
Marsh et al. 2005. Nomenclature for factors of the HLA system. Tissue Antigens. 65, 301-69.
Martinez-Vicente et al. (2010) Cargo recognition failure is responsible for inefficient autophagy in Huntington's disease. Nature Neuroscience 13:567-576.
Martínez-Vicente et al. 2008. Dopamine-modified alphasynuclein blocks chaperone-mediated autophagy. J. Clin. Invest. 118, 777-788.
McGeer et al. (1988) Expression of the histocompatibility glycoprotein HLA-DR in neurological disease. Acta Neuropathol 76:550-557.
McGeer et al. (2003) Presence of reactive microglia in monkey substantia nigra years after 1-methyl-4-phenyl-1,2,3,6-tetrahydropyridine administration. Ann Neurol 54:599-604.
McGeer et al. 1988. Reactive microglia are positive for HLA-DR in the substantia nigra of Parkinson's and Alzheimer's disease brains. Neurology. 38, 1285-91.
McKeith I G et al. (2005) Diagnosis and management of dementia with Lewy bodies: third report of the DLB Consortium. Neurology 65:1863-1872.
McKhann et al. (2011) The diagnosis of dementia due to Alzheimer's disease: recommendations from the National Institute on Aging-Alzheimer's Association workgroups on diagnostic guidelines for Alzheimer's disease. Alzheimer's & dementia: the journal of the Alzheimer's Association 7:263-269.Pmc3312024.
Medana et al. 2000. MHC class I-restricted killing of neurons by virus-specific CD8+ T lymphocytes is effected through the Fas/FasL, but not the perforin pathway. Eur. J. Immunol. 30, 3623-33.
Meuth et al. 2009. Cytotoxic CD8+ T cell-neuron interactions: perforin-dependent electrical silencing precedes but is not causally linked to neuronal cell death. J. Neurosci. 29, 15397-409.
Moehle M S, West A B (2014) M1 and M2 immune activation in Parkinson's Disease: Foe and ally? Neuroscience.
Mogi et al. 2007. Nagatsu, p53 protein, interferon-gamma, and NF-kappaB levels are elevated in the parkinsonian brain. Neurosci. Lett. 414, 94-97.
Mori I, Kimura Y (2000) Apoptotic neurodegeneration induced by influenza A virus infection in the mouse brain. Microbes and infection/Institut Pasteur 2:1329-1334.
Mori I, Kimura Y (2001) Neuropathogenesis of influenza virus infection in mice. Microbes Infect 3:475-479.
Mosharov et al. (2014) Presynaptic Effects of Levodopa and Their Possible Role in Dyskinesia. Mov Disord.
Mosharov et al. (2009) Interplay between cytosolic dopamine, calcium, and alpha-synuclein causes selective death of substantia nigra neurons. Neuron 62:218-229.
Muñoz, P., S. Huenchuguala, I. Paris, and J. Segura-Aguilar. 2012. Dopamine oxidation and autophagy. Parkinsons Dis. 2012: 920953. doi: 10.1155/2012/920953.
Needleman et al. 2010. MHC class I molecules are present both pre-a and postsynaptically in the visula cortex during postnatal development and in adulthood. Proc. Natl. Acad. Sci. USA. 107, 16999-7004.
Nelson et al. 2013. MHC class I immune proteins are critical for hippocampus-dependent memory and gate NMDAR-dependent hippocampal long-term depression. Learn Mem. 20(9):505-17.
Neumann et al. 1995. Induction of MHC class I genes in neurons. Science. 28; 269(5223):549-52. J Exp Med. 20; 185(2):305-16.
Neumann et al. 1997. Major histocompatibility complex (MHC) class I gene expression in single neurons of the central nervous system: differential regulation by interferon (IFN)-gamma and tumor necrosis factor (TNF)-alpha. J Exp Med. 185(2):305-16.
Nisbet et al. 1981. The complete amino-acid sequence of hen ovalbumin. Eur J Biochem. 1981 April; 115(2):335-45.
Norris et al. 2005. Reversible inhibition of alpha-synuclein fibrillization by dopaminochrome-mediated conformational alterations. J Biol Chem. 280(22):21212-9.
Oliveira et al. 2004. A role for MHC class I molecules in synaptic plasticity and regeneration of neurons after axotomy. Proc. Natl. Acad. Sci. USA. 101, 17843-8.
Oliveras-Salva et al. (2014) Alpha-synuclein-induced neurodegeneration is exacerbated in PINK1 knockout mice. Neurobiol Aging 35:2625-2636.
Orenstein et al. (2013) Interplay of LRRK2 with chaperone-mediated autophagy. Nat Neurosci 16:394-406.3609872.
Oseroff et al. (2010) Molecular determinants of T cell epitope recognition to the common Timothy grass allergen. J Immunol 185:943-955.3310373.
Pardo et al. 1995. Toxic effects of L-DOPA on mesencephalic cell cultures: protection with antioxidants. Brain Res. 682(1-2):133-43.
Paul et al. (2013) HLA class I alleles are associated with peptide-binding repertoires of different size, affinity, and immunogenicity. J Immunol 191:5831-5839.Pmc3872965.
Peakman M (2008) CD8 and cytotoxic T cells in type 1 diabetes. Novartis Found Symp 292:113-119; discussion 119-129, 202-113.
Petersen et al. (2001) Expanded CAG repeats in exon 1 of the Huntington's disease gene stimulate dopamine-mediated striatal neuron autophagy and degeneration. Hum Mol Genet 10:1243-1254.
Petrich et al. (2010) IFN-gamma and IL-10 islet-antigen-specific T cell responses in autoantibody-negative first-degree relatives of patients with type 1 diabetes. Diabetologia 53:1451-1460.
Phani et al. 2012. Neurodegeneration and inflammation in Parkinson's disease. Parkinsonism Relat Disord. 18 Suppl 1:S207-9.
Polymeropoulos et al. 1997. Mutation in the alpha-synuclein gene identified in families with Parkinson's disease. Science. 276(5321):2045-7.
Prabowo et al. 2013. Differential expression of major histocompatibility complex class I in developmental glioneuronal lesions. J. Neuroinflammation. 24 (10): 12.
Rall et al. 1995. Consequences of cytotoxic T lymphocyte interaction with major histocompatibility complex class I-expressing neurons in vivo. J Exp Med. 182(5):1201-12.
Richter et al. 2009. Interferon-gamma prevents death of bystander neurons during CD8 T cell responses in the brain. Am J Pathol. 174(5):1799-807.
Robinson et al. 2011. The IMGT/HLA database. Nucleic Acids Res. 39, D1171-6.
Roep B O, Peakman M (2012) Antigen targets of type 1 diabetes autoimmunity. Cold Spring Harbor perspectives in medicine 2:a007781.Pmc3312399.
Rothenberg, B. E., and J. R. Voland. 1996. beta2 knockout mice develop parenchymal iron overload: A putative role for class I genes of the major histocompatibility complex in iron metabolism. Proc. Natl. Acad. Sci. USA. 93, 1529-34.
Sabha et al. 2008. MHC I expression and synaptic plasticity in different mice strains after axotomy. Synapse. 62(2):137-48.
Sahu et al. (2011) Microautophagy of cytosolic proteins by late endosomes. Dev Cell 20:131-139.3025279.
Sarikonda et al. (2014) CD8 T-cell reactivity to islet antigens is unique to type 1 while CD4 T-cell reactivity exists in both type 1 and type 2 diabetes. J Autoimmun 50:77-82.
Sawla et al. (2012) Regulatory T cells in systemic lupus erythematosus (SLE); role of peptide tolerance. Autoimmunity reviews 11:611-614.
Schmitz et al. (2013) Glycine transporter-1 inhibition promotes striatal axon sprouting via NMDA receptors in dopamine neurons. J Neurosci 33:16778-16789.3797382.
Sengupta et al. (2015) Pathological Interface between Oligomeric Alpha-Synuclein and Tau in Synucleinopathies. Biol Psychiatry.
Shannon et al. (2012) Is alpha-synuclein in the colon a biomarker for premotor Parkinson's disease? Evidence from 3 cases. Mov Disord 27:716-719.
Sharma M et al. (2012) Large-scale replication and heterogeneity in Parkinson disease genetic loci. Neurology 79:659-667.Pmc3414661.
Shatz, C. J. 2009. MHC class I: an unexpected role in neuronal plasticity. Neuron. 64(1):40-5.
Simunovic et al. 2009. Gene expression profiling of substantia nigra dopamine neurons: further insights into Parkinson's disease pathology. Brain. 132, 1795-1809.
Singleton et al. 2003. alpha-Synuclein locus triplication causes Parkinson's disease. Science. 302(5646):841.
Solerte et al. 2000. Overproduction of IFNgamma and TNF-alpha from natural killer (NK) cells is associated with abnormal NK reactivity and cognitive derangement in Alzheimer's disease. Ann. N.Y. Acad. Sci. 917, 331-40.
Spillantini et al. (1998a) Alpha-synuclein in filamentous inclusions of Lewy bodies from Parkinson's disease and dementia with Lewy bodies. Proc Natl Acad Sci USA 95:6469-6473.
Spillantini et al. (1997) Alpha-synuclein in Lewy bodies. Nature 388:839-840.
Spillantini et al. (1998b) Filamentous alpha-synuclein inclusions link multiple system atrophy with Parkinson's disease and dementia with Lewy bodies. Neurosci Lett 251:205-208.
St Martin et al. (2007) Dopaminergic neuron loss and up-regulation of chaperone protein mRNA induced by targeted over-expression of alpha-synuclein in mouse substantia nigra. J Neurochem 100:1449-1457.
Strawbridge A B, Blum J S (2007) Autophagy in MHC class II antigen processing. Curr Opin Immunol 19:87-92.
Sulzer et al. 2000. Neuromelanin biosynthesis is driven by excess cytosolic catecholamines not accumulated by synaptic vesicles. Proc. Natl. Acad. Sci. USA. 97, 11869-11874.
Sulzer et al., in Parkinson's Disease: Molecular and Therapeutic Insights from Model Systems, R. Nass, Ed. (Academic Press, 2008), 491-504.
Sulzer D (2007) Multiple hit hypotheses for dopamine neuron loss in Parkinson's disease. Trends Neurosci 30:244-250.
Sulzer et al. (2008) Neuronal pigmented autophagic vacuoles: lipofuscin, neuromelanin, and ceroid as macroautophagic responses during aging and disease. J Neurochem.
Takahashi et al. (1995) The substantia nigra is a major target for neurovirulent influenza A virus. J Exp Med 181:2161-2169.Pmc2192055.
Tallóczy et al. 2008. Methamphetamine inhibits antigen processing, presentation, and phagocytosis. PLoS Pathog. 4, e28.
Tanner et al. (1999) Parkinson disease in twins: an etiologic study. Jama 281:341-346.
Taymans J M, Baekelandt V (2014) Phosphatases of alpha-synuclein, LRRK2, and tau: important players in the phosphorylation-dependent pathology of Parkinsonism. Frontiers in genetics 5:382.Pmc4224088.
ten Broeke T, Wubbolts R, Stoorvogel W (2013) MHC class II antigen presentation by dendritic cells regulated through endosomal sorting. Cold Spring Harbor perspectives in biology 5:a016873.
Teoh, C. Y., and K. J. Davies. 2004. Potential roles of protein oxidation and the immunoproteasome in MHC class I antigen presentation: the ‘PrOxI’ hypothesis. Arch. Biochem. Biophys. 423, 88-96.
Theodore et al. (2008) Targeted overexpression of human alpha-synuclein triggers microglial activation and an adaptive immune response in a mouse model of Parkinson disease. J Neuropathol Exp Neurol 67:1149-1158.Pmc2753200.
Tooyama et al. 1990. Reactive microglia express class I and class II major histocompatibility complex antigens in Alzheimer's disease. Brain. Res. 523, 273-280.
Traugott U (1987) Multiple sclerosis: relevance of class I and class II MHC-expressing cells to lesion development. J Neuroimmunol 16:283-302.
van den Elsen et al. 2004. Transcriptional regulation of antigen presentation. Curr. Opin. Immunol. 16, 67-75. (2004).
Velthuis et al. (2010) Simultaneous detection of circulating autoreactive CD8+ T-cells specific for different islet cell-associated epitopes using combinatorial MHC multimers. Diabetes 59:1721-1730.Pmc2889772.
Volpicelli-Daley et al. 2011. Exogenous α-synuclein fibrils induce Lewy body pathology leading to synaptic dysfunction and neuron death. Neuron. 72, 57-71.
Wang et al. (2009) Tau fragmentation, aggregation and clearance: the dual role of lysosomal processing. Hum Mol Genet 18:4153-4170.2758146.
Willing, A., and M. A. Friese. 2012. CD8-mediated inflammatory central nervous system disorders. Curr. Opin. Neurol. 25, 316-21.
Wissemann et al. (2013) Association of Parkinson disease with structural and regulatory variants in the HLA region. Am J Hum Genet 93:984-993.Pmc3824116.
Wong et al. 1984. Inducible expression of H-2 and Ia antigens on brain cells. Nature. 310, 688-91.
Wu et al. (2012) Prediction of HLA class I-restricted T-cell epitopes of islet autoantigen combined with binding and dissociation assays. Autoimmunity 45:176-185.
Yamada et al. (2004) Overexpression of alpha-synuclein in rat substantia nigra results in loss of dopaminergic neurons, phosphorylation of alpha-synuclein and activation of caspase-9: resemblance to pathogenetic changes in Parkinson's disease. J Neurochem 91:451-461.
Yin et al. (2013) Recognition of self and altered self by T cells in autoimmunity and allergy. Protein Cell 4:8-16.Pmc3951410.
Zecca et al. 2008. New melanic pigments in the human brain that accumulate in aging and block environmental toxic metals. Proc. Natl. Acad. Sci. USA. 105, 17567-72.
Zecca et al. 2004. The role of iron and copper molecules in the neuronal vulnerability of locus coeruleus and substantia nigra during aging. Proc. Natl. Acad. Sci. USA. 101, 9843-8.
Zetterberg et al. (2013) Plasma tau levels in Alzheimer's disease. Alzheimer's research & therapy 5:9.Pmc3707015.
Zhao et al. (2013) Association of HLA locus variant in Parkinson's disease. Clinical genetics 84:501-504.
Zhang et al. 2007. Microglial PHOX and Mac-1 are essential to the enhanced dopaminergic neurodegeneration elicited by A30P and A53T mutant alphasynuclein. Glia. 55, 1178-1188.
Zhang et al. 2011. Neuromelanin activates microglia and induces degeneration of dopaminergic neurons: implications for progression of Parkinson's disease. Neurotox. Res. 19, 63-72.
Zhang et al. 2005. Aggregated alpha-synuclein activates microglia: a process leading to disease progression in Parkinson's disease. FASEB J. 19(6):533-42.
Zhang et al. 2013a. The expression patterns of MHC class I molecules in the developmental human visual system. Neurochem Res. 38(2):273-81.
Zhang et al. 2013b. The spatio-temporal expression of MHC class I molecules during human hippocampal formation development. Brain Res. In press. doi:pii: S0006-8993(13)00950-5.
Zhang et al. 2013c. Human neuromelanin: an endogenous microglial activator for dopaminergic neuron death. Front Biosci (Elite Ed). 5:1-11.
Zhao et al. 2013. Activated microglia are implicated in cognitive deficits, neuronal death, and successful recovery following intermittent ethanol exposure. Behav Brain Res. 236(1):270-82.
Zheng et al. 2010. Global PD Gene Expression (GPEX) Consortium, PGC-1α, a potential therapeutic target for early intervention in Parkinson's disease. Sci. Transl. Med. 52, 52ra73.
Zolotukhin et al. (1999) Recombinant adeno-associated virus purification using novel methods improves infectious titer and
yield. Gene therapy 6:973-985.

What is claimed is:

1. A method for assessing whether a subject is at risk of developing an α-synucleinopathy, Parkinson's disease (PD), Lewy Body dementia (LBD), or Alzheimer's disease (AD) comprising
(a)
i) obtaining leukocytes from the subject;
ii) contacting the leukocytes with an epitope;
iii) determining whether the leukocytes have increased activation after contact with the epitope; and
iv) identifying the subject as at risk of developing PD, LBD or AD if in step iii) the leukocytes are determined to have increased activation after contact with the epitope, and identifying the subject as not at risk of developing the α-synucleinopathy, PD, LBD or AD if in step iii) the leukocytes are determined to not have increased activation after contact with the epitope, or
(b)
i) obtaining leukocytes from the subject;
ii) separating the leukocytes into 2 or more pools of leukocytes and contacting each pool with an epitope, wherein each pool is contacted with a different epitope;
iii) determining whether each pool has increased activation after contact with the epitope; and
iv) identifying the subject as at risk of developing the α-synucleinopathy, PD, LBD, or AD if and only if in step iii) 1 or more pools is determined to have increased activation after contact with an epitope.

2. The method of claim 1, for for assessing whether a subject is at risk of developing PD.

3. The method of claim 1 or 2, wherein in step ii) the leukocytes are separated into 2, 3, 4, 5, 6, 7, 8, 9, 10, 11-50 or more pools, and in step iv) the subject is identified as at risk of developing the α-synucleinopathy, PD, LBD or AD if and only if in step iii) 1, 2, 3, 4, 5, 6, 7, 8, 9, 10, 11-50 or more pools is determined to have increased activation after contact with an epitope.

4. The method of any one of claims 1-3, wherein the subject
a) is at least about 35, 40, 45, 50, 55, 60, 65, 70, 75 or 80 years of age;
b) is less than about 35, 40, 45, 50, 55, 60, 65, 70, 75 or 80 years of age;
c) has a symptom that has preceded the onset of the α-synucleinopathy, PD, LBD or AD in subjects who have developed the α-synucleinopathy, PD, LBD or AD;
d) has a symptom that has preceded the onset of the α-synucleinopathy, PD, LBD or AD in subjects who have developed the α-synucleinopathy, PD, LBD or AD, wherein the symptom has preceded the onset of the α-synucleinopathy, PD, LBD or AD in the subjects by at least about 5, 10, 15, 20, 25, 30 or 5-30 years;
e) is afflicted with cognitive decline, constipation or orthostatic hypotension
f) is afflicted with cognitive decline, and the cognitive decline is reduced spatial reasoning ability and/or reduced memory ability.

5. The method of any one of claims 1-4, further comprising directing the subject to
(a) be monitored more frequently for the α-synucleinopathy, PD, LBD, or AD; or
(b) receive additional diagnostic testing for the α-synucleinopathy, PD, LBD, or AD,
if the subject is identified as at risk of developing the α-synucleinopathy, PD, LBD, or AD.

6. A method for diagnosing or confirming whether a subject is afflicted with an α-synucleinopathy, Parkinson's disease (PD), Lewy Body dementia (LBD), or Alzheimer's disease (AD) comprising
(a)
i) obtaining leukocytes from the subject;
ii) contacting the leukocytes with an epitope;
iii) determining whether the leukocytes have increased activation after contact with the epitope; and
iv) identifying the subject as afflicted with PD if in step iii) the leukocytes are determined to have increased activation after contact with the epitope, and identifying the subject as not afflicted with the α-synucleinopathy, PD, LBD, or AD if in step iii) the leukocytes are determined to not have increased activation after contact with the epitope, or
(b)
i) obtaining leukocytes from the subject;
ii) separating the leukocytes into 1 or more pools of leukocytes and contacting each pool with an epitope, wherein each pool is contacted with a different epitope;
iii) determining whether each pool has increased activation after contact with the epitope; and
iv) identifying the subject as afflicted with the α-synucleinopathy, PD, LBD, or AD if and only if in step iii) 1 or more pools is determined to have increased activation after contact with an epitope.

7. The method of claim 6, for diagnosing or confirming whether a subject is afflicted with PD.

8. The method of claim 6 or 7, wherein in step ii) the leukocytes are separated into 2, 3, 4, 5, 6, 7, 8, 9, 10, 11-50 or more pools, and in step iv) the subject is identified as afflicted with the α-synucleinopathy, PD, LBD or AD if and only if in step iii) 1, 2, 3, 4, 5, 6, 7, 8, 9, 10, 11-50 or more pools is determined to have increased activation after contact with an epitope.

9. The method of any one of claims 1-8, further comprising determining the presence of at least one human leukocyte antigen (HLA) allele in the subject.

10. The method of claim 10, wherein the subject is identified as at risk of developing the α-synucleinopathy, PD, LBD, or AD or identified as afflicted with the α-synucleinopathy, PD, LBD, or AD if
(a) the leukocytes are determined to have increased activation after contact with the epitope, or 1 or more pools is determined to have increased activation after contact with an epitope, and
(b) the subject has the HLA allele DRB5*01:01 or DRB1*15:01.

11. A method for assessing whether an α-synucleinopathy, Parkinson's disease (PD), Lewy Body dementia (LBD), or Alzheimer's disease (AD) has progressed in a subject afflicted with the α-synucleinopathy, PD, LBD, or AD comprising
(a)
performing each of the following steps i) to iii):
i) obtaining leukocytes from the subject;
ii) contacting the leukocytes with an epitope that was previously identified to increase activation of the leukocytes; and
iii) determining the level of activation of the leukocytes after contact with the epitope,
at a first and a second point in time, and then
iv) concluding that the α-synucleinopathy, PD, LBD, or AD has progressed in the subject if the leukocytes are determined to be more activated in step iii) performed at the second point in time compared to the level of activation in step iii) performed at the first point in time, or
(b)
performing each of the following steps i) to iii):
i) obtaining leukocytes from the subject;
ii) separating the leukocytes into two or more pools of leukocytes and contacting each pool with an epitope, wherein each pool is contacted with a different epitope;
iii) determining whether each pool has increased activation after contact with the epitope;
at a first and a second point in time, and then
iv) concluding that the α-synucleinopathy, PD, LBD, or AD has progressed in the subject if more pools of leukocytes are determined to be activated in step iii) performed at the second point in time compared to the number of pools that are determined to be activated in step iii) performed at the first point in time.

12. The method of claim 13, for assessing whether PD has progressed in a subject afflicted with PD.

13. A method for treating a subject afflicted with an α-synucleinopathy, Parkinson's disease (PD), Lewy Body dementia (LBD), or Alzheimer's disease (AD), comprising
(a) administering to the subject a compound that is approved for use in treating subjects afflicted with the α-synucleinopathy, PD, LBD, or AD, wherein the subject has been diagnosed or confirmed to be afflicted with PD according to the method of any one of claims 7-12; or
(b) diagnosing or confirming the subject to be afflicted with the α-synucleinopathy, PD, LBD, or AD according to the method of any one of claims 7-12, and administering to the subject a compound that is approved for use in treating subjects afflicted with PD, LBD, or AD.

14. A method for assessing whether an α-synucleinopathy, Parkinson's disease (PD), Lewy Body dementia (LBD), or Alzheimer's disease (AD) is developing in a subject who has been identified as being at risk of developing the α-synucleinopathy, PD, LBD, or AD comprising
(a)
performing each of the following steps i) to iii):
i) obtaining leukocytes from the subject;
ii) contacting the leukocytes with an epitope that was previously identified to increase activation of the leukocytes; and
iii) determining the level of activation of the leukocytes after contact with the epitope,
at a first and a second point in time, and then
iv) concluding that the α-synucleinopathy, PD, LBD, or AD is developing in the subject if the leukocytes are determined to be more activated in step iii) performed at the second point in time compared to the level of activation in step iii) performed at the first point in time, or
(b)
performing each of the following steps i) to iii):
i) obtaining leukocytes from the subject;
ii) separating the leukocytes into two or more pools of leukocytes and contacting each pool with an epitope, wherein each pool is contacted with a different epitope;
iii) determining whether each pool has increased activation after contact with the epitope;
at a first and a second point in time, and then
iv) concluding that that PD, LBD, or AD is developing in the subject if more pools of leukocytes are determined to be activated in step iii) performed at the second point in time compared to the number of pools that are determined to be activated in step iii) performed at the first point in time.

15. The method of claim 16, for assessing whether PD is developing in a subject who has been identified as being at risk of developing PD.

16. A method for assessing whether a subject afflicted with an α-synucleinopathy, Parkinson's disease (PD), Lewy Body dementia (LBD), or Alzheimer's disease (AD) is likely to benefit from a therapy, wherein the therapy is directed to leukocytes that are activated by an epitope, the method comprising
(a)
i) obtaining leukocytes from the subject;
ii) contacting the leukocytes with the epitope;
iii) determining whether the leukocytes have increased activation after contact with the epitope; and
iv) identifying the subject as likely to benefit from the therapy if in step iii) the leukocytes are determined to have increased activation after contact with the epitope, and identifying the subject as unlikely to benefit from the therapy if in step iii) the leukocytes are determined to not have increased activation after contact with the epitope, or
(b)
i) obtaining leukocytes from the subject;
ii) contacting the leukocytes with the epitope;
iii) determining whether the leukocytes have increased activation after contact with the epitope; and
iv) identifying the subject as having benefited from the therapy if in step iii) if the leukocytes are determined to have increased activation after contact with the epitope, and identifying the subject as not having benefited from the therapy if in step iii) the leukocytes are determined to not have increased activation after contact with the epitope.

17. The method of claim 16, for assessing whether a subject afflicted with PD is likely to benefit from a therapy.

18. A method for treating a subject afflicted with an α-synucleinopathy, Parkinson's disease (PD), Lewy Body dementia (LBD), or Alzheimer's disease (AD) comprising administering to the subject a therapy that is directed to leukocytes that are activated by an epitope, wherein leukocytes of the subject have been determined to have increased activation after contact with the epitope.

19. The method of claim 18, wherein the therapy is tolerization therapy, and the tolerization therapy is specific for leukocytes that are activated by the epitope.

20. The method of claim 19, wherein administering the tolerization therapy comprises administering to the subject the epitope in an amount that is effective to reduce activation of leukocytes in the subject by the epitope.

21. The method of claim 18, wherein the therapy comprises selectively killing the leukocytes that are activated by the epitope in the subject.

22. The method of claim 21, wherein selectively killing the leukocytes that are activated by the epitope in the subject comprises administering to the subject an effective amount of a compound comprising a major histocompatibility complex (MHC) Tetramer and a toxin to the subject, wherein the MHC Tetramer comprises the epitope.

23. A method for treating a subject afflicted with an α-synucleinopathy, Parkinson's disease (PD), Lewy Body dementia (LBD), or Alzheimer's disease (AD), comprising administering an immunosuppressant therapy to the subject, wherein the subject has been identified as being likely to benefit from a therapy directed to leukocytes that are activated by an epitope according to the method of claim 16.

24. The method of any one of claims 16-23, wherein the immunosuppressant therapy comprises tolerization therapy, selectively killing the leukocytes that are activated by an epitope in the subject, or administering an effective amount of an immunosuppressive compound to the subject.

25. The method of claim 24, wherein the immunosuppressive compound is a calcineurin inhibitor, a compound that blocks a chemokine receptor that is expressed by a leukocyte, a glucocorticoid, a mTOR inhibitor, an anti-metabolic compound, a phosphodiesterase-5 inhibitor, an antibody, or a leukocyte function antigen-3 (LFA-3)/Fc fusion protein.

26. A method for assessing whether leukocytes of a subject afflicted with an α-synucleinopathy, Parkinson's disease (PD), Lewy Body dementia (LBD), or Alzheimer's disease (AD) are activated by an epitope, comprising
i) obtaining leukocytes from the subject;
ii) contacting the leukocytes with the epitope;
iii) determining whether the leukocytes have increased activation after contact with the epitope; and
iv) identifying the leukocytes of the subject as activated by the epitope if in step iii) the leukocytes are determined to have increased activation after contact with the epitope, and identifying the leukocytes of the subject as not activated by the epitope if in step iii) the leukocytes are determined to not have increased activation after contact with the epitope.

27. The method of claim 26, for assessing whether leukocytes of a subject afflicted with PD are activated by an epitope.

28. The method of any one of claims 1-27, wherein the epitope
a) is or comprises part of a compound that is produced by neurons in subjects afflicted with the α-synucleinopathy, PD, LBD, or AD;
b) is or comprises part of a compound that is produced by neurons in subjects afflicted with the α-synucleinopathy, PD, LBD, or AD, wherein the neurons are in the ventral midbrain, the substantia nigra, the locus coeruleus, or the ventral tegmental area;
c) is or comprises part of a compound that is produced by neurons in subjects afflicted with the α-synucleinopathy, PD, LBD, or AD, wherein the neurons are catecholamine neurons;
d) comprises consecutive amino acids that are identical to a stretch of consecutive amino acids in a protein that is produced by the neurons;
e) comprises consecutive amino acids that are identical to a stretch of consecutive amino acids in α-synuclein (α-syn), tau, leucine-rich repeat kinase 2 (LRRK2) or glucocerebrosidase;
f) comprises consecutive amino acids that are identical to a stretch of consecutive amino acids in an α-syn, tau, leucine-rich repeat kinase 2 (LRRK2) or glucocerebrosidase mutant
g) comprises consecutive amino acids that are identical to a stretch of consecutive amino acids in an α-syn mutant;
h) comprises consecutive amino acids that are identical to a stretch of consecutive amino acids in an α-syn mutant, wherein the α-syn mutant is an α-syn A53T or A30P mutant;
i) comprises about 15, at least 15, 5-50, 8-11, or 8-14 amino acids;
j) is phosphorylated, nitrated, or dopamine modified;
k) comprises a phosphorylated serine or a phosphorylated tyrosine;
l) comprises a phosphorylated serine or a phosphorylated tyrosine, wherein the phosphorylated serine or phosphorylated tyrosine is within a stretch of consecutive amino acids that is identical to a stretch of consecutive amino acids comprising the serine at position 129 of α-syn or the tyrosine at position 39 of α-syn;
m) comprises consecutive amino acids in the sequence set forth as VFMKGLSKA (SEQ ID NO: 10), DVFMKGLSKA (SEQ ID NO: 11), GVVAAAEKTK (SEQ ID NO: 12), VAAAEKTKQGVAEAP (SEQ ID NO: 13), VAAAEKTKQGVAEAA (SEQ ID NO: 14), AGKTKEGVL (SEQ ID NO: 15), PGKTKEGVL (SEQ ID NO: 16), AGKTKEGVLY (SEQ ID NO: 17), APGKTKEGVL (SEQ ID NO: 18), GVAEAAGKTK (SEQ ID NO: 19), KQGVAEAPGKTKEGV (SEQ ID NO: 20), PGKTKEGVLYVGSKT (SEQ ID NO: 21), KTKEGVLYVGSKTKK (SEQ ID NO: 22), KQGVAEAAGKTKEGV (SEQ ID NO: 23), AGKTKEGVLYVGSKT (SEQ ID NO: 24), VLYVGSKTK (SEQ ID NO: 25), LYVGSKTKK (SEQ ID NO: 26), YVGSKTKEGV (SEQ ID NO: 27), VLYVGSKTKK (SEQ ID NO: 28), GVLYVGSKTK (SEQ ID NO: 29), LYVGSKTKEG (SEQ ID NO: 30), KTKKGVVHGV (SEQ ID NO: 31), KTKKGVVHG (SEQ ID NO: 32), YVGSKTKKGVVHGVA (SEQ ID NO: 33), KTKEGVLYVGSKTKE (SEQ ID NO: 34), VTNVGGAVV (SEQ ID NO: 35), GVVHGVTTV (SEQ ID NO: 36), EEGAPQEGI (SEQ ID NO: 37), GSIAAATGFV (SEQ ID NO: 38), SIAAATGFVK (SEQ ID NO: 39), AGSIAAATGF (SEQ ID NO: 40), IAAATGFVKK (SEQ ID NO: 41), APQEGILEDM (SEQ ID NO: 42), EEGAPQEGIL (SEQ ID NO: 43), VFMKGLSKAK (SEQ ID NO: 44), AEAAGKTKEG (SEQ ID NO: 45), YVGSKTKEGVVHGVT (SEQ ID NO: 46), or IAAATGFVK (SEQ ID NO: 47);
n) comprises at least 8 consecutive amino acids having a sequence within the amino acid sequence set forth as KTKEGVLYVGSKTKE (SEQ ID NO: 34), GKTKEGVLYVGSKTK (SEQ ID NO: 59) or DNEAYEMPSEEGYQDY (SEQ ID NO: 48);
o) comprises at least 8 consecutive amino acids having a sequence within the amino acid sequence set forth as DNEAYEMPSEEGYQDY (SEQ ID NO: 48);
p) comprises consecutive amino acids in the sequence set forth as PSEEGYQDY (SEQ ID NO: 49), YEMPSEEGY (SEQ ID NO: 50), MPSEEGYQD (SEQ ID NO: 51), AYEMPSEEGY (SEQ ID NO: 52), MPSEEGYQDY (SEQ ID NO: 53), EMPSEEGYQD (SEQ ID NO: 54), DNEAYEMPSE (SEQ ID NO: 55), YEMPSEEGYQ (SEQ ID NO: 56), or SEEGYQDYEP (SEQ ID NO: 57);
q) comprises consecutive amino acids in the sequence set forth as PSEEGYQDY (SEQ ID NO: 49), YEMPSEEGY (SEQ ID NO: 50), MPSEEGYQD (SEQ ID NO: 51), AYEMPSEEGY (SEQ ID NO: 52), MPSEEGYQDY (SEQ ID NO: 53), EMPSEEGYQD (SEQ ID NO: 54), DNEAYEMPSE (SEQ ID NO: 55), YEMPSEEGYQ (SEQ ID NO: 56), or SEEGYQDYEP (SEQ ID NO: 57), wherein the serine in the sequence set forth as PSEEGYQDY (SEQ ID NO: 49), YEMPSEEGY (SEQ ID NO: 50), MPSEEGYQD (SEQ ID NO: 51), AYEMPSEEGY (SEQ ID NO: 52), MPSEEGYQDY (SEQ ID NO: 53), EMPSEEGYQD (SEQ ID NO: 54), DNEAYEMPSE (SEQ ID NO: 55), YEMPSEEGYQ (SEQ ID NO: 56), or SEEGYQDYEP (SEQ ID NO: 57) is phosphorylated;
r) comprises a non-amino acid polymer that is produced by the neurons;
s) is neuromelanin or a portion thereof.

29. The method of any one of claims 1-28, wherein in step iii) the leukocytes are determined to have increased activation after contact with the epitope
a) if the leukocytes express or release more of at least one cytokine compared to corresponding leukocytes not contacted with the epitope;
b) if the leukocytes release at least one cytokine;
c) if the leukocytes release at least one cytokine, wherein in step iii) the leukocytes are determined to have released the at least one cytokine if there are over 20 spot-forming cells (SFC) per million cells as measured by an ELISpot assay comprising the colorimetric detection of the at least one cytokine.

30. The method of any one of claims 1-29, wherein
a) the leukocytes are T cells;
b) the at least one cytokine is at least interferon-gamma (IFN-γ) or IL-5;
c) the at least one cytokine is at least TNFα, IL-4, IL-17, IL-10, or IL-21;
d) the at least one cytokine is two or more cytokines, wherein the two or more cytokines are at least IFN-γ and IL-5;
e) the leukocytes are T cells are CD4+ T cells, CD8+ T cells, and/or CD4+CD8+ T cells

31. The method of claim 29 or 30, wherein in step iii) the level of the at least one cytokine that is expressed or released from the leukocytes is assayed through a process comprising an enzyme-linked immunosorbent assay (ELISA), intracellular cytokine staining (ICS), or quantitative RT-PCR.

32. The method of any one of claims 1-31, wherein determining whether the leukocytes have increased activation comprises
(a) contacting the leukocytes with compound comprising a major histocompatibility complex (MHC) Tetramer having four MHC molecules, wherein each MHC molecule is associated with an epitope;
(b) identifying leukocytes that become bound to the compound as activated.

33. A method for assessing whether a test compound is an epitope to which leukocytes of a subject suffering from a neurological disorder are responsive comprising
i) obtaining leukocytes from the subject;
ii) contacting the leukocytes with the test compound;
iii) determining whether the leukocytes has increased activation after contact with the test compound; and
iv) identifying the test compound as an epitope to which the leukocytes are responsive if in step iii) the leukocytes are determined to have increased activation after contact with the test compound, and identifying the test compound as not an epitope to which the leukocytes are responsive if in step iii) the leukocytes are determined to not have increased activation after contact with the test compound.

34. The method of claim 33, wherein the test compound is or comprises part of a compound that is produced by neurons in subjects afflicted with an α-synucleinopathy, Parkinson's disease (PD), Lewy Body dementia (LBD), or Alzheimer's disease (AD).

35. A kit comprising an epitope as in any one of claims 1-34.

36. A compound for treating an α-synucleinopathy, Parkinson's disease (PD), Lewy Body dementia (LBD), or Alzheimer's disease (AD), comprising i) a major histocompatibility complex (MHC) Tetramer having four MHC molecules, wherein each MHC molecule is associated with an epitope, and ii) a toxin.

37. In a process for assessing whether a subject is at risk of developing an α-synucleinopathy, Parkinson's disease (PD), Lewy Body dementia (LBD), or Alzheimer's disease (AD), which involves an array of testing, the improvement comprising including in the array of testing the steps of:
(a)
i) obtaining leukocytes from the subject;
ii) contacting the leukocytes with an epitope;
iii) determining whether the leukocytes have increased activation after contact with the epitope; and
iv) identifying the subject as at risk of developing PD, LBD or AD if in step iii) the leukocytes are determined to have increased activation after contact with the epitope, and identifying the subject as not at risk of developing the α-synucleinopathy, PD, LBD or AD if in step iii) the leukocytes are determined to not have increased activation after contact with the epitope, or
(b)
i) obtaining leukocytes from the subject;
ii) separating the leukocytes into 2 or more pools of leukocytes and contacting each pool with an epitope, wherein each pool is contacted with a different epitope;
iii) determining whether each pool has increased activation after contact with the epitope; and
iv) identifying the subject as at risk of developing the α-synucleinopathy, PD, LBD, or AD if in step iii) 1 or more pools is determined to have increased activation after contact with an epitope.

38. In a process for diagnosing or confirming whether a subject is afflicted with an α-synucleinopathy, Parkinson's disease (PD), Lewy Body dementia (LBD), or Alzheimer's disease (AD), which involves an array of testing, the improvement comprising including in the array of testing the steps of:
(a)
i) obtaining leukocytes from the subject;
ii) separating the leukocytes into 1 or more pools of leukocytes and contacting each pool with an epitope, wherein each pool is contacted with a different epitope;
iii) determining whether each pool has increased activation after contact with the epitope; and
iv) identifying the subject as afflicted with the α-synucleinopathy, PD, LBD, or AD if and only if in step iii) 1 or more pools is determined to have increased activation after contact with an epitope, or
(b)
i) obtaining leukocytes from the subject;
ii) separating the leukocytes into 1 or more pools of leukocytes and contacting each pool with an epitope, wherein each pool is contacted with a different epitope;
iii) determining whether each pool has increased activation after contact with the epitope; and
iv) identifying the subject as afflicted with the α-synucleinopathy, PD, LBD, or AD if and only if in step iii) 1 or more pools is determined to have increased activation after contact with an epitope.


SEQUENCE LISTING (121 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 112 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKEGYQDYEPEA

SEQ ID NO: 3 - DNA, 3215 bp, Homo sapiens
aggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 4 - DNA, 3127 bp, Homo sapiens
gccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 5 - protein, 2527 aa, Homo sapiens
MASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 6 - DNA, 9239 bp, Homo sapiens
gcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcacgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactttaaaaaaaaaaaaaaaaa

SEQ ID NO: 7 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 8 - DNA, 2259 bp, Homo sapiens
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 9 - protein, 8 aa, Homo sapiens
SIINFEKL

SEQ ID NO: 10 - protein, 9 aa, Homo sapiens
VFMKGLSKA

SEQ ID NO: 11 - protein, 10 aa, Homo sapiens
DVFMKGLSKA

SEQ ID NO: 12 - protein, 10 aa, Homo sapiens
GVVAAAEKTK

SEQ ID NO: 13 - protein, 15 aa, Homo sapiens
VAAAEKTKQGVAEAP

SEQ ID NO: 14 - protein, 15 aa, Homo sapiens
VAAAEKTKQGVAEAA

SEQ ID NO: 15 - protein, 9 aa, Homo sapiens
AGKTKEGVL

SEQ ID NO: 16 - protein, 9 aa, Homo sapiens
PGKTKEGVL

SEQ ID NO: 17 - protein, 10 aa, Homo sapiens
AGKTKEGVLY

SEQ ID NO: 18 - protein, 10 aa, Homo sapiens
APGKTKEGVL

SEQ ID NO: 19 - protein, 10 aa, Homo sapiens
GVAEAAGKTK

SEQ ID NO: 20 - protein, 15 aa, Homo sapiens
KQGVAEAPGKTKEGV

SEQ ID NO: 21 - protein, 15 aa, Homo sapiens
PGKTKEGVLYVGSKT

SEQ ID NO: 22 - protein, 15 aa, Homo sapiens
KTKEGVLYVGSKTKK

SEQ ID NO: 23 - protein, 15 aa, Homo sapiens
KQGVAEAAGKTKEGV

SEQ ID NO: 24 - protein, 15 aa, Homo sapiens
AGKTKEGVLYVGSKT

SEQ ID NO: 25 - protein, 9 aa, Homo sapiens
VLYVGSKTK

SEQ ID NO: 26 - protein, 9 aa, Homo sapiens
LYVGSKTKK

SEQ ID NO: 27 - protein, 10 aa, Homo sapiens
YVGSKTKEGV

SEQ ID NO: 28 - protein, 10 aa, Homo sapiens
VLYVGSKTKK

SEQ ID NO: 29 - protein, 10 aa, Homo sapiens
GVLYVGSKTK

SEQ ID NO: 30 - protein, 10 aa, Homo sapiens
LYVGSKTKEG

SEQ ID NO: 31 - protein, 10 aa, Homo sapiens
KTKKGVVHGV

SEQ ID NO: 32 - protein, 9 aa, Homo sapiens
KTKKGVVHG

SEQ ID NO: 33 - protein, 15 aa, Homo sapiens
YVGSKTKKGVVHGVA

SEQ ID NO: 34 - protein, 15 aa, Homo sapiens
KTKEGVLYVGSKTKE

SEQ ID NO: 35 - protein, 9 aa, Homo sapiens
VTNVGGAVV

SEQ ID NO: 36 - protein, 9 aa, Homo sapiens
GVVHGVTTV

SEQ ID NO: 37 - protein, 9 aa, Homo sapiens
EEGAPQEGI

SEQ ID NO: 38 - protein, 10 aa, Homo sapiens
GSIAAATGFV

SEQ ID NO: 39 - protein, 10 aa, Homo sapiens
SIAAATGFVK

SEQ ID NO: 40 - protein, 10 aa, Homo sapiens
AGSIAAATGF

SEQ ID NO: 41 - protein, 10 aa, Homo sapiens
IAAATGFVKK

SEQ ID NO: 42 - protein, 10 aa, Homo sapiens
APQEGILEDM

SEQ ID NO: 43 - protein, 10 aa, Homo sapiens
EEGAPQEGIL

SEQ ID NO: 44 - protein, 10 aa, Homo sapiens
VFMKGLSKAK

SEQ ID NO: 45 - protein, 10 aa, Homo sapiens
AEAAGKTKEG

SEQ ID NO: 46 - protein, 15 aa, Homo sapiens
YVGSKTKEGVVHGVT

SEQ ID NO: 47 - protein, 9 aa, Homo sapiens
IAAATGFVK

SEQ ID NO: 48 - protein, 16 aa, Homo sapiens
DNEAYEMPSEEGYQDY

SEQ ID NO: 49 - protein, 9 aa, Homo sapiens
PSEEGYQDY

SEQ ID NO: 50 - protein, 9 aa, Homo sapiens
YEMPSEEGY

SEQ ID NO: 51 - protein, 9 aa, Homo sapiens
MPSEEGYQD

SEQ ID NO: 52 - protein, 10 aa, Homo sapiens
AYEMPSEEGY

SEQ ID NO: 53 - protein, 10 aa, Homo sapiens
MPSEEGYQDY

SEQ ID NO: 54 - protein, 10 aa, Homo sapiens
EMPSEEGYQD

SEQ ID NO: 55 - protein, 10 aa, Homo sapiens
DNEAYEMPSE

SEQ ID NO: 56 - protein, 10 aa, Homo sapiens
YEMPSEEGYQ

SEQ ID NO: 57 - protein, 10 aa, Homo sapiens
SEEGYQDYEP

SEQ ID NO: 58 - protein, 19 aa, Homo sapiens
NTQTDRESLRNLRCYYNQS

SEQ ID NO: 59 - protein, 15 aa, Homo sapiens
GKTKEGVLYVGSKTK

SEQ ID NO: 60 - protein, 18 aa, Homo sapiens
YWDLQTRNVKAHSQTDRA

SEQ ID NO: 61 - protein, 23 aa, Homo sapiens
RNTQIFKTNTQTHRENLRIALRY

SEQ ID NO: 62 - protein, 16 aa, Homo sapiens
FLPTGGKGGSCSQAAS

SEQ ID NO: 63 - protein, 18 aa, Homo sapiens
QRMEPRAPWIEQERPAYW

SEQ ID NO: 64 - protein, 28 aa, Homo sapiens
VYSRHPAENGKSNFLNCYVSGFHPSDIE

SEQ ID NO: 65 - protein, 16 aa, Homo sapiens
PDGRLLRGYQQDAYDG

SEQ ID NO: 66 - protein, 15 aa, Homo sapiens
GHVRGVAPQIPGERE

SEQ ID NO: 67 - protein, 24 aa, Homo sapiens
HQAQVGGGPCGGAVESLPGGHVRG

SEQ ID NO: 68 - protein, 16 aa, Homo sapiens
FLPTGGKGGSCSQAAS

SEQ ID NO: 69 - protein, 21 aa, Homo sapiens
RKLEAAGVAEQLRAYLEGECV

SEQ ID NO: 70 - protein, 10 aa, Homo sapiens
DVGSDGRFLR

SEQ ID NO: 71 - protein, 19 aa, Homo sapiens
NTQTDRESLRNLRCYYNQS

SEQ ID NO: 72 - protein, 17 aa, Homo sapiens
SLPGGRVRGVAPQIPGE

SEQ ID NO: 73 - protein, 26 aa, Homo sapiens
KWEAARVAEQLRAYLEGLCVEWLRRH

SEQ ID NO: 74 - protein, 13 aa, Homo sapiens
DRETRDLTGNGKD

SEQ ID NO: 75 - protein, 21 aa, Homo sapiens
LSSWTAADTAAQITQRKLEAA

SEQ ID NO: 76 - protein, 23 aa, Homo sapiens
ILRWEPSSQPTIPIVGIIAGLVL

SEQ ID NO: 77 - protein, 15 aa, Homo sapiens
GHVRGVAPQIPGERE

SEQ ID NO: 78 - protein, 24 aa, Homo sapiens
HQAQVGGGPCGGAVESLPGGHVRG

SEQ ID NO: 79 - protein, 16 aa, Homo sapiens
FLPTGGKGGSCSQAAS

SEQ ID NO: 80 - protein, 24 aa, Homo sapiens
FLPTGGKGGSCSQAASSNSAQGSD

SEQ ID NO: 81 - protein, 9 aa, Homo sapiens
KTKQGVAEA

SEQ ID NO: 82 - protein, 9 aa, Homo sapiens
DVFMKGLSK

SEQ ID NO: 83 - protein, 9 aa, Homo sapiens
FMKGLSKAK

SEQ ID NO: 84 - protein, 10 aa, Homo sapiens
FMKGLSKAKE

SEQ ID NO: 85 - protein, 10 aa, Homo sapiens
KTKQGVAEAA

SEQ ID NO: 86 - protein, 10 aa, Homo sapiens
KAKEGVVAAA

SEQ ID NO: 87 - protein, 9 aa, Homo sapiens
KAKEGVVAA

SEQ ID NO: 88 - protein, 9 aa, Homo sapiens
KTKEGVLYV

SEQ ID NO: 89 - protein, 9 aa, Homo sapiens
APGKTKEGV

SEQ ID NO: 90 - protein, 10 aa, Homo sapiens
GVAEAPGKTK

SEQ ID NO: 91 - protein, 9 aa, Homo sapiens
KTKEGVVHG

SEQ ID NO: 92 - protein, 15 aa, Homo sapiens
YVGSKTKEGVVHGVA

SEQ ID NO: 93 - protein, 9 aa, Homo sapiens
AVVTGVTAV

SEQ ID NO: 94 - protein, 9 aa, Homo sapiens
QVTNVGGAV

SEQ ID NO: 95 - protein, 9 aa, Homo sapiens
KTKEQVTNV

SEQ ID NO: 96 - protein, 10 aa, Homo sapiens
NVGGAVVTGV

SEQ ID NO: 97 - protein, 10 aa, Homo sapiens
GAVVTGVTAV

SEQ ID NO: 98 - protein, 10 aa, Homo sapiens
GVTAVAQKTV

SEQ ID NO: 99 - protein, 10 aa, Homo sapiens
EQVTNVGGAV

SEQ ID NO: 100 - protein, 10 aa, Homo sapiens
VATVAEKTKE

SEQ ID NO: 101 - protein, 9 aa, Homo sapiens
GVVHGVATV

SEQ ID NO: 102 - protein, 15 aa, Homo sapiens
EGVVHGVTTVAEKTK

SEQ ID NO: 103 - protein, 15 aa, Homo sapiens
TTVAEKTKEQVTNVG

SEQ ID NO: 104 - protein, 15 aa, Homo sapiens
KGVVHGVATVAEKTK

SEQ ID NO: 105 - protein, 15 aa, Homo sapiens
EGVVHGVATVAEKTK

SEQ ID NO: 106 - protein, 15 aa, Homo sapiens
ATVAEKTKEQVTNVG

SEQ ID NO: 107 - protein, 9 aa, Homo sapiens
SIAAATGFV

SEQ ID NO: 108 - protein, 9 aa, Homo sapiens
AAATGFVKK

SEQ ID NO: 109 - protein, 9 aa, Homo sapiens
KTVEGAGSI

SEQ ID NO: 110 - protein, 9 aa, Homo sapiens
GSIAAATGF

SEQ ID NO: 111 - protein, 10 aa, Homo sapiens
NEEGAPQEGI

SEQ ID NO: 112 - protein, 10 aa, Homo sapiens
AATGFVKKDQ

SEQ ID NO: 113 - protein, 9 aa, Homo sapiens
FVKKDQLGK

SEQ ID NO: 114 - protein, 9 aa, Homo sapiens
PVDPDNEAY

SEQ ID NO: 115 - protein, 9 aa, Homo sapiens
MPVDPDNEA

SEQ ID NO: 116 - protein, 9 aa, Homo sapiens
DPDNEAYEM

SEQ ID NO: 117 - protein, 10 aa, Homo sapiens
EGILEDMPVD

SEQ ID NO: 118 - protein, 10 aa, Homo sapiens
MPVDPDNEAY

SEQ ID NO: 119 - protein, 10 aa, Homo sapiens
QEGILEDMPV

SEQ ID NO: 120 - protein, 266 aa, Homo sapiens
MVCLKLPGGSYMAKLTVTLMVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS

SEQ ID NO: 121 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS